(19) United States
(12) Patent Application Publication
(10) Pub. No.: US 2024/0409618 A1
(43) Pub. Date: Dec. 12, 2024

(54) DNA ENCODED ANTIBODIES WITH Fc MODIFICATIONS

(71) Applicants: David Weiner, Merion, PA (US); Elizabeth Parzych, Philadelphia, PA (US); Trevor Smith, San Diego, CA (US); Ami Patel, Philadelphia, PA (US); Daniel Kulp, Moorestown, NJ (US); The Wistar Institute of Anatomy and Biology, Philadelphia, PA (US)

(72) Inventors: David Weiner, Merion, PA (US); Elizabeth Parzych, Philadelphia, PA (US); Trevor Smith, San Diego, CA (US); Ami Patel, Philadelphia, PA (US); Daniel Kulp, Moorestown, NJ (US)

(21) Appl. No.: 18/701,492

(22) PCT Filed: Oct. 13, 2022
(86) PCT No.: PCT/US2022/078033

§ 371 Date: Apr. 15, 2024

Related U.S. Application Data
(60) Provisional application No. 63/255,197, filed on Oct. 13, 2021.
(60) Provisional application No. 63/313,112, filed on Feb. 23, 2022.
(60) Provisional application No. 63/323,469, filed on Mar. 24, 2022.
(60) Provisional application No. 63/343,759, filed on May 19, 2022.
(60) Provisional application No. 63/355,009, filed on Jun. 23, 2022.
(60) Provisional application No. 63/375,795, filed on Sep. 15, 2022.
(60) Provisional application No. 63/375,912, filed on Sep. 16, 2022.

(51) Int. Cl. C07K 16/10; A61K 39/00; A61K 39/42

(52) U.S. Cl. CPC C07K 16/1003; A61K 39/42; A61K 2039/53; C07K 2317/52; C07K 2317/76; C07K 2317/94

(57) ABSTRACT

Disclosed herein are nucleic acid encoded Fc-modified antibodies and methods of use of the same for the treatment of diseases and disorders.

### CROSS REFERENCE TO RELATED APPLICATIONS

This application claims priority to U.S. Provisional Application No. 63/255,197, filed Oct. 13, 2021, U.S. Provisional Application No. 63/313,112, filed Feb. 23, 2022, U.S. Provisional Application No. 63/323,469, filed Mar. 24, 2022, U.S. Provisional Application No. 63/343,759, filed May 19, 2022, U.S. Provisional Application No. 63/355,009, filed Jun. 23, 2022, U.S. Provisional Application No. 63/375,795, filed Sep. 15, 2022, and U.S. Provisional Application No. 63/375,912, filed Sep. 16, 2022, each of which is hereby incorporated by reference herein in its entirety.

### STATEMENT REGARDING FEDERALLY SPONSORED RESEARCH OR DEVELOPMENT

This invention was made with government support under HR0011-21-9-0001 awarded by the Defense Advanced Research Projects Agency (DARPA). The government has certain rights in the invention.

### TECHNICAL FIELD

The present invention relates to optimized recombinant nucleic acid molecules for generating one or more synthetic antibodies having high expression, stability or longer half-life, functional fragments thereof, and compositions comprising the optimized synthetic antibodies, and optimized recombinant nucleic acid molecules, as well as methods of treating or preventing a disease or disorder by administering said optimized recombinant nucleic acid molecules.

### BACKGROUND

Targeted monoclonal antibodies (mAbs) represent one of the most important medical therapeutic advances of the last 25 years. This type of immune based therapy is now used routinely against a host of autoimmune diseases, treatment of cancer as well as infectious diseases.

The clinical impact of mAb therapy is impressive. However, issues remain that limit the use and dissemination of this therapeutic approach. Some of these include the high cost of production of these complex biologics that can limit their use in the broader population, particularly in the developing world where they could have a great impact. For example, the dramatic cost, slow development, and requirement for several high-dose administrations (mg/kg) represent a significant challenge for protein mAb delivery, especially during a possible outbreak. Furthermore, the frequent requirement for repeat administrations of the mAbs to attain and maintain efficacy can be an impediment in terms of logistics and patient compliance.

To address some of these problems, methods for nucleic acid-based delivery of antibodies have been developed, however the stability or half-life of these antibody formulations is frequently short and less than optimal.

Thus, there is a need in the art for compositions and methods for DNA delivery of antibodies that have increased stability or longer half-life. The present invention addresses this unmet need.

### SUMMARY OF THE INVENTION

In one embodiment, the invention relates to a nucleic acid molecule encoding an antibody or fragment thereof wherein the Fc domain of the heavy chain has been modified to include a half-life extending variation, a stability increasing variation, a variation to alter complement binding, a variation to alter dimerization or a variation to alter the level of Fc-FcγR interaction.

In one embodiment, the nucleic acid molecule encodes a Fc domain of the heavy chain comprising at least of M252Y, S254T, T256E, L234F, L235E, P331S, E430G, S239D, I332E, G236A, A330L, G236R, L328R, L235Q, or K322Q.

In one embodiment, the nucleic acid molecule encodes an Fc domain of the heavy chain comprising a combination of M252Y/S254T/T256E (YTE); L234F/L235E/P331S (TM); S239D/I332E (DE); S239D/I332E/E430G (DEG); G236A/I332E (AE); A330L/I332E (ALIE); G236A/A330L/I332E (GAALIE); S239D/A330L (SDAL); G236A/S239D/A330L (GASDAL); or G236R/L328R (GRLR).

In one embodiment, the nucleic acid molecule further comprises a nucleotide sequence encoding a cleavage domain. In one embodiment, the nucleic acid molecule further comprises a nucleotide sequence encoding a leader sequence.

In one embodiment, the nucleic acid molecule is an expression vector.

In one embodiment, the nucleic acid molecule comprises a nucleotide sequence encoding a heavy chain of an anti-SARS-CoV-2 spike antigen synthetic antibody. In one embodiment, the nucleic acid molecule encodes SEQ ID NO:6, SEQ ID NO: 12, SEQ ID NO: 18, SEQ ID NO:24, SEQ ID NO:26, SEQ ID NO:28, SEQ ID NO: 60, SEQ ID NO:62, SEQ ID NO:68, SEQ ID NO: 72, SEQ ID NO: 74, SEQ ID NO: 88, SEQ ID NO: 90, SEQ ID NO:92, SEQ ID NO:94, SEQ ID NO:96 or SEQ ID NO: 98. In one embodiment, the nucleic acid molecule comprises SEQ ID NO:5, SEQ ID NO: 11, SEQ ID NO:17, SEQ ID NO:23, SEQ ID NO:25, SEQ ID NO:27, SEQ ID NO: 59, SEQ ID NO:61, SEQ ID NO:67, SEQ ID NO: 71, SEQ ID NO: 73, SEQ ID NO: 87, SEQ ID NO: 89, SEQ ID NO:91, SEQ ID NO:93, SEQ ID NO:95, or SEQ ID NO: 97.

In one embodiment, the invention relates to a composition comprising at least one nucleic acid molecule encoding an antibody comprising an Fc variation or fragment thereof, wherein the Fc domain of the heavy chain has been modified to include a half-life extending variation, a stability increasing variation, a variation to alter complement binding, a variation to alter dimerization or a variation to alter the level of Fc-FcγR interaction.

In one embodiment, the nucleic acid molecule encodes a Fc domain of the heavy chain comprising at least of M252Y, S254T, T256E, L234F, L235E, P331S, E430G, S239D, I332E, G236A, A330L, G236R, L328R, L235Q, or K322Q.

In one embodiment, the nucleic acid molecule encodes an Fc domain of the heavy chain comprising a combination of M252Y/S254T/T256E (YTE); L234F/L235E/P331S (TM); S239D/I332E (DE); S239D/I332E/E430G (DEG); G236A/I332E (AE); A330L/I332E (ALIE); G236A/A330L/I332E (GAALIE); S239D/A330L (SDAL); G236A/S239D/A330L (GASDAL); or G236R/L328R (GRLR).

In one embodiment, the composition comprises a first nucleic acid molecule comprising a nucleotide sequence encoding a heavy chain of a synthetic antibody comprising at least one Fc variation; and a second nucleic acid molecule comprising a nucleotide sequence encoding a light chain of a synthetic antibody.

In one embodiment, the composition further comprises a pharmaceutically acceptable excipient.

In one embodiment, the invention relates to a method of preventing or treating a disease in a subject, the method comprising administering to the subject at least one nucleic acid molecule encoding an antibody comprising an Fc variation or fragment thereof, wherein the Fc domain of the heavy chain has been modified to include a half-life extending variation, a stability increasing variation, a variation to alter complement binding, a variation to alter dimerization or a variation to alter the level of Fc-FcγR interaction or a composition comprising the same.

In one embodiment, the nucleic acid molecule encodes a Fc domain of the heavy chain comprising at least of M252Y, S254T, T256E, L234F, L235E, P331S, E430G, S239D, I332E, G236A, A330L, G236R, L328R, L235Q, or K322Q.

In one embodiment, the nucleic acid molecule encodes an Fc domain of the heavy chain comprising a combination of M252Y/S254T/T256E (YTE); L234F/L235E/P331S (TM); S239D/I332E (DE); S239D/I332E/E430G (DEG); G236A/I332E (AE); A330L/I332E (ALIE); G236A/A330L/I332E (GAALIE); S239D/A330L (SDAL); G236A/S239D/A330L (GASDAL); or G236R/L328R (GRLR).

In one embodiment, the disease is COVID-19.

In one embodiment, the invention relates to a method of inducing an immune response against a disease or disorder in a subject, the method comprising administering to the subject at least one nucleic acid molecule encoding an antibody comprising an Fc variation or fragment thereof, wherein the Fc domain of the heavy chain has been modified to include a half-life extending variation, a stability increasing variation, a variation to alter complement binding, a variation to alter dimerization or a variation to alter the level of Fc-FcγR interaction or a composition comprising the same.

In one embodiment, the nucleic acid molecule encodes a Fc domain of the heavy chain comprising at least of M252Y, S254T, T256E, L234F, L235E, P331S, E430G, S239D, I332E, G236A, A330L, G236R, L328R, L235Q, or K322Q.

In one embodiment, the nucleic acid molecule encodes an Fc domain of the heavy chain comprising a combination of M252Y/S254T/T256E (YTE); L234F/L235E/P331S (TM); S239D/I332E (DE); S239D/I332E/E430G (DEG); G236A/I332E (AE); A330L/I332E (ALIE); G236A/A330L/I332E (GAALIE); S239D/A330L (SDAL); G236A/S239D/A330L (GASDAL); or G236R/L328R (GRLR).

### BRIEF DESCRIPTION OF THE DRAWINGS

FIG. 1A through FIG. 1D depict mAb characteristics and construct designs for selected SARS-CoV-2 DMAbs 2196, 2130 and 2381. FIG. 1A depicts summary profiles detailing the family, antibody class, epitope specificity and reported potency of each mAb against the indicated SARS-CoV-2 viral variants. FIG. 1B and FIG. 1C depict DMAb plasmid designs. The heavy (VH) and light (VL) domains of clones 2196-2130 and 2381 were grafted onto wildtype (WT) human IgG1 constant domain framework (CH and CL, respectively; allotype G1m1) using a (FIG. 1B) single plasmid (pHC/LC) or (FIG. 1C) dual plasmid (pLC+pHC_FcWT) approaches. FIG. 1D depicts modified pHCs constructs containing Fc mutations L234F, L235E and P331S (pHC_FcTM) to ablate effector functions. Yellow=leader sequences; red=flexible linker connecting Ab chains in single plasmid design.

FIG. 2A through FIG. 2H depict exemplary experimental results demonstrating a rapid evaluation of wildtype (FcWT) DMAbs 2196, 2130 and 2381 using single plasmid systems. FIG. 2A through FIG. 2C depicts an analysis of the indicated DMAbs following in vitro expression. FIG. 2A depicts the quantification using an anti-human IgG ELISA; bars represent the average titer (+/−SEM) of transfection duplicates for each construct. FIG. 2B depicts neutralization against pseudotyped virus expressing the spike protein from SARS-CoV-2 strain WA1/2020; curves represent the best-fit lines. FIG. 2C depicts neutralizing ID50 against authentic SARS-CoV-2 virus (WA1/2020); LOD=limit of detection. FIG. 2D depicts acute serum DMAb levels (anti-human IgG ELISA) in BALB/c mice (n=5/group) following in vivo plasmid delivery (100 ug/animal); bars represent the average titer for each group (+/−SEM) at the indicated timepoints. FIG. 2E depicts representative neutralization activities of in vivo-launched DMAbs against pseudotyped SARS-CoV-2 (WA1/2020); best-fit curves represent two independent serum samples. Calculated IC50s are shown above each graph. Naïve sera served as a control. FIG. 2F through FIG. 2H depicts the efficacy of in vivo-launched DMAbs against SARS-CoV-2 (WA1/2020) infection using an AAV-ACE2 challenge model. FIG. 2F depicts a schematic of challenge study conducted in BALB/c mice (n=8/group). FIG. 2G depicts endpoint titers in the sera of DMAb-treated mice at the time of challenge (D21 post-plasmid-delivery). FIG. 2H depicts the viral load (copies/g) in the lungs of DMAb-treated and control mice at D4 post-challenge as determined qPCR. Group differences determined by Kruskal-Wallis Test followed by Dunn's post hoc analysis (* P<0.05, **P<0.01, ***P<0.001, and ****P<0.0001).

FIG. 3A through FIG. 3H depict exemplary experimental results demonstrating the characterization of in vivo-launched 2196, 2130 and 2381-based DMAb variants (FcWT and FcTM) using dual plasmid approaches. FIG. 3A through FIG. 3C depict the expression and functionality of wildtype DMAbs in the sera of plasmid-treated BALB/c mice following in vivo dual plasmid delivery (100 ug/animal). FIG. 3A depicts the extended expression kinetics (anti-human IgG ELISA) in sera over time; graphs depict the average titer for each group (+/−SEM) at the indicated timepoints. FIG. 3B depicts neutralization curves (best-fit lines) of pseudotyped SARS-CoV-2 (WA1/2020) using pooled sera (D6). Calculated ID50s and IC50s are displayed. FIG. 3C depicts the neutralizing ID50 of pooled sera (D6) against authentic SARS-CoV-2 virus (WA1/2020). FIG. 3D depicts the quantification of DMAb titers in the lung bronchiolar lavage (BAL) of DMAb-treated BALB/c mice (n=13/14) harvested at D14 post-plasmid delivery. Graph depicts titers in individual animals, with the horizontal bar indicating the mean titer (+/−SEM). (e-i) Expression kinetics and characterization of DMAb Fc variants (FcWT and FcTM) in K-18 mice. FIG. 3E depicts DMAb titers (group average+/−SEM) of FcWT variants over time. FIG. 3F depicts DMAb titers (group average+/−SEM) of FcTM variants over time. FIG. 3G depicts neutralizing ID50 of pooled sera (D8) from K-18 mice against authentic SARS-CoV-2 virus (WA1/2020); LOD=limit of detection. FIG. 3H depicts the reactivity of pooled sera (D19) from DMAb-treated animals against indicated epitope-specific mutant RBD recombinant proteins via ELISA; curves indicate the average OD values (+/−SEM) of individual animals within each group.

FIG. 4A and FIG. 4B depict exemplary experimental data demonstrating that the dual plasmid systems enhance DMAb in vitro expression relative single construct approaches while maintaining neutralizing activity. Parallel in vitro expression and evaluation of single and dual plasmid systems for the indicated DMAbs. FIG. 4A depicts the quantification using an anti-human IgG ELISA; bars represent the average titer (+/−SEM) of transfection duplicates for each construct. FIG. 4B depict the neutralization against pseudotyped virus expressing the spike protein from SARS-CoV-2 strain WA1/2020; curves represent the best-fit lines. Calculated IC50s are shown.

FIG. 5A through FIG. 5H depict exemplary experimental results demonstrating that prophylactic administration 2196_FcTM and 2130_FcTM protect mice in a lethal SARS-CoV-2 challenge model. FIG. 5A depicts a schematic of lethal challenge conducted in K-18 mice. FIG. 5B depicts DMAb titers (anti-human IgG ELISA) in the sera of individual challenge mice at the indicated times post plasmid-delivery. Group averages+/−SEM are indicated. FIG. 5C and FIG. 5D depict measurements of viral control at D4 post-challenge in a subset of each treatment group (n=4); viral loads (TCID50/g tissue) in the (FIG. 5C) nasal turbinates (NT) and (FIG. 5D) lungs of challenged mice. Significant reductions relative to control animals were determined using Mann-Whitney U Tests (* P<0.05. **P<0.01, ***P<0.001, and ****P<0.0001). LOD=Limit of detection. FIG. 5E and FIG. 5F depict the histopathology in the lung at D4 post-challenge using H&E-stained lung sections. FIG. 5E depicts that pathology scores, ranging from 0-5, were assigned to blinded samples based on visual evaluation at the gross and microscopic levels. Graph depicts the individual scores and the group averages (+/−SD). FIG. 5F depicts representative images from each group used for histopathology scoring. FIG. 5G and FIG. 5H depict the challenge outcome through D14 post-infection. FIG. 5G depicts the percentage of weight change over time for each mouse relative to starting weights (prior to challenge). FIG. 5H depicts the survival (%) in DMAb treated groups relative to control animals, compared using a log-rank test (* P<0.05, **P<0.01, ***P<0.001, and ****P<0.0001).

FIG. 6A through FIG. 6H depict exemplary experimental results demonstrating that prophylactic delivery of DMAb cocktails results in complete viral elimination, protecting mice against morbidity and mortality following lethal SARS-CoV-2 challenge. FIG. 6A depicts a schematic of lethal challenge conducted in K-18 mice. FIG. 6B depicts the DMAb titers (anti-human IgG ELISA) in the sera of individual challenge mice at the indicated times post plasmid-delivery. Group averages+/−SEM are indicated. FIG. 6C depicts the reactivity of sera harvested from cocktail-treated mice (D19) against the indicated epitope-specific mutant RBD recombinant proteins via ELISA; individual OD curves values for each animal are shown. FIG. 6D and FIG. 6E depict measurements of viral control at D4 post-challenge in a subset of each treatment group (n=4); viral loads (TCID50/g tissue) in the (FIG. 6D) nasal turbinates (NT) and (FIG. 6E) lungs of challenged mice. Significant reductions relative to control animals were determined using Mann-Whitney U Tests (* P<0.05, **P<0.01, ***P<0.001, and ****P<0.0001). LOD=Limit of detection. FIG. 6F depicts the histopathology in the lung at D4 post-challenge using H&E-stained lung sections. Pathology scores, ranging from 0-5, were assigned to blinded samples based on visual evaluation at the gross and microscopic levels. Graph depicts the individual scores and the group averages. FIG. 6G and FIG. 6H depict the challenge outcome through D14 post-infection. FIG. 6G depicts the percentage of weight change over time for each mouse relative to starting weights (prior to challenge). FIG. 6H depicts the survival (%) in DMAb treated groups relative to control animals, compared using a log-rank test (* P<0.05, **P<0.01, ***P<0.001, and ****P<0.0001).

FIG. 7A through FIG. 7G depict exemplary experimental results demonstrating that in vivo-launched 2196- and 2130-based DMAbs retain antiviral activity against major variants of concern. FIG. 7A through FIG. 7E depict the neutralizing activity of sera (n=3-5/group; D19 or D9) from animals administered the indicated FcTM DMAb(s) against pseudoviruses expressing the spike protein from (FIG. 7A) wildtype SARS-CoV-2 (WA1/2020) and viral variants (FIG. 7B) B.1.1.7, (FIG. 7C) B.1.251, (FIG. 7D) B.1.526 and (FIG. 7E) B.1.617.2. Individual neutralization curves (best-fit lines) and matched ID50s against each variant compared to WA1/2020 are shown. The fold change (x) in ID50 is indicated above each graph. FIG. 7F and FIG. 7G depict the relative activity of indicated in vivo-launched DMAb(s) against WA1/2020 as (FIG. 7F) serum ID50s and (FIG. 7G) serum IC50s). Values for individual mice as well as the group mean (+/−SEM) are shown. Differences between groups were measured using Kruskal-Wallis Test followed by Dunn's post hoc analysis (* P<0.05, **P<0.01, ***P<0.001, and ****P<0.0001).

FIG. 8A through FIG. 8E depict exemplary experimental results demonstrating the antiviral activity of 2196_FcWT and 2130_FcWT DMAbs against SARS-CoV-2 variants of concern (related to FIG. 7). Neutralizing activity of sera (n=3-5/group; D19 or D9) from animals administered the indicated FcWT DMAb(s) against pseudoviruses expressing the spike protein from (FIG. 8A) wildtype SARS-CoV-2 (WA1/2020) and viral variants (FIG. 8B) B.1.1.7, (FIG. 8C) B.1.351, (FIG. 8D) B.1.526 and (FIG. 8E) B.1.617.2. Individual neutralization curves (best-fit lines) and matched ID50s against each variant compared to WA1/2020 are shown. The fold change (x) in ID50 is indicated above each graph.

FIG. 9 depicts a table of the expanded DMAb panel with selected SARS-CoV-2 mAb clones.

FIG. 10A through FIG. 10D depict exemplary experimental results demonstrating the design, rapid expression and antiviral activity of an expanded SARS-CoV-2 DMAb panel following in vivo delivery. FIG. 10A depicts a representation of the DMAb panel as a function of Ab class. FIG. 10B depicts the in vivo expression levels (anti-human IgG ELISA) of the indicated DMAbs at D14 post-plasmid administration. Group averages+/−SEM are indicated. FIG. 10C depicts the antiviral activity of sera pools (D6) containing clinically-relevant combinations against WA1/2020, B.1.351 and B.1.617.2 pseudoviruses. Neutralization curves (best-fit lines) are shown. FIG. 10D depicts the heat map summarizing the relative IC50 values against WA1/2020 measured for each DMAb when administered individually or in combinations as well as the fold change in IC50 against indicated compared to WA1/2020. Blue=decreased activity.

FIG. 11A and FIG. 11B depict exemplary experimental results demonstrating the relative binding of in vivo-launched 2196 and 2130-based DMAbs to recombinant RBD (or full-length S protein) containing key mutations found in SARS-CoV-2 variants of concern. FIG. 11A depicts a summary of mutations identified throughout the SARS-CoV-2 S protein from the indicated variants of concern. Colored squares (green, pink, blue, purple) indicate key residues within the RBD that are linked to viral resistance. S1=subdomain 1; S2=Subdomain 2; RBD=Receptor binding domain. FIG. 11B depicts the relative binding of the indicated in vivo-launched DMAbs to various mutant RBDs via ELISA. Naïve serum was used as a control. Graphs depict the average OD values (+/−SEM).

FIG. 12 depicts a table presenting engineering strategies to optimize the efficacy of in vivo-launched antibodies using synthetic DNA.

FIG. 13 depicts a diagram of the unique combination of strategies used to enhance specific DMAb properties.

FIG. 14 depicts exemplary experimental results demonstrating that combinations of Fc engineering strategies enhance effector functions.

FIG. 15 depicts exemplary experimental results demonstrating the in vitro expression and antiviral activity of Fc-engineered DMAbs with multiple effector function enhancement.

FIG. 16 depicts exemplary experimental results demonstrating the in vivo expression of the S309 Fc variants.

FIG. 17A through FIG. 17C depict exemplary experimental results demonstrating the effects of allotype modification. FIG. 17A depicts an analysis of allotype-matched clinical constructs. FIG. 17B depicts the quantification of DMAb variants' in vitro-expression. FIG. 17C depicts the antiviral activity of indicated DMAb variants following in-vitro expression (WA1/2020 vs. B.1.351 pseudoviruses)

FIG. 18 depicts exemplary experimental results demonstrating the expression optimization of the single plasmid system.

FIG. 19 depicts exemplary experimental results demonstrating that reversing the orientation of HC and LC results in 1.5-2× fold increase in expression for DMAB-AZD1061.

FIG. 20 depicts the cryo-EM data processing specifications.

FIG. 21A through FIG. 21E: mAb characteristics and construct designs for selected SARS-CoV-2 DMAbs 2196, 2130 and 2381. (FIG. 21A) Summary profiles detailing the family, antibody class, epitope specificity and reported potency of each mAb against the indicated SARS-CoV-2 viral variants. (FIG. 21B-D) DMAb plasmid designs. The heavy (VH) and light (VL) domains of clones 2196-2130 and 2381 were grafted onto wildtype (WT) human IgG1 constant domain framework (CH and CL, respectively; allotype G1m1) (FIG. 21B) single plasmid (pHC/LC). (FIG. 21C) dual plasmid (pLC+pHC_WT) approaches. (FIG. 21D) Modified pHCs constructs containing Fc mutations L234F, L235E and P331S (pHC_TM) to ablate effector functions. (FIG. 21E) Modified pHCs containing Fc mutations M252Y, S254T, T256E for in vivo half-life extension. Yellow=leader sequences; red=flexible linker connecting Ab chains in single plasmid design.

FIG. 22A through FIG. 22H: Expression and characterization of in vivo-launched SARS-CoV-2 DMAb constructs. (FIG. 22A-B) WT DMAb expression in the sera of BALB/c mice (100 μg dose; 5/group). Group geometric mean titer (GMT) (+/−SD)) is represented following administration of (FIG. 22A) single plasmid system or (FIG. 22B) dual plasmid systems. (FIG. 22C) Neutralization curves (best-fit lines) of pseudotyped SARS-CoV-2 (USA-WA1/2020) using pooled sera from (FIG. 22B); ID50/IC50 values are displayed. (FIG. 22D) DMAb levels in the lung bronchoalveolar lavage (BAL) of BALB/c mice at D14 post-plasmid delivery (100 μg dose; 13-14/group). GMT (+/−SD) is indicated. (FIG. 22E-H) Expression and characterization of WT and TM DMAb constructs in K-18 mice (100 μg dose; 5/group). (FIG. 22E) DMAb expression levels (GMT+/−SD) of WT and TM constructs. (FIG. 22F) Neutralizing activity of pooled sera against authentic SARS-CoV-2 virus (USA-WA1/2020); LOD=limit of detection. Graph depicts ID50 and calculated IC50 for each pool. (FIG. 22G) Reactivity of pooled sera against indicated epitope-specific mutant RBDs; curves indicate the average OD values (+/−SEM) of technical replicates. (FIG. 22H) ACE2 receptor blocking activity (% of control wells) relative to control wells (left) and corresponding blocking DMAb titer (right) for individual serum samples. Horizontal bars indicate group means.

FIG. 23A through FIG. 23B: Dual plasmid systems enhance DMAb in vitro expression relative single construct approaches while maintaining neutralizing activity. Parallel in vitro expression and evaluation of single and dual plasmid systems was performed for the indicated DMAbs. (FIG. 23A) DMAb quantification using an anti-human IgG ELISA following single (solid bars) or dual plasmid (hatched bars) delivery; bars represent the group geometric mean titer+/−SD of transfection duplicates for each construct. (FIG. 23B) Neutralization of dual plasmid constructs against pseudotyped virus expressing the spike protein from SARS-CoV-2 (USA-WA/2020); curves represent the best-fit lines. Calculated IC50s are shown.

FIG. 24A through FIG. 24C: Antiviral activity of 2196_FcWT and 2130_FcWT DMAbs against SARS-CoV-2 viral variants (related to FIG. 25). (FIG. 24A) Summary of mutations identified throughout the SARS-CoV-2 S protein from the indicated variants. Colored squares (green, blue, orange) indicate key residues within the RBD that are linked to viral resistance. S1=subdomain 1; S2=subdomain 2; RBD=receptor binding domain. (FIG. 24B) Relative binding of sera pools containing the indicated in vivo-launched DMAbs to various mutant RBDs via ELISA. Naïve serum was used as a control. Graphs depict the average OD values (+/−SEM). (FIG. 24C) Neutralizing activity of sera (n=3-5/group; D19 or D9) from animals administered the indicated WT DMAb(s) against pseudoviruses expressing the spike protein from wildtype SARS-CoV-2 (WA1/2020) and viral variants B.1.1.7, B.1.351, B.1.526 and B.1.617.2. Individual neutralization curves (best-fit lines) and matched ID50s against each variant compared to WA1/2020 are shown in FIG. 25.

FIG. 25A through FIG. 25G: In vivo-launched 2196- and 2130-based DMAbs retain activity against major SARS-CoV-2 viral variants. Neutralizing activity of sera samples from DMAb-administered mice (n=3-5/group) against (FIG. 25A) USA-WA1/2020 SARS-CoV-2 pseudovirus (Individual neutralization curves (best-fit lines) displayed) or (FIG. 25B-E) the indicated variant pseudoviruses for which matched ID50s compared to USA-WA1/2020 are shown. Average fold change (x) in ID50 for each group is indicated: (FIG. 25B) B.1.1.7 variant. (FIG. 25C) B.1.351 variant. (FIG. 25D) B.1.526 variant. (FIG. 25E) B.1.617.2 variant. (FIG. 25F) Comparison of serum ID50 values for individual samples against USA-WA1/2020 (geometric mean+/−SD depicted). Differences between groups were measured using Kruskal-Wallis Test followed by Dunn's post hoc analysis. (FIG. 25G) Comparison of serum IC50 values for individual samples against USA-WA1/2020 (geometric mean+/−SD depicted). Differences between groups were measured using Kruskal-Wallis Test followed by Dunn's post hoc analysis. P-values indicated.

FIG. 26A through FIG. 26Q: Prophylactic delivery of 2196(TM) and 2130(TM) DMAbs, protect mice against lethal SARS-CoV-2 challenge. (FIG. 26A-G) DMAb prophylaxis against lethal SARS-CoV-2 (USA-WA1/2020) (monotherapy). (FIG. 26A) Schematic of challenge in K-18 mice. (FIG. 26B) Serum DMAb levels following plasmid delivery (GMT (+/−SD)). (FIG. 26C-D) Measurements of viral control (TCID50/g tissue) at D4 (4/group). Viral load (GMT (+/−SD)) in the (FIG. 26C) nasal turbinate (NT) and (FIG. 26D) lung were compared using a Kruskal-Wallis Test followed by Dunn's post hoc analysis. P-values indicated. Horizontal lines indicate LOD. (FIG. 26E) Histopathology scores for H&E-stained lung sections (D4; group averages (+/−SD). (FIG. 26F) Body weight change (%) following challenge. (FIG. 26G) Survival (%) in each group compared to control animals using a log-rank test. P-values indicated. (FIG. 26H-O) DMAb prophylaxis against lethal SARS-CoV-2 (USA-WA1/2020) following co-administration. (FIG. 26H) Schematic of lethal challenge in K-18 mice. (FIG. 26I) Total serum DMAb (hIgG) levels following plasmid delivery (GMT (+/−SD)). (FIG. 26J) Sera reactivity against epitope-specific mutant RBDs; individual OD450 curves values for each animal. (FIG. 26K-L) Viral load (GMT (+/−SD)) in the (FIG. 26K) NT and (FIG. 26L) lung were compared using a Kruskal-Wallis Test followed by Dunn's post hoc analysis. P-values indicated. Horizontal lines indicate LOD. (FIG. 26M) Histopathology scores for H&E-stained lung sections (D4; group averages (+/−SD). (FIG. 26N) Body weight change (%) following challenge. (FIG. 26O) Survival (%) in each group compared to control animals using a log-rank test. P-values indicated. (FIG. 26P) Relative binding (OD450 curves) of pooled sera from cocktail-expressing challenge mice to the indicated mutant RBDs relative to parental D614G RBD. (FIG. 26Q) Neutralizing activity of this sera (3-5/group) against variant pseudoviruses. Individual neutralization curves against USA-WA1/2020 (best-fit lines) and matched ID50s against the other variants are shown. The fold change (x) in ID50 is indicated.

FIG. 27A through FIG. 27D: Evaluation of wildtype (WT) DMAbs 2196, 2130 and 2381 in a non-lethal AAV6.2FF-hACE-2-transduced murine challenge model. (FIG. 27A) Schematic of efficacy study conducted in an AAV-ACE2 challenge model (n=8/group). (FIG. 27B) Endpoint titers in the sera of DMAb-treated mice at the time of harvest (D4 post-challenge group geometric mean titer+/−SD). (FIG. 27C) Neutralization activity of in vivo-launched DMAbs against pseudotyped SARS-CoV-2 (USA-WA1/2020); best-fit curves represent two representative serum samples per group. Calculated IC50s are shown above each graph. Naïve sera served as a control. (FIG. 27D) Viral load (copies/g) in the lungs of DMAb-treated and control mice at D4 post-challenge as determined qPCR (group geometric mean titer (+/−SD)). Group differences determined by Kruskal-Wallis Test followed by Dunn's post hoc analysis (*P<0.05, **P<0.01, ***P<0.001, and ****P<0.0001).

FIG. 28A through FIG. 28C: In vitro expression and validation of 2130- and 2196-based DMAbs with Fc-modified framework for extended in vivo half-life (YTE variants). (FIG. 28A-C) Analysis of the indicated DMAbs following in vitro expression. (FIG. 28A) Quantification using an anti-human IgG ELISA; bars represent the group geometric mean titer (+/−SD) of transfection duplicates for each construct. (FIG. 28B) Transfection supernatants containing the indicated DMAbs (200 ng/lane) were analyzed via western blot using anti-human IgG (h+l)-HRP (top blots) or anti-YTE IgG (bottom blots). Beta actin was visualized on all blots as a loading control (asterisks); LC=light chain, HC=heavy chain. (FIG. 28C) Neutralizing activity of transfection supernatants against pseudotyped SARS-CoV-2 (USA-WA1/2020) virus; curves represent the best-fit lines and calculated IC50s are displayed. Gray lines indicate naïve sera control.

FIG. 29A through FIG. 29N: Fc-engineered DMAb cocktail(s) confer equivalent protection in both murine (K-18; FIG. 29A-C) and Syrian golden hamsters (H-M) models of SARS-CoV-2 infection, comparable to bioprocessed rIgG. (FIG. 29A-G) Efficacy of DMAb cocktails (TM or WT) compared to rIgG cocktail (WT) benchmark. (FIG. 29A) Schematic of lethal challenge in K-18 mice. (FIG. 29B) Serum antibody levels (GMT+/−SD) following plasmid or rIgG administration. (FIG. 29C-D) Measurements of viral control (TCID50/g tissue) at D4 (4/group). Viral load (GMT(+/−SD)) in the (c) NT and (d) lung were compared using a Kruskal-Wallis Test followed by Dunn's post hoc analysis. P-values indicated. Horizontal lines indicate LOD. (FIG. 29E) Histopathology scores for H&E-stained lung sections (D4; group averages (+/−SD). (FIG. 29F) Body weight change (%) following challenge for each animal relative to starting weights. (FIG. 29G) Survival (%) in each group compared to control animals using a log-rank test. P-value indicated. (FIG. 29H-N) Efficacy of DMAb cocktails (TM or WT) in Syrian golden hamsters. (FIG. 29H) Schematic of non-lethal challenge conducted in Syrian golden hamsters (6/group). (FIG. 29I) Serum DMAb levels (GMT+/−SD) pre-challenge (D18 post-delivery). (FIG. 29J) Antiviral activity of hamster sera (D18) against live SARS-CoV-2 (USA-WA1/2020); ID50 values (GMT (+/−geometric SD)) are displayed. (FIG. 29K-L) Measurements of viral control (TCID50/g tissue) at D4 post-challenge in the (FIG. 29K) lung and (FIG. 29L) NT were compared using a Kruskal-Wallis Test followed by Dunn's post hoc analysis. P-values indicated. Horizontal lines indicate LOD. (FIG. 29M) Cumulative lung histopathology score for each animal in the indicated treatment group. Lung sections were scored for microscopic indications of edema, hemorrhage, hyperplasia, hypertrophy, metaplasia, mineralization and syncytial cells. Group scores were compared using a Kruskal-Wallis Test followed by Dunn's post hoc analysis. P-values. (FIG. 29N) Body weight change (%) following challenge for each animal relative to starting weights.

FIG. 30: Prophylactic delivery of DMAb or rIgG cocktails prevents lung pathology following SARS-CoV-2 challenge in K-18 mice (related to FIG. 4e). Representative images of H&E-stained lung sections from each group that were assessed for SARS-CoV-2-induced pathology. SARS-CoV-2-related mononuclear cell vascular/perivascular inflammation (arrows) and mononuclear cell alveolar/interstitial inflammation (arrowhead) were observed in naïve control animals only.

FIG. 31A through FIG. 31F: In vivo delivery and half-life engineering (YTE) contribute to improved durability of functional DMAbs compared to bioprocessed rIgG in hFcRn mice. hFcRn mice (4-5/group) were administered plasmids encoding the indicated DMAb cocktails (100 μg/animal) or rIgG cocktails (100 μg protein/animal; IP). (FIG. 31A) Serum levels (group geometric mean titer+/−SD) of the indicated DMAbs or rIgG mAbs. (FIG. 31B) Neutralizing activity of hFcRn sera (4-5/group) against wildtype (USA-WA1/2020) or B.1.351 and B.1.617.2 variant pseudoviruses; neutralization curves against WA1-2020 (best-fit lines) and matched ID50s against other variants are shown. (FIG. 31C) Relative reactivity of pooled sera (average OD values (+/−SEM) of technical replicates) from hFcRn mice against recombinant B.1.1.529 spike trimer. (FIG. 31D-F) BALB/c mice were administered the indicated DMAb cocktails and harvested sera was pooled for evaluation: (FIG. 31D) Relative reactivity of pooled sera against recombinant B.1.1.529/BA. 1 spike trimer (dashed) or the parental D614G (solid) spike trimer. Naïve serum was used as a control. (FIG. 31E) Individual neutralization curves of pooled sera (best-fit lines) against pseudotyped virus expressing the B.1.1.529/BA.1 spike; calculated IC50 values depicted. (FIG. 31F) Individual neutralization curves of pooled sera (best-fit lines) against pseudotyped virus expressing the BA.2 spike; calculated IC50 values depicted.

FIG. 32: Cryo-EM data processing workflow. Cryo-EM data processing workflow used in data processing. Various density segments are displayed for quality assurance. FSC curves for resolution assessment are shown.

FIG. 33A through FIG. 33F: Cryo-EM in vivo-produced DMAb Fabs complexed with stabilized SARS-CoV-2 (WA1/2020) spike trimer. (FIG. 33A-C) Cryo-EM density map of 2196 DMAb Fab (salmon) complexed to SARS-CoV-2 spike (gray); spike RBD indicated (blue). (FIG. 33A) Side view. (FIG. 33B) Top view. (FIG. 33C) Top view with Fab density removed. (FIG. 33D-F) Cryo-EM density map of 2196 (salmon) and 2130 DMAb (green) Fabs complexed to SARS-CoV-2 spike (gray); spike RBD indicated (blue). (FIG. 33D) Side view. (FIG. 33E) Top view. (FIG. 33F) Top view with Fab densities removed.

FIG. 34A through FIG. 34B: Distance between neighboring Fabs in 2130 and 2196 DMAb complex. (FIG. 34A) The distance between centers of neighboring 2196 Fabs bound to RBD in ‘out’ configuration is ˜48 Å. Distance between neighboring 2130 and 2196 Fabs bound to RBD in ‘out’ configuration is ˜29 Å. For the latter, hydrogen bonding was observed between the Fabs in the structure. Taking into consideration the flexibility of this macromolecular complex, it is entirely possible for additional stabilizing interactions to occur between IgGs bound to spike. (FIG. 34B) Model showing the expected distance of ˜50 Å between centers of 2130 Fabs bound to RBD in ‘in’ configuration. This distance likewise enables neighboring 2130 IgGs bound to RBD in ‘in’ configuration to engage in IgG-to-IgG stabilizing interactions.

FIG. 35A through FIG. 35I: Structural details of the diverse interactions between in vivo-produced DMAb Fabs and the SARS-CoV-2 (WA1/2020) spike trimer. (FIG. 35A) Structural overview of RBD (blue) in complex with both 2196 (VL in pink; VH in salmon) and 2130 DMAb Fab (VL in gold; VH in green). (FIG. 35B) 2130 interactions with RBD main chain partners (CDRH3 T102 to RBD R346 peptide bond; RBD 346 to CDRH3 Y100 peptide bond; CDRH3 Y98 top to RBD V445 peptide bond; RBD N450 to CDRH3 Y100 peptide bond) and side chain partners (CHRL1 N30 to RBD S494 and CDRL1 to S30B to RBD 484E). (FIG. 35C) 2196 interactions with RBD (Q493 engages CDRH2 S54. RBD N481 engages CDRL1 Y32, RBD N487 engages CHRL3 D104 and RBD T478 engages CHRL3 D104). (FIG. 35D-E) 2130 interactions with RBD via hydrophobic interactions. (FIG. 35D) CDRL2 W50 packs against RBD G446. G447 and Y449. (FIG. 35E) CDRH3 G104-P105 packs against RBD L441 and P499. (FIG. 35F-G) 2196 interacts with RBD via hydrophobic interactions; (FIG. 35F) hydrophobic cage with RBD F486 formed by CDRL1 Y32. CDRL3 Y91 and W96. CDRH3 P95 and F106. (FIG. 35G) additional hydrophobic contacts include CDRH1 M30. CDRH2 G53 and RBD L455 and L456. (FIG. 35H-I) Cation-pi interactions between 2130 and RBD; (FIG. 35H) CDRL1 30F to RBD Y449. (FIG. 35I) CDRH3 Y98 to RBD K444.

FIG. 36A through FIG. 36B: Predictive modeling against B.1.617.2 (Delta) and B.1.1.529/BA.1 (Omicron). (FIG. 36A) The Delta variant exhibits one mutation relevant for 2196 binding (and none for 2130 binding). This T478K Delta mutation is tolerated well and while a hydrogen bonding is broken between T478 and 2196 CDRH3 D104. D104 remains hydrogen bonded with RBD. (FIG. 36B) Omicron likewise exhibits the T478K substitution in identical fashion to Delta. In addition. Omicron exhibits Q493R. The hydrogen bond between Q493 and 2196 CDRH2 S54 is simply replaced by a similar hydrogen bond between R493 and S54. In addition. R493 is in hydrogen bond distance to 2196 CDRH2 N56. Omicron also exhibits E484A. This breaks E484 hydrogen bonding to 2130 CDRL1 S30B; however, the shorter side chain of A484 may hydrophobically pack against 2196 CDRH2 V52-G53.

FIG. 37A-37C: COVID-19 DMAb Fc-WT and Fc-YTE cocktails express in rhesus macaques and neutralize SARS-CoV-2. (A) DMAb challenge experimental design. (B) DMAb serum expression. (C) Serum from DMAb administered macaques in SARSCoV2 authentic live virus and Spike pseudotyped virus.

FIG. 38A-38B: Rhesus macaques receiving the DMAb cocktail have fewer signs of COVID-19 associated disease. (A) DMAb challenge experimental design. (B) Clinical scores.

FIG. 39A-39B: The presence of gross pathological lesions was significantly lower for DMAb-administered animals. (A) Necropsy Score. (B) Gross Pathology Scores upon Necropsy (Day 7 post-challenge).

FIG. 40: Lung histopathology scores indicated significantly fewer signs of SARS-CoV-2 associated pneumonia in DMAb-administered animals.

FIG. 41 depicts a table of strategies to improve functionality/efficacy of antibodies.

FIG. 42 depicts a table of selected Fc modifications and predicted functionality.

FIG. 43 depicts the design of plasmid constructs encoding the indicated 2130 DMAb Fc variants.

FIG. 44 depicts data demonstrating the expression of the indicated 2130 DMAb variants in vitro.

FIG. 45 depicts data demonstrating the in vivo expression kinetics of Fc-engineered 2130 DMAb variants.

FIG. 46 depicts data demonstrating the in vivo-launched 2130 DMAb variants retain comparable antiviral/neutralizing activity against SARSpCoV-2 pseudotyped virus (strains WA1/2020 and B.1.351).

FIG. 47 depicts data demonstrating that the effector-enhanced 2130 DMAb variants demonstrate improved in vitro elimination of HEK293 cells expressing SARS-CoV-2 spike protein compared the parental IgG1 WT construct. This is particularly pronounced for the SDAL variant.

FIG. 48 depicts data demonstrating that the effector-enhanced 2130 DMAb variants demonstrate improved in vitro elimination of Hela cells expressing SARS-CoV-2 spike protein compared the parental IgG1 WT construct. This is particularly pronounced for the SDAL variant.

### DETAILED DESCRIPTION

The present invention relates to compositions comprising a recombinant nucleic acid sequence encoding an antibody, a fragment thereof, a variant thereof, or a combination thereof. The composition can be administered to a subject in need thereof to facilitate in vivo expression and formation of a synthetic antibody.

In particular, the heavy chain and light chain polypeptides expressed from the recombinant nucleic acid sequences can assemble into the synthetic antibody. The heavy chain polypeptide and the light chain polypeptide can interact with one another such that assembly results in the synthetic antibody being capable of binding the antigen, being more immunogenic as compared to an antibody not assembled as described herein, and being capable of eliciting or inducing an immune response against the antigen.

Additionally, these synthetic antibodies are generated more rapidly in the subject than antibodies that are produced in response to antigen induced immune response. The synthetic antibodies are able to effectively bind and neutralize a range of antigens. The synthetic antibodies are also able to effectively protect against and/or promote survival of disease.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising.” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein may mean refers to the nucleic acid (RNA or DNA molecule) that comprise a nucleotide sequence which encodes an antibody as set forth herein. The coding sequence may also comprise a DNA sequence which encodes an RNA sequence. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The genetic construct may also refer to a DNA molecule which transcribes an RNA. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, RSV-LTR promoter, tac promoter, SV40 early promoter or SV40 late promoter and the CMV IE promoter.

“Sample” or “biological sample” as used herein means a biological material isolated from an individual. The biological sample may contain any biological material suitable for detecting the desired biomarkers, and may comprise cellular and/or non-cellular material obtained from the individual.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tm may be the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering a vaccine of the present invention to a subject prior to onset of the disease. Suppressing the disease involves administering a vaccine of the present invention to a subject after induction of the disease but before its clinical appearance. Repressing the disease involves administering a vaccine of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of +2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Compositions

The instant invention relates to the design and development of a nucleic acid encoded monoclonal antibody sequences comprising at least one Fc modification as compared to a parental antibody.

In one embodiment, the nucleic acid molecule encodes at least one Fc modification in the heavy chain to increase the stability or half-life of the encoded antibody, to alter (e.g. increase or decrease) complement activation, to alter (e.g. increase or decrease) Fcγ receptor binding or to alter the dimerization capability (e.g., prevent homodimerization or promote heterdimerization) of the encoded antibody.

Exemplary Fc modifications that can be encoded by the nucleic acid molecule of the invention include, but are not limited to, M252Y/S254T/T256E (YTE), L234F, L235E, and P331S (TM), H268F/S324T (FT), E430G, S239D/I332E (DE), S239D/I332E/E430G (DEG), G236A (GA), G236A/I332E (AE), S267E/H268F/S324T (EFT), A330L/I332E (ALIE), G236A/A330L/I332E (GAALIE), (S239D/A330L) SDAL, (G236A/S239D/A330L) GASDAL, G236A/S239D/A330L/I332E (GASDALIE), G236A/S239D/I332E (ADE), G236R/L328R (GRLR), L234F/L235Q/K322Q/M252T/S254T/T256E (FQQ-YTE) and any combination thereof.

In one embodiment, the invention provides a composition comprising a first nucleic acid molecule comprising a nucleotide sequence encoding a heavy chain of an antibody comprising at least one Fc variant and a second nucleic acid molecule comprising a nucleotide sequence encoding a light chain of an antibody.

In one embodiment, the composition comprises a nucleic acid molecule comprising a first nucleotide sequence encoding a heavy chain region of an antibody comprising at least one Fc variant and a second nucleotide sequence encoding a light chain of a synthetic antibody. In one embodiment, the nucleic acid molecule comprises a nucleotide sequence encoding a cleavage domain.

In one embodiment, the nucleotide sequence encoding the heavy chain is located 5′ to the nucleotide sequence encoding the light chain of the synthetic antibody with a cleavage domain located between the sequence encoding the heavy chain and the sequence encoding the light chain.

In one embodiment, the nucleotide sequence encoding the heavy chain is located 3′ to the nucleotide sequence encoding the light chain of the synthetic antibody with a cleavage domain located between the sequence encoding the heavy chain and the sequence encoding the light chain.

In one embodiment, the invention provides a composition comprising a first nucleic acid molecule comprising a nucleotide sequence encoding a variable heavy chain region of an anti-SARS-CoV-2 antibody comprising at least one Fc variant and a second nucleic acid molecule comprising a nucleotide sequence encoding a light chain region of an anti-SARS-CoV-2 antibody.

In one embodiment, the invention provides a composition comprising a single nucleic acid molecule comprising a nucleotide sequence encoding a variable heavy chain region of an anti-SARS-CoV-2 antibody comprising at least one Fc variant and a nucleotide sequence encoding a light chain region of an anti-SARS-CoV-2 antibody.

Exemplary anti-SARS-CoV-2 antibodies comprising Fc variants include, but are not limited to, antibodies comprising a heavy chain as set forth in SEQ ID NO:6, SEQ ID NO: 12, SEQ ID NO: 18, SEQ ID NO:24, SEQ ID NO:26, SEQ ID NO:28, SEQ ID NO: 60, SEQ ID NO:62, SEQ ID NO:68, SEQ ID NO:72, SEQ ID NO:74, SEQ ID NO: 88, SEQ ID NO:90, SEQ ID NO:92, SEQ ID NO:94, SEQ ID NO:96 and SEQ ID NO: 98.

Exemplary nucleic acid molecules encoding a heavy chain of an anti-SARS-CoV-2 antibodies comprising Fc variants include, but are not limited to, SEQ ID NO: 5, SEQ ID NO:11, SEQ ID NO:17, SEQ ID NO:23, SEQ ID NO:25, SEQ ID NO: 27, SEQ ID NO:59, SEQ ID NO:61, SEQ ID NO:67, SEQ ID NO: 71, SEQ ID NO: 73, SEQ ID NO:87, SEQ ID NO:89, SEQ ID NO:91, SEQ ID NO:93, SEQ ID NO: 95, and SEQ ID NO:97.

The composition of the invention can treat, prevent and/or protect against any disease, disorder, in a subject in need thereof. In certain embodiments, the composition can treat, prevent, and or/protect against cancer, or a disease or disorder associated with an infection. In certain embodiments, the composition can treat, prevent, and or/protect against condition associated with SARS-CoV-2 infection. In certain embodiments, the composition can treat, prevent, and or/protect against COVID-19.

The composition can result in the generation of the Fc modified synthetic antibody in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the Fc modified synthetic antibody in the subject within at least about 1 day, 2 days, 3 days, 4 days, 5 days, 6 days, 7 days, 8 days, 9 days, or 10 days of administration of the composition to the subject. The composition can result in generation of the Fc modified synthetic antibody in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the generation of the Fc modified synthetic antibody in the subject more quickly than the generation of an endogenous antibody in a subject who is administered an antigen to induce a humoral immune response. The composition can result in the generation of the Fc modified synthetic antibody at least about 1 day, 2 days, 3 days, 4 days, 5 days, 6 days, 7 days, 8 days, 9 days, or 10 days before the generation of the endogenous antibody in the subject who was administered an antigen to induce a humoral immune response.

The composition of the present invention can have features required of effective compositions such as being safe so that the composition does not cause illness or death; being protective against illness; and providing ease of administration, few side effects, biological stability and low cost per dose.

In some embodiments, the SARS-CoV-2 spike protein binding molecules (e.g., antibodies, etc.) of the present invention, exhibit a high capacity to detect and bind SARS-CoV-2 spike protein in a complex mixture of salts, compounds and other polypeptides, e.g., as assessed by any one of several in vitro and in vivo assays known in the art. The skilled artisan will understand that the SARS-CoV-2 spike protein binding molecules (e.g., antibodies, etc.) described herein as useful in the methods of diagnosis and treatment and prevention of disease, are also useful in procedures and methods of the invention that include, but are not limited to, an immunochromatography assay, an immunodot assay, a Luminex assay, an ELISA assay, an ELISPOT assay, a protein microarray assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, a protein chip assay, separation and purification processes, and affinity chromatography (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

In some embodiments, the SARS-CoV-2 spike protein binding molecules (e.g., antibodies, etc.) of the present invention, exhibit a high capacity to reduce or to neutralize SARS-CoV-2 spike protein activity (e.g., receptor binding activity, etc.) as assessed by any one of several in vitro and in vivo assays known in the art. For example, these SARS-CoV-2 spike protein binding molecules (e.g., antibodies, etc.) neutralize SARS-CoV-2-associated or SARS-CoV-2-mediated disease or disorder.

As used herein, a SARS-CoV-2 antigen binding molecule (e.g., antibody, etc.) that “specifically binds to a SARS-CoV-2 antigen” binds to a SARS-CoV-2 spike protein with a KD of 1×10⁻⁶ M or less, more preferably 1×10⁻⁷ M or less, more preferably 1×10⁻⁸ M or less, more preferably 5×10⁻⁹ M or less, more preferably 1×10⁻⁹ M or less or even more preferably 3×10⁻¹⁰ M or less. The term “does not substantially bind” to a protein or cells, as used herein, means does not bind or does not bind with a high affinity to the protein or cells, i.e., binds to the protein or cells with a KD of greater than 1×10⁶ M or more, more preferably 1×10⁵ M or more, more preferably 1×10⁴ M or more, more preferably 1×10³ M or more, even more preferably 1×10² M or more. The term “KD”, as used herein, is intended to refer to the dissociation constant, which is obtained from the ratio of Kd to Ka (i.e., Kd/Ka) and is expressed as a molar concentration (M). KD values for a SARS-CoV-2 spike protein binding molecule (e.g., antibody, etc.) can be determined using methods well established in the art. A preferred method for determining the KD of a binding molecule (e.g., antibody, etc.) is by using surface plasmon resonance, preferably using a biosensor system such as a Biacore® system.

As used herein, the term “high affinity” for an IgG antibody refers to an antibody having a KD of 1×10⁻⁷ M or less, more preferably 5×10⁻⁸ M or less, even more preferably 1×10⁻⁸ M or less, even more preferably 5×10⁻⁹ M or less and even more preferably 1×10⁻⁹ M or less for a target binding partner molecule. However, “high affinity” binding can vary for other antibody isotypes. For example, “high affinity” binding for an IgM isotype refers to an antibody having a KD of 10⁻⁶ M or less, more preferably 10⁻⁷ M or less, even more preferably 10⁻⁸ M or less.

In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

### Recombinant Nucleic Acid Sequence

As described above, the composition can comprise a recombinant nucleic acid sequence. The recombinant nucleic acid sequence can encode the antibody, a fragment thereof, a variant thereof, or a combination thereof. The antibody is described in more detail below.

The recombinant nucleic acid sequence can be a heterologous nucleic acid sequence. The recombinant nucleic acid sequence can include one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence for increased translation; addition of an immunoglobulin (Ig) leader sequence encoding a signal peptide; addition of an internal IRES sequence and eliminating to the extent possible cis-acting sequence motifs (i.e., internal TATA boxes).

### Recombinant Nucleic Acid Sequence Construct

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or an eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

### (1) Heavy Chain Polypeptide

The recombinant nucleic acid sequence construct can include the heterologous nucleic acid encoding the heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

### (2) Light Chain Polypeptide

The recombinant nucleic acid sequence construct can include the heterologous nucleic acid sequence encoding the light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

### (3) Protease Cleavage Site

The recombinant nucleic acid sequence construct can include heterologous nucleic acid sequence encoding a protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

### (4) Linker Sequence

The recombinant nucleic acid sequence construct can include one or more linker sequences. The linker sequence can spatially separate or link the one or more components described herein. In other embodiments, the linker sequence can encode an amino acid sequence that spatially separates or links two or more polypeptides.

### (5) Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

### (6) Intron

The recombinant nucleic acid sequence construct can include one or more introns. Each intron can include functional splice donor and acceptor sites. The intron can include an enhancer of splicing. The intron can include one or more signals required for efficient splicing.

### (7) Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

### (8) Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

### (9) Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

### (10) Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

### (11) Leader Sequence

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and a IgE signal peptide.

### Arrangement of the Recombinant Nucleic Acid Sequence Construct

As described above, the recombinant nucleic acid sequence can include one or more recombinant nucleic acid sequence constructs, in which each recombinant nucleic acid sequence construct can include one or more components. The one or more components are described in detail above. The one or more components, when included in the recombinant nucleic acid sequence construct, can be arranged in any order relative to one another. In some embodiments, the one or more components can be arranged in the recombinant nucleic acid sequence construct as described below.

### (12) Arrangement 1

In one arrangement, a first recombinant nucleic acid sequence construct can include the heterologous nucleic acid sequence encoding the heavy chain polypeptide and a second recombinant nucleic acid sequence construct can include the heterologous nucleic acid sequence encoding the light chain polypeptide. The first recombinant nucleic acid sequence construct can be placed in a vector. The second recombinant nucleic acid sequence construct can be placed in a second or separate vector. Placement of the recombinant nucleic acid sequence construct into the vector is described in more detail below.

The first recombinant nucleic acid sequence construct can also include the promoter, intron, transcription termination region, initiation codon, termination codon, and/or polyadenylation signal. The first recombinant nucleic acid sequence construct can further include the leader sequence, in which the leader sequence is located upstream (or 5′) of the heterologous nucleic acid sequence encoding the heavy chain polypeptide. Accordingly, the signal peptide encoded by the leader sequence can be linked by a peptide bond to the heavy chain polypeptide.

The second recombinant nucleic acid sequence construct can also include the promoter, initiation codon, termination codon, and polyadenylation signal. The second recombinant nucleic acid sequence construct can further include the leader sequence, in which the leader sequence is located upstream (or 5′) of the heterologous nucleic acid sequence encoding the light chain polypeptide. Accordingly, the signal peptide encoded by the leader sequence can be linked by a peptide bond to the light chain polypeptide.

Accordingly, one example of arrangement 1 can include the first vector (and thus first recombinant nucleic acid sequence construct) encoding the heavy chain polypeptide that includes VH and CH1, and the second vector (and thus second recombinant nucleic acid sequence construct) encoding the light chain polypeptide that includes VL and CL. A second example of arrangement 1 can include the first vector (and thus first recombinant nucleic acid sequence construct) encoding the heavy chain polypeptide that includes VH, CH1, hinge region, CH2, and CH3, and the second vector (and thus second recombinant nucleic acid sequence construct) encoding the light chain polypeptide that includes VL and CL.

### (13) Arrangement 2

In a second arrangement, the recombinant nucleic acid sequence construct can include the heterologous nucleic acid sequence encoding the heavy chain polypeptide and the heterologous nucleic acid sequence encoding the light chain polypeptide. The heterologous nucleic acid sequence encoding the heavy chain polypeptide can be positioned upstream (or 5′) of the heterologous nucleic acid sequence encoding the light chain polypeptide. Alternatively, the heterologous nucleic acid sequence encoding the light chain polypeptide can be positioned upstream (or 5′) of the heterologous nucleic acid sequence encoding the heavy chain polypeptide.

The recombinant nucleic acid sequence construct can be placed in the vector as described in more detail below.

The recombinant nucleic acid sequence construct can include the heterologous nucleic acid sequence encoding the protease cleavage site and/or the linker sequence. If included in the recombinant nucleic acid sequence construct, the heterologous nucleic acid sequence encoding the protease cleavage site can be positioned between the heterologous nucleic acid sequence encoding the heavy chain polypeptide and the heterologous nucleic acid sequence encoding the light chain polypeptide. Accordingly, the protease cleavage site allows for separation of the heavy chain polypeptide and the light chain polypeptide into distinct polypeptides upon expression. In other embodiments, if the linker sequence is included in the recombinant nucleic acid sequence construct, then the linker sequence can be positioned between the heterologous nucleic acid sequence encoding the heavy chain polypeptide and the heterologous nucleic acid sequence encoding the light chain polypeptide.

The recombinant nucleic acid sequence construct can also include the promoter, intron, transcription termination region, initiation codon, termination codon, and/or polyadenylation signal. The recombinant nucleic acid sequence construct can include one or more promoters. The recombinant nucleic acid sequence construct can include two promoters such that one promoter can be associated with the heterologous nucleic acid sequence encoding the heavy chain polypeptide and the second promoter can be associated with the heterologous nucleic acid sequence encoding the light chain polypeptide. In still other embodiments, the recombinant nucleic acid sequence construct can include one promoter that is associated with the heterologous nucleic acid sequence encoding the heavy chain polypeptide and the heterologous nucleic acid sequence encoding the light chain polypeptide.

The recombinant nucleic acid sequence construct can further include two leader sequences, in which a first leader sequence is located upstream (or 5′) of the heterologous nucleic acid sequence encoding the heavy chain polypeptide and a second leader sequence is located upstream (or 5′) of the heterologous nucleic acid sequence encoding the light chain polypeptide. Accordingly, a first signal peptide encoded by the first leader sequence can be linked by a peptide bond to the heavy chain polypeptide and a second signal peptide encoded by the second leader sequence can be linked by a peptide bond to the light chain polypeptide.

Accordingly, one example of arrangement 2 can include the vector (and thus recombinant nucleic acid sequence construct) encoding the heavy chain polypeptide that includes VH and CH1, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A second example of arrangement of 2 can include the vector (and thus recombinant nucleic acid sequence construct) encoding the heavy chain polypeptide that includes VH and CH1, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

A third example of arrangement 2 can include the vector (and thus recombinant nucleic acid sequence construct) encoding the heavy chain polypeptide that includes VH, CH1, hinge region, CH2, and CH3, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A forth example of arrangement of 2 can include the vector (and thus recombinant nucleic acid sequence construct) encoding the heavy chain polypeptide that includes VH, CH1, hinge region, CH2, and CH3, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

Expression from the Recombinant Nucleic Acid Sequence Construct

As described above, the recombinant nucleic acid sequence construct can include, amongst the one or more components, the heterologous nucleic acid sequence encoding the heavy chain polypeptide and/or the heterologous nucleic acid sequence encoding the light chain polypeptide. Accordingly, the recombinant nucleic acid sequence construct can facilitate expression of the heavy chain polypeptide and/or the light chain polypeptide.

When arrangement 1 as described above is utilized, the first recombinant nucleic acid sequence construct can facilitate the expression of the heavy chain polypeptide and the second recombinant nucleic acid sequence construct can facilitate expression of the light chain polypeptide. When arrangement 2 as described above is utilized, the recombinant nucleic acid sequence construct can facilitate the expression of the heavy chain polypeptide and the light chain polypeptide.

Upon expression, for example, but not limited to, in a cell, organism, or mammal, the heavy chain polypeptide and the light chain polypeptide can assemble into the Fc modified synthetic antibody. In particular, the heavy chain polypeptide and the light chain polypeptide can interact with one another such that assembly results in the Fc modified synthetic antibody being capable of binding the antigen. In other embodiments, the heavy chain polypeptide and the light chain polypeptide can interact with one another such that assembly results in the Fc modified synthetic antibody being more immunogenic as compared to an antibody not assembled as described herein. In still other embodiments, the heavy chain polypeptide and the light chain polypeptide can interact with one another such that assembly results in the Fc modified synthetic antibody being capable of eliciting or inducing an immune response against the antigen.

### Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. In some embodiments, the vector includes linear DNA, enzymatic DNA or synthetic DNA. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

### (14) Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

### (15) Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extra-chromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif.), which may be used for protein production in Escherichia coli (E. coli). The plasmid may also be pYES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

### (16) RNA

In one embodiment, the nucleic acid is an RNA molecule. In one embodiment, the RNA molecule is transcribed from a DNA sequence described herein. Accordingly, in one embodiment, the invention provides an RNA molecule encoding one or more of the DMAbs. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

### (17) Circular and Linear Vector

The one or more vectors may be circular plasmid, which may transform a target cell by integration into the cellular genome or exist extra-chromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be pcrM2. The LEC can be pcrNP. pcrNP and pcrMR can be derived from pNP (Puerto Rico/34) and pM2 (New Caledonia/99), respectively.

### (18) Viral Vectors

In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

### (19) Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described below in more detail.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### 3. Antibody

As described above, the recombinant nucleic acid sequence can encode the antibody, a fragment thereof, a variant thereof, or a combination thereof. The antibody can bind or react with the antigen, which is described in more detail below. The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F (ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antibody can be the Fab or F(ab′)₂. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody as described below in more detail. The antibody can be a bifunctional antibody as also described below in more detail.

As described above, the antibody can be generated in the subject upon administration of the composition to the subject. The antibody may have a half-life within the subject. In some embodiments, the antibody may be modified to extend or shorten its half-life within the subject. Such modifications are described below in more detail.

The antibody can be defucosylated as described in more detail below.

In one embodiment, the antibody binds a SARS-CoV-2 antigen. In one embodiment, the antibody binds at least one epitope of a SARS-CoV-2 Spike protein. In one embodiment, the antibody binds a SARS-CoV-2 RBD.

The antibody may be modified to reduce or prevent antibody-dependent enhancement (ADE) of disease associated with the antigen as described in more detail below.

### Bispecific Antibody

The recombinant nucleic acid sequence can encode a bispecific antibody, a fragment thereof, a variant thereof, or a combination thereof. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker.

The invention provides novel bispecific antibodies comprising a first antigen-binding site that specifically binds to a first target and a second antigen-binding site that specifically binds to a second target, with particularly advantageous properties such as producibility, stability, binding affinity, biological activity, specific targeting of certain T cells, targeting efficiency and reduced toxicity. In some instances, there are bispecific antibodies, wherein the bispecific antibody binds to the first target with high affinity and to the second target with low affinity. In other instances, there are bispecific antibodies, wherein the bispecific antibody binds to the first target with low affinity and to the second target with high affinity. In other instances, there are bispecific antibodies, wherein the bispecific antibody binds to the first target with a desired affinity and to the second target with a desired affinity.

In one embodiment, the bispecific antibody is a bivalent antibody comprising a) a first light chain and a first heavy chain of an antibody specifically binding to a first antigen, and b) a second light chain and a second heavy chain of an antibody specifically binding to a second antigen.

A bispecific antibody molecule according to the invention may have two binding sites of any desired specificity. In some embodiments one of the binding sites is capable of binding a tumor associated antigen. In some embodiments, the binding site included in the Fab fragment is a binding site specific for a SARS-CoV-2 antigen. In some embodiments, the binding site included in the single chain Fv fragment is a binding site specific for a SARS-CoV-2 antigen such as a SARS-CoV-2 spike antigen.

In some embodiments, one of the binding sites of a bispecific antibody according to the invention is able to bind a T-cell specific receptor molecule and/or a natural killer cell (NK cell) specific receptor molecule. A T-cell specific receptor is the so called “T-cell receptor” (TCRs), which allows a T cell to bind to and, if additional signals are present, to be activated by and respond to an epitope/antigen presented by another cell called the antigen-presenting cell or APC. The T cell receptor is known to resemble a Fab fragment of a naturally occurring immunoglobulin. It is generally monovalent, encompassing α- and β-chains, in some embodiments it encompasses γ-chains and 8-chains. Accordingly, in some embodiments the TCR is TCR (alpha/beta) and in some embodiments it is TCR (gamma/delta). The T cell receptor forms a complex with the CD3 T-Cell co-receptor. CD3 is a protein complex and is composed of four distinct chains. In mammals, the complex contains a CD3γ chain, a CD36 chain, and two CD3E chains. These chains associate with a molecule known as the T cell receptor (TCR) and the ζ-chain to generate an activation signal in T lymphocytes. Hence, in some embodiments a T-cell specific receptor is the CD3 T-Cell co-receptor. In some embodiments, a T-cell specific receptor is CD28, a protein that is also expressed on T cells. CD28 can provide co-stimulatory signals, which are required for T cell activation. CD28 plays important roles in T-cell proliferation and survival, cytokine production, and T-helper type-2 development. Yet a further example of a T-cell specific receptor is CD134, also termed Ox40. CD134/OX40 is being expressed after 24 to 72 hours following activation and can be taken to define a secondary costimulatory molecule. Another example of a T-cell receptor is 4-1 BB capable of binding to 4-1 BB-Ligand on antigen presenting cells (APCs), whereby a costimulatory signal for the T cell is generated. Another example of a receptor predominantly found on T-cells is CD5, which is also found on B cells at low levels. A further example of a receptor modifying T cell functions is CD95, also known as the Fas receptor, which mediates apoptotic signaling by Fas-ligand expressed on the surface of other cells. CD95 has been reported to modulate TCR/CD3-driven signaling pathways in resting T lymphocytes.

An example of a NK cell specific receptor molecule is CD16, a low affinity Fc receptor and NKG2D. An example of a receptor molecule that is present on the surface of both T cells and natural killer (NK) cells is CD2 and further members of the CD2-superfamily. CD2 is able to act as a co-stimulatory molecule on T and NK cells.

In some embodiments, the first binding site of the bispecific antibody molecule binds a SARS-CoV-2 antigen and the second binding site binds a T cell specific receptor molecule and/or a natural killer (NK) cell specific receptor molecule. In some embodiments, the first binding site of the antibody molecule binds the SARS-CoV-2 spike antigen, and the second binding site binds a T cell specific receptor molecule and/or a natural killer (NK) cell specific receptor molecule. In some embodiments, the first binding site of the antibody molecule binds a SARS-CoV-2 spike antigen and the second binding site binds one of CD3, the T cell receptor (TCR), CD28, CD16, NKG2D, Ox40, 4-1BB, CD2, CD5 and CD95.

In some embodiments, the first binding site of the antibody molecule binds a T cell specific receptor molecule and/or a natural killer (NK) cell specific receptor molecule and the second binding site binds a SARS-CoV-2 antigen. In some embodiments, the first binding site of the antibody binds a T cell specific receptor molecule and/or a natural killer (NK) cell specific receptor molecule and the second binding site binds the SARS-CoV-2 spike antigen. In some embodiments, the first binding site of the antibody binds one of CD3, the T cell receptor (TCR), CD28, CD16, NKG2D, Ox40, 4-1BB, CD2, CD5 and CD95, and the second binding site binds the SARS-CoV-2 spike antigen.

### CAR Molecules

In one embodiment, the invention provides a chimeric antigen receptor (CAR) comprising a binding domain comprising a SARS-CoV-2 antibody of the invention. In one embodiment, the CAR comprises an antigen binding domain. In one embodiment, the antigen binding domain is a targeting domain, wherein the targeting domain directs the T cell expressing the CAR to a SARS-CoV-2 viral particle. For example, in one embodiment, the targeting domain comprises an antibody, antibody fragment, or peptide that specifically binds to a SARS-CoV-2 antigen.

In various embodiments, the CAR can be a “first generation,” “second generation,” “third generation,” “fourth generation” or “fifth generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

“First generation” CARs for use in the invention comprise an antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD35-chain, which is the primary transmitter of signals from endogenous T cell receptors (TCRs). “First generation” CARs can provide de novo antigen recognition and cause activation of both CD4+ and CD8+ T cells through their CD3 chain signaling domain in a single fusion molecule, independent of HLA-mediated antigen presentation.

“Second-generation” CARs for use in the invention comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3ζ signaling domain. Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1(9):1577-1583 (2012)).

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD3 {signaling domain in addition to a constitutive or inducible chemokine component.

“Fifth generation” CARs provide co-stimulation, for example, by CD28 or 4-1BB domains, and activation, for example, by a CD3ζ signaling domain, a constitutive or inducible chemokine component, and an intracellular domain of a cytokine receptor, for example, IL-2Rβ.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above. In a particular non-limiting embodiment, the antigen-binding domain is a SARS-CoV-2 antibody of the invention or a variant thereof, such as an scFV fragment of a SARS-CoV-2 antibody of the invention specific for binding to a surface antigen of SARS-CoV-2.

### Bifunctional Antibody

The recombinant nucleic acid sequence can encode a bifunctional antibody, a fragment thereof, a variant thereof, or a combination thereof. The bifunctional antibody can bind or react with the antigen described below. The bifunctional antibody can also be modified to impart an additional functionality to the antibody beyond recognition of and binding to the antigen. Such a modification can include, but is not limited to, coupling to factor H or a fragment thereof. Factor His a soluble regulator of complement activation and thus, may contribute to an immune response via complement-mediated lysis (CML).

### Immune Cells

In various embodiments, the invention relates to a composition comprising an immune cell engineered for expression or endogenous secretion of an anti-SARS-CoV-2 antibody of the invention. In one embodiment, the anti-SARS-CoV-2 antibody is a bi-specific T cell engaging antibody comprising a domain for binding to a SARS-CoV-2 antigen and a domain for activating an immune cell. Examples of immune cells that can be engineered for expression or secretion of an anti-SARS-CoV-2 antibody of the invention include, but are not limited to, T cells, B cells, natural killer (NK) cells, or macrophages. In some embodiments, the immune cell further comprises a chimeric antigen receptor (CAR). Therefore, in some embodiments, the invention relates to the use of CAR T-cells for expression or delivery of an anti-SARS-CoV-2 antibody of the invention.

In various embodiments, the invention relates to compositions for endogenous secretion of a T cell-redirecting bispecific antibody (T-bsAb) by engineered T cells (STAb-T cells), which have been engineered to express the anti-SARS-CoV-2 antibody of the invention. In various embodiments, the method comprises administering to a subject in need thereof a composition comprising a STAb-T cell, wherein the STAb-T cell has been engineered to express a bispecific immune cell engaging anti-SARS-CoV-2 antibody of the invention. In some embodiments, the STAb-T cell further comprises a chimeric antigen receptor (CAR). Therefore, in some embodiments, the invention relates to the use of CAR T-cells for expression or delivery of an anti-SARS-CoV-2 antibody of the invention.

### Extension of Antibody Half-Life

As described above, the antibody may be modified to extend or shorten the half-life of the antibody in the subject. The modification may extend or shorten the half-life of the antibody in the serum of the subject.

The modification may be present in a constant region of the antibody. The modification may be one or more amino acid substitutions in a constant region of the antibody that extend the half-life of the antibody as compared to a half-life of an antibody not containing the one or more amino acid substitutions. The modification may be one or more amino acid substitutions in the CH2 domain of the antibody that extend the half-life of the antibody as compared to a half-life of an antibody not containing the one or more amino acid substitutions.

In some embodiments, the one or more amino acid substitutions in the constant region may include replacing a methionine residue in the constant region with a tyrosine residue, a serine residue in the constant region with a threonine residue, a threonine residue in the constant region with a glutamate residue, or any combination thereof, thereby extending the half-life of the antibody.

In other embodiments, the one or more amino acid substitutions in the constant region may include replacing a methionine residue in the CH2 domain with a tyrosine residue, a serine residue in the CH2 domain with a threonine residue, a threonine residue in the CH2 domain with a glutamate residue, or any combination thereof, thereby extending the half-life of the antibody.

### Defucosylation

The recombinant nucleic acid sequence can encode an antibody that is not fucosylated (i.e., a defucosylated antibody or a non-fucosylated antibody), a fragment thereof, a variant thereof, or a combination thereof. Fucosylation includes the addition of the sugar fucose to a molecule, for example, the attachment of fucose to N-glycans, O-glycans and glycolipids. Accordingly, in a defucosylated antibody, fucose is not attached to the carbohydrate chains of the constant region. In turn, this lack of fucosy lation may improve FcγRIIIa binding and antibody directed cellular cytotoxic (ADCC) activity by the antibody as compared to the fucosylated antibody. Therefore, in some embodiments, the non-fucosylated antibody may exhibit increased ADCC activity as compared to the fucosylated antibody.

The antibody may be modified so as to prevent or inhibit fucosylation of the antibody. In some embodiments, such a modified antibody may exhibit increased ADCC activity as compared to the unmodified antibody. The modification may be in the heavy chain, light chain, or a combination thereof. The modification may be one or more amino acid substitutions in the heavy chain, one or more amino acid substitutions in the light chain, or a combination thereof.

### 4. Antigen

In one embodiment, the Fc modified synthetic antibody of the invention is directed to an antigen or fragment or variant thereof. The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

The antigen can be a tumor antigen. The antigen can be associated with increased risk of cancer development or progression.

The antigen can be a pathogen antigen. The antigen can be associated with infection or a disease or disorder associated with an infectious agent.

### Tumor Antigen

The antigen binding domain of the Fc modified synthetic antibody of the invention can interact with a tumor antigen. In the context of the present invention, “tumor antigen” or “hyperproliferative disorder antigen” or “antigen associated with a hyperproliferative disorder,” refers to antigens that are common to specific hyperproliferative disorders such as cancer.

The type of tumor antigen referred to in the invention may be a tumor-specific antigen (TSA) or a tumor-associated antigen (TAA). A TSA is unique to tumor cells and does not occur on other cells in the body. A TAA antigen is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

The antigens discussed herein are merely included by way of example. The list is not intended to be exclusive and further examples will be readily apparent to those of skill in the art.

Tumor antigens are proteins that are produced by tumor cells that elicit an immune response, particularly T-cell mediated immune responses. The selection of the antigen binding moiety of the invention will depend on the particular type of cancer to be treated. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, Her2, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

Illustrative examples of a tumor associated surface antigen are CD10, CD19, CD20, CD22, CD33, CD123, B-cell maturation antigen (BCMA), Fms-like tyrosine kinase 3 (FLT-3, CD135), chondroitin sulfate proteoglycan 4 (CSPG4, melanoma-associated chondroitin sulfate proteoglycan), Epidermal growth factor receptor (EGFR), Her2, Her3, IGFR, CD133, IL3R, fibroblast activating protein (FAP), CDCP1, Derlin1, Tenascin, frizzled 1-10, the vascular antigens VEGFR2 (KDR/FLK1), VEGFR3 (FLT4, CD309), PDGFR-α (CD140a), PDGFR-.beta. (CD140b) Endoglin, CLEC14, Tem1-8, and Tie2. Further examples may include A33, CAMPATH-1 (CDw52), Carcinoembryonic antigen (CEA), Carboanhydrase IX (MN/CA IX), CD21, CD25, CD30, CD34, CD37, CD44v6, CD45, CD133, de2-7 EGFR, EGFRVIII, EpCAM, Ep-CAM, Folate-binding protein, G250, Fms-like tyrosine kinase 3 (FLT-3, CD135), follicle stimulating hormone receptor (FSHR), c-Kit (CD117), CSFIR (CD115), HLA-DR, IGFR, IL-2 receptor, IL3R, MCSP (Melanoma-associated cell surface chondroitin sulphate proteoglycane), Muc-1, Prostate-specific membrane antigen (PSMA), Prostate stem cell antigen (PSCA), Prostate specific antigen (PSA), and TAG-72. Examples of antigens expressed on the extracellular matrix of tumors are tenascin and the fibroblast activating protein (FAP).

In one embodiment, the tumor antigen is a hormone or fragment thereof which can be used to target a specific receptor. Examples include, but are not limited to, FSH hormone, LH hormone, TSH hormone or fragments thereof.

Non-limiting examples of TSA or TAA antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG16, TA-90\Mac-2 binding protein\cyclophilin C-associated protein, TAAL6, TAG72, TLP, and TPS.

### Pathogen Antigen

The antigen binding domain of the Fc modified synthetic antibody of the invention can interact with a pathogen antigen. In one embodiment, the pathogen is pathogenic to humans. In one embodiment, the pathogen is pathogenic to non-humans (e.g., a non-human mammal pathogen, a plant pathogen, a marine animal pathogen or an insect pathogen.) A pathogenic microbe can be a virus, a bacterium, and/or a fungus. In certain aspects, a device of the invention can be configured to detect a variety of microbes including viruses, bacteria, and fungi simultaneously.

In certain aspects, a microbe includes a virus. The virus can be from the Adenoviridae, Coronaviridae, Filoviridae, Flaviviridae, Hepadnaviridae, Herpesviridae, Orthomyxoviridae, Paramyxovirinae, Pneumovirinae, Picornaviridae, Poxyiridae, Retroviridae, or Togaviridae family of viruses; and/or Parainfluenza, Influenza, H5N1, Marburg, Ebola, Severe acute respiratory syndrome coronavirus, Yellow fever virus, Human respiratory syncytial virus, Hantavirus, or Vaccinia virus. In some embodiments, the virus is Severe acute respiratory syndrome coronavirus 2 (SARS-CoV-2), smallpox, influenza, mumps, measles, chickenpox, Ebola, HIV, or rubella.

In yet another aspect, the pathogenic or potentially pathogenic microbe can be a bacteria. A bacterium can be an intracellular, a gram positive, or a gram-negative bacteria. In a further aspect, bacteria include, but is not limited to a Neisseria meningitidis (N. meningitidis), Streptococcus pneumoniae (S. pneumoniae), and Haemophilus influenzae type B (Hib), B. pertussis, B. parapertussis, B. holmesii, Escherichia, a Staphylococcus, a Bacillus, a Francisella, or a Yersinia bacteria. In still a further aspect, the bacteria is Bacillus anthracis, Yersinia pestis, Francisella tularensis, Pseudomonas aerugenosa, or Staphylococcus aureas. In still a further aspect, a bacteria is a drug resistant bacteria, such as a multiple drug resistant Staphylococcus aureas (MRSA). Representative medically relevant Gram-negative bacilli include Hemophilus influenzae, Klebsiella pneumoniae, Legionella pneumophila, Pseudomonas aeruginosa, Escherichia coli, Proteus mirabilis, Enterobacter cloacae, Serratia marcescens, Helicobacter pylori, Salmonella enteritidis, and Salmonella typhi. Representative gram-positive bacteria include, but are not limited to Bacillus, Listeria, Staphylococcus, Streptococcus, Enterococcus, Actinobacteria and Clostridium Mycoplasma that lack cell walls and cannot be Gram stained, including those bacteria that are derived from such forms.

In still another aspect, the pathogenic or potentially pathogenic microbe is a fungus, such as members of the family Aspergillus, Candida, Crytpococus, Histoplasma, Coccidioides, Blastomyces, Pneumocystis, or Zygomyces. In still further embodiments a fungus includes, but is not limited to Aspergillus fumigatus, Candida albicans, Cryptococcus neoformans, Histoplasma capsulatum, Coccidioides immitis, or Pneumocystis carinii. The family zygomycetes includes Basidiobolales (Basidiobolaceae), Dimargaritales (Dimargaritaceae), Endogonales (Endogonaceae), Entomophthorales (Ancylistaceae, Completoriaceae, Entomophthoraceae, Meristacraceae, Neozygitaceae), Kickxellales (Kickxellaceae), Mortierellales (Mortierellaceae), Mucorales, and Zoopagales.

### Substrates

In one embodiment, the present invention provides a scaffold, substrate, or device comprising a Fc modified synthetic antibody of the invention or a fragment thereof, or nucleic acid molecule encoding the same. For example, in some embodiments, the present invention provides a tissue engineering scaffold, including but not limited to, a hydrogel, electrospun scaffold, polymeric matrix, or the like, comprising the modulator. In certain embodiments, a bispecific immune cell engager, fragment thereof, or nucleic acid molecule encoding the same, may be coated along the surface of the scaffold, substrate, or device. In certain embodiments, the Fc modified synthetic antibody of the invention, fragment thereof, or nucleic acid molecule encoding the same is encapsulated within the scaffold, substrate, or device.

### 5. Excipients and Other Components of the Composition

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition may further comprise a genetic facilitator agent as described in U.S. Ser. No. 021,579 filed Apr. 1, 1994, which is fully incorporated by reference.

In some embodiments, the composition comprises hyaluronidase. In some embodiments, the composition comprises recombinant human hyaluronidase.

The composition may comprise DNA at quantities of from about 1 nanogram to 100 milligrams; about 1 microgram to about 10 milligrams; or preferably about 0.1 microgram to about 10 milligrams; or more preferably about 1 milligram to about 2 milligrams. In some preferred embodiments, composition according to the present invention comprises about 5 nanograms to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligrams, from about 5 nanograms to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

In some embodiments, the composition can be formulated for administration of a dosage of 0.5 mg of DNA. In some embodiments, the composition can be formulated for administration of a dosage of 1.0 mg of DNA.

### 6. Nanoparticle Formulations

In one embodiment, the immunogenic composition of the invention may comprise a nanoparticle, including but not limited to a lipid nanoparticle (LNP), comprising a SARS-CoV-2 antibody of the invention, or a LNP comprising a nucleic acid encoding a SARS-CoV-2 antibody of the invention. In some embodiments, the composition comprises or encodes all or part of a SARS-CoV-2 antigen binding molecule of the invention, or an immunogenically functional equivalent thereof. In some embodiments, the composition comprises an mRNA molecule that encodes all or part of a SARS-CoV-2 antigen binding molecule of the invention.

In one embodiment, the immunogenic composition of the invention may comprise a composition comprising a combination of SARS-CoV-2 antibodies of the invention, or a LNP comprising one or more nucleic acid molecules encoding a combination of SARS-CoV-2 antibodies of the invention. In one embodiment, the immunogenic composition of the invention may comprise a composition comprising a combination of LNP, wherein the combination of LNP comprises one or more nucleic acid molecules encoding a combination of SARS-CoV-2 antibodies of the invention.

In one embodiment, the LNP comprises or encapsulates an RNA molecule encoding at least one amino acid sequence of SEQ ID NO:2, SEQ ID NO:4, SEQ ID NO:6, SEQ ID NO:8, SEQ ID NO: 10, SEQ ID NO: 12, SEQ ID NO: 14, SEQ ID NO: 16, SEQ ID NO: 18, SEQ ID NO:20, SEQ ID NO:22, SEQ ID NO:24, SEQ ID NO: 26, SEQ ID NO:28, SEQ ID NO: 30, SEQ ID NO:32, SEQ ID NO:34, SEQ ID NO: 36, SEQ ID NO:38, SEQ ID NO: 40, SEQ ID NO:42, SEQ ID NO:44, SEQ ID NO: 46, SEQ ID NO:48, SEQ ID NO:50, SEQ ID NO:52, SEQ ID NO:54, SEQ ID NO: 56, SEQ ID NO:58, SEQ ID NO:60, SEQ ID NO:62, SEQ ID NO:64, SEQ ID NO: 66, SEQ ID NO:68, SEQ ID NO: 70, SEQ ID NO: 72, SEQ ID NO:74, SEQ ID NO: 76, SEQ ID NO: 78, SEQ ID NO: 80, SEQ ID NO:88, SEQ ID NO:90, SEQ ID NO: 92, SEQ ID NO:94, SEQ ID NO:96, or SEQ ID NO:98 or a fragment or variant thereof.

In one embodiment, the LNP comprises or encapsulates an RNA molecule encoding at least one amino acid sequence of SEQ ID NO:2. SEQ ID NO:4. SEQ ID NO:6. SEQ ID NO:8. SEQ ID NO: 10. SEQ ID NO: 12. SEQ ID NO: 14. SEQ ID NO: 16. SEQ ID NO: 18. SEQ ID NO:20. SEQ ID NO:22. SEQ ID NO:24. SEQ ID NO: 26. SEQ ID NO:28. SEQ ID NO:30. SEQ ID NO:32. SEQ ID NO:34. SEQ ID NO: 36. SEQ ID NO:38. SEQ ID NO:40. SEQ ID NO:42. SEQ ID NO:44. SEQ ID NO: 46. SEQ ID NO:48. SEQ ID NO:50. SEQ ID NO:52. SEQ ID NO:54. SEQ ID NO: 56. SEQ ID NO:58. SEQ ID NO:60. SEQ ID NO:62. SEQ ID NO:64. SEQ ID NO: 66. SEQ ID NO:68. SEQ ID NO: 70. SEQ ID NO: 72. SEQ ID NO: 74. SEQ ID NO: 76, SEQ ID NO: 78. SEQ ID NO:80. SEQ ID NO:88. SEQ ID NO:90. SEQ ID NO: 92. SEQ ID NO:94. SEQ ID NO:96, or SEQ ID NO:98 or a fragment or variant thereof.

In one embodiment, the invention relates to a combination of LNPs comprising or encapsulating a combination of at least four RNA molecules encoding at least four of SEQ ID NO:2. SEQ ID NO:4. SEQ ID NO:6. SEQ ID NO:8. SEQ ID NO: 10. SEQ ID NO: 12. SEQ ID NO: 14, SEQ ID NO: 16. SEQ ID NO: 18. SEQ ID NO: 20. SEQ ID NO:22. SEQ ID NO:24. SEQ ID NO:26. SEQ ID NO:28. SEQ ID NO: 30. SEQ ID NO:32. SEQ ID NO:34. SEQ ID NO:36. SEQ ID NO:38. SEQ ID NO: 40, SEQ ID NO:42. SEQ ID NO:44. SEQ ID NO:46. SEQ ID NO:48. SEQ ID NO: 50. SEQ ID NO:52. SEQ ID NO:54. SEQ ID NO:56. SEQ ID NO:58. SEQ ID NO: 60. SEQ ID NO:62. SEQ ID NO:64. SEQ ID NO:66. SEQ ID NO:68. SEQ ID NO: 70. SEQ ID NO:72. SEQ ID NO: 74. SEQ ID NO: 76. SEQ ID NO: 78 or SEQ ID NO: 80, or a fragment or variant thereof.

In one embodiment, the invention relates to a combination of LNPs comprising or encapsulating a combination of at least four RNA molecules encoding the combination of SEQ ID NO:2. SEQ ID NO:4. SEQ ID NO:8, and SEQ ID NO: 10 or fragments or variants thereof. In one embodiment, the invention relates to a combination of LNPs comprising or encapsulating a combination of at least four RNA molecules encoding the combination of SEQ ID NO:2. SEQ ID NO:4. SEQ ID NO:8 and SEQ ID NO: 12 or fragments or variants thereof. In one embodiment, the invention relates to a combination of LNPs comprising or encapsulating a combination of at least four RNA molecules encoding the combination of SEQ ID NO:2. SEQ ID NO:6. SEQ ID NO:8 and SEQ ID NO: 10 or fragments or variants thereof. In one embodiment, the invention relates to a combination of LNPs comprising or encapsulating a combination of at least four RNA molecules encoding the combination of SEQ ID NO:2, SEQ ID NO:6, SEQ ID NO: 8 and SEQ ID NO: 12 or fragments or variants thereof.

In one embodiment, the invention relates to a combination of LNPs comprising or encapsulating a combination of at least four RNA molecules encoding the combination of a light chain of SEQ ID NO:2, and a heavy chain of SEQ ID NO:4 or SEQ ID NO:6, and a light chain of SEQ ID NO:8 and a heavy chain of SEQ ID NO:88, SEQ ID NO:90, SEQ ID NO:92, SEQ ID NO:94, SEQ ID NO:96 or SEQ ID NO:98 or fragments or variants thereof.

In one embodiment, the invention relates to a combination of LNPs comprising or encapsulating a combination of at least four RNA molecules encoding the combination of SEQ ID NO:58, SEQ ID NO:62, SEQ ID NO:66; and SEQ ID NO:72 or fragments or variants thereof. In one embodiment, the invention relates to a combination of LNPs comprising or encapsulating a combination of at least four RNA molecules encoding the combination of SEQ ID NO:58, SEQ ID NO:64, SEQ ID NO:66; and SEQ ID NO: 70 or fragments or variants thereof. In one embodiment, the invention relates to a combination of LNPs comprising or encapsulating a combination of at least four RNA molecules encoding the combination of SEQ ID NO:58, SEQ ID NO: 74, SEQ ID NO: 66; and SEQ ID NO:76 or fragments or variants thereof.

In one embodiment, the composition further comprises one or more additional immunostimulatory agents. Immunostimulatory agents include, but are not limited to, an additional antigen or antigen binding molecule, an immunomodulator, or an adjuvant.

### 7. Generation of Antibodies

The present invention also relates a method of generating the Fc modified synthetic antibody. The method can include administering the composition to the subject in need thereof by using the method of delivery described in more detail below. Accordingly, the Fc modified synthetic antibody is generated in the subject or in vivo upon administration of the composition to the subject.

The method can also include introducing the composition into one or more cells, and therefore, the Fc modified synthetic antibody can be generated or produced in the one or more cells. The method can further include introducing the composition into one or more tissues, for example, but not limited to, skin and muscle, and therefore, the Fc modified synthetic antibody can be generated or produced in the one or more tissues.

### 8. Methods of Identifying or Screening for the Antibody

The present invention further relates to a method of identifying or screening for the antibody described above, which is reactive to or binds the antigen described above. The method of identifying or screening for the antibody can use the antigen in methodologies known in those skilled in art to identify or screen for the antibody. Such methodologies can include, but are not limited to, selection of the antibody from a library (e.g., phage display) and immunization of an animal followed by isolation and/or purification of the antibody.

### 9. Methods of Diagnosing a Disease or Disorder

The present invention further relates to a method of diagnosing a subject as having a disease or disorder using an antibody, fragment thereof, or nucleic acid molecule encoding the same as described herein. In some embodiments, the present invention features methods for identifying subjects who are at risk of spreading SARS-CoV-2 infection or COVID-19, including those subjects who are asymptomatic or only exhibit non-specific indicators of SARS-CoV-2 infection or COVID-19. In some embodiments, the present invention is also useful for monitoring subjects undergoing treatments and therapies for SARS-CoV-2 infection or COVID-19, and for selecting or modifying therapies and treatments that would be efficacious in subjects having SARS-CoV-2 infection or COVID-19, wherein selection and use of such treatments and therapies promote immunity to SARS-CoV-2, or prevent infection by SARS-CoV-2.

In one embodiment, the antibody, fragment thereof, or nucleic acid molecule encoding the same can be used in an immunoassay for diagnosing a subject as having an active SARS-CoV-2 infection, having COVID-19, or having immunity to SARS-CoV-2 infection, or for monitoring subjects undergoing treatments and therapies for SARS-CoV-2 infection or COVID-19. Non-limiting exemplary immunoassay's include, for example, immunohistochemistry assays, immunocytochemistry assays, ELISA, capture ELISA, sandwich assays, enzyme immunoassay, radioimmunoassay, fluorescent immunoassay, and the like, all of which are known to those of skill in the art. See e.g. Harlow et al., 1988, Antibodies: A Laboratory Manual, Cold Spring Harbor, New York; Harlow et al., 1999, Using Antibodies: A Laboratory Manual, Cold Spring Harbor Laboratory Press, NY.

In some embodiments the methods include obtaining a sample from a subject and contacting the sample with an antibody of the invention or a cell expressing an antibody of the invention and detecting binding of the antibody to an antigen present in the sample.

In some embodiments, samples can be provided from a subject undergoing treatment regimens or therapeutic interventions, e.g., drug treatments, vaccination, etc. for SARS-CoV-2 infection or COVID-19. Samples can be obtained from the subject at various time points before, during, or after treatment.

The SARS-CoV-2 antibodies of the present invention, or nucleic acid molecules encoding the same, can thus be used to generate a risk profile or signature of subjects: (i) who are expected to have immunity to SARS-CoV-2 infection or COVID-19 and/or (ii) who are at risk of developing SARS-CoV-2 infection or COVID-19. The antibody profile of a subject can be compared to a predetermined or reference antibody profile to diagnose or identify subjects at risk for developing SARS-CoV-2 infection or COVID-19, to monitor the progression of disease, as well as the rate of progression of disease, and to monitor the effectiveness of SARS-CoV-2 infection or COVID-19 treatments. Data concerning the antibodies of the present invention can also be combined or correlated with other data or test results for SARS-CoV-2 infection or COVID-19, including but not limited to age, weight, BMI, imaging data, medical history, smoking status and any relevant family history.

The present invention also provides methods for identifying agents for treating SARS-CoV-2 infection or COVID-19 that are appropriate or otherwise customized for a specific subject. In this regard, a test sample from a subject, exposed to a therapeutic agent, drug, or other treatment regimen, can be taken and the level of one or more SARS-CoV-2 antibody can be determined. The level of one or more SARS-CoV-2 antibody can be compared to a sample derived from the subject before and after treatment, or can be compared to samples derived from one or more subjects who have shown improvements in risk factors as a result of such treatment or exposure.

In one embodiment, the invention is a method of diagnosing SARS-CoV-2 infection or COVID-19. In one embodiment, the method includes determining immunity to infection or reinfection by SARS-CoV-2. In some embodiments, these methods may utilize at least one biological sample (such as urine, saliva, blood, serum, plasma, amniotic fluid, or tears), for the detection of one or more SARS-CoV-2 antibody of the invention in the sample. Frequently the sample is a “clinical sample” which is a sample derived from a patient. In one embodiment, the biological sample is a blood sample.

In one embodiment, the method comprises detecting one or more SARS-CoV-2 antigen in at least one biological sample of the subject. In various embodiments, the level of one or more SARS-CoV-2 antigen of the invention in the biological sample of the subject is compared to a comparator. Non-limiting examples of comparators include, but are not limited to, a negative control, a positive control, an expected normal background value of the subject, a historical normal background value of the subject, an expected normal background value of a population that the subject is a member of, or a historical normal background value of a population that the subject is a member of.

### 10. Methods of Delivery of the Composition

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

### 11. Methods of Treatment

Also provided herein is a method of treating, protecting against, and/or preventing disease in a subject in need thereof by generating the Fc modified synthetic antibody in the subject. The method can include administering the composition to the subject. Administration of the composition to the subject can be done using the method of delivery described above.

The method can include administering a combination of antibodies or nucleic acid molecules encoding the same to the subject. In one embodiment, the method of the invention provides for administration of an antibody cocktail to the subject. In one embodiment, the cocktail is administered as a single formulation comprising multiple antibodies or nucleic acid molecules encoding the same. In one embodiment, the cocktail is administered as multiple formulations, either sequentially or concurrently. Administration of the composition to the subject can be done using the method of delivery described above. In one embodiment, the method of administration is intramuscular administration.

In certain embodiments, the invention provides a method of treating protecting against, and/or preventing a SARS-CoV-2 virus infection. In one embodiment, the method treats, protects against, and/or prevents a disease or disorder associated with SARS-CoV-2 virus infection. In one embodiment, the method treats, protects against, and/or prevents COVID-19.

In one embodiment the subject has, or is at risk of, SARS-CoV-2 virus infection.

Upon generation of one or more Fc modified synthetic antibody in the subject, the one or more Fc modified synthetic antibody can bind to or react with the antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The one or more Fc modified synthetic antibody can treat, prevent, and/or protect against disease in the subject administered the composition. The one or more Fc modified synthetic antibody by binding the antigen can treat, prevent, and/or protect against disease in the subject administered the composition. The Fc modified synthetic antibody can promote survival of the disease in the subject administered the composition. In one embodiment, the Fc modified synthetic antibody can provide increased survival of the disease in the subject over the expected survival of a subject having the disease who has not been administered the Fc modified synthetic antibody. In various embodiments, the Fc modified synthetic antibody can provide at least about a 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or a 100% increase in survival of the disease in subjects administered the composition over the expected survival in the absence of the composition or over the expected survival of a subject administered a parental antibody. In one embodiment, the Fc modified synthetic antibody can provide increased protection against the disease in the subject over the expected protection of a subject who has not been administered the Fc modified synthetic antibody or who is administered a parental antibody. In various embodiments, the Fc modified synthetic antibody can protect against disease in at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% of subjects administered the composition over the expected protection in the absence of the composition or over the expected protection in a subject administered a parental antibody.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

In one embodiment, immunotherapy with the Fc modified antibodies of the invention will have a direct therapeutic effect. In one embodiment, immunotherapy with the Fc modified antibodies of the invention can be used as immune “adjuvant” treatment to reduce viral protein load, in order to provide host immunity and optimize the effect of antiviral drugs.

### 12. Use in Combination

In some embodiments, the invention provides a cocktail of antibodies comprising at least two, at least three, at least four or more than four Fc modified synthetic antibodies of the invention or fragments thereof.

The present invention also provides a method of treating, protecting against, and/or preventing disease in a subject in need thereof by administering a combination of the synthetic antibody and one or more additional agent. In one embodiment, the additional agent is an additional therapeutic agent. In one embodiment the additional therapeutic agent is an additional therapeutic antibody. In one embodiment, the therapeutic agent is an antiviral agent. In one embodiment, the therapeutic is an antibiotic agent. In one embodiment, the therapeutic agent is a SARS-CoV-2 vaccine. In one embodiment, the therapeutic agent is a small-molecule drug or biologic.

The present invention also provides a method of treating, protecting against, and/or preventing disease in a subject in need thereof by administering a combination of the Fc modified synthetic antibody and at least one additional therapeutic agent. In one embodiment, the additional therapeutic agent is an additional Fc modified synthetic antibody or nucleic acid molecule encoding the same. In one embodiment, the therapeutic agent is an antiviral agent. In one embodiment, the therapeutic is an antibiotic agent. In one embodiment, the therapeutic is chemotherapeutic agent. In one embodiment, the therapeutic agent is a small-molecule drug or biologic.

In some embodiments, the present invention provides a method of treating, protecting against, and/or preventing disease in a subject in need thereof by administering a combination of a) a nucleic acid molecule encoding Fc modified synthetic antibody heavy chain; and b) a nucleic acid molecule encoding a synthetic antibody light chain. In some embodiments, a) and b) are administered concurrently, simultaneously, essentially simultaneously or within the same treatment protocol.

In some embodiments, the present invention provides a method of treating, protecting against, and/or preventing disease in a subject in need thereof by administering a combination of a) a nucleic acid molecule encoding Fc modified synthetic antibody heavy chain; and b) a nucleic acid molecule encoding a synthetic antibody light chain and c) one or more additional therapeutic agent. In some embodiments, a), b) and c) are administered concurrently, simultaneously, essentially simultaneously or within the same treatment protocol. In some embodiments, a), b), and c) are administered consecutively. In some embodiments, a) and b) are administered at least once as part of a treatment protocol.

One or more Fc modified synthetic antibody and one or more additional therapeutic agent, or the Fc modified synthetic antibody cocktail, may be administered using any suitable method such that a combination of one or more Fc modified synthetic antibody and therapeutic agent are both present in the subject. In one embodiment, the method may comprise administration of a first composition comprising a Fc modified synthetic antibody or cocktail of the invention by any of the methods described in detail above and administration of a second composition comprising a therapeutic agent less than 1, less than 2, less than 3, less than 4, less than 5, less than 6, less than 7, less than 8, less than 9 or less than 10 days following administration of the Fc modified synthetic antibody or cocktail. In one embodiment, the method may comprise administration of a first composition comprising a Fc modified synthetic antibody or cocktail of the invention by any of the methods described in detail above and administration of a second composition comprising a therapeutic agent more than 1, more than 2, more than 3, more than 4, more than 5, more than 6, more than 7, more than 8, more than 9) or more than 10 days following administration of the Fc modified synthetic antibody or cocktail. In one embodiment, the method may comprise administration of a first composition comprising a therapeutic agent and administration of a second composition comprising a Fc modified synthetic antibody or cocktail of the invention by any of the methods described in detail above less than 1, less than 2, less than 3, less than 4, less than 5, less than 6, less than 7, less than 8, less than 9 or less than 10 days following administration of the therapeutic agent. In one embodiment, the method may comprise administration of a first composition comprising a therapeutic agent and administration of a second composition comprising a Fc modified synthetic antibody or cocktail of the invention by any of the methods described in detail above more than 1, more than 2, more than 3, more than 4, more than 5, more than 6, more than 7, more than 8, more than 9 or more than 10 days following administration of the therapeutic agent. In one embodiment, the method may comprise administration of a first composition comprising a Fc modified synthetic antibody or cocktail of the invention by any of the methods described in detail above and a second composition comprising a therapeutic agent concurrently. In one embodiment, the method may comprise administration of a first composition comprising a Fc modified synthetic antibody or cocktail of the invention by any of the methods described in detail above and a second composition comprising a Fc modified synthetic antibody or cocktail of the invention by any of the methods described in detail above and a third composition comprising a therapeutic agent concurrently. In one embodiment, the method may comprise administration of a single composition comprising a Fc modified synthetic antibody or cocktail of the invention and a therapeutic agent.

In some embodiments, the invention provides a method of treating or preventing a disease or disorder associated with SARS-CoV-2 infection, the method comprising administering a combination of a first composition comprising a combination of nucleic acid molecules encoding a first anti-SARS-CoV-2 antibody and a second composition comprising a combination of nucleic acid molecules encoding a second anti-SARS-CoV-2 antibody, wherein
a) the first combination of nucleic acid molecules comprises a first nucleic acid molecule comprising a nucleotide sequence of SEQ ID NO:73, comprising a nucleotide sequence encoding a heavy chain amino acid sequence, and a second nucleic acid molecule comprising a nucleotide sequence of SEQ ID NO:57, comprising a nucleotide sequence encoding a light chain amino acid sequence; and
b) the second combination of nucleic acid molecules comprises a first nucleic acid molecule comprising a nucleotide sequence of SEQ ID NO:75, comprising a nucleotide sequence encoding a heavy chain amino acid sequence, and a second nucleic acid molecule comprising a nucleotide sequence of SEQ ID NO:65, comprising a nucleotide sequence encoding a light chain amino acid sequence.

In some embodiments, the method comprises a total administration of 0.5 mg nucleic acid from the first composition and 0.5 mg nucleic acid from the second composition, for a total administration of 1.0 mg nucleic acid.

In some embodiments, the method comprises a total administration of 1.0 mg nucleic acid from the first composition and 1.0 mg nucleic acid from the second composition, for a total administration of 2.0 mg nucleic acid.

In some embodiments, each of the first and second composition are administered concurrently at two different injection sites on day 0 and again on day 3 of a treatment regimen.

In some embodiments, the method comprises a total administration of 0.5 mg nucleic acid from the first composition and 0.5 mg nucleic acid from the second composition, for a total administration of 1.0 mg nucleic acid on each of days 0 and 3.

In some embodiments, the method comprises a total administration of 1.0 mg nucleic acid from the first composition and 1.0 mg nucleic acid from the second composition, for a total administration of 2.0 mg nucleic acid on each of days 0 and 3.

In some embodiments, the method further comprises administering a single dose of 120 mg methylprednisolone following the final administration of the combination of the first composition and the second composition.

In some embodiments, a Fc modified synthetic antibody or antibody cocktail of the invention can be administered in combination with one or more additional antibiotic agent. Non-limiting examples of antibiotics that can be used in combination with the Fc modified synthetic antibody of the invention include aminoglycosides (e.g., gentamicin, amikacin, tobramycin), quinolones (e.g., ciprofloxacin, levofloxacin), cephalosporins (e.g., ceftazidime, cefepime, cefoperazone, cefpirome, ceftobiprole), antipseudomonal penicillins: carboxypenicillins (e.g., carbenicillin and ticarcillin) and ureidopenicillins (e.g., mezlocillin, azlocillin, and piperacillin), carbapenems (e.g., meropenem, imipenem, doripenem), polymyxins (e.g., polymyxin B and colistin) and monobactams (e.g., aztreonam).

### 13. Generation of Synthetic Antibodies In Vitro and Ex Vivo

In one embodiment, one or more Fc modified synthetic antibody is generated in vitro or ex vivo. For example, in one embodiment, a nucleic acid encoding a Fc modified synthetic antibody can be introduced and expressed in an in vitro or ex vivo cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome or lipid nanoparticle. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

The present invention has multiple aspects, illustrated by the following non-limiting examples.

### 14. Examples

The present invention is further illustrated in the following Examples. It should be understood that these Examples, while demonstrating some embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: In Vivo Delivery of an Engineered Antibody Cocktail Confers Protection Against Lethal SARS-CoV-2 Challenge

The synthetic DNA-encoded monoclonal antibody (DMAb) platform has shown preclinical promise as a strategy for the in vivo delivery of functional antibodies against infectious diseases, offering numerous potential advantages in terms of production, distribution and in vivo durability compared to traditional mAb therapy. This is achieved through the facilitated delivery of optimized, synthetic plasmids that drive the in vivo expression, assembly and systemic distribution of encoded immunoglobulin (Patel et al., 2020, BioDrugs, 34(3):273-293). While initial studies documented the ability to deliver modest levels of DMAbs against several pathogens (Muthumani et al., 2016, J Infect Dis, 214:369-78; Flingai et al., 2015, Sci Rep, 5:12616; Elliott et al., 2017, NPJ Vaccines, 2:18; Esquivel et al., 2019, Mol Ther, 27:974-85), recent studies aimed to improve in vivo expression and potency using a number of approaches (Patel et al., 2017. Nat Commun, 8:637; Patel et al., 2018, Cell Rep, 25:1982-1993 e4; Wise et al . . . 2020, J Clin Invest, 130:827-37; Parzych et al., 2021, MBio, 12:1-13). In vivo-launched DMAbs demonstrate functional activity and potency equivalent to their recombinant counterparts, conferring protection against challenge in both small and large animal models (Esquivel et al., 2019, Mol Ther, 27:974-85; Wise et al., 2020, J Clin Invest, 130:827-37; McNee et al., 2020, J Immunol, 205:648-60). Due to continuous in vivo production, serum DMAb can often be detected at or above protective levels for a prolonged period of time in small animal models compared to recombinant (protein) IgG (Patel et al., 2018, Cell Rep, 25:1982-1993 e4; Wise et al., 2020, J Clin Invest, 130:827-37). Importantly, the ability to concurrently deliver and express multiple functional DMAbs in vivo has also been established (Wise et al., 2020, J Clin Invest, 130:827-37). DMAbs targeting SARS-CoV-2 could serve as a potentially important strategy to bypass the hurdles associated with bioprocessing, distribution and delivery of traditional mAb therapy, providing an additional tool for impacting COVID-19.

The COVID-19 pandemic has shown the ability of nucleic-based approaches to rapidly develop and deliver clinically effective interventions against infectious pathogens. These hold potential as novel avenues to administer a diverse set of biologically functional molecules to vulnerable and at-risk populations. Validated mAb clones 2196, 2130 and 2381 were focused on due to their striking cross-strain potency, and a series of plasmids was generated which were designed to optimize in vivo expression levels. A series pK studies conducted in both BALB/c and K-18 mice demonstrated the successful expression of functional antibodies using single plasmid constructs, the titers of which were further improved with dual plasmid approaches and additional Fc-engineering without detectible loss in potency. Continued in vivo mAb production is a unique advantage of the DNA platform relative to traditional recombinant wildtype IgG that has a serum half-life of approximately 21 days. Peak in vivo titers of Fc variants ranged from 13-50 μg/mL which were maintained at significant levels for at least 80 days. With continued platform development and engineering modifications to improve half-life, this could potentially lower the need for repeated drug delivery.

Here, it is demonstrated that plasmid-launched DMAbs possess molecular and functional profiles comparable to their recombinant counterparts both in vitro and in vivo. Antiviral activity of serum-derived 2130 and 2196-based DMAbs against pseudotyped and authentic SARS-CoV-2 (WA1/2020) was extremely high, with IC50 values in the low ng/ml range. Moreover, in vivo efficacy of was evaluated using two established murine models of SARS-CoV-2. In an AAV6.2FF-hACE-2 model, DMAbs 2196, 2130 and 2381 (FcWT variants) reduced lung viral burden by 1-2 logs, a similar degree of reduction as achieved with a 200 g dose of the recombinant mAbs using a similar Ad5-hACE2 model (Zost et al., 2020, Nature, 584:443-9). Infection of transgenic K-18 mice results in a more stringent, lethal challenge. When administered as prophylactic monotherapies, 2196- and 2130-based DMAbs (FcTM variants) significantly reduced viral burden at mucosal sites, preventing major weight-loss, pathology and providing 100% protection compared to the naïve group in which all animals succumbed to infection. Additionally, co-administration and expression of 2196 and 2130-based DMAbs facilitated complete viral control in all cocktail-treated mice following lethal challenge. This not only conferred 100% survival, but also protected the lungs from all evidence of SARS-CoV-2-mediated pathology and clinical disease. Recent studies reported similar protection against lethal challenge with SARS-CoV-2 (WA1/2020) as well as numerous variant strains in K-18 mice using a 40 μg dose of recombinant 2196/2130 mAb cocktail (Chen et al., 2021, Nature, 596:103-8). Given the above data, it is likely that comparable protection against variant strains would be achieved by the DMAb cocktail following plasmid delivery. Interestingly, similar protection from disease and pathology was mediated by both FcWT and FcTM variants, indicating that Fc-mediated effector mechanisms are not detrimental to their function or safety profiles. This is consistent with preclinical (Winkler et al., 2021. Cell, 184:1804-1820.e16; Suryadevara et al., 2021, Cell, 184:2316-2331.e15; Schäfer et al., 2020, J Exp Med, 218; Ullah et al., 2021, Immunity, 54:2143-2158.e15) and clinical studies which have reported no indication of ADE in SARS-CoV-2 (Gottlieb et al., 2021, JAMA, 325:632-44; Dougan et al., 2021, N Engl J Med, 385(15):1382-1392; Wu et al., 2021, Antimicrob Agents Chemother, 65(8):e0035021). Effector function antibodies may provide potential benefits in immune clearance especially in the therapeutic setting. The data supports that such modifications do not have negative impact in this animal model supporting their further study in this context.

As SARS-CoV-2 continues to evolve, it is imperative that interventions remain effective against viral variants. Similar to recent reports of recombinant 2196 and 2130 mAbs, in vivo-launched DMAbs largely maintain activity against the current major variants of interest; 2130-based DMAbs demonstrate comparable neutralizing capacity (<3-fold reduction) against all SARS-CoV-2 variant pseudotyped viruses while DMAbs 2196 and 2381, Class I mAbs, show a modest reduction in the binding to and neutralization (<3 to 9-fold reduction) of spike variants containing mutations at position 486, a key residue commonly found within the Class I binding epitopes. Despite this reduction, the serum functionality following plasmid delivery of 2196-based DMAbs remained high (ID₅₀>10³) against all viral strains due to its exceptional molecular potency and significant expression profile. When administered in combination, the 2196 and 2130 DMAb pair achieves optimal activity against the parental WA1/2020 strain that is maintained (<3-fold reduction) against all strains tested, including the highly transmissible and rampant Delta (B.1.617.2) variant currently responsible for the vast majority of global cases. When compared to additional DMAb clones, 2196 and 2130 demonstrated superior cross-strain potency which supports their continued development. Together, these studies demonstrate the flexibility and promise of DNA-based approaches for the rapid design, evaluation and delivery of effective immunotherapeutics. Nucleic acid approaches, including DNA-encoded alternatives, may broaden the applications of traditional bioprocessed products, potentially expanding their indications and improving accessibility to life-saving therapeutics.

### The Materials and Methods Used for the Experiments are Now Described

### DNA Expression Constructs (DMAbs)

The mature variable heavy (VH) and light (VL) domains of the selected mAb clones were optimized at the DNA and RNA levels. Synthetic inserts encoding the heavy chain (HC) and light chain (LC) genes for each clone were designed, containing a leader sequence(s) and the optimized VH or VL sequences followed by the corresponding constant domains (CH and CL, respectively) of wildtype human IgG1 (hFcWT). These were and inserted into a modified mammalian expression vector (pVax) under the human cytomegalovirus (hCMV) promoter between an IgG leader sequence and a bovine grown hormone (BGH) poly A signal using single or dual plasmid approaches. In single plasmid constructs (pHC/LC), matching genes were encoded in cis and separated by a porcine teschovirus-1 2A peptide/furin cleavage site. For dual plasmid systems, separate light chain plasmids (pLC) and heavy chain plasmids (pHC_hFcWT) were generated for each clone. An additional HC variant, pHC_hFcTM, was generated for selected clones containing a triple mutation (L234F/L235E/P331S) known to nullify effector functions of hIgG1.

### Mammalian Cell Culture and In Vitro Transfections

In vitro expression of DNA plasmids was performed in Expi293F™ suspension cells (A14527; Thermo). Cells suspension was maintained in Expi293™ Expression Medium (A1435101) at 37° C./8% CO₂ conditions and transfected using the Expi293F™ Expression System Kit (A14635; Thermo). All transfection parameters (cell concentrations, culture volumes, DNA dilutions, incubation times, reagent preparations, etc.) were determined according to the manufacturer's guidelines. Briefly, cells were seeded 6-well culture plates at 1×10⁶ cells/mL. DMAb plasmid(s) were diluted in OPTI-MEM media (1 μg/mL) and mixed with EpiFectamine. DNA:lipid mixtures were incubated for 20 minutes at room temperature (RT) to allow for complex formation and then added, dropwise, to cells. All constructs were tested in duplicate. Enhancers were added 18-22 hrs later, as instructed. Clarified culture supernatants were harvested 4-5 day's post-transfection and stored at −20° C. prior to analysis.

### Animals and In Vivo DMAb Delivery

These studies were performed in five-to-eight-week-old BALB/C (pK studies, AAV-hACE2 challenge/Exp1) and K-18 (pK studies, lethal challenges/Exp 2+3) mouse strains. Transgenic K-18 mice (B6.Cg-Tg (K18-ACE2) 2Prlmn/J; 034860) express the gene for human angiotensin 1 converting enzyme (hACE2) in the airway epithelia under a human keratin 18 (KRT18) mice. These were purchased from The Jackson Laboratory and housed in The Wistar Institute animal facility. All procedures were performed in accordance with the guidelines from the Wistar Institute Animal Care and Use Committee (IACUC) under approved protocols 201399 and 201464. For all DMAb administrations, 50-200 μg of total plasmid DNA was formulated in water supplemented with hyaluronidase (12 U/injection; Sigma) and injected into the tibialis anterior(s) and/or quadricep muscle(s). Injections were followed by the delivery of two 0.1 Amp electric constant current square-wave pulses by the CELLECTRA-3P electroporation device (Inovio Pharmaceuticals) to facilitate plasmid uptake. To prevent xenogenic responses against human DMAbs, T cell depletion (Anti-CD4⁺/CD8⁺ mAbs, 200 μg/mouse, given intraperitoneally) was performed at the time of plasmid injection. For pK studies, sera were periodically collected via submandibular bleed to determine expression levels, durability and functionality. For bronchoalveolar lavage isolation (BAL), animals were euthanized and lungs were flushed by with 900 μl of PBS supplemented with 0.05% NaN₃, 0.05% Tween-20, 2% 0.5M EDTA and protease inhibitor using a 20G blunt ended needle. BAL fluid was heat-inactivated for 20 m at 56° C. and stored at −20° C. prior to analysis. For efficacy studies, DMAb-treated mice were shipped to collaborators at Public Health Agency of Canada (PHAC) or transferred to BioQual, Inc. for challenge with SARS-CoV-2 (see SARS COV-2 Challenge methods below). Further experimental details for individual in vivo pk and efficacy studies are indicated in the appropriate figure(s).

### IgG Quantification (Anti-Human IgG ELISA)

For quantification of DMAb in supernatant or mouse serum, NUNC 96-well MaxiSorp plates (Sigma M9410-ICS) were coated with 5 μg/ml goat anti-human IgG-Fc (Bethyl Cat #A80-104A) diluted in 1×PBS overnight at 4° C. The following day, plates were washed 4 times with 0.05% PBS-T and were blocked with 5% non-fat dry milk in PBS for 1 hr at room temperature (RT). Plates were washed and incubated with duplicate samples, diluted in 1% newborn calf serum (NCS) in 0.2% PBS-T for 1 hr at RT. Plates were washed and incubated with 1:10000 HRP-conjugated goat anti-human IgG-Fc (Bethyl Cat #A80-104P) diluted in 1% NCS in 0.2% PBS-T for 1 hr at RT. Finally, washed plates were developed with 1-Step™ Ultra TMB-ELISA Substrate Solution (Thermo Scientific Cat #34028) for 2 min followed by quenching with 2N H₂SO₄. Plates were read at 450 nm on the BioTek Synergy 2 (Biotek) plate reader. Blank wells were included on each plate and subtracted as background. Purified human IgG/Lambda (Bethyl Cat #P80-112) was used to create a standard curve for quantification (μg/mL). Positive control sample was included on each plate and used to standardize values across assays. Data were subsequently exported to Microsoft Excel and analyzed using Graphpad Prism version 8.

### IgG Epitope Specificity Variant Recognition (RBD-Binding ELISAs)

Binding ELISAs were used to confirm the epitope specificities of DMAbs 2130, 2196 and 2381 and evaluate their relative reactivity to recombinant RBDs containing common mutations found within arising variants of concern. For epitope specificity, NUNC 96-well MaxiSorp plates were coated with recombinant RBD proteins (3 μg/mL in 1×PBS) containing mutations at residues F444A (RBD-F444A) or F486 (RBD-F486A) (kindly provided by AstraZeneca), which are key residues required for the binding of clones 2130 and 2196/2381, respectively. To evaluate the relative binding to RBDs from VoC, the following coating antigens were used (3 μg/mL in 1×PBS): SARS-CoV-2 Spike RBD-His Recombinant Protein (Sino Biologicals Cat #40592-V08B), Spike S1 (D614G)-His Recombinant Protein (40591-V08H3), RBD-His K417N (Sino Biologicals 40592-V08H59), RBD-His E484K (Sino Biologicals 40592-V08H84), RBD-His N501Y (Sino Biologicals 40592-V08H82), Spike S1 K417N E484K N501Y D614G (Sino Biologicals 40591-V08H10). ELISA procedure was completed as described above (see IgG Quantification section).

### ACE-2 Inhibition Assays

96-well Flat-Bottom Half-Area plates (Corning) were coated at room temperature for 8 hours with 1 μg/mL 6×-His tag polyclonal antibody (PA1-983B, ThermoFisher), followed by overnight blocking with blocking buffer containing 5% milk/1×PBS/0.01% Tween-20 at 4° C. The plates were then incubated with RBD at 1 μg/mL at room temperature for 1-2 hours. Sera harvested from DMAb-treated mice either were serially diluted 3-fold starting at 1:20 with dilution buffer (5% milk/1×PBS/0.01% Tween-20), added to the plate and incubated at RT for 1-2 hrs. Human Angiotensin-converting enzyme 2 (ACE2-IgHu) antibody was biotinylated using Novus Biologicals Lightning-Link rapid type A Biotin antibody labeling kit (NovusBio, 370-0010) according to protocol. The biotinylated ACE2-IgHu was added to wells at a constant concentration of 0.5 μg/ml diluted with the dilution buffer and incubated at RT for 1 hour. The plates were further incubated at room temperature for 1 hour with native streptavidin-HRP (Abcam, ab7403) at 1:15,000 dilution followed by addition of TMB substrates (ThermoFisher), and then quenched with 1M H₂SO₄. Absorbances at 450 nm and 570 nm were recorded with a BioTek plate reader. Four washes were performed between every incubation step using PBS and 0.05% Tween-20. The assay was performed in triplicates and the average of the absorbance value was determined. The average absorbance of the lowest dilutions with saturating ACE2 signals was calculated to get a maximum ACE2 binding and no blocking. Each average absorbance value was subtracted from the maximum to get an ACE2 blocking curve. The blocking titer is defined as the reciprocal of the highest dilution where two consecutive dilutions have readings below zero. The maximum area under the curve is determined by calculating the Area Under the Curve (AUC) of full ACE2 binding without the competitor. The AUC of the competitor is then subtracted from the maximum AUC to get the area between the curves (blocking Area) and is the measure of ACE2 blocking. The fraction ACE2 blocking is defined as the fraction of the blocking area to the maximum AUC.

### Neutralization Assay: Pseudotyped Virus

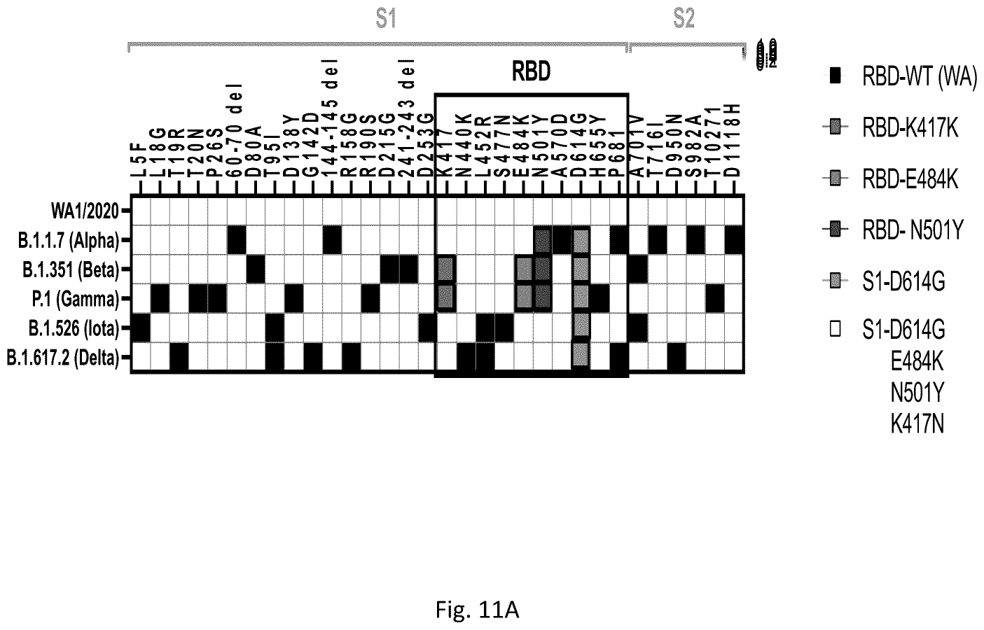

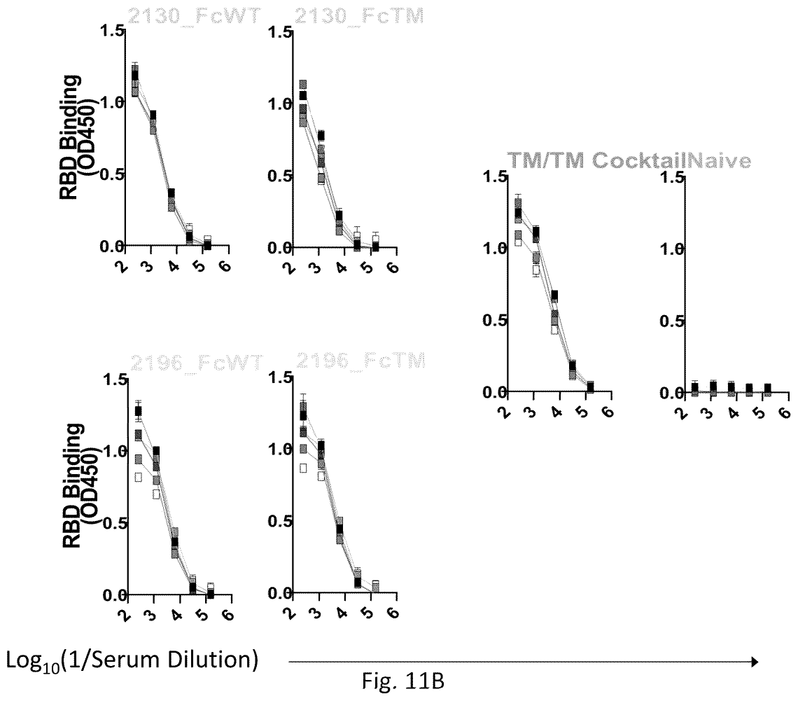

HEK293T (Vendor?) were maintained in DMEM supplemented with 10% fetal bovine serum (FBS) and 1% penicillin streptomycin (P/S) antibiotic in 37° C./5% CO2 conditions. To create SARS-CoV-2 pseudoviruses, Gene jammer (Agilent) was used to transfect cells with 1:1 ratio of pNL4-3.Luc.R-E-plasmid (NIH AIDS reagent) along with various of synthetic plasmids (Genscript) expressing the wildtype spike protein (derived from isolate USA-WA1/2020) or mutated spikes derived from variants B.1.1.7 (Alpha), B.1.351 (Beta), P.I (Gamma), B.1.617.2 (Delta) or B.1.526 (Iota); see FIG. 11 for specific mutations. Forty-eight hours post-transfection, culture supernatants were collected, enriched with FBS to 12% final volume, and stored at −80_C. SARS-Cov-2 pseudovirus neutralization assays were established using huCHOAce2 cells (Creative Biolabs, Catalog No. VCeL-Wyb019) plated in a 96-well plate format. Cells were resuspended in D10 media (DMEM supplemented with 10% FBS and 1× Penicillin-Streptomycin), plated (10,000 cells/well) and rested overnight in 37° C./5% CO2 conditions. The following day, transfection supernatant or sera from DMAb-treated animals were heat-inactivated and serially diluted in duplicate as desired. Supernatant from non-transfected cells or sera from naïve animals served as controls, respectively. Diluted samples were incubated with the indicated SARS-Cov-2 pseudovirus for 90 minutes at RT and then transferred to rested huCHOAce2 cells. Plates were incubated in 37° C./5% CO2 conditions for 72 hrs and then lysed using the britelite plus luminescence reporter gene assay system (Perkin Elmer Catalog no. 6066769). RLUs were measured using the Biotek plate reader. Using GraphPad Prism 8, nonlinear regressions were applied to duplicate RLU values for each sample to determine the best fit line. Neutralization titers (ID50) were then calculated, defined as the reciprocal dilution that yielded a 50% reduction in RLU compared to sample control wells; RLUs from cell-only control wells on each plate were subtracted as background prior to analysis. To assess the relative activity against mutant pseudoviruses, the same dilution series was tested in parallel against the indicated variants. The calculated ID50s were used to calculate a fold change relative to WA1/2020 (ID50_{WA1/2020}/ID50ᵥₐᵣᵢₐₙₜ). ID50s for each sample were also used along with the corresponding DMAb titer (ng/mL) to calculate inhibitory concentrations (IC50s=DMAb titer/ID50) that reflect the individual molecular potency of a test sample while controlling for expression levels.

### Neutralization Assay: Authentic SARS-CoV-2 Viruses

Live SARS-Related Coronavirus 2, Isolate USA-WA1/2020, was obtained through BEI Resources (NIAID, NIH; NR-52281) and contained within the BSL-3 facility at the Wistar Institute. Vero cells (ATCC CCL-81) were maintained in DMEM supplemented with 10% fetal bovine serum (FBS). Viral propagation and titration were achieved as previously described. Briefly, the USA-WA1/2020 virus stock was serially diluted in DMEM with 1% FBS and transferred in replicates of 8 to previously seeded Vero cells and incubated for five days under 37° C./5% CO2 conditions. Individual wells were then scored positive or negative for the presence of cytopathic effect (CPE) by examination under a light microscope. The virus titer (TCID50/mL) was calculated using the Reed-Munch method and the published Microsoft Excel-based calculator. For neutralization assays, Vero cells were seeded in DMEM with 1% FBS at 20,000 cells/well in 96 well flat bottom plates and incubated overnight. Samples were heat-inactivated at 56° C. for 30 minutes and then serially diluted in triplicates. These were incubated for Ihr at RT with 300 TCID50/mL of virus before the mixture was transferred to previously seeded Vero cells and incubated for 5 days. Viral titer (TCID50) was determined as described above.

SARS-CoV-2 Challenges: hACE2-AAV Model

Female BALB/C mice (n=10/group) were treated with the indicated DMAb plasmid formulations (200 μg/mouse; see Animals and in vivo DMAb delivery methods) and shipped to PHAC for evaluation using the previously validated hACE2-AAV model (Gary et al., 2021, IScience, 24:102699). Mice were anesthetized with isoflurane 14 days post-plasmid delivery and administered 1×10¹¹ viral copies of AAV6.2FF-hACE2 intranasally (50 μL) to facilitate expression of hACE2 in the lungs of recipient mice. One week later (D21 post-plasmid delivery), pre-challenge blood samples were collected by prior to intranasal challenge with 1×10⁵ PFU (50 μL) of SARS-CoV-2 virus (hCoV-19/Canada/ON-VIDO-01/2020; GISAID #EPI_ISL_425177). Controls include a group of non-AAV transduced animals (insusceptible; negative control) and a group of AAV-transduced/non-DMAb treated animals (susceptible; positive control). Following challenge, animals were monitored daily for signs of clinical disease and euthanized 4 days post-infection, at which time lung tissue was collected for viral quantification. Levels of viral RNA (copies/g lung tissue) for each animal were determined via qPCR.

### SARS-CoV-2 Challenges: Lethal K-18 Model

Male or female K-18 mice (n=12/group) were treated with the indicated DMAb plasmid formulations (200 μg/mouse; see Animals and in vivo DMAb delivery methods) and transferred to BioQual Inc. for evaluation in a lethal SARS-CoV-2 challenge model. Baseline sera samples and body weights were collected prior to challenge. Mice were anesthetized and intranasally (50 μL) infected with 2.8×10³ PFU (SARS-CoV-2/human/USA/WA-CDC-WA1/2020USA_WA1/2020; GenBank Acc: MN985325). Animals were monitored daily following challenge for clinical signs of disease (visual scoring, weight-loss, etc.); euthanasia criteria included moribund scoring and/or weight-loss of >20% (vs. pre-challenge starting weight). At D4 post-challenge, a subset of each group (n=4) were sacrificed to assess viral titers in the lungs and nasal turbinates of challenged mice via TCID₅₀ assay. Lung tissue was also collected and processed for histopathology (H&E staining). Gross and microscopic scoring of lung sections was conducted, using the following scale that reflects the intensity and pervasiveness of observed histopathological change: Grade 1 (1+): minimal, <10%; Grade 2 (2+): mild, 10-25%; Grade 3 (3+): moderate, 25-75%; Grade 4 (4+): marked, 75-95%; Grade 5 (5+): severe >95%. Additional experimental details individual K-18 challenges are provided in the appropriate figure(s).

### Statistical Analysis

Statistical analyses were conducted using GraphPad Prism 9 software. Nonparametric tests were performed due to small group sizes. Mann-Whitney U tests were used when comparing means of two groups and Kruskal-Wallis nonparametric rank-sum tests followed by Dunn's post hoc analysis were conducted to compare three or more groups. Survival curves were analyzed using Mantel-Cox log-rank test tests. In all cases, P values <0.05 were considered significant and denoted as * P<0.05, **P<0.01, ***P<0.001, and ****P<0.0001.

### The Experimental Results are Now Described

### Engineering and In Vitro Expression of 2196, 2130 and 2381 DMAbs Using Single Plasmid Approaches

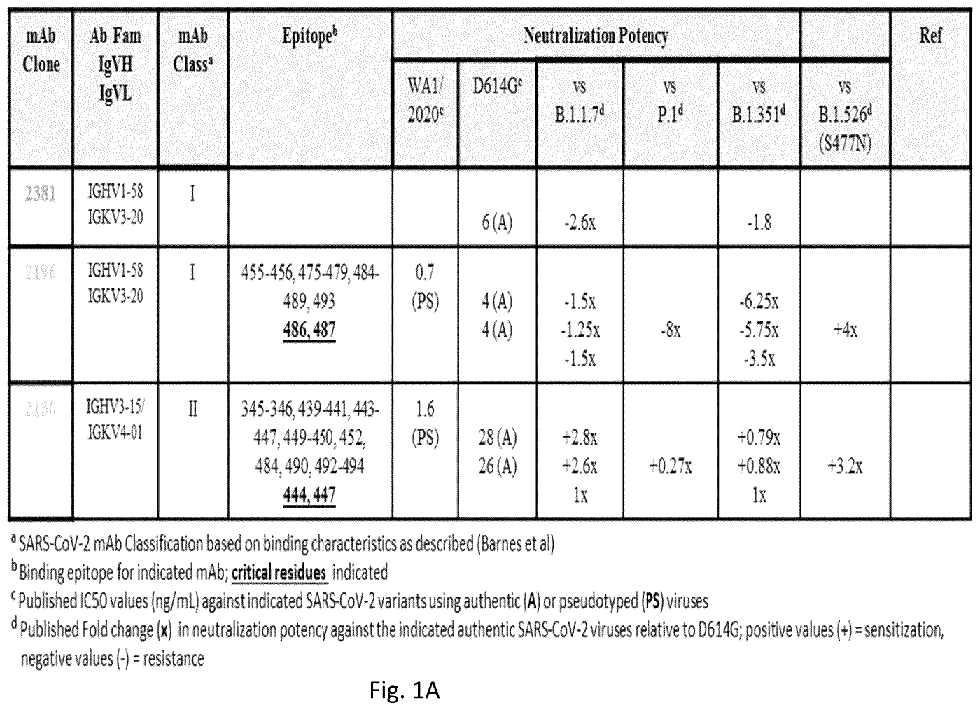

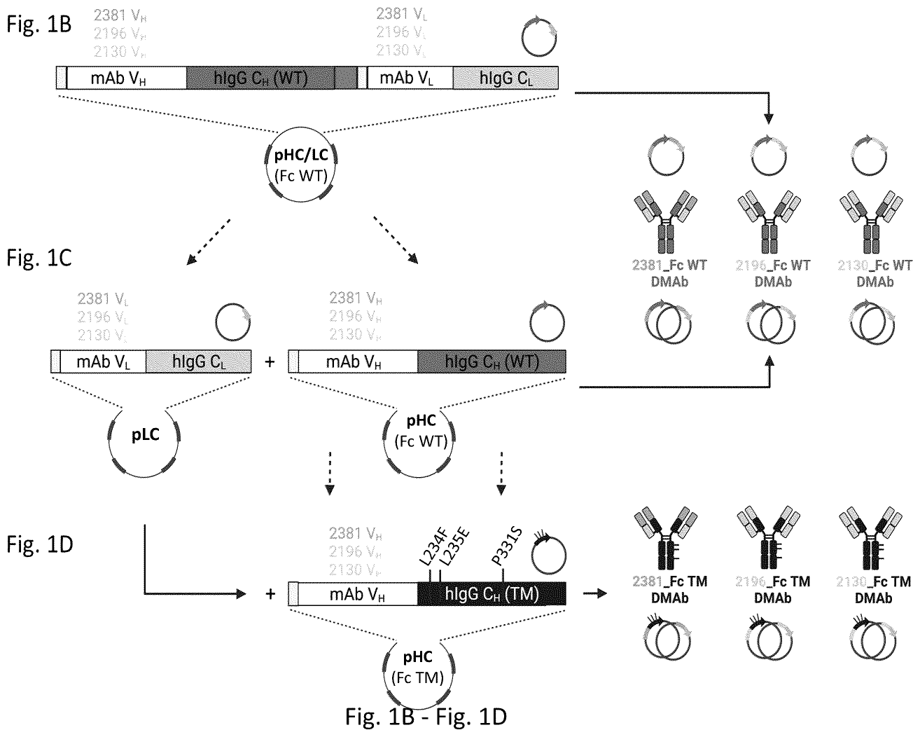

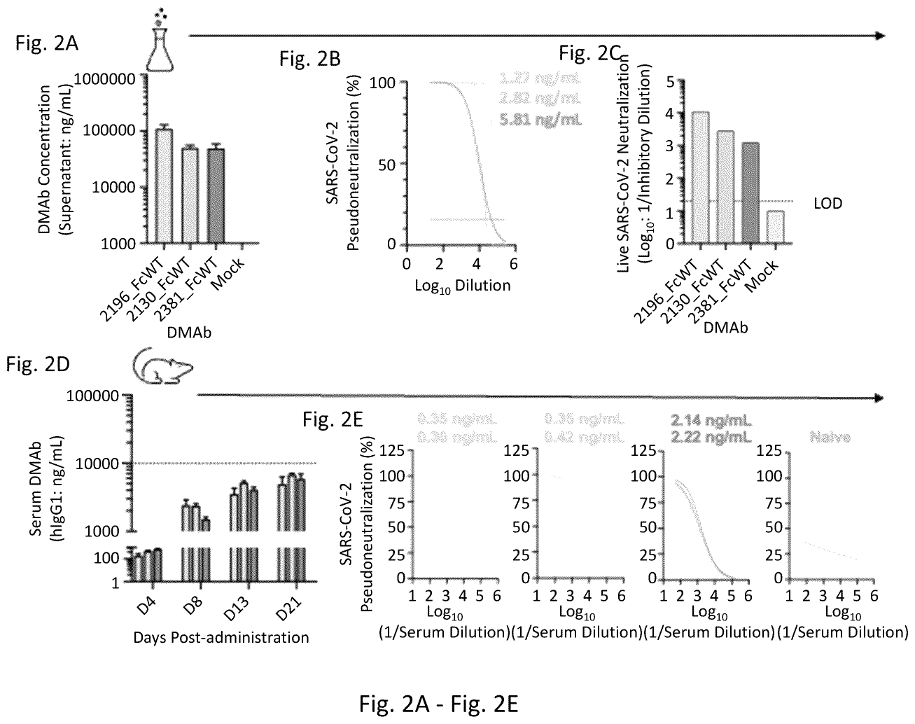

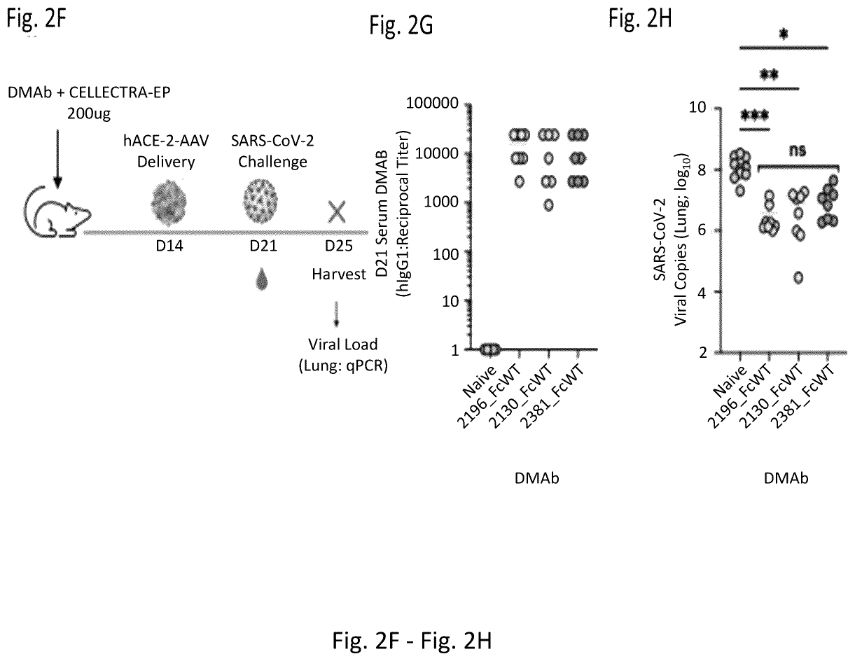

Anti-SARS-CoV-2 clones 2130, 2196 and 2381 are three highly potent nAbs originally described at Vanderbilt Medical Center prior to licensure by AstraZeneca (Zost et al., 2020, Nature, 584:443-9). Derived from different SARS-CoV-2 mAb classes, clones 2196 and 2130 target non-redundant and complementary epitopes within the receptor binding domain of SARS-CoV-2 spike protein (S-RBD) that overlap with the ACE-2 binding site to meditate neutralization (FIG. 1A) (Zost et al., 2020, Nature, 584:443-9; Dong et al., 2021, Nat Microbiol 6, 1233-1244). They were highly effective in multiple SARS-CoV-2 challenge models in small and large animals (Zost et al., 2020, Nature, 584:443-9; Chen et al., 2021, Nature, 596:103-8). mAbs 2130 and 2196 were further developed into clinical candidates AZD1061 and AZD8895, respectively, and are currently under evaluation as a cocktail combination (AZD7442) in a number of phase III clinical trials (NCT04723394, NCT04625972, NCT04625725). Indeed, recent results from the PROVENT trial (NCT04625725) demonstrated that prophylactic delivery of AZD7442 reduces the risk of symptomatic COVID-19 by 77% in a study cohort composed largely of high-risk participants (>75%). This speaks to the remarkable antiviral potency of these mAbs, both of which exhibit IC50 values in the low ng/mL range that appear to be retained against the major SARS-CoV-2 circulating strains (Zost et al., 2020, Nature, 584:443-9; Chen et al., 2021, NatureCom, 27:717-26; Chen et al., 2021, Nature, 596:103-8; Wang et al., 2021, Nature, 593:130-135). For adaptation to the DMAb platform, sequences encoding the mature variable heavy (V_{H}) and light (V_{L}) domains of mAbs 2130, 2196 and 2381 were optimized at the DNA and RNA levels and encoded, along with a wildtype human IgG1 framework (FcWT), into a custom mammalian expression vector (FIG. 1B-FIG. 1D). DMAbs 2196_FcWT, 2130 FcWT and 2381_FcWT, initially designed as single plasmid constructs (pHC/LC; FIG. 1B), were successfully expressed in vitro (FIG. 2A). Potent functionality against SARS-CoV-2 (WA1/2020) was confirmed in neutralization assays with both pseudotyped (FIG. 2B) and authentic (FIG. 2C) viruses.

### Rapid In Vivo Expression and Efficacy in a SARS-CoV-2 AAV-hACE2 Challenge Model

To determine the in vivo expression levels of initial DMAb constructs, plasmids were delivered intramuscularly to wildtype mice (BALB/c) followed by electroporation (CELLECTRA-EP) at the site of injection to facilitate uptake (Broderick et al., 2014, Expert Rev Vaccines, 14:195-204). Serum DMAb levels were detected over time following administration. Levels consistently increased, peaking at 6-10 μg/mL by D21 (FIG. 2D). The functional activity of sera harvested from DMAb-treated mice was evaluated against SARS-CoV-2 (WA1/2020) pseudotyped virus, confirming that in vivo-launched DMAbs possess high neutralization potencies (IC50<5 ng/ml; FIG. 2E) similar to those previously reported for the bioprocessed mAbs (Zost et al., 2020, Nature, 584:443-9). Next, the ability of DMAbs 2196_FcWT, 2130 FcWT and 2381_FcWT to provide protection against live SARS-CoV-2 infection was evaluated in vivo using an established non-lethal AAV6.2FF-hACE-2 murine challenge model (Gary et al., 2021, IScience, 24:102699) (FIG. 2F). Wildtype mice (BALB/c) were administered constructs encoding the indicated DMAbs (DO) before intranasal inoculation with AAV6.2FF-hACE2 (D14) to transduce human ACE2 receptor into the lungs. At D21, sera were collected and expression was detected in all DMAb-treated animals (FIG. 2G). Viral titers in the lungs of DMAb-treated and control mice were measured 4 days post-challenge with SARS-CoV-2 (WA1/2020) by qPCR (FIG. 2H). Animals expressing DMAbs 2196_FcWT, 2130_FcWT and 2381_FcWT had similar and significant reductions in lung viral burden (1-2 logs) compared to naïve animals (Kruskal-Wallis Test; P<0.05). These initial data confirm that functionally active 2196, 2130 and 2381 bearing the wildtype human IgG1 Fc, can be expressed in vivo at sufficient levels to mediate viral control of SARS-CoV-2.

### Design and Functional Characterization of Optimized 2196, 2130 and 2382-Based DMAbs

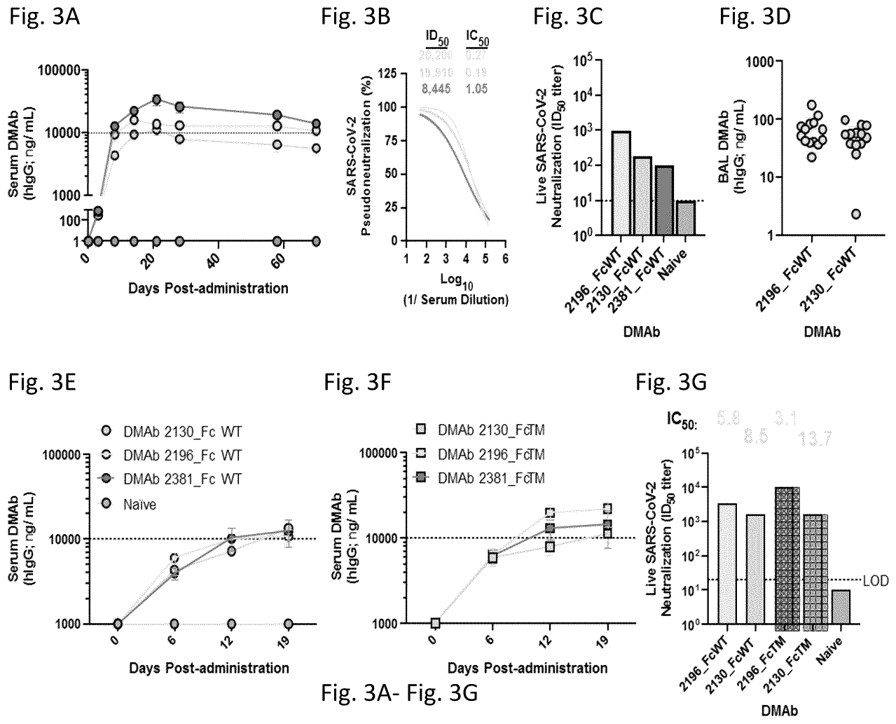

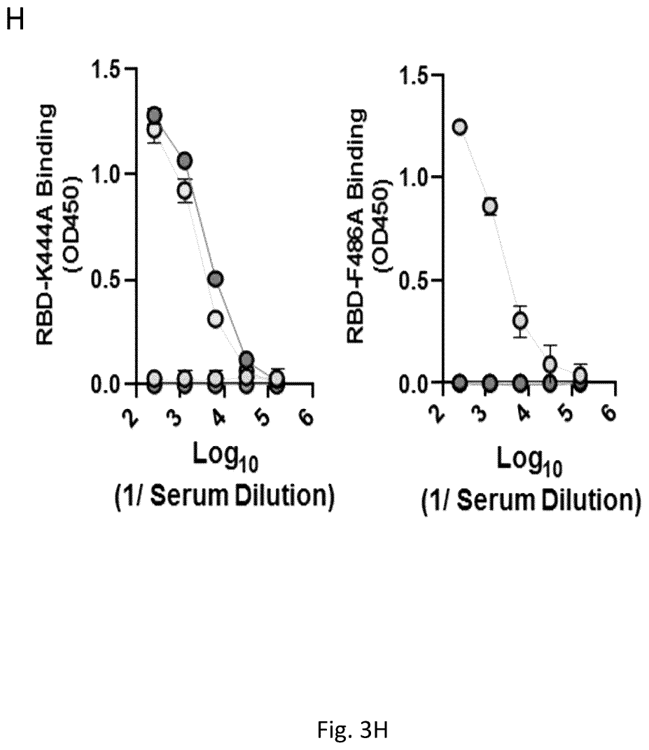

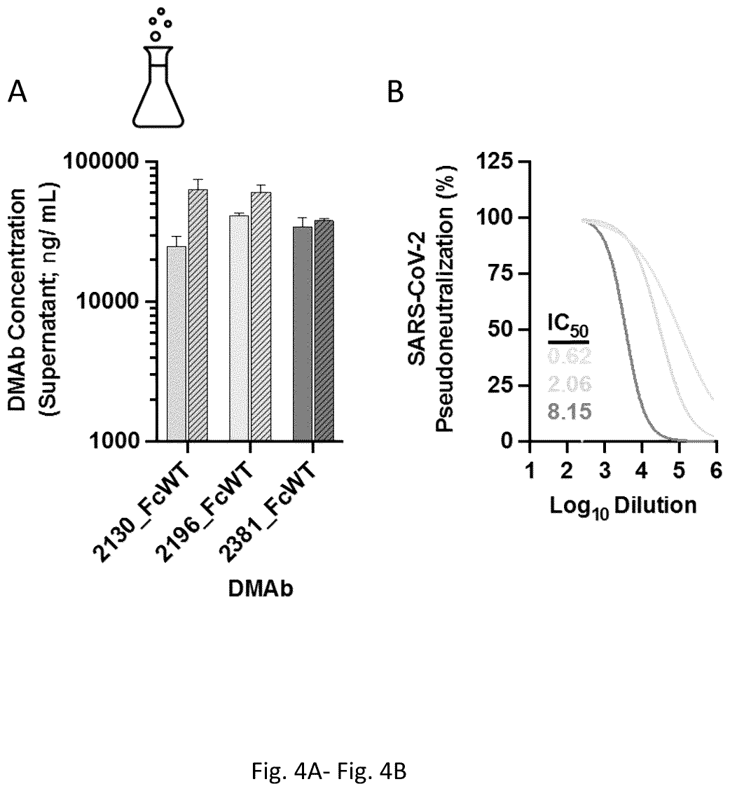

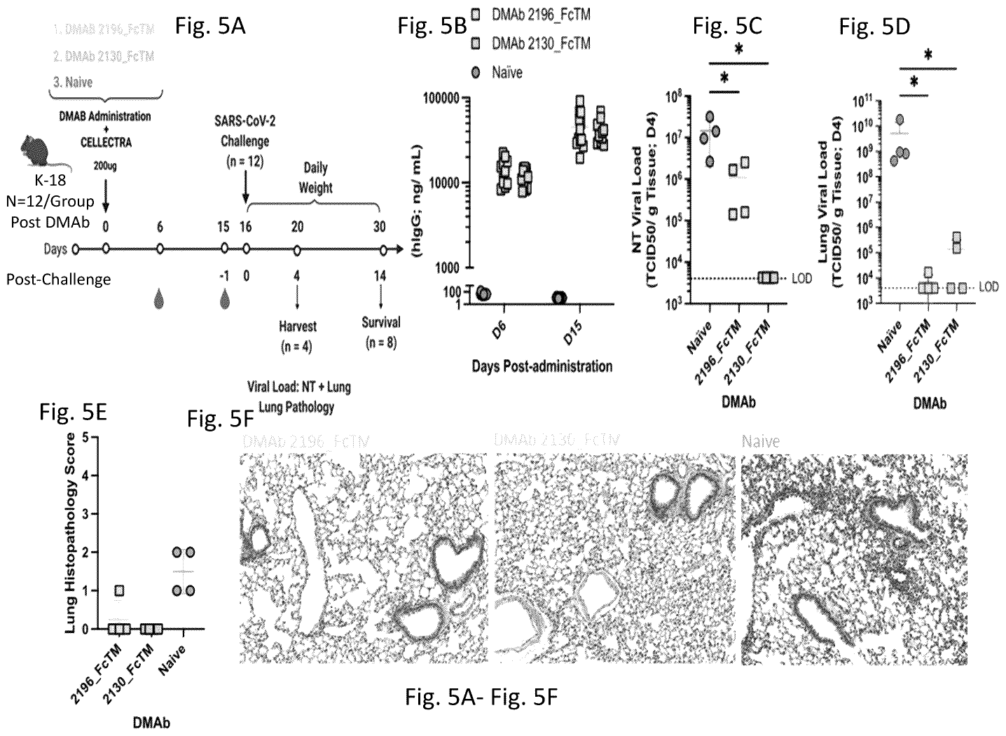

Various engineering approaches have been studied to enhance in vivo DMAb expression, focusing on strategic plasmid modifications that promote translation, processing and assembly of IgG in vivo (Elliott et al., 2017, PJ Vaccines, 2:18; Patel et al., 2018, Cell Rep, 25:1982-1993 e4). It was sought to further improve SARS-CoV-2 DMAb titers and optimize in vivo efficacy through construct reconfiguration using a dual plasmid system wherein each Ig chain is encoded separately (FIG. 1C). In vivo-delivery 2196_FcWT, 2130_FcWT and 2381_FcWT DMAbs using the dual system format led to a 2-4-fold increase in peak serum DMAb titers that were maintained for months (FIG. 3A). Functional assays confirmed that enhanced expression did not compromise antiviral activity against pseudotyped or authentic SARS-CoV-2 virus (FIG. 3B-FIG. 3C). Similar results were found following side-by-side in vitro expression of single and dual plasmid systems (FIG. 4). Of note, DMAbs 2196 and 2130 were also detected in the bronchiolar lavage (BAL) collected from parallel groups of mice at D14 post administration, indicating that in vivo-launched DMAbs are present the site of infection prior to challenge (FIG. 3D).

In addition to constructs bearing wildtype IgG (FcWT), which is capable of Fc-mediated effector engagement, an additional variant of each DMAb was generated containing triple-residue modifications (“TM”; L234F/L235E/P331S) in the Fc domain that abrogate these immune effector functions (Oganesyan et al., 2008, Acta Crystallogr Sect D Biol Crystallogr, 64:700-4) (FIG. 1D). Several clinical candidates, including AZD7442, incorporated such modifications. Parallel pK studies were conducted in K-18-hACE2, demonstrating that the FcTM variants of each DMAb clone are expressed at comparable, and in some cases significantly higher, levels than their WT counterparts (FIG. 3E-FIG. 3F). Side-by-side functional assessment of Fc variants showed similar potency against authentic SARS-CoV-2 (WA1/2020) virus (FIG. 3G). The epitope specificity of each DMAb clone was confirmed using modified RBDs containing point mutations K444A and F486A which are known to abrogate binding of mAb clones 2130 and 2196/2381, respectively (Zost et al., 2020, Nature, 584:443-9; Dong et al., 2021, Nat Microbiol 6, 1233-1244) (FIG. 3H). Furthermore, an established ACE-2 inhibition assay (Walker et al., 2020, J Clin Microbiol, 58) was utilized to demonstrate the ability of in vivo-launched DMAB variants to efficiently block RBD-binding to the hACE-2 receptor at very low ng/mL concentrations, consistent with their reported antiviral potencies.

### Prophylactic Delivery of Optimized 2196 and 2130-Based DMAbs Protects Mice Against SARS-CoV-2 Lethal Challenge

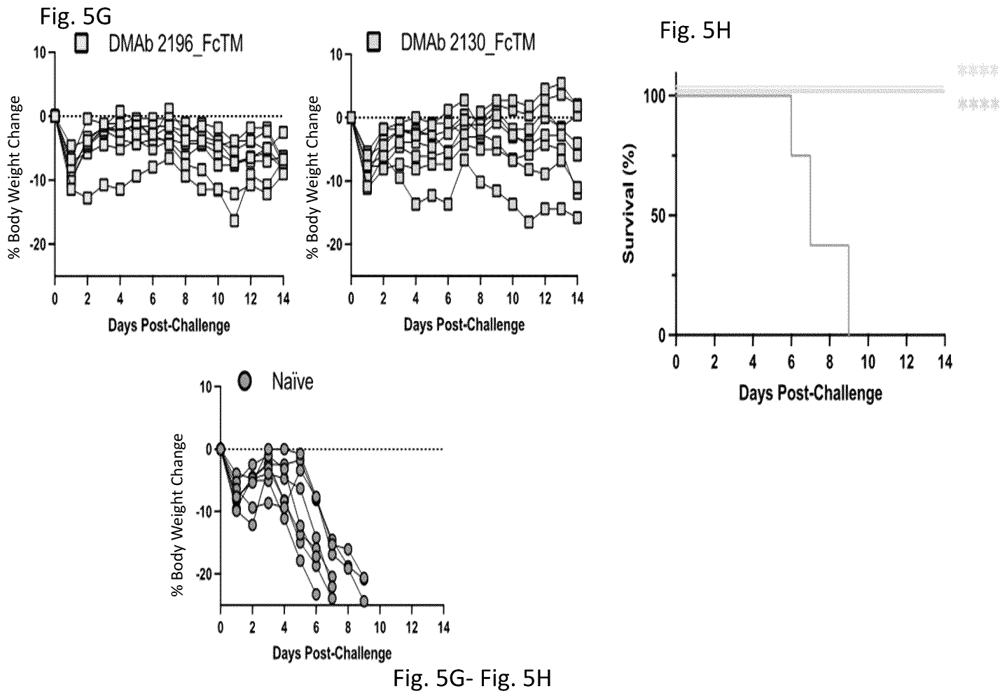

The in vivo efficacy of optimized DMAbs was next evaluated using a more stringent, lethal challenge model of transgenic mice (K-18 strain) that express human ACE2 under the cytokeratin 18 (K18) promotor, allowing the development of clinical symptoms, progressive disease, lung pathology and mortality following challenge (McCray et al., 2007, J Virol, 81:813-21; Winkler et al., 2020, Nat Immunol, 21(11):1327-35) (FIG. 5A). DMAbs 2196_FcTM and 2130_FcTM were administered individually to female K-18 mice (12/group) and serum expression was confirmed (FIG. 5B). Just prior to challenge (D15), both DMAbs were detected at high and comparable titers averaging 30-40 μg/mL. Following challenge with SARS-CoV-2 (WA1/2020), a subset (4/group) of each group were sacrificed at D4 to evaluate DMAb-mediated viral control. Viral burdens in the nasal turbinates (NT; FIG. 5C) and lungs (FIG. 5D) were significantly reduced in DMAb-treated groups compared to control mice (Mann-Whitney U; P<0.05); this reduction was particularly striking in the lung, where it was lower by a magnitude of 4-6 logs. Consistent with these findings, DMAb-treated animals also had reduced levels of lung pathology relative to controls (FIG. 5E and FIG. 5F). The remaining animals (8/group) were monitored daily for weight loss and survival. As expected, the majority (88%, 14/16) of mice in both DMAb-administered groups were protected against the severe (>10%) and progressive weight loss observe in control mice (FIG. 5G). This conferred 100% survival in these groups at D14 post-challenge, a stark contrast from the control group in which all mice succumb to infection by D9 (Log-rank test; P<0.0001) (FIG. 5H). These data demonstrate the ability of each in vivo-launched neutralizing antibody to mediate viral reduction, prevent lung inflammation/pathology and protect animals from severe disease and death when administered as monotherapies.

In Vivo-Launched DMAb Cocktails Mediate Superior Viral Control and Protection from Lethal SARS-CoV-2 Infection

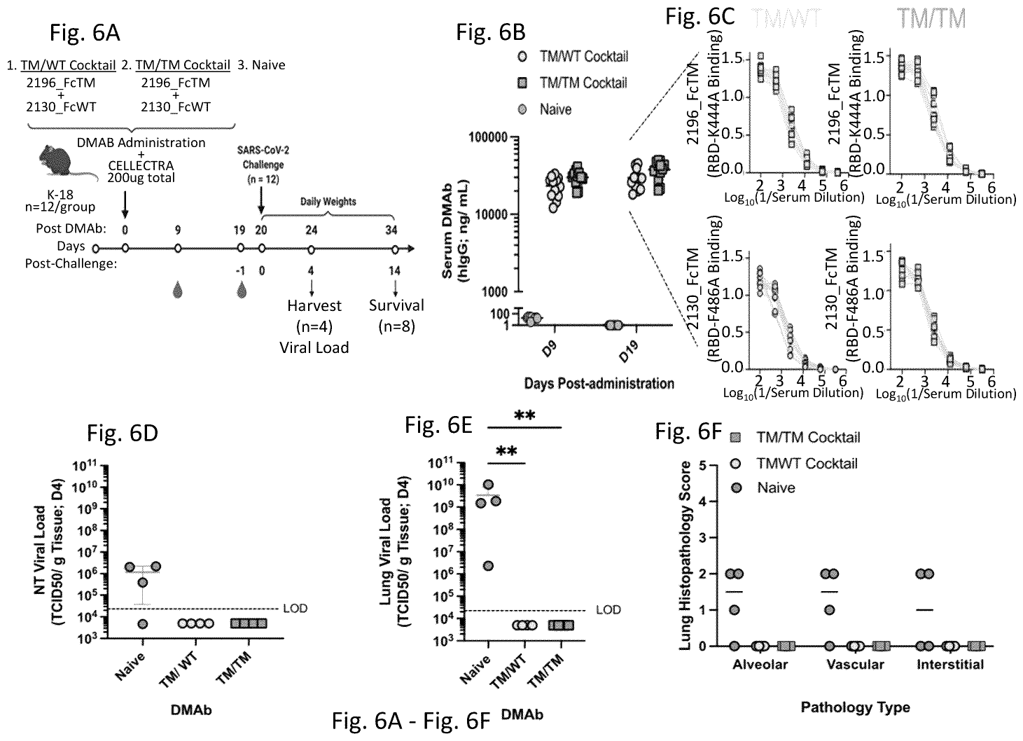

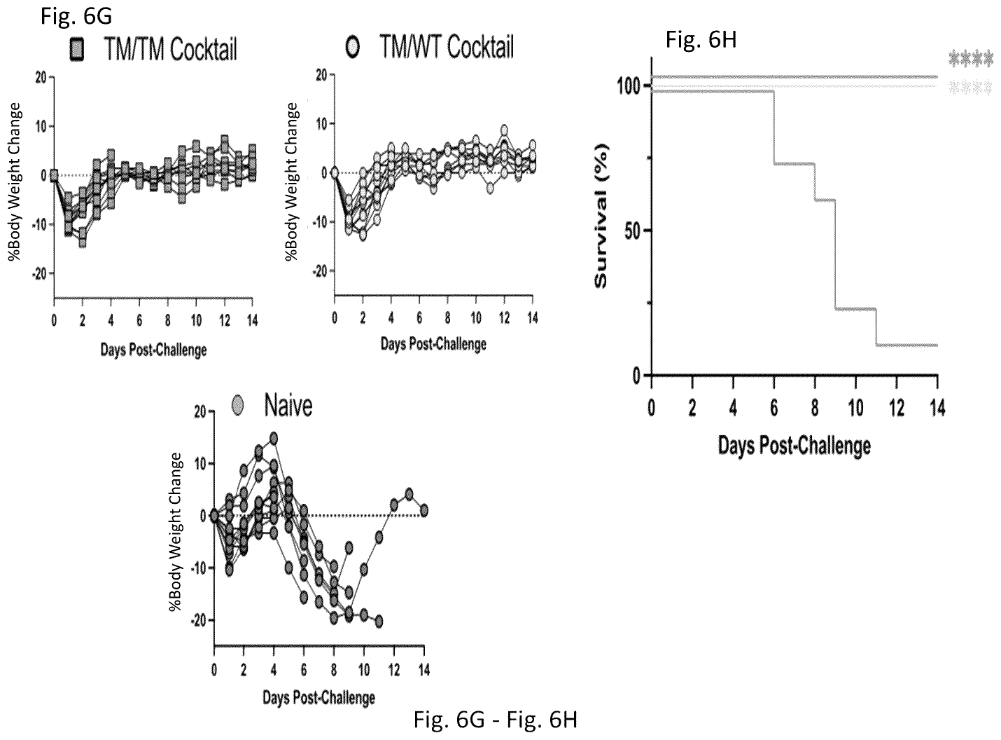

Previous reports demonstrated the complimentary and synergistic nature of mAbs 2130 and 2196 (Zost et al., 2020, Nature, 584:443-9; Dong et al., 2021, Nat Microbiol 6, 1233-1244). The protection afforded by co-delivery of DMAbs 2196_FcTM and 2130_FcTM (TM/TM cocktail) was evaluated, similar to the AZD7442 clinical candidate. Further, an alternate cocktail containing DMAbs 2196_FcTM and 2130_FcWT (TM/WT cocktail) was tested to assess the effect of Fc-mediated effector function engagement on in vivo efficacy against lethal challenge (FIG. 6A). Delivery of DMAb cocktails to male K-18 mice (n=12/group) resulted in robust expression of plasmid-derived hIgG the sera, averaging 30-40 μg/mL in both groups by D19 (FIG. 6B). Of note, sera to from all DMAb-administered animals exhibited strong and consistent recognition of both epitope-specific RBD mutants (K444A and F486A), indicating concurrent in vivo expression of both 2196 and 2130-derived DMAbs (FIG. 6C). Following SARS-CoV02 challenge, all DMAb-treated animals (n=4/group) exhibited complete viral control in the nasal turbinates (FIG. 6D) and lungs (FIG. 6E) at D4 post-infection where viral titers were below the limit of detection; this represented >5-log reduction in lung viral loads compared to control animals (Mann-Whitney-U; P<0.01). Complete protection against SARS-CoV-2-induced lung pathology in DMAb-treated mice was also observed in these animals while most control mice (75%) exhibited one or more forms of inflammation (FIG. 6F). Consistent with robust viral control, DMAb-treated animals exhibited minimal weight-loss (FIG. 6G), and complete survival (FIG. 6H) while progressive weight-loss was observed in all control mice leading to a significant (88%) death (Log-rank Test; P<0.0001). Similar to previous reports, these data suggest that co-delivery of 2130 and 2196-based DMAb provides superior viral control and protection compared to corresponding monotherapies. This was true regardless of the status of effector function engagement.

### 2196 and 2130-Based DMAbs Maintain Functionality Against SARS-CoV-2 Variants of Concern

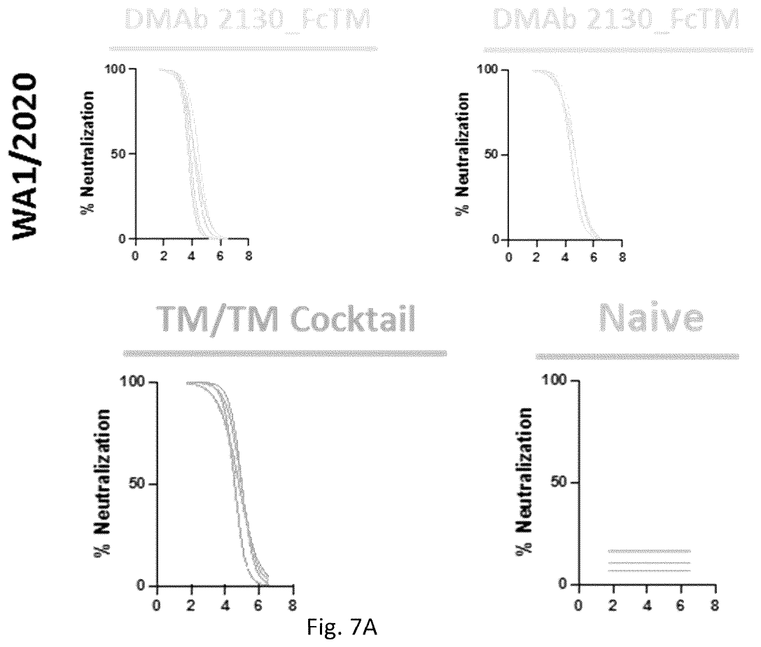

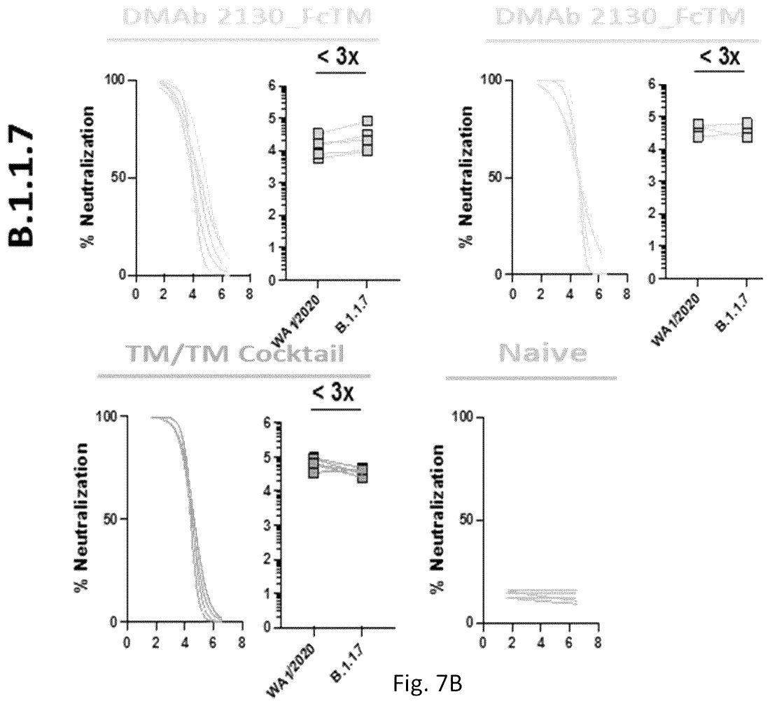

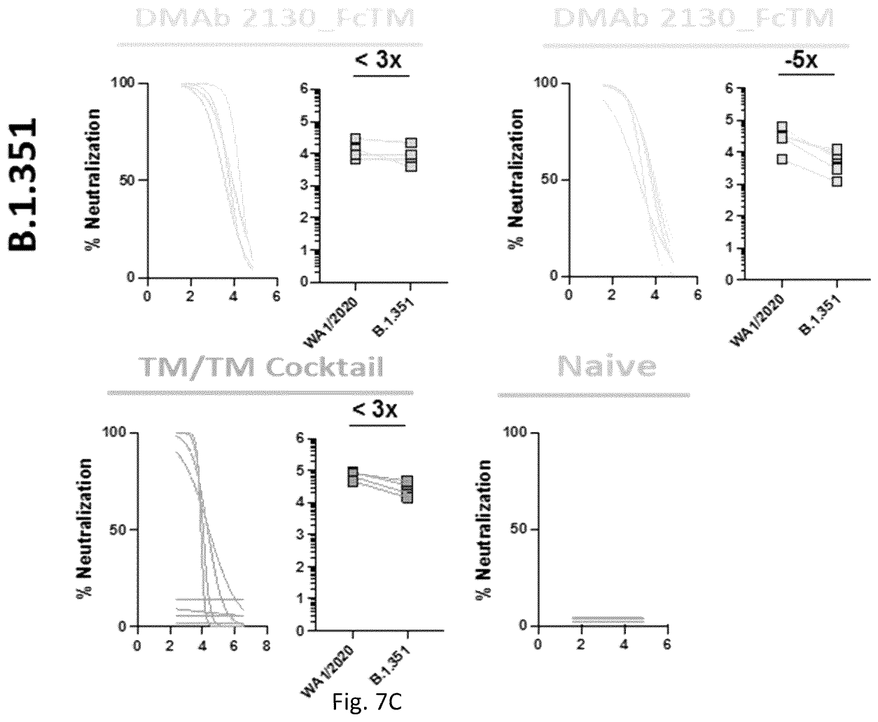

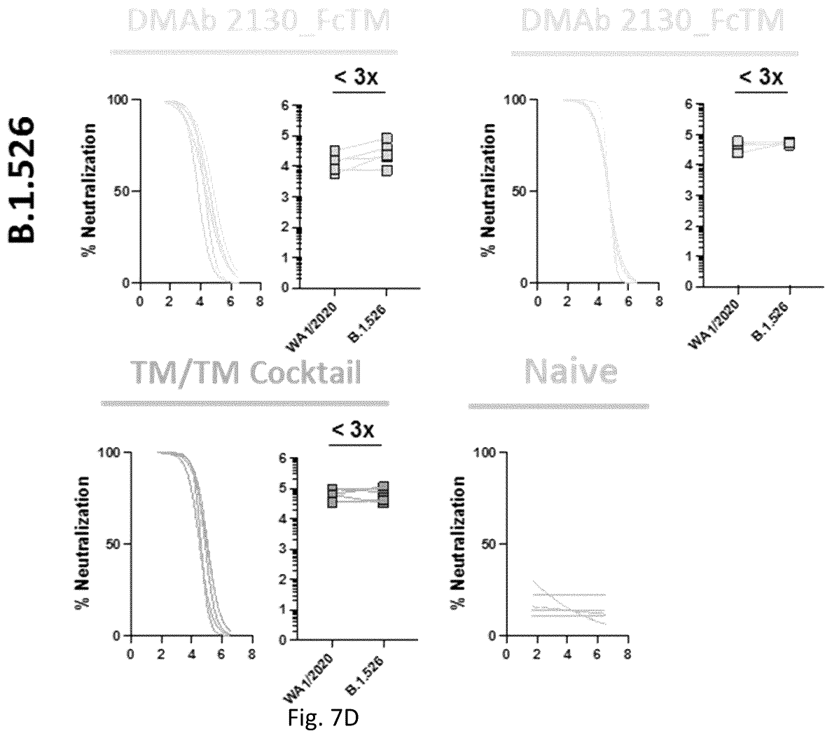

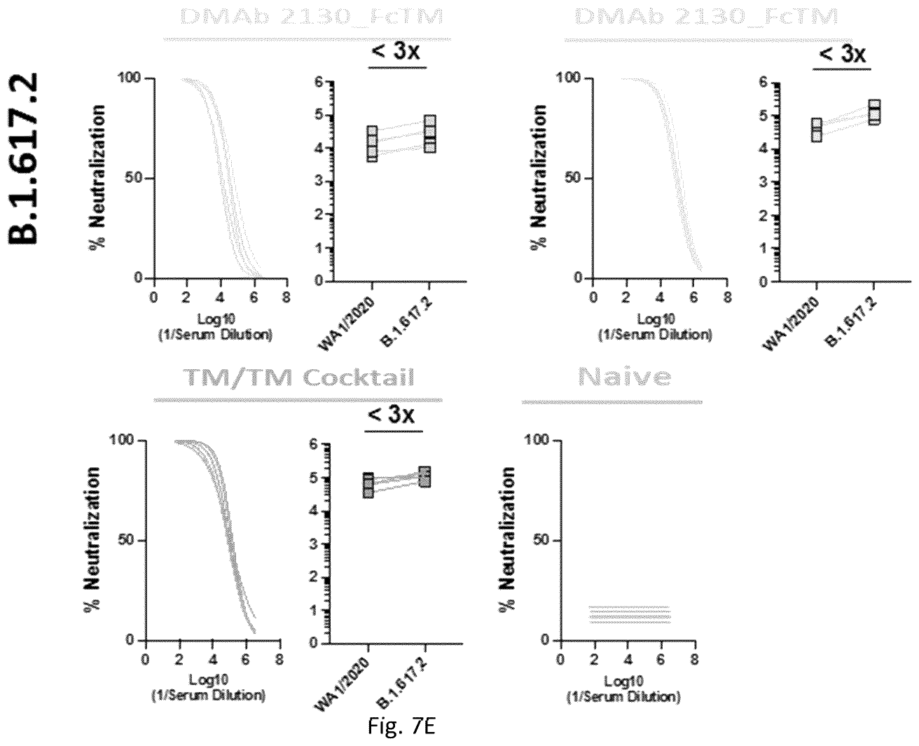

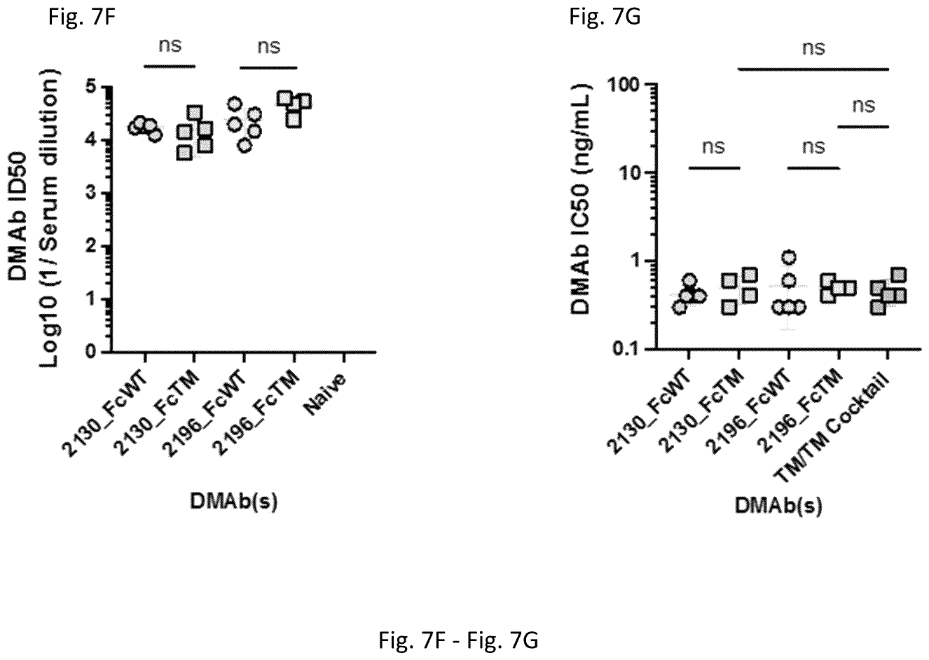

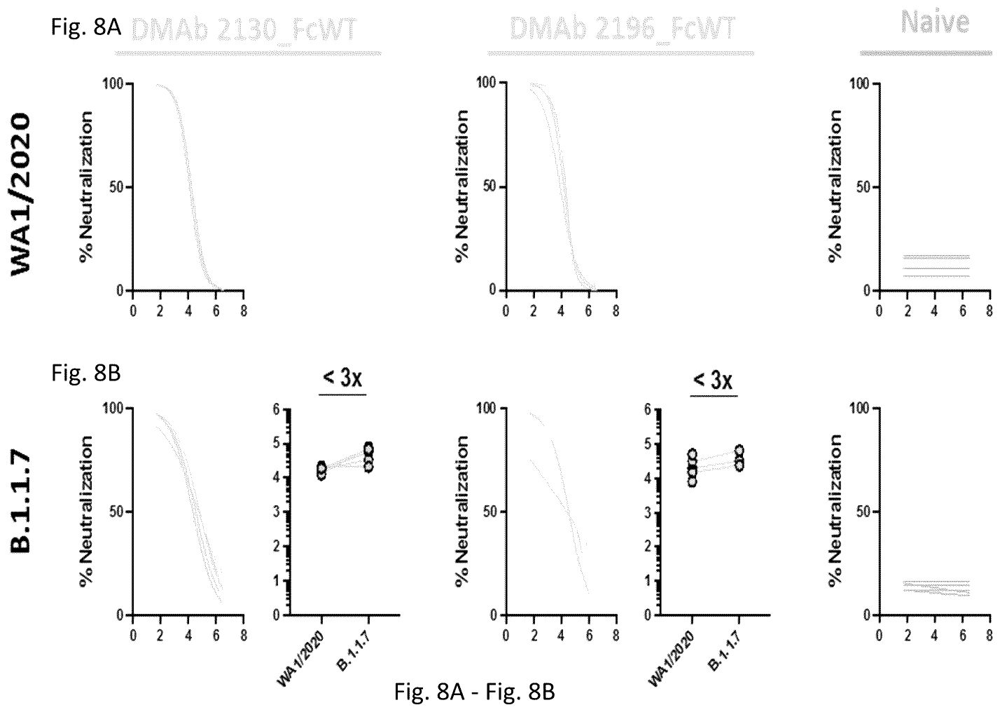

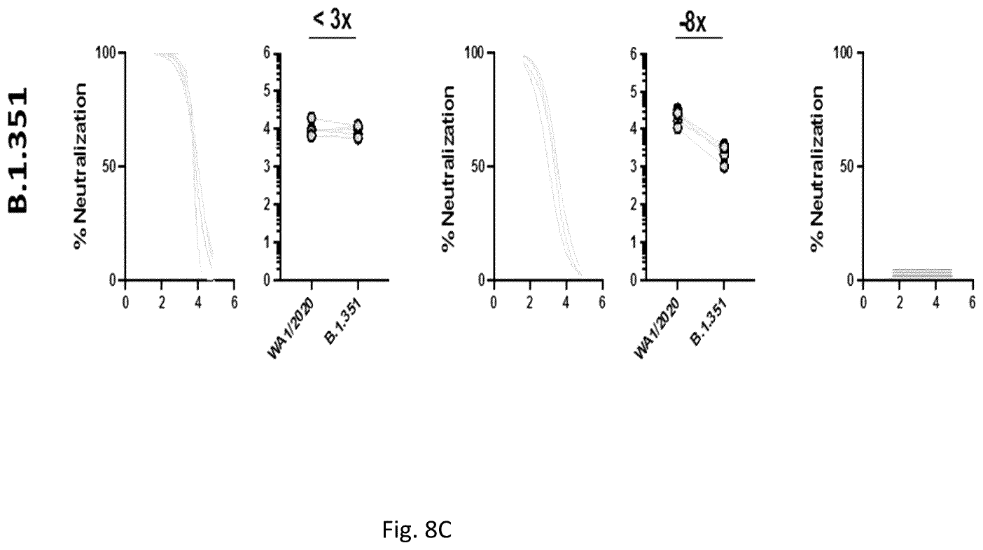

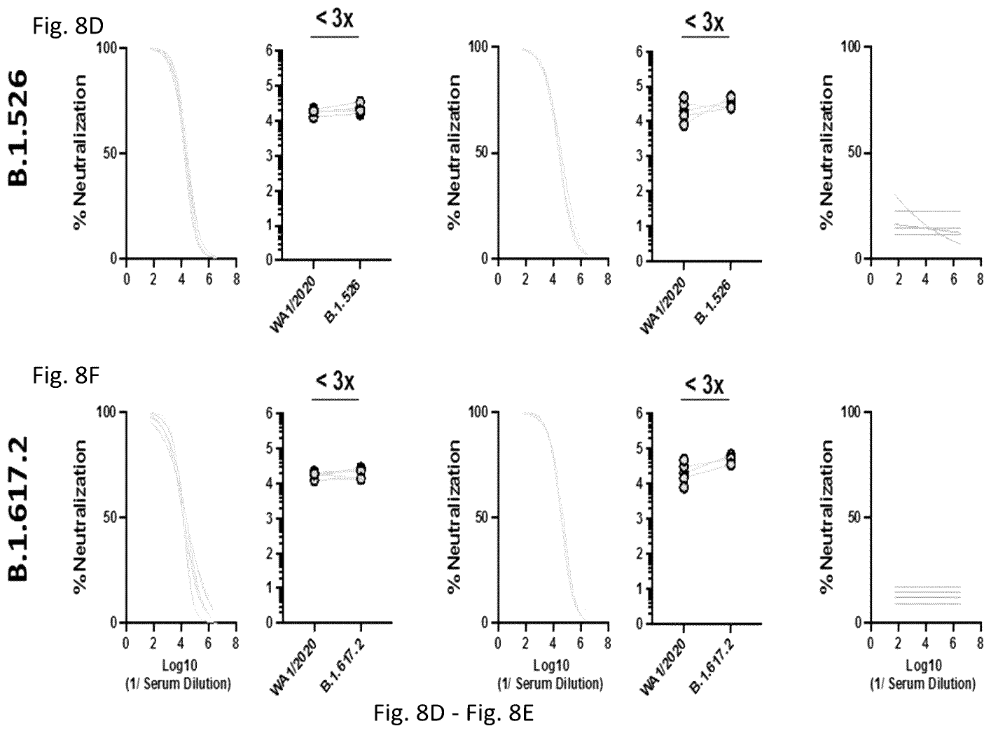

A number of dominant SARS-CoV-2 variants bearing mutations in the spike protein have been identified, including B.1.1.7 (Rambaut et al., 2020, VirologicalOrg, 2020:1-9), B.1.351 (Tegally et al., 2021, Nature, 592:438-43), P.1 (Naveca et al., VirologicalOrg, 2021), B.1.526 (Annavajhala et al., 2021, Nature, 597:703-708), B.1.617.2 (European Centre for Disease Prevention and Control, Threat assessment brief: emergence of SARS-CoV-2 B.1.617 variants in India and situation in the EU/EEA. 2021) among others. Substitutions that confer increased resistance to therapeutics are largely clustered in the RBD where they are more likely to interrupt epitope recognition by neutralizing mAbs (Starr et al., 2021, Cell Reports Med, Volume 2, Issue 4, 20 Apr. 2021, 100255; Greaney et al., 2021, Cell Host Microbe, 29:44-57.e9; Starr et al., 2021, Science, 371:850-4) (FIG. 7A). To confirm activity against viral variants, the relative binding of in vivo-launched DMAbs was first assessed against mutant RBDs with modifications at position(s) 501, 417, 484 and/or 614 compared to the parental RBD-WA1/2020 (FIG. 7B). Consistent with recent reports, 2130-based DMAbs retained recognition of all mutant RBDs while 2196-derived DMAbs demonstrated reduced binding to the E484K single RBD mutant. This binding was further reduced when additional mutations were incorporated. Importantly, the relative binding against E484K-containing RBDs was largely restored in sera from animals that received both DMAbs (TM/TM). The effect on antiviral activity was measured against pseudotyped variant viruses bearing the B.1.1.7 (Alpha), B.1.351 (Beta), B.1.526 (Iota) or B.1.617.2 (Delta) spike mutations (FIG. 7A through FIG. 7E and FIG. 8). Consistent with binding assays, 2130-based DMAbs maintained activity against all variants (<3-fold reduction) while 2196-derived DMAbs exhibited a mild (5-to-8 fold) reduction relative to the parental strain. Importantly, sera containing the DMAb cocktail restored activity (<3-fold reduction) to variant spikes containing mutations at the 484 position. Reproducible serum potency of 1-2 ng/mL was observed in all individual sera samples tested, which was unaffected by the Fc framework (FcWT vs FcTM) (FIG. 7F and FIG. 7G). These data verify the functional activity of plasmid-launched DMAbs against multiple variants of concern, supporting further expansion of the DMAb platform to additional promising clones.

### Expanding the SARS-CoV-2 DMAb Panel

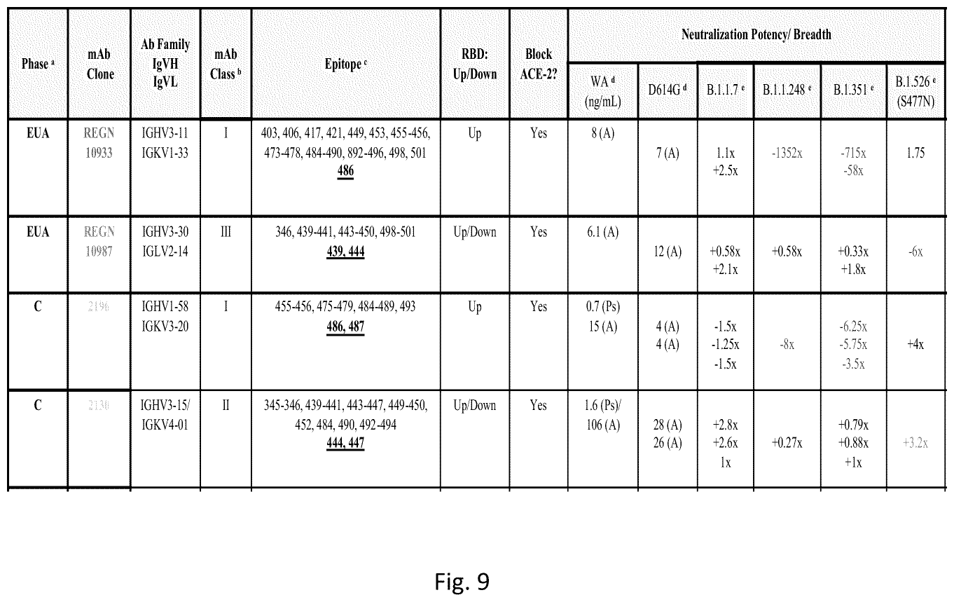

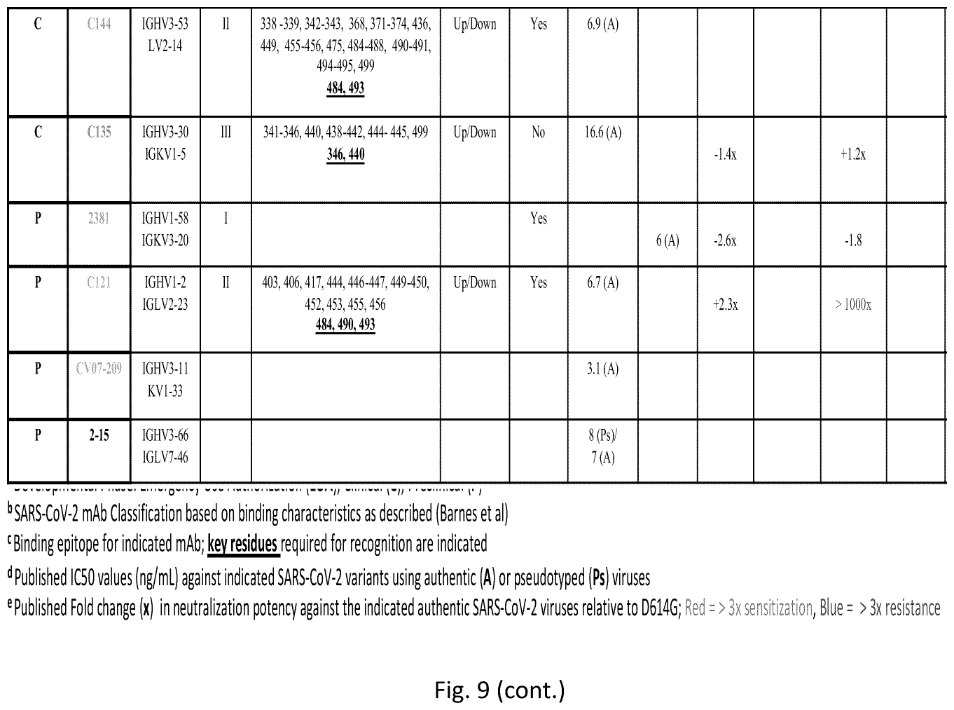

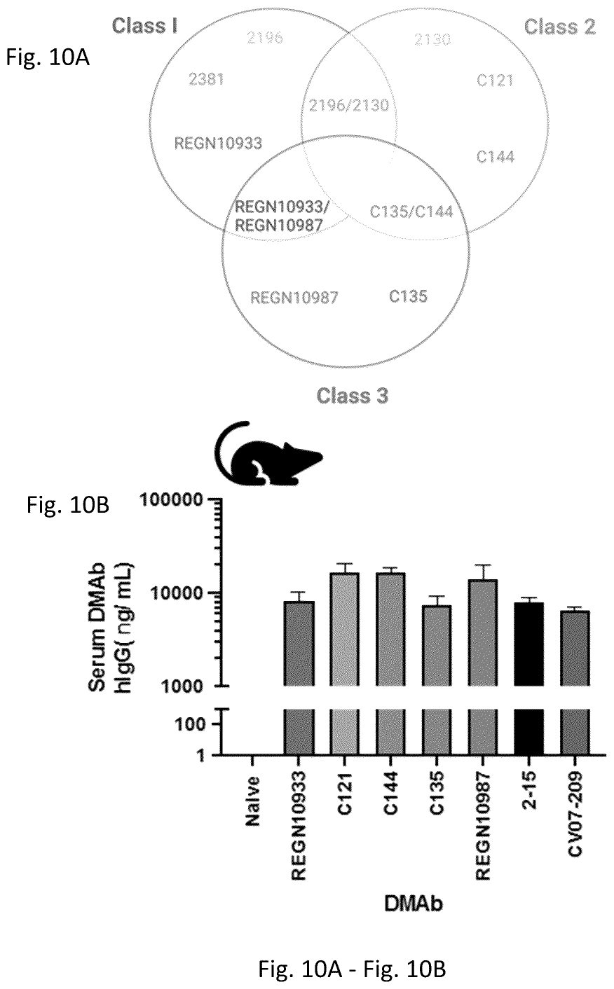

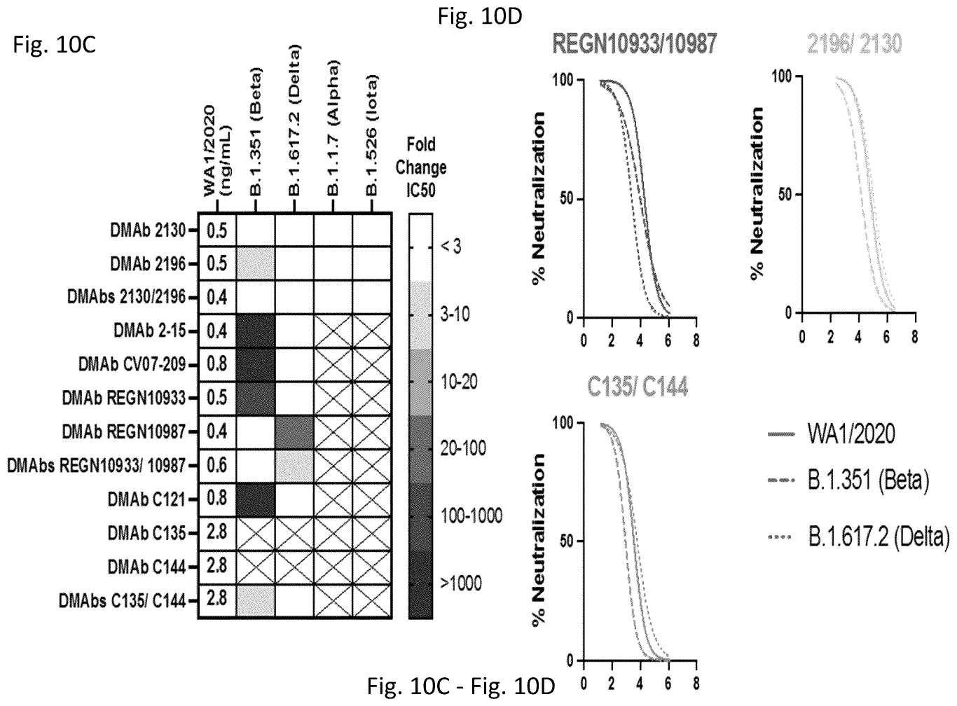

Finally, it was sought to engineer additional DMAbs in order to broaden and diversify the SARS-CoV-2 DMAb portfolio (FIG. 9). Seven additional promising RBD-specific clones were selected, including authorized mAb pair REGN10933/REGN10987 (REGN-CoV-2) (Hansen et al., 2020, Science, 369:1010-4) as well as promising clinical candidate pair C135/C144 (Robbiani et al., 2020, Nature, 584:437-442) and preclinical clones C121 (Robbiani et al., 2020, Nature, 584:437-442), CV07-209 (Kreye et al., 2020, Cell, 183(4): 1058-1069.e19) and 2-15 (Liu et al., 2020, Nature, 584:450-6). Together with DMAbs 2196, 2130 and 2381, this panel possesses a range of binding specificities and functional characteristics. They span the three main SARS-CoV-2 antibody classes (FIG. 10A), cocktails of which represent all possible class combinations (I/II, I/III and II/III). They also contain 8 unique IgHV/IgLV immunoglobulin gene pairings which, similar to the reported VH-gene signatures of patient-derived SARS-CoV-2 clones, are heavily enriched for IgGVH1 and IgGVH3 (IgGHV3-66 and IgGHV3-53) families (Robbiani et al., 2020, Nature, 584:437-442; Cao et al., 2020, Cell, 182:73-84.e16). Constructs encoding publicly available variable sequences were engineered and inserted on a FcWT framework using dual plasmid systems as previously described (see FIG. 1C). All DMAbs were expressed in K-18 mice with D14 average titers ranging from 6-20 μg/mL (FIG. 10B). Antiviral activities of all in vivo-launched DMAbs were assessed side-by-side against SARS-CoV-2 (WA1/2020) pseudotyped viruses, with IC50 potencies similar to those reported for their recombinant counterparts (FIG. 10C). Neutralization against the B.1.351 were as expected, with the class I clones from each from clinical pair (REGN10933 and 2196) showing some level of decreased activity which was much more severe for REGN10933 (>200-fold reduction) compared to 2196 (˜8-fold reduction). Their partners, REGN10987 and 2130, maintained activity against this variant (<3× change). Interestingly, REGN10987 exhibited somewhat reduced activity against the B. 1.617.2 variant which contains a mutation at residue 440 that is not as common in other variants to date. As this residue lies within the binding epitope of REGN10987 (FIG. 9), some reduction in potency expected. Importantly, antiviral activity in sera was completely (2130/2196) or almost completely (REGN10933/10987 and C135/C144) restored in cocktail-treated animals (FIG. 10C and FIG. 10D).

### Example 2: Antibodies Against SARS-CoV-2

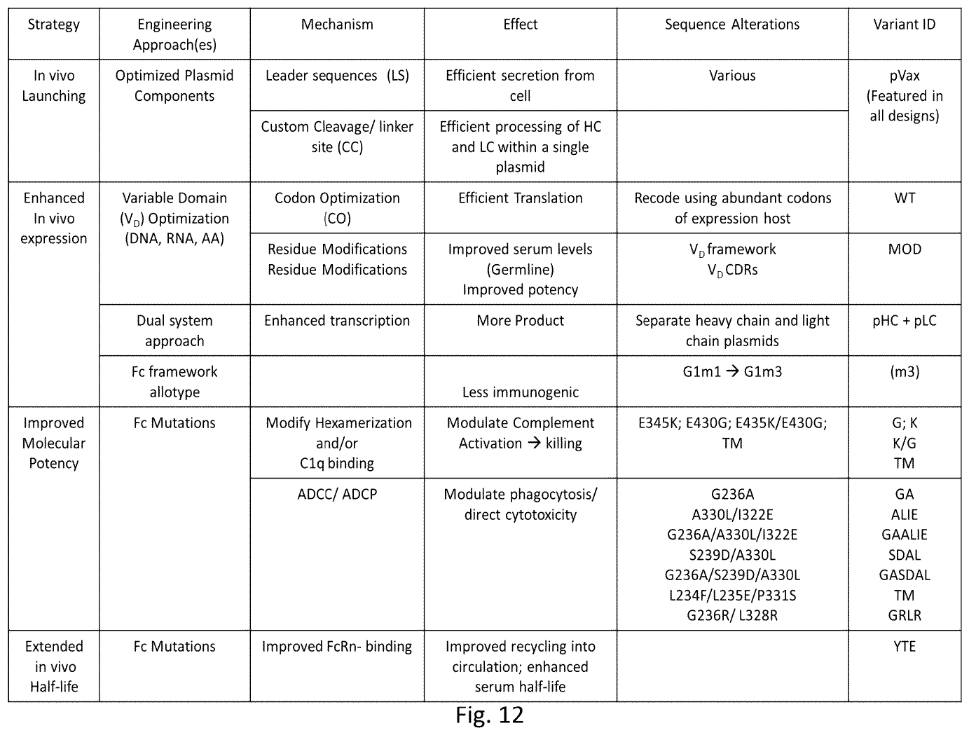

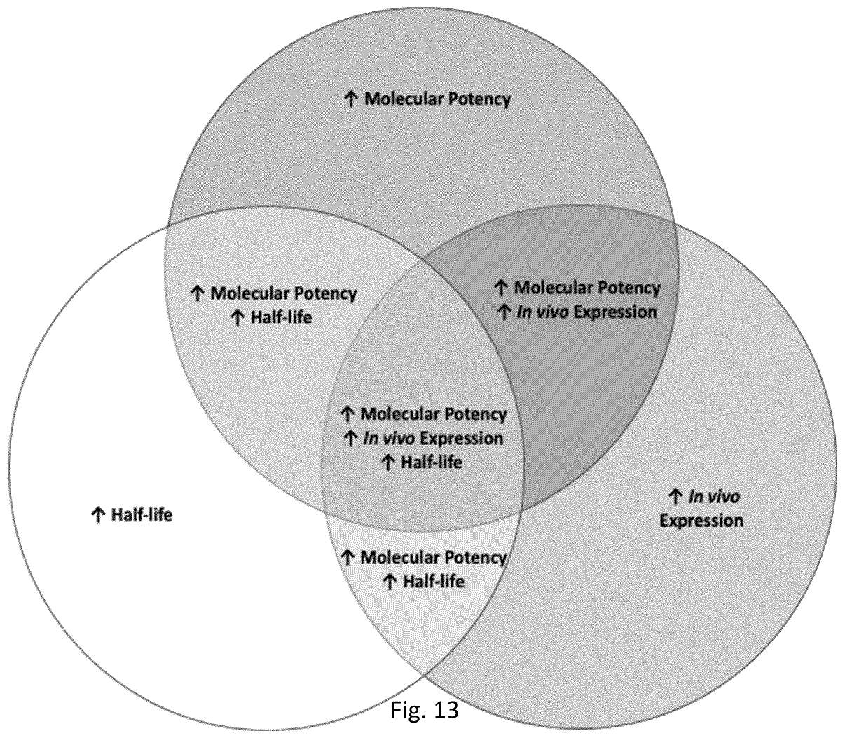

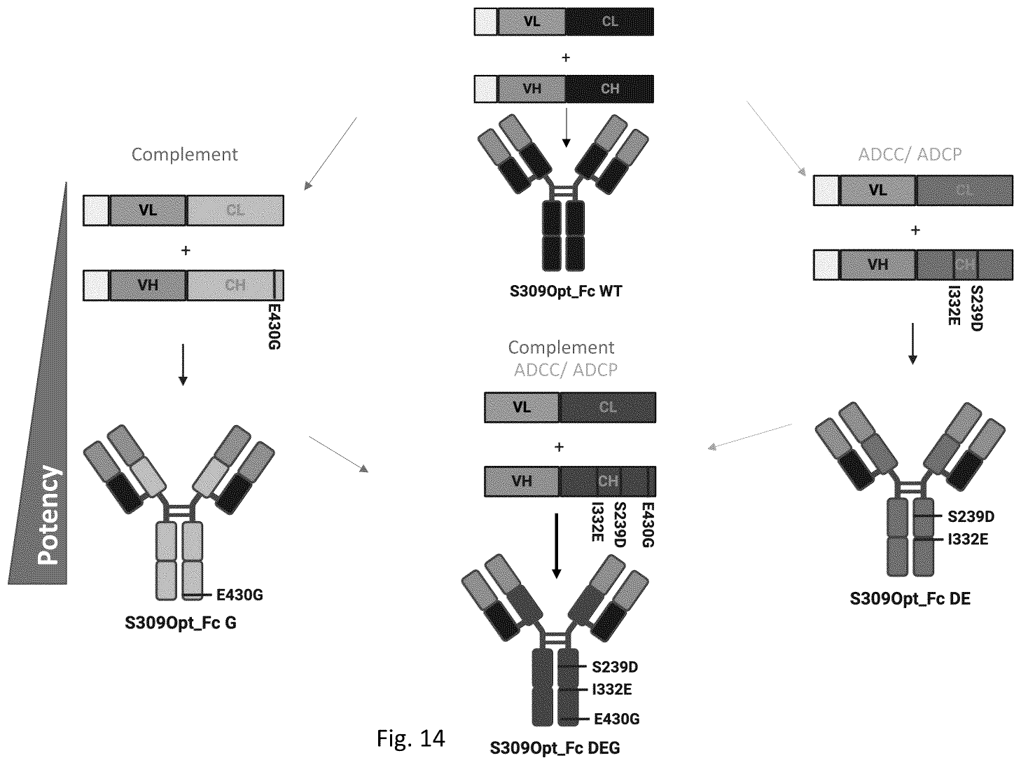

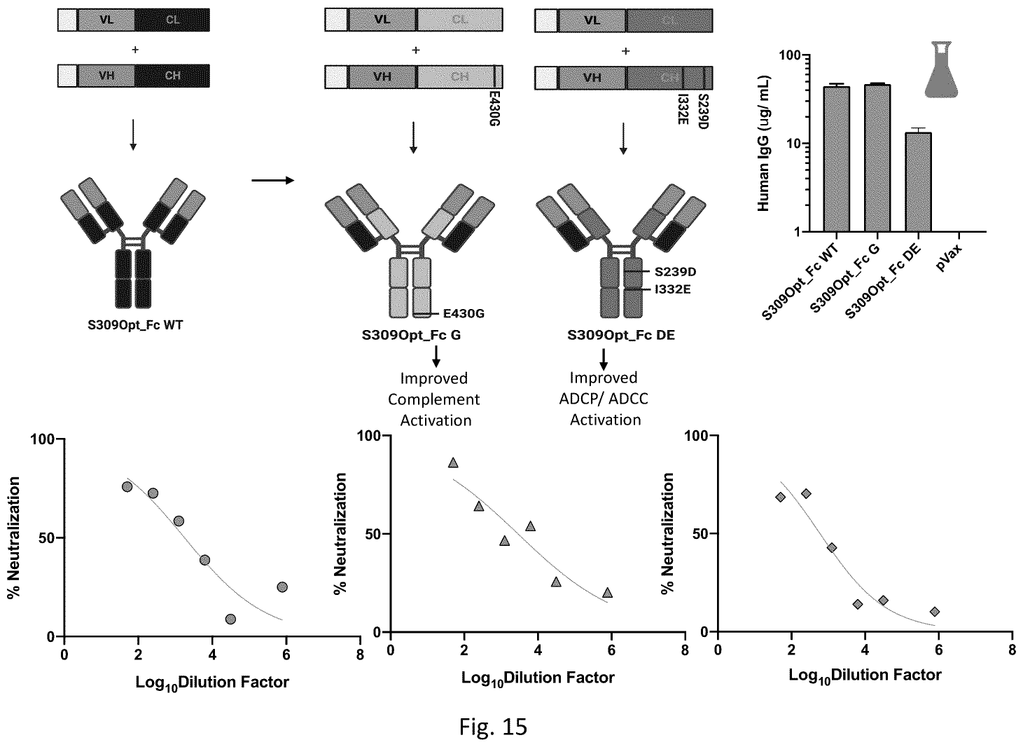

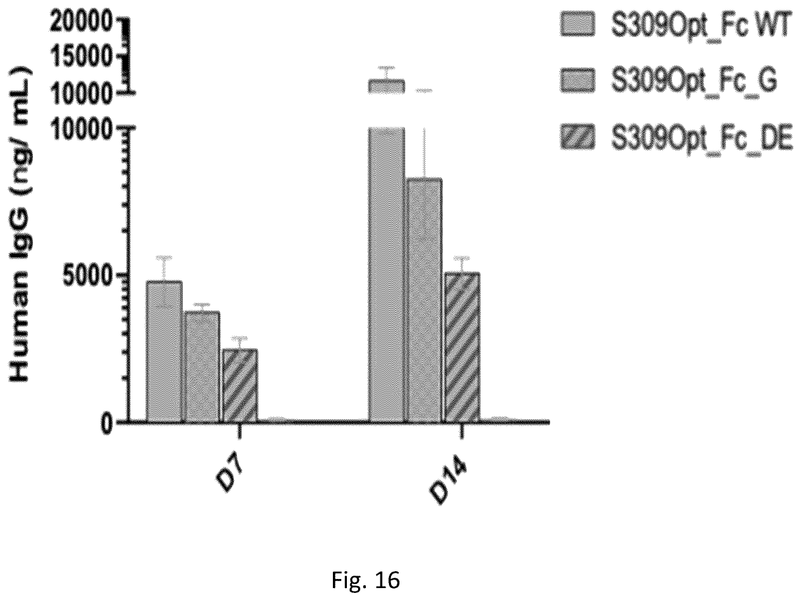

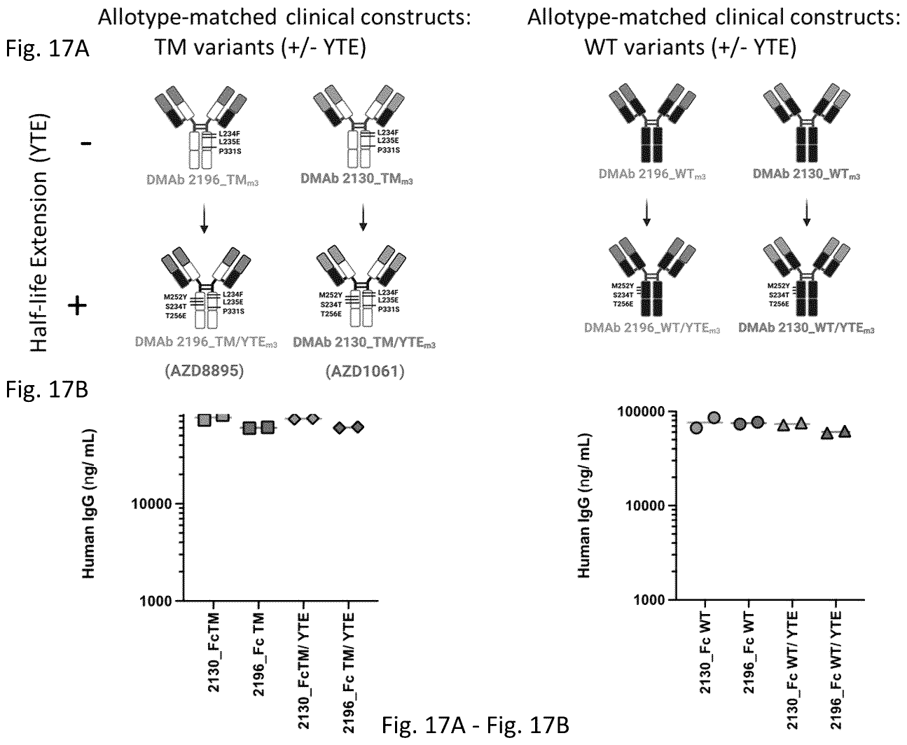

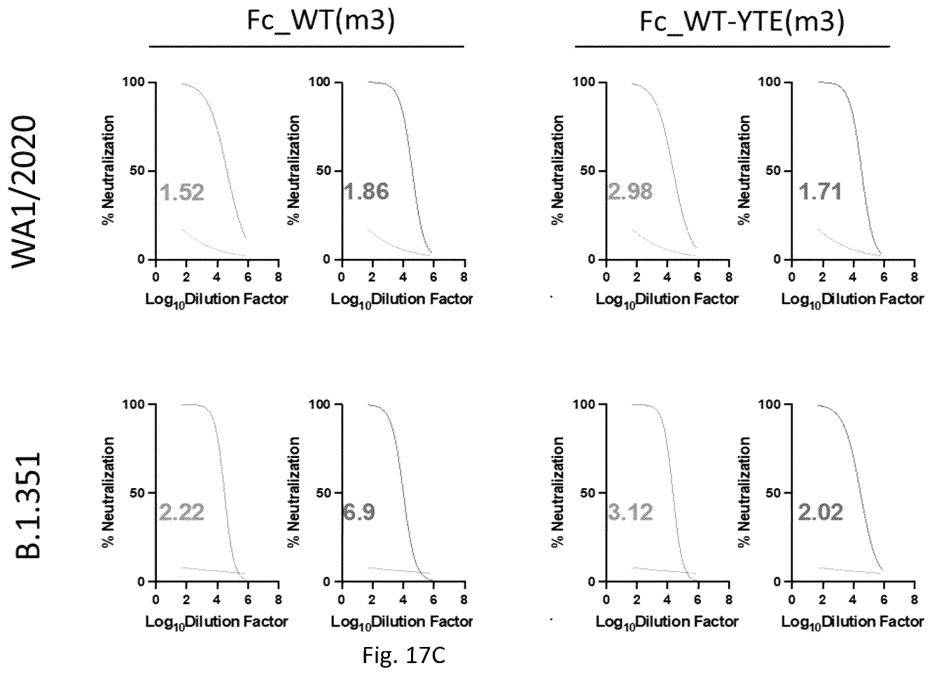

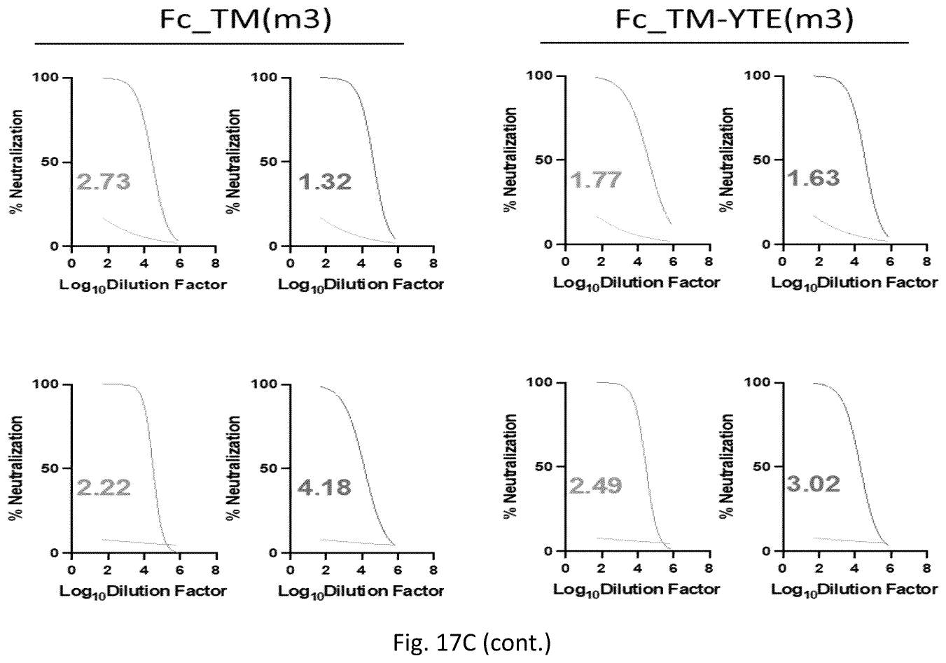

Optimized DNA-encoded monoclonal antibodies (dMABs) were developed against SARS-CoV-2 virus for prevention, treatment and/or diagnostic use, including treatment or prevention of a SARS-CoV-2-mediated disease (e.g., COVID-19). Novel engineering approaches were used to develop dMABs for in vivo delivery with enhanced production including enhanced expression, processing, assembly, secretion and half-life (FIG. 12-FIG. 13). The optimizations included use of a two-plasmid system (FIG. 2-FIG. 3), variation of the Fc region (FIG. 2, FIG. 3 and FIG. 14-FIG. 16), variation of allotype (FIG. 17), and combinations (FIG. 10).

TABLE 1
Antibody and DMAb Sequences

| SEQ ID | | | | | | | |
|---|---|---|---|---|---|---|---|
| NOs: | | | | | | | |
| (DNA/ | Clone | | WT or | Ig | | | |
| AA) | (Allotype) | Variable Domain | MOD | Isotype | Allotype | Fc Variant | PLASMID |
| 1/2 | 2196(m1) | 2196 LC | WT | | | | DMAb-2196_kLC_pVax1 |
| 3/4 | 2196(m1) | 2196 | WT | IgG1 | G1m1 | WT | DMAb-2196MOD_IgG1HC |
| | | | | | | | WT_pVax1 |
| 5/6 | 2196(m1) | 2196 | WT | IgG1 | G1m1 | TM | 11. DMAb- |
| | | | | | | | 2196MOD_IgG1HC_MOD/ |
| | | | | | | | TM_pVax1 |
| 7/8 | 2130(m1) | 2130 LC | WT | | G1m1 | | DMAB-2130_kLC_pVax1 |
| 9/10 | 2130(m1) | 2130 HC | WT | IgG1 | G1m1 | WT | DMAb-2130_IgG1HC |
| | | | | | | | WT_pVax1 |
| 11/12 | 2130(m1) | 2130 HC | WT | IgG1 | G1m1 | TM | 1. DMAb- |
| | | | | | | | 2130_IgG1HC_WT/TM_pVax1 |
| 13/14 | 2381(m1) | 2381 LC | WT | | G1m1 | | DMAb-2381_kLC_pVax1 |
| 15/16 | 2381(m1) | 2381 HC | WT | IgG1 | G1m1 | WT | DMAb-2381_IgG1HC |
| | | | | | | | WT_pVax1 |
| 17/18 | 2381(m1) | 2381 HC | WT | IgG1 | G1m1 | TM | 6. DMAb- |
| | | | | | | | 2381_IgG1HC_WT/TM_pVax1 |
| 19/20 | S309 (m1) | S309 LC_k | WT | | G1m1 | | S309_LC(k) |
| 21/22 | S309 (m1) | S309 HC | WT | IgG1 | G1m1 | WT | S309_IgG1_HC_WT_pVax1 |
| 23/24 | S309 (m1) | S309 HC | WT | IgG1 | G1m1 | E430G | S309_HC_E430G_pVax1 |
| 25/26 | S309 (m1) | S309 HC | WT | IgG1 | G1m1 | DE | S309_HC_D/E_pVax1 |
| 27/28 | S309 (m1) | S309 HC | WT | IgG1 | G1m1 | DEG | S309_HC_D/E/G_pVax1 |
| 29/30 | 2-15(m1) | 215_(k) LC | WT | | G1m1 | | DMAB 215_(K)LC |
| | | | | | | | WT_pVax1 |
| 31/32 | 2-15(m1) | 2-15 HC | WT | IgG1 | G1m1 | WT | DMAB |
| | | | | | | | 215_IgG1HC_WT_pVax1 |
| 33/34 | REGN87(m1) | REGN87_(L) LC | WT | | G1m1 | | 6.REGN87_L_LC_WT_pVax1 |
| 35/36 | REGN87(m1) | REGN87_HC_{—} | WT | IgG1 | G1m1 | WT | 5.REGN87_IgG1HC_WT_pVax1 |
| 37/38 | REGN33(m1) | REGN33_(k) LC | WT | | G1m1 | | 4.REGN33_k_LC_WT_pVax1 |
| 39/40 | REGN33(m1) | REGN33_HC_{—} | WT | IgG1 | G1m1 | WT | 3.REGN33_IgG1HC_WT_pVax1 |
| 41/42 | C121(m1) | C121_(L) LC_{—} | WT | | G1m1 | | 10.C121_L_LC_WT_pVax1 |
| 43/44 | C121(m1) | C121_HC_{—} | WT | IgG1 | G1m1 | WT | 9.C121_IgG1HC_WT_pVax1 |
| 45/46 | C135(m1) | C135_(k) LC_{—} | WT | | G1m1 | | 12.C135_k_LC_WT |
| 47/48 | C135(m1) | C135_HC_{—} | WT | IgG1 | G1m1 | WT | 11.C135_IgG1HC_WT_pVax1 |
| 49/50 | C144(m1) | C144_(L) LC | WT | | G1m1 | | 14.C144_L_LC_WT_pVax1 |
| 51/52 | C144(m1) | C144_HC_{—} | WT | IgG1 | G1m1 | WT | 13.C144_IgG1HC_WT |
| 53/54 | CV07- | CV07-209_(k) | WT | | G1m1 | | 8.CV07- |
| | 209(m1) | LC_{—} | | | | | 209_k_LC_WT_pVax1 |
| 55/56 | CV07- | C135_HC_{—} | WT | IgG1 | G1m1 | WT | 7.CV07- |
| | 209(m1) | | | | | | 209_IgG1HC_WT_pVax1 |
| 57/58 | 2130(m3) | DMAB | WT | | G1m3 | | 18. DMAB |
| | | AZ1061CLIN_LC | | | | | AZ1061CLIN_LC |
| 59/60 | 2130(m3) | DMAB | WT | IgG1 | G1m3 | TM/YTE | 19. DMAB |
| | | AZD1061CLIN HC | | | | | AZD1061CLIN_HC_TM_YTE |
| 61/62 | 2130(m3) | DMAB | WT | IgG1 | G1m3 | TM | 20. DMAb |
| | | AZD1061CLIN HC | | | | | AZD1061_HC_TM |
| 63/64 | 2130(m3) | DMAB | WT | IgG1 | G1m3 | WT | 21. DMAb |
| | | AZD1061CLIN HC | | | | | AZD1061_HC_WT |
| 65/66 | 2196(m3) | DMAb | WT | | G1m3 | WT | 22. DMAb |
| | | AZD8895CLIN_LC | | | | | AZD8895CLIN_LC |
| 67/68 | 2196(m3) | DMAb | WT | IgG1 | G1m3 | TM/YTE | 23. DMAb |
| | | AZD8895CLIN_HC | | | | | AZD8895CLIN_HC_TM_YTE |
| 69/70 | 2196(m3) | DMAb | WT | IgG1 | G1m3 | WT | 24. DMAb |
| | | AZD8895CLIN_HC | | | | | AZD8895CLIN_HC_WT |
| 71/72 | 2196(m3) | DMAb | WT | IgG1 | G1m3 | TM | 26. DMAb |
| | | AZD8895CLIN_HC | | | | | AZD8895CLIN_HC_TM.2 |
| 73/74 | 2130(m3) | DMAb | WT | IgG1 | G1m3 | YTE | 35. DMAb |
| | | AZD1061_HC_WT/YTE | | | | | AZD1061_HC_WT/YTE |
| 75/76 | 2196(m3) | DMAb | WT | IgG1 | G1m3 | YTE | 36. DMAb |
| | | AZD8895_HC_WT/YTE | | | | | AZD8895_HC_WT/YTE |
| 87/88 | | DMAb-2130_HC | | | | GA | |
| | | GA_pVax1 | | | | | |
| 89/90 | | DMAb-2130_HC | | | | ALIE | |
| | | ALIE_pVax1 | | | | | |
| 91/92 | | DMAb-2130_HC | | | | GAALIE | |
| | | GAALIE_pVax1 | | | | | |
| 93/94 | | DMAb-2130_HC | | | | SDAL | |
| | | SDAL_pVax1 | | | | | |
| 95/96 | | DMAb-2130_HC | | | | GASDAL | |
| | | GASDAL_pVax1 | | | | | |
| 97/98 | | DMAb-2130_HC | | | | GRLR | |
| | | GRLR_pVax1 | | | | | |

### Example 3: Additional DMAb Sequences

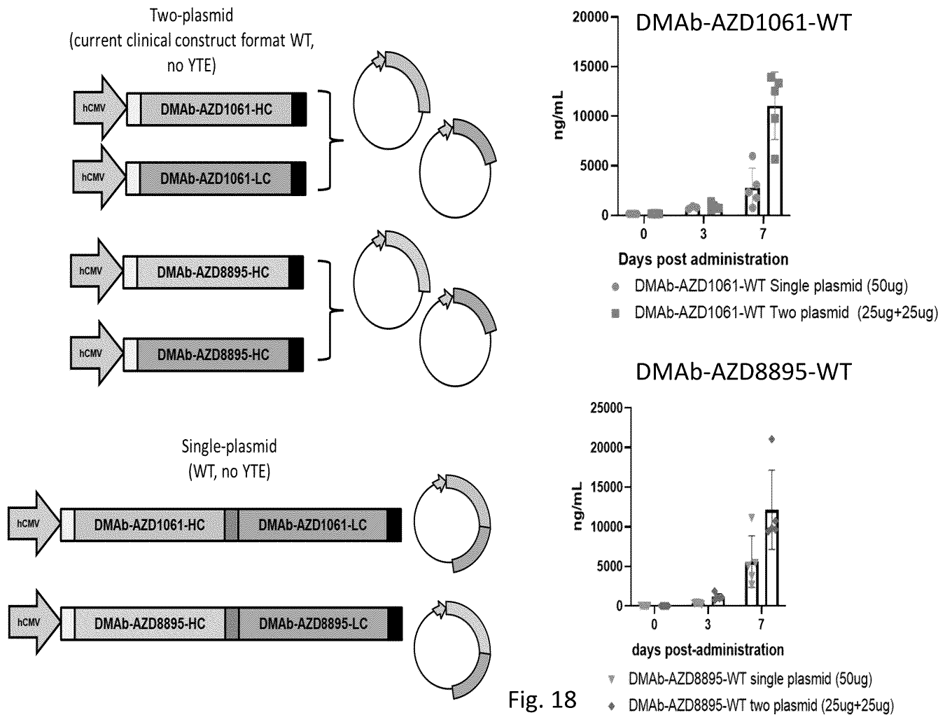

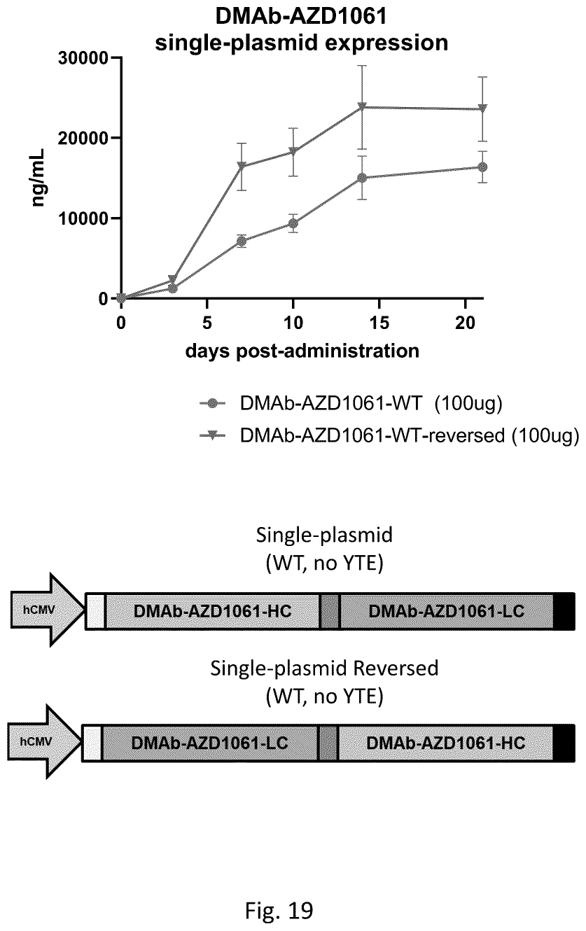

Reversal of the Heavy Chain and Light Chains increased expression using a single plasmid system. FIG. 18 and FIG. 19 provided data demonstrating the optimization of a single plasmid system through reversing the orientation of HC and LC (SEQ ID NO:79/SEQ ID NO:80).
2196 HC/LC (m1) (SEQ ID NO:81/SEQ ID NO:82)
2130 HC/LC (m1) (SEQ ID NO:83/SEQ ID NO:84)
2381 HC/LC (m1) (SEQ ID NO:85/SEQ ID NO:86)
SEQ ID NO:87 DMAb-2130_HC GA_pVax1
SEQ ID NO:88 DMAb-2130_HC GA_pVax1
SEQ ID NO:89 DMAb-2130_HC ALIE_pVax1
SEQ ID NO:90 DMAb-2130_HC ALIE_pVax1
SEQ ID NO:91 DMAb-2130_HC GAALIE_pVax1
SEQ ID NO:92 DMAb-2130_HC GAALIE_pVax1
SEQ ID NO:93 DMAb-2130_HC SDAL_pVax1
SEQ ID NO:94 DMAb-2130_HC SDAL_pVax1
SEQ ID NO:95 DMAb-2130_HC GASDAL_pVax1
SEQ ID NO:96 DMAb-2130_HC GASDAL_pVax1
SEQ ID NO:97 DMAb-2130_HC GRLR_pVax1
SEQ ID NO:98 DMAb-2130_HC GRLR_pVax1

### Example 4: DNA-Delivered SARS-CoV-2 Antibody Cocktail Exhibits Improved Pharmacokinetics, Confers Prophylactic Protection and Extends Structural Insight into Cooperativity

The COVID-19 pandemic highlighted the value of nucleic acid approaches for the timely development and large-scale deployment of life-saving vaccines. However, the versatility of such platforms extends beyond antigen delivery, potentially allowing the administration of biologically functional therapeutics. Here, DMAb technology was utilized to induce in vivo expression of validated anti-SARS-CoV-2 clones COV2-2196 and COV2-2130 and compared them to the biologic forms (Dong et al., 2021, Nat. Microbiol. 6, 1233-1244; Zost et al., 2020, Nature, 584:443-9; Zost et al., 2020, Nat Med, 26:1422-7). Pharmacokinetic studies conducted in both BALB/c and K-18 mice demonstrated that optimized expression was achieved with the dual plasmid system, resulting in higher peak DMAb serum titers and long-term expression exceeding 6 months. In a side-by-side evaluation, DMAbs exhibited prolonged kinetics relative to protein IgG which is a unique advantage of the DNA platform. Incorporation of the YTE modification, which improved the PK of clinical candidate AZD7442 in humans, also contributed to DMAb durability in hFcRn mice.

The molecular and functional profiles were characterized and it was found that the DMAbs were comparable to their protein counterparts both in vitro and in vivo (Dong et al., 2021, Nat. Microbiol. 6, 1233-1244; Zost et al., 2020), Nature, 584:443-9; Zost et al., 2020, Nat Med, 26:1422-7). The potency of in vivo-launched DMAbs against pseudotyped and infectious SARS-CoV-2 (USA-WA1/2020) was extremely high, with IC₅₀ values in the low ng/ml range as previously described (Zost et al., 2020, Nature, 584:443-9). In an AAV6.2FF-hACE-2 model, DMAbs administration reduced lung viral burden by 1-2 logs, a similar degree of control as achieved with a 200 μg dose of the recombinant mAbs in a similar Ad5-hACE2 model (Zost et al., 2020, Nature, 584:443-9). Moreover, prophylactic delivery of DMAbs 2196(TM) and 2196(TM), individually or in combination, conferred complete protection in a lethal mouse model and reduced viral burden in a hamster challenge model by >4 logs. Similar protection from disease and pathology was mediated by both WT(m3) and TM(m3) DMAb cocktails, indicating that Fc-mediated effector mechanisms are not detrimental to their function or safety profiles in these models. Rather, effector functionalities of antibodies may provide potential benefits in immune clearance, particularly at lower levels (Winkler et al., 2021, Cell, 184:1804-1820.e16; Suryadevara et al., 2021, Cell, 184:2316-2331.e15; Schäfer et al., 2020, J Exp Med, 218; Ullah et al., 2021, Immunity, 54:2143-2158.e15). Importantly, efficacy of the WT(m3) cocktail against lethal challenge was remarkably similar following protein or DNA-delivery, demonstrating equivalency of DMAbs in vivo. These combined strategies of sequence optimization, plasmid engineering and Fc modifications to enhance durability and potency could potentially lower the need for repeated drug delivery.

In addition to kinetic and functional evaluation, the first-ever structures of nucleic acid-delivered, in vivo-produced antibodies were produced. Overall, in vivo analysis supports many of the in vitro structural studies for their protein counterparts. Consistent with initial electron microscopy studies (Zost et al., 2020, Nature, 584:443-9), these complexes confirmed that DMAb 2130 can recognize its epitope regardless of RBD positioning (‘in’ or ‘out’) while DMAb 2196 is restricted to the “out” confirmation. Epitope chemistry of each DMAb in the 2130/2196 cocktail was characterized, recapitulating essential structural features/interactions previously defined in crystal structures of 2130/2196/RBD complexes (Dong et al., 2021, Nat. Microbiol. 6, 1233-1244). These include the formation of a hydrophobic cage around RBD residue F486 by DMAb 2196 involving both heavy and light chain contacts. DMAb 2130 demonstrated extensive interactions with key RBD residue K444 with additional interactions noted. Fab-to-Fab h-bonding was also observed between 2196 and 2130 light chains that supported potential interactions previously described (Dong et al., 2021, Nat. Microbiol. 6, 1233-1244).

Moreover, additional evidence was found that the two antibodies interact in vivo in a potentially cooperative fashion. High-resolution cryo-EM of the full trimeric 2130/2196/S complex revealed not only simultaneous binding of 2130 and 2196 to a single spike protein in vivo, but their concurrent binding to multiple spikes within a trimer. This allowed for the visualization and measurement of the proximity of bound DMAbs at nearly full trimer occupancy (5/6 binding sites). Measurements of physical distances support a basis for IgG-to-IgG interactions within and between spikes of the same trimer. These data revealed that the cocktail greatly benefits from cooperative binding effect via Fab-to-Fab and IgG-to-IgG interactions, helping to visualize mechanisms that explain their striking potency. This, combined with their potential to form compensatory interactions with highly mutated SARS-CoV-2 spike variants B.1.617.2 and B.1.1.529/BA.1, could explain their retained activity against all SARS-CoV-2 variants to date. Thus, a comprehensive understanding of the in vivo-produced 2196 and 2130 DMAb cocktail is provided that reveals broader insight into the properties of this valuable clinical mAb pair. Collectively, this rigorous interrogation of the DMAb approach supports its further development as a prophylactic/immunotherapeutic tool for SARS-CoV-2 and future pandemic preparedness.

### The Materials and Methods Used for the Experiments are Now Described

### DNA Expression Constructs (DMAbs)

The mature variable heavy (VH) and light (VL) domains of the selected mAb clones were optimized at the DNA and RNA levels. Synthetic inserts encoding the heavy chain (HC) and light chain (LC) genes for each clone were designed, containing a leader sequence(s) and the optimized VH or VL sequences followed by the corresponding constant domains (CH and CL, respectively) of wildtype human IgG1 (WT). These were inserted into a modified mammalian expression vector (pVax) under the human cytomegalovirus (hCMV) promoter between an IgG leader sequence and a bovine grown hormone (BGH) poly A signal using single or dual plasmid approaches. In single plasmid constructs (pHC/LC), matching genes were encoded in cis and separated by a porcine teschovirus-1 2A peptide/furin cleavage site. For dual plasmid systems, separate light chain plasmids (pLC) and heavy chain plasmids (pHC_WT) were generated for each clone. An additional HC variant, pHC_TM, was generated for selected clones containing a triple mutation (L234F/L235E/P331S) known to nullify effector functions of hIgG1.

### Mammalian Cell Culture and In Vitro Transfections

In vitro expression of DNA plasmids was performed in Expi293F™ suspension cells (Thermo Fisher Scientific; A14527). Cells suspension was maintained in Expi293™ Expression Medium (Thermo; A1435101) at 37° C./8% CO₂ conditions and transfected using the Expi293F™ Expression System Kit (Thermo; A14635). All transfection parameters (cell concentrations, culture volumes, DNA dilutions, incubation times, reagent preparations, etc.) were determined according to the manufacturer's guidelines. For in vitro transfection, cells were seeded in 6-well culture plates at 1×10⁶ cells/mL. HC/LC plasmid(s) encoding the indicated DMAbs were diluted in OPTI-MEM media (1 μg/mL; 1:1 ratio) and mixed with EpiFectamine transfection reagent. All constructs were tested in duplicate. DNA:lipid mixtures were incubated for 20 minutes at room temperature (RT) to allow for complex formation and then added, dropwise, to plated cells. Enhancers were added 18-22 hrs later, as instructed. Clarified culture supernatants were harvested via centrifugation 4-5 days post-transfection and stored at −20° C. prior to analysis.

### Animals, In Vivo DMAb Delivery and Sample Collection

Animal studies were performed in five-to-eight-week-old BALB/c, K-18 or hFcRn mice. Transgenic K-18 mice (B6.Cg-Tg (K18-ACE2) 2Prlmn/J; 034860; The Jackson Laboratory) express the gene for human angiotensin 1 converting enzyme (hACE2) in the airway epithelia under a human keratin 18 (KRT18) promotor and are susceptible to SARS-CoV-2 infection. hFcRn mice (B6.Cg-Fcgrt^{tm1Der}Tg (FCGRT) 32Dcr/DcrJ; 014565; The Jackson Laboratory) carry a knock-out mutation for mouse Fogrt and express the gene for human FCGRT under its native hTg32 promotor. This allows a more accurate evaluation of the in vivo kinetics of human IgG. All mice were purchased from certified vendors and housed in The Wistar Institute animal facility. All procedures were performed in accordance with the guidelines from the Wistar Institute Animal Care and Use Committee (IACUC) under approved protocols 201399 or 201464. For all DMAb administrations, 50-200 μg of total plasmid DNA was formulated in water supplemented with hyaluronidase (12 U/injection; Sigma) and injected into the tibialis anterior(s) and/or quadricep muscle(s). Injections were followed by the delivery of two 0.1 Amp electric constant current square-wave pulses by the CELLECTRA-3P electroporation device (Inovio Pharmaceuticals) to facilitate plasmid uptake. Recombinant 2196 and 2130 mAbs (100-200 μg/dose) were administered intraperitoneally. To prevent xenogenic responses against human DMAbs, T cell depletion (Anti-CD4/CD8 mAbs, 200 μg/mouse, given intraperitoneally) was performed at the time of plasmid/rIgG injection. For PK studies, sera were periodically collected via submandibular bleed to determine expression levels, durability and functionality. For bronchoalveolar lavage isolation (BAL), animals were euthanized and lungs were flushed with 900 μl of PBS supplemented with 0.05% NaN₃, 0.05% Tween-20, 2% 0.5M EDTA and protease inhibitor using a 20G blunt ended needle. BAL fluid was heat-inactivated for 20 min at 56° C. and stored at −20° C. prior to analysis. For efficacy studies, DMAb-treated mice were shipped to collaborators at Public Health Agency of Canada (PHAC) or transferred to BioQual, Inc. for challenge with SARS-CoV-2 (see SARS COV-2 Challenge methods below). Further experimental details for individual in vivo PK and efficacy studies are indicated in the appropriate Figure(s).

### Western Blot

Culture supernatants were probed by Western blot for human IgG expression and presence of the YTE Fc modification. Sample lanes on two identical NuPAGE™ 4-12% Bris-Tris gels (Thermo) were loaded with supernatants containing the indicated DMAbs (200 ng/lane based on ELISA quantification). All samples were reduced with NuPAGE™ Sample Reducing Agent (10×) (Thermo) for 10 minutes at 70° C. prior to loading. After gel electrophoresis, samples were transferred to PVDF membrane Immobilon-FL (EMD Millipore; IPFL07810) using iBlot™ 2 system (Thermo). Membranes were blocked in OBB (Odyssey® Blocking Buffer; LI-COR) for 1 hour and washed with PBS-T (1% Tween-20) and probed with the indicated antibodies. The first gel was probed with mouse anti-beta actin IgG (Sigma; A5316-1000UL; diluted 1:5000 in OBB) as a loading control for 1 hour at RT and washed. hIgG DMAbs were visualized using Goat Anti-hIgG-IRDye-800CW secondary antibody (LI-COR; diluted 1:10,000 in OBB) and bound mouse anti-beta actin was detected with anti-mouse IgG-IRDye-680RD (LI-COR; diluted 1:10,000 in OBB). The second gel was also probed with mouse anti-beta actin IgG as a loading control as well as a rabbit anti-YTE IgG monoclonal antibody (AstraZeneca; diluted 1:5000 in OBB) to detect HCs containing the YTE modification for 1 hour at RT and washed. YTE-containing HCs and beta actin were visualized with Goat anti-rabbit IgG-IRDye-RD680 (LI-COR; diluted 1:10,000 in OBB) and Goat anti-mouse IgG-IRDye-RD680 (LI-COR; diluted 1:10,000 in OBB), respectively, for 1 hour at RT. Finally, membranes were washed three times and scanned using Odyssey® CLx Imager (LI-COR).

### IgG Quantification (Anti-Human IgG ELISA)

For quantification of DMAb in culture supernatants, NUNC 96-well MaxiSorp plates (Sigma; M9410-ICS) were coated with 5 μg/ml goat anti-human IgG-Fc (Bethyl; A80-104A) diluted in 1×PBS overnight at 4° C. The following day, plates were washed 4 times with 0.05% PBS-T and were blocked with 5% non-fat dry milk in PBS for 1 hr at room temperature (RT). Plates were washed and incubated with duplicate samples, diluted in 1% newborn calf serum (NCS) in 0.2% PBS-T for 1 hr at RT. Plates were washed and incubated with 1:10000 HRP-conjugated goat anti-human IgG-Fc (Bethyl; A80-104P) diluted in 1% NCS in 0.2% PBS-T for 1 hr at RT. Finally, washed plates were developed with 1-Step™ Ultra TMB-ELISA Substrate Solution (Thermo; 34028) and quenched with 2N H₂SO₄. Plates were read at 450 nm on the BioTek Synergy 2 (Biotek) plate reader. Blank wells were included on each plate and subtracted as background. Purified human IgG (Bethyl; P80-112) was used to create a standard curve for quantification (μg/mL). Positive control sample was included on each plate and used to standardize values across assays. Data were subsequently exported to Microsoft Excel and analyzed using GraphPad Prism 9. Negative OD values (following background correction) were represented by zero for graphing purposes.

### IgG Epitope Specificity/Variant Recognition (Antigen-Binding ELISAs)

Binding ELISAs were used to confirm the epitope specificities of DMAbs 2130, 2196 and 2381. NUNC 96-well MaxiSorp plates were coated with recombinant RBD proteins (3 μg/mL in 1×PBS) containing mutations at residues F444A (RBD-F444A) or F486 (RBD-F486A) (AstraZeneca), which are key residues required for the binding of clones 2130 and 2196/2381, respectively. To evaluate the relative binding of each construct to different VoC, the following coating antigens were used (0.5-1 μg/mL in 1×PBS); SARS-CoV-2 Spike RBD-His Recombinant Protein (Sino Biologicals; 40592-V08B), Spike S1 (D614G)-His Recombinant Protein (Sino Biologicals; 40591-V08H3), RBD-His K417N Recombinant Protein (Sino Biologicals; 40592-V08H59), RBD-His E484K Recombinant Protein (Sino Biologicals; 40592-V08H84), RBD-His N501Y (Sino Biologicals; 40592-V08H82), Spike S1-K417N/E484K/N501Y/D614G Recombinant Protein (Sino Biologicals; 40591-V08H10), B.1.1.529 (BA.1) S1+S2 Trimer-His Recombinant Protein (Sino Biologicals; 40589-V08H26). ELISA procedure was completed as described above (see IgG Quantification section).

### ACE-2 Inhibition Assays

An established ACE-2 inhibition assay was performed (Walker et al., 2020, J. Clin. Microbiol, 58, e01533-20). Briefly, the ability of biotinylated, recombinant ACE2-IgHu to bind plate-bound SARS-CoV-2 RBD protein in the presence of the indicated DMAb(s) was determined. 96-well Flat-Bottom Half-Area plates (Corning) were coated at room temperature for 8 hours with 1 μg/mL 6×-His tag polyclonal antibody (Thermo; PA1-983B) followed by overnight blocking with blocking buffer containing 5% milk/1×PBS/0.01% Tween-20 at 4° C. The plates were then incubated with RBD at 1 μg/mL at room temperature for 1-2 hours. Sera harvested from DMAb-treated mice either were serially diluted 3-fold starting at 1:20 with dilution buffer (5% milk/1×PBS/0.01% Tween-20), added to the plate and incubated at RT for 1-2 hrs. Human Angiotensin-converting enzyme 2 (ACE2-IgHu) antibody was biotinylated using Novus Biologicals Lightning-Link rapid type A Biotin antibody labeling kit (NovusBio; 370-0010) according to protocol. The biotinylated ACE2-IgHu was added to wells at a constant concentration of 0.5 g/ml diluted with the dilution buffer and incubated at RT for 1 hour. The plates were further incubated at room temperature for 1 hour with native streptavidin-HRP (Abcam; ab7403) at 1:15,000 dilution followed by addition of TMB substrates (Thermo), and then quenched with 1M H₂SO₄. Absorbances at 450 nm and 570 nm were recorded with a BioTek plate reader. Four washes were performed between every incubation step using PBS and 0.05% Tween-20. The assay was performed in triplicates and the average of the absorbance value was determined. The average absorbance of the lowest dilutions with saturating ACE2 signals was calculated to get a maximum ACE2 binding and no blocking. Each average absorbance value was subtracted from the maximum to get an ACE2 blocking curve. The blocking titer is defined as the reciprocal of the highest dilution where two consecutive dilutions have readings below zero. The maximum area under the curve is determined by calculating the Area Under the Curve (AUC) of full ACE2 binding without the competitor. The AUC of the competitor is then subtracted from the maximum AUC to get the area between the curves (blocking Area) and is the measure of ACE2 blocking. The fraction ACE2 blocking is defined as the fraction of the blocking area to the maximum AUC.

### Neutralization Assay: Pseudotyped Virus

HEK293T were maintained in DMEM supplemented with 10% fetal bovine serum (FBS) and 1% penicillin streptomycin (P/S) antibiotic in 37° C./5% CO2 conditions. To create SARS-CoV-2 pseudoviruses, Gene jammer (Agilent) was used to transfect cells with 1:1 ratio of pNL4-3. Luc. R-E-plasmid (NIH AIDS reagent) along with various of synthetic plasmids (Genscript) expressing the wildtype spike protein (derived from isolate USA-WA1/2020) or mutated spikes derived from variants B.1.1.7 (Alpha), B.1.351 (Beta), P.1 (Gamma), B.1.617.2 (Delta) or B.1.526 (Iota), B.1.1.529/BA. 1 (Omicron sublineage BA.1), B.1.1.529/BA.2 (Omicron sublineage BA.2). Forty-eight hours post-transfection, culture supernatants were collected, enriched with FBS to 12% final volume, and stored at −80° C. SARS-CoV-2 pseudovirus neutralization assays were established using huCHOAce2 cells (Creative Biolabs; VCeL-Wyb019) plated in a 96-well plate format. Cells were resuspended in D10 media (DMEM supplemented with 10% FBS and 1× Penicillin-Streptomycin), plated (10,000 cells/well) and rested overnight in 37° C./5% CO₂ conditions. The following day, transfection supernatant or sera from DMAb-treated animals were heat-inactivated and serially diluted in duplicate as desired. Supernatant from non-transfected cells or sera from naïve animals served as controls, respectively. Diluted samples were incubated with the indicated SARS-CoV-2 pseudovirus for 90 minutes at RT and then transferred to rested huCHOAce2 cells. Plates were incubated in 37° C./5% CO2 conditions for 72 hrs and then lysed using the britelite plus luminescence reporter gene assay system (Perkin Elmer; 6066769). RLUs were measured using the Biotek plate reader. Using GraphPad Prism 9, nonlinear regressions were applied to duplicate RLU values for each sample to determine the best fit line. Neutralization titers (ID50) were then calculated, defined as the reciprocal dilution that yielded a 50% reduction in RLU compared to sample control wells; RLUs from cell-only control wells on each plate were subtracted as background prior to analysis. To assess the relative activity against mutant pseudoviruses, the same dilution series was tested in parallel against the indicated variants. The calculated ID50s were used to calculate a fold change relative to WA1/2020 (ID50_{WA1/2020}/ID50ᵥₐᵣᵢₐₙₜ). ID50s for each sample were also used along with the corresponding DMAb titer (ng/ml) to calculate inhibitory concentrations (IC50s=DMAb titer/ID50) that reflect the individual molecular potency of a test sample while controlling for expression levels.

### Neutralization Assay: Authentic SARS-CoV-2 Viruses

Live SARS-Related Coronavirus 2, Isolate USA-WA1/2020, was obtained through BEI Resources (NIAID, NIH; NR-52281) and contained within the BSL-3 facility at the Wistar Institute. Vero cells (ATCC; CCL-81) were maintained in DMEM supplemented with 10% fetal bovine serum (FBS). Viral propagation and titration were achieved as previously described. Briefly, the USA-WA1/2020 virus stock was serially diluted in DMEM with 1% FBS and transferred in replicates of 8 to previously seeded Vero cells and incubated for five days under 37° C./5% CO2 conditions. Individual wells were then scored positive or negative for the presence of cytopathic effect (CPE) by examination under a light microscope. The virus titer (TCID50/mL) was calculated using the Reed-Munch method and the published Microsoft Excel-based calculator. For neutralization assays, Vero cells were seeded in DMEM with 1% FBS at 20,000 cells/well in 96 well flat bottom plates and incubated overnight. Samples were heat-inactivated at 56° C. for 30 minutes and then serially diluted in triplicates. These were incubated for Ihr at RT with 300 TCID50/mL of virus before the mixture was transferred to previously seeded Vero cells and incubated for 5 days. Neutralizing titers and inhibitory concentration (ID50 and IC50) were determined as described above.

SARS-CoV-2 Challenge: hACE2-AAV model (murine)

Female BALB/C mice (n=10/group) were treated with the indicated DMAb plasmid formulations (200 μg/mouse; see Animals and in vivo DMAb delivery methods) and shipped to PHAC for evaluation using the previously validated hACE2-AAV model (Gary et al., 2021, IScience, 24:102699). All procedures were performed in accordance with PHAC-approved protocols. Mice were anesthetized with isoflurane 14 days post-plasmid delivery and administered 1×10¹¹ viral copies of AAV6.2FF-hACE2 intranasally (50 μL) to facilitate expression of hACE2 in the lungs of recipient mice. Two weeks later (D21 post-plasmid delivery), mice were given an intranasal challenge with 1×10⁵ TCID50 (50 μL) of SARS-CoV-2 virus (hCoV-19/Canada/ON-VIDO-01/2020; GISAID #EPI_ISL_425177). Controls include a group of non-AAV transduced animals (insusceptible; negative control) and a group of AAV-transduced/non-DMAb treated animals (susceptible; positive control). Following challenge, animals were monitored daily for signs of clinical disease and euthanized 4 days post-infection, at which time lung tissue was collected for viral quantification and blood was collected for evaluation of DMAb levels. Levels of viral RNA (copies/g lung tissue) for each animal were determined were determined via qPCR (Gary et al., 2021, IScience, 24:102699).

### SARS-CoV-2 Challenge: Lethal K-18 Model (Murine)

Male or female K-18 mice (n=12/group) were treated with the indicated DMAb plasmid formulations (200 μg/mouse; see Animals and in vivo DMAb delivery methods) and transferred to BioQual Inc. for evaluation in a lethal SARS-CoV-2 challenge model. Baseline sera samples and body weights were collected prior to challenge. Mice were anesthetized and intranasally (50 μL) infected with 2.8×10³ PFU (SARS-CoV-2/human/USA/WA-CDC-WA1/2020USA_WA1/2020; GenBank Acc: MN985325) (BioQual, Inc.). Animals were monitored daily following challenge for clinical signs of disease (visual scoring, weight-loss, etc.); euthanasia criteria included moribund scoring and/or weight-loss of >20% (vs. pre-challenge starting weight). At D4 post-challenge, a subset of each group (n=4) was sacrificed to assess viral titers in the lungs and nasal turbinates of challenged mice via a validated TCID50 assay (BioQual, Inc.). Left lung was collected and placed in 10% neutral buffered formalin for histopathologic analysis. Tissues were processed to hematoxylin and eosin (H&E) stained slides and examined by a board-certified pathologist. Gross and microscopic scoring was conducted, using the following scale that reflects the intensity and pervasiveness of observed histopathological change: Grade 1 (1+): minimal, <10%; Grade 2 (2+): mild, 10-25%: Grade 3 (3+): moderate, 25-75%; Grade 4 (4+): marked, 75-95%: Grade 5 (5+): severe >95%. Additional experimental details individual K-18 challenges are provided in the appropriate figure legend(s).

### SARS-CoV-2 Challenge: Hamster Model

Syrian golden hamsters (n=6/group) were administered the WT(m3) DMAb cocktail (1:1 ratio of DMAb 2130_FcWT(m3)+DMAb 2130_FcWT(m3); 1.6 μg total) or TM(m3) DMAb cocktail (1:1 ratio of DMAb 2130_FcTM(m3)+DMAb 2130_FcTM(m3); 1.6 μg total) intramuscularly followed by CELLECTRA-EP. To prevent xenogenic responses against human DMAbs, T cell depletion (500 μL of 0.7 mg/mL anti-CD4/CD8 mAbs per hamster, given intraperitoneally) was performed 3 days prior to plasmid injection. 18 days post-DMAb delivery, sera were collected and animals were challenged intranasally with SARS-CoV-2 (USA-WA1/2020; 6000 PFU) (BioQual, Inc.). Hamsters were weighed over time and sacrificed 4 days post-challenge (D22) for analysis of viral load in the lung and nasal turbinate tissues via a validated TCID₅₀ assay (BioQual, Inc.).

### Cryo-Electron Microscopy

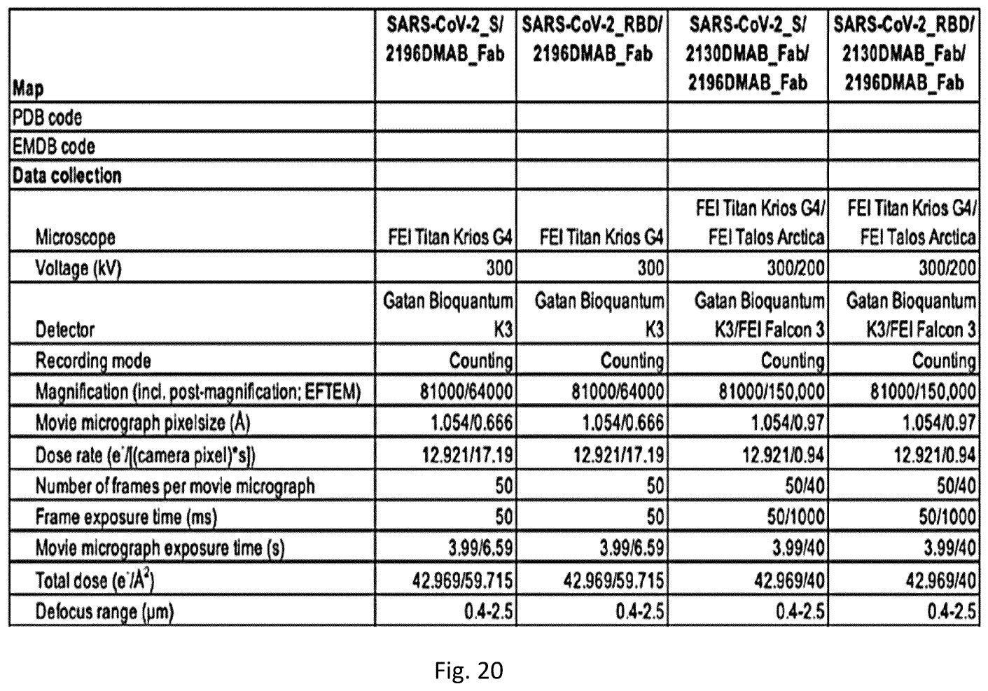

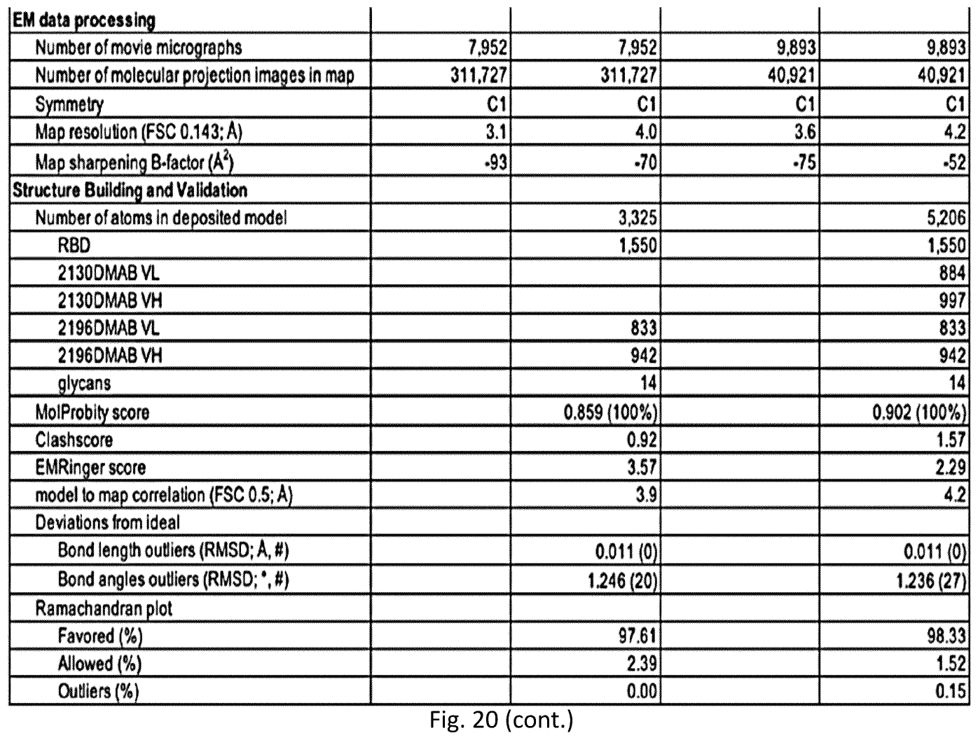

Serum IgG was recovered from mice that had been administered constructs for in vivo production of either 2196 DMAb or a cocktail of 2130 and 2196 DMAbs. Serum IgG was digested with papain (Sigma; P3125) and Fab was recovered. SARS-CoV-2 6P spike ectodomain peplomers (SARS-CoV-2/human/USA/WA-CDC-WA1/2020USA_WA1/2020; GenBank Acc: MN985325) were expressed in expi293 culture (Gibco) and affinity purified via a double strep tag followed by gel filtration using a 10/300 S6I column (Cytiva). Fab and spike peplomer were incubated on ice and complexes purified by S6I gel filtration. Complexes were concentrated in centrifugal filters (Amicon) and vitrified on 1.2/1.3 gold cryo-electron microscopy grids (Protochips) by use of a Vitrobot Mark IV (Thermo). EFTEM data was collected using a Titan Krios G4 instrument (Thermo) equipped with a Bioquantum K3 detector (Gatan) in electron counting mode. A subset of TEM data was collected on a Talos Arctica equipped with a Falcon 3 detector (Thermo) (FIG. 20). Data collection was automated by use of EPU in AFIS mode (Thermo Fisher Scientific). Dose fractionated movies were recorded of each of the two samples; 7,952 movies of Spike/2196DMAb_Fab and 9,893 movies of Spike/2130DMAb_Fab/2196DMAb_Fab. The former data was recorded at nominal magnification of 81,000× (Krios/K3) or 150,000× (Arctica/F3); the latter data at either 81,000× or 64,000× (super resolution). The Spike/2196 DMAb_Fab data treatment was performed in Relion 3.1.2 (Scheres, 2012, Struct. Biol, 180:519-530). Movie frame alignment and weighted integration (Relion) were followed by CTF estimation (ctffind4) (Rohou et al., 2015, J. Struct. Biol, 192:216-221). LoG picking was followed by 2D image classification. Suitable 2D classes were identified and underlying molecular projection images were selected for further processing. A low-pass filtered in-house unliganded SARS-CoV-2 density map was used for initial Euler angle assignment and 3D refinement was conducted. CTF refinement, beam tilt correction and Bayesian polishing were performed resulting in a global density map with a resolution of 3.1 Å (FSC 0.143 criterion). The global density map has two RBDs in the ‘in’ position and one in the ‘out’ position; only the ‘out’ position is occupied by one copy of 2196DMAb Fab. Density subtraction and local refinement focusing on the ‘out’ RBD/2196DMAb Fab was performed (Bai et al., 2015, Elife, 4:e11182; Pallesen et al., 2017, Proc. Natl Acad. Sci. USA, 114:E7348-E7357) resulting in a local density map with a resolution of 4.0 Å (FSC 0.143 criterion). The Spike/2130DMAb_Fab/2196DMAb_Fab data was processed in similar fashion using cryosparc (Punjani, et al., 2017, Nat. Methods, 14:290-296). The global density map has all three RBDs in the ‘out’ position and each RBD is bound by one copy of 2130DMAb_Fab and one copy of 2196DMAb_Fab. Resolution of this global density map is 3.6 Å and resolution of the local density map is 4.2 Å (FSC 0.143 criterion). Model building was initiated using the SARS-CoV-2 Spike RBD from 7E23. Models of 2130 Fab and 2196 Fab were obtained using Rosetta's antibody application (Weitzner et al., 2017, Nat. Protoc, 12:401-416) (Chotia numbering); models were adjusted and CDR loops rebuilt manually in Coot (Emsley et al., 2004, Acta Crystallogr. D. Biol. Crystallogr, 60:2126-2132) guided by the local density maps. Atomic models were refined with Rosetta FastRelax and geometry evaluated with molprobity online analysis (Chen et al., 2010, Acta Crystallogr. Sect. D. Biol. Crystallogr, 66:12-21), fit-to-map evaluated with command line implementation of EMRinger (Barad et al., 2015, Nat. Methods, 12:943-946) and glycan geometry evaluated with Privateer (Agirre et al., 2015, Nat. Struct. Mol. Biol, 22:833-834). Hydrogen bonding patterns were identified using hbonds in UCSF Chimera (Pettersen et al., 2004, J. Comput. Chem, 25:1605-1612).

### Statistical Analysis

Statistical analyses were conducted using GraphPad Prism 9 software. Nonparametric tests were performed due to small group sizes. Mann-Whitney U test was used when comparing means of two groups and Kruskal-Wallis nonparametric rank-sum test followed by Dunn's post hoc analysis were conducted to compare three or more groups. Survival curves were analyzed using Mantel-Cox log-rank test tests. In all cases, P values <0.05 were considered significant and denoted as * P<0.05, **P<0.01, ***P<0.001, and ****P<0.0001 where applicable.

### The Experimental Results are Now Described

Plasmid Optimization Combined with Fc-Engineering Induces the In Vivo Production of Functionally Potent 2130- and 2196-Based DMAbs

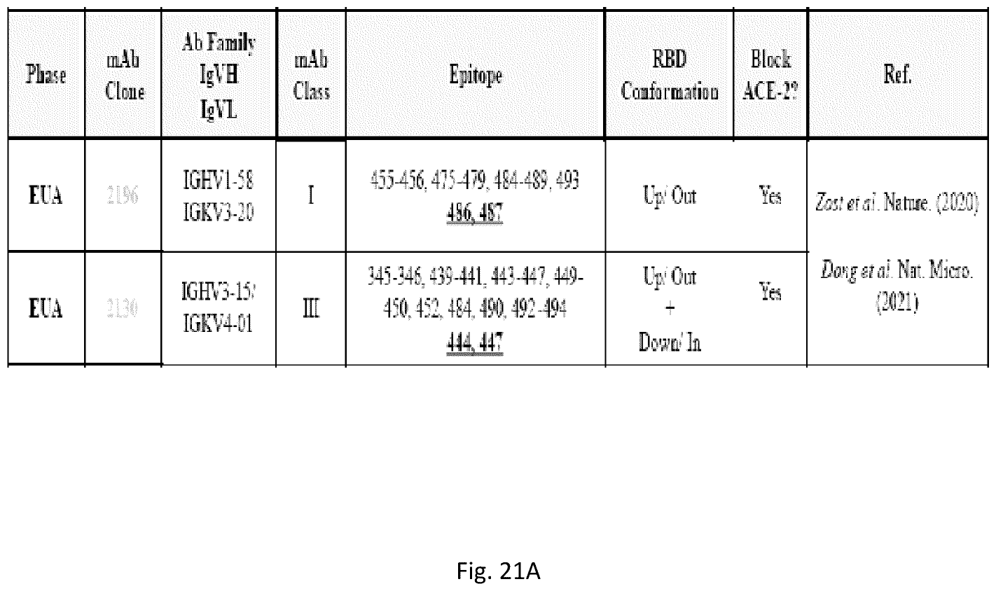

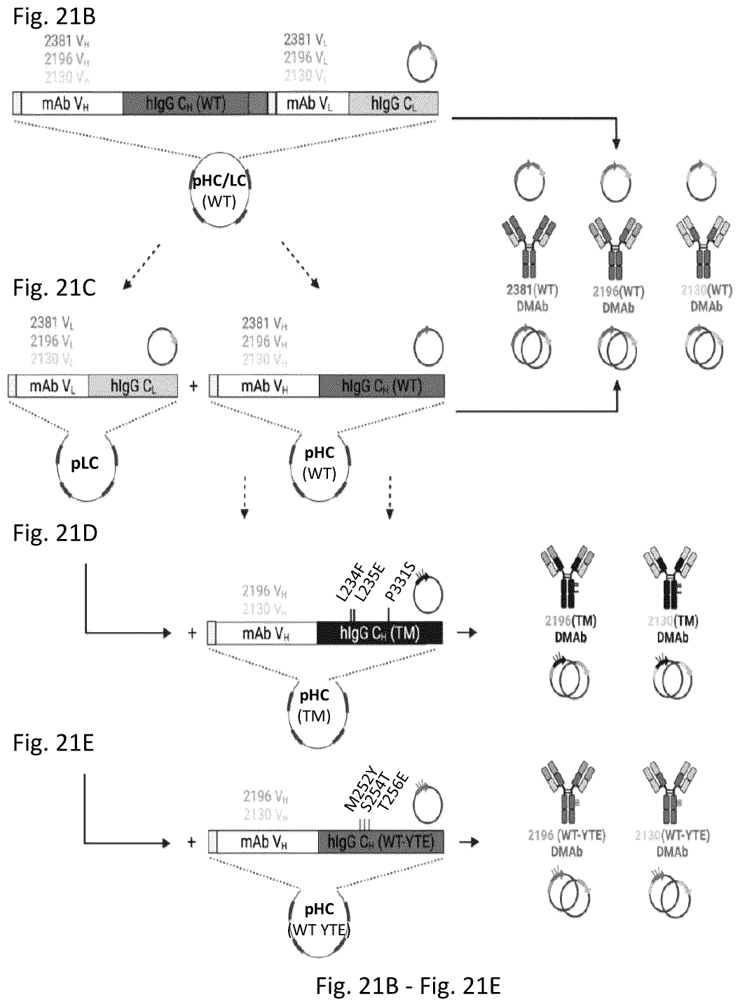

Anti-SARS-CoV-2 mAb pair COV2-2196 (class I) and COV2-2130 (class III) are human neutralizing Abs (nAbs) that target non-redundant, complementary epitopes within the receptor binding domain of SARS-CoV-2 spike protein (S-RBD). Both epitopes overlap with the ACE-2 binding site to mediate viral neutralization (FIG. 21A) (Dong et al., 2021, Nat. Microbiol., 6:1233-1244; Zost et al., 2020, Nature 584:443-449). They exhibit high antiviral potency and were effective in multiple preclinical SARS-CoV-2 challenge models (Loo et al., 2022, Sci. Transl. Med. 14:ABL8124; Chen et al., 2021, Nature, 596:103-108; Zost et al., 2020, Nature 584:443-449). DMAb constructs were designed using publicly available sequences for the variable heavy (VH) and light (VL) domains of mAbs 2130, 2196 and an additional clone, COV2-2381 (2381) (Zost et al., 2020, Nature 584:443-449). These were DNA and RNA optimized to promote in vivo transcript production/processing and inserted, along with a wildtype human IgG1 framework (WT), into a verified custom mammalian expression vector using single or dual plasmid systems (FIG. 21B-C).

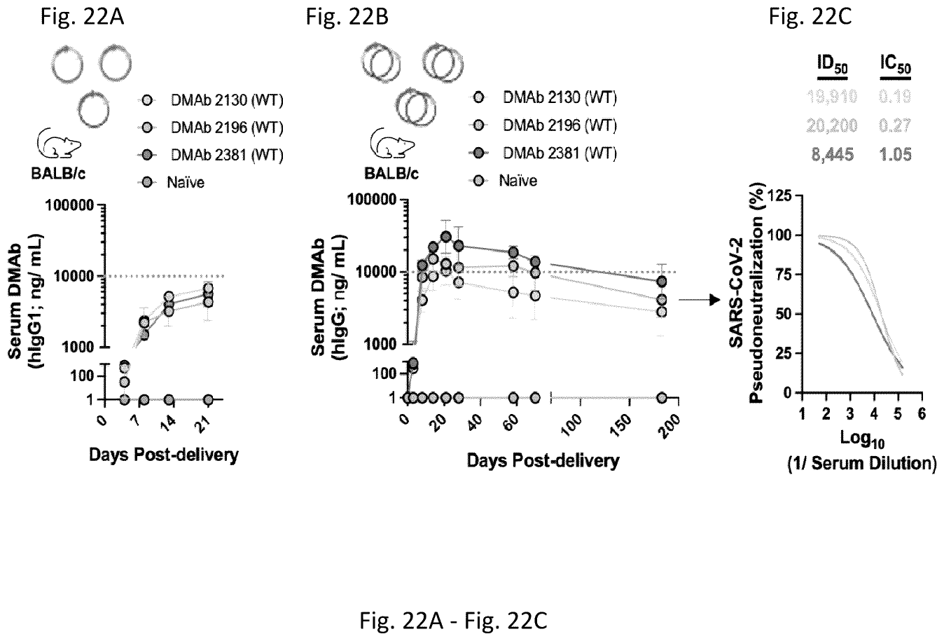

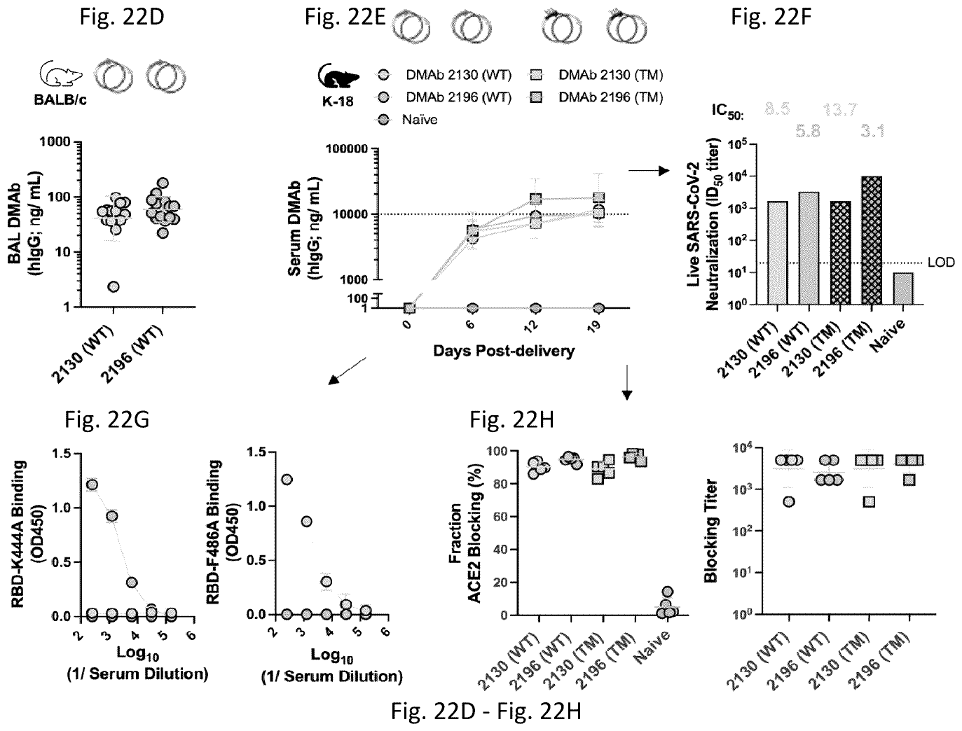

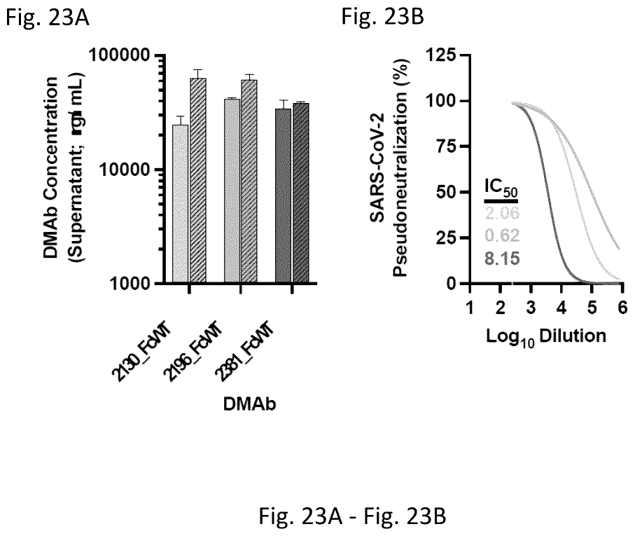

Studies were conducted to determine the relative expression profiles of single vs dual plasmid constructs following facilitated in vivo delivery to wildtype mice via intramuscular injection and electroporation (CELLECTRA-EP) (Broderick et al., 2014, Expert Review of Vaccines, 14:195-204) (FIG. 22A-B). Consistent with in vitro studies (FIG. 23), dual-plasmid in vivo-delivery led to a 2-4× increase in peak serum DMAb levels compared to single plasmid constructs. These were maintained for at least 6 months post DMAb administration (FIG. 22B) and displayed potent antiviral activity against SARS-CoV-2 pseudotyped virus (USA-WA1/2020) (FIG. 22C). DMAbs 2196 (WT) and 2130 (WT) were also detected in the bronchoalveolar lavage (BAL) collected from parallel groups of mice at D14 post-administration, indicating that in vivo-launched DMAbs are present the site of infection prior to challenge (FIG. 22D).

In addition to WT constructs, variants of each DMAb were generated that contain triple-residue modifications (“TM”; L234F/L235E/P331S) in the Fc domain that ablate FcR and C1q binding as found in AZD7442 (FIG. 21D) (Oganesyan et al., 2008, Acta Crystallogr. Sect. D Biol. Crystallogr., 64:700-704). Corresponding Fc variants showed strong and similar expression profiles in vivo (FIG. 22E) and comparable activity against authentic SARS-CoV-2 (USA-WA1/2020) virus (FIG. 22F). The epitope specificity of each DMAb construct was confirmed using modified RBDs containing point mutations K444A and F486A which abrogate binding of mAb clones 2130 and 2196/2381, respectively (FIG. 22G) (Dong, J. et al. 2021, Nat. Microbiol. 6:1233-1244; Zost et al., 2020, Nature 584:443-449). An established ACE-2 inhibition assay was used to demonstrate the ability of in vivo-launched DMAbs to efficiently block the binding of spike to hACE-2 (FIG. 22H) (Walker et al., 2020, J. Clin. Microbiol., 58).

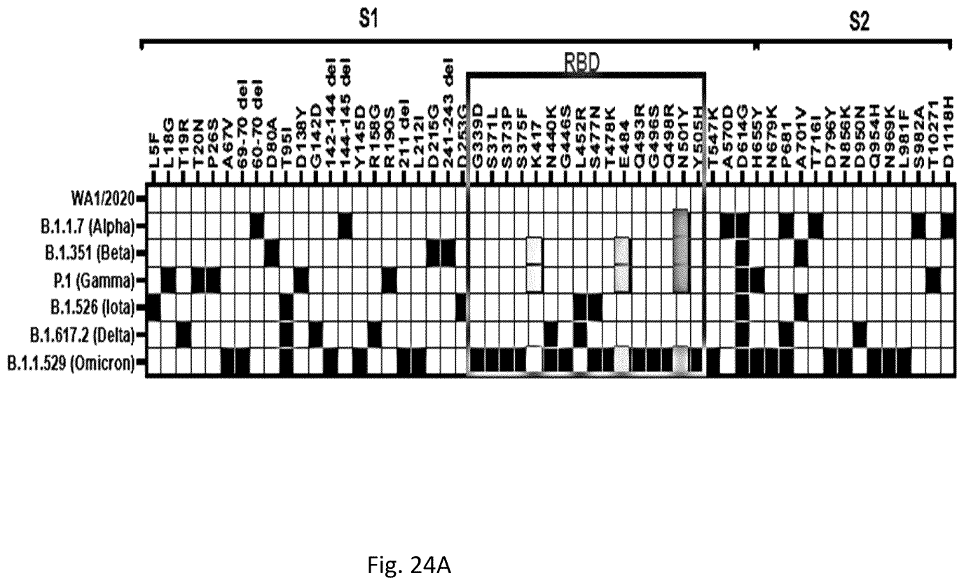

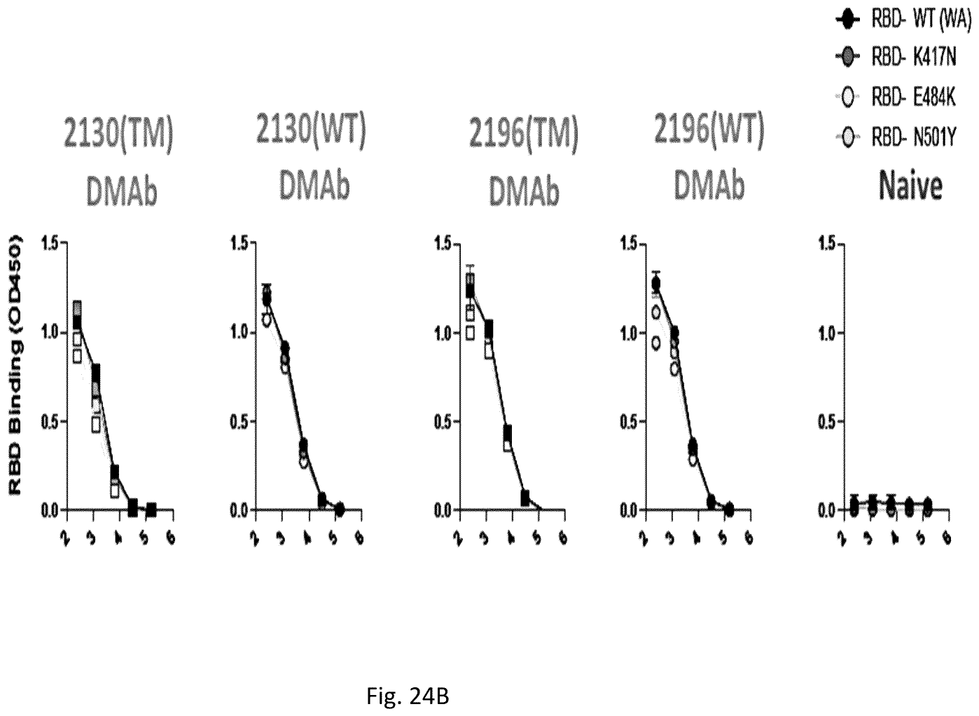

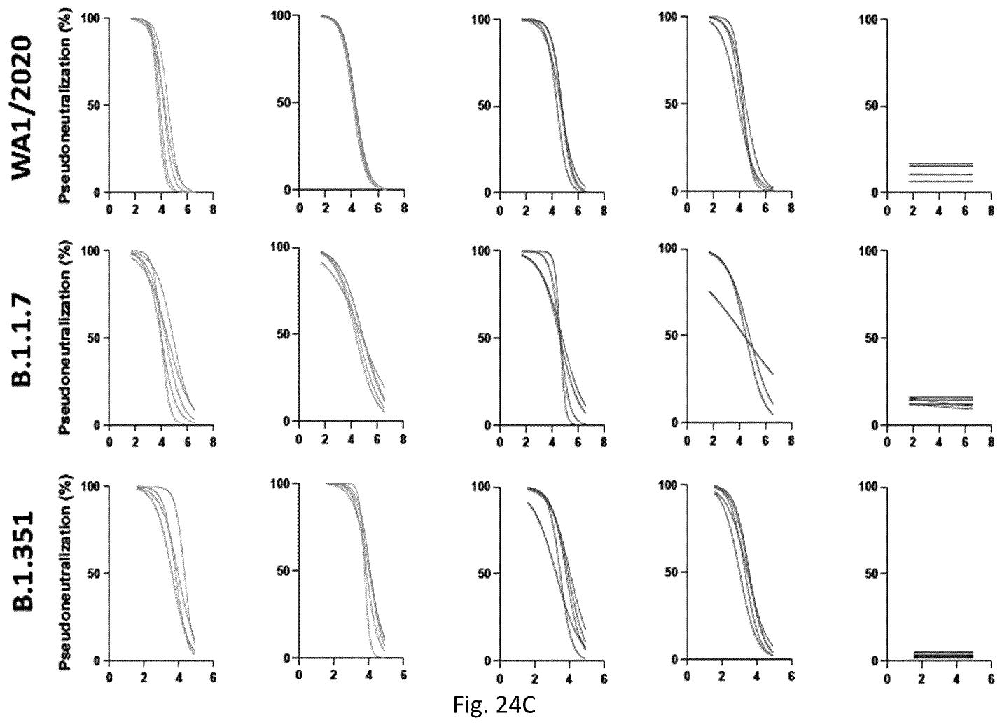

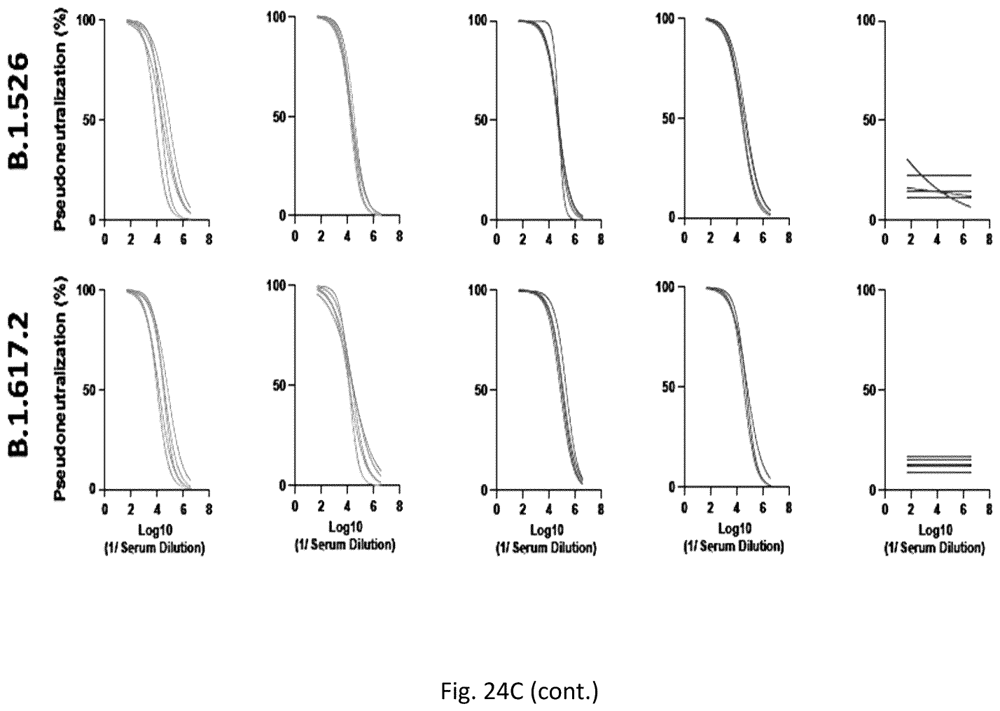

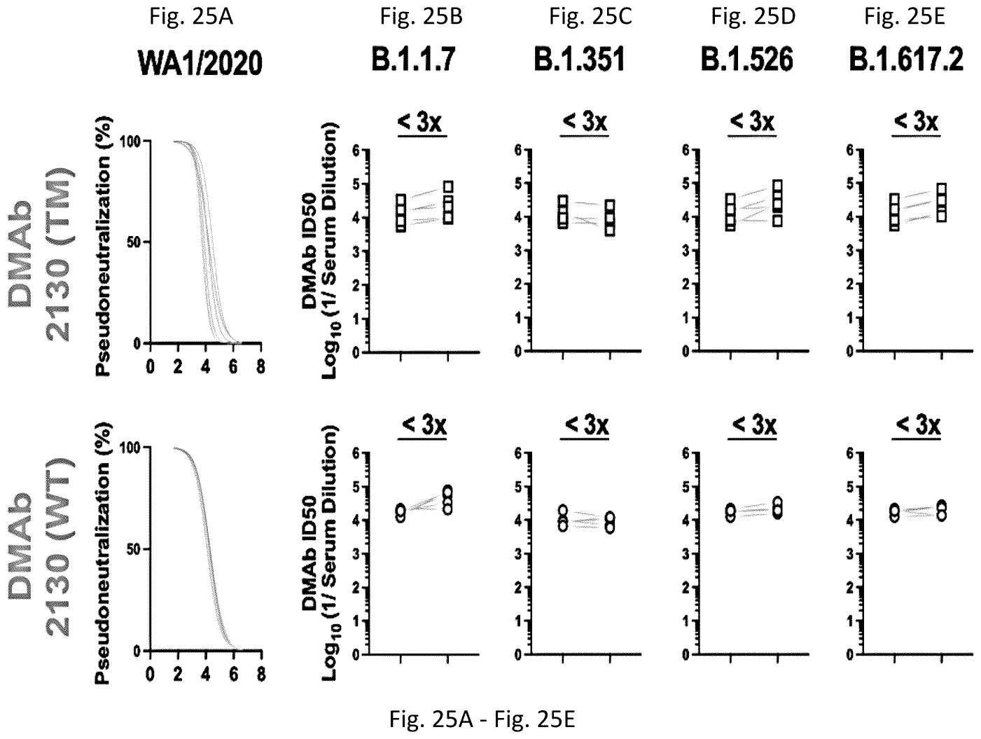

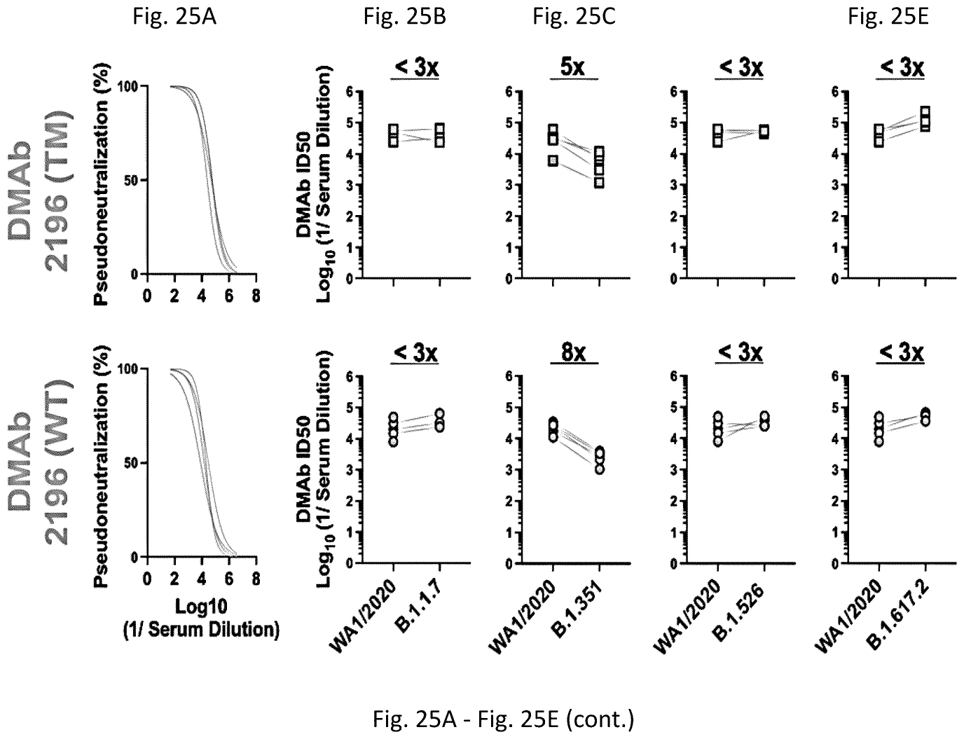

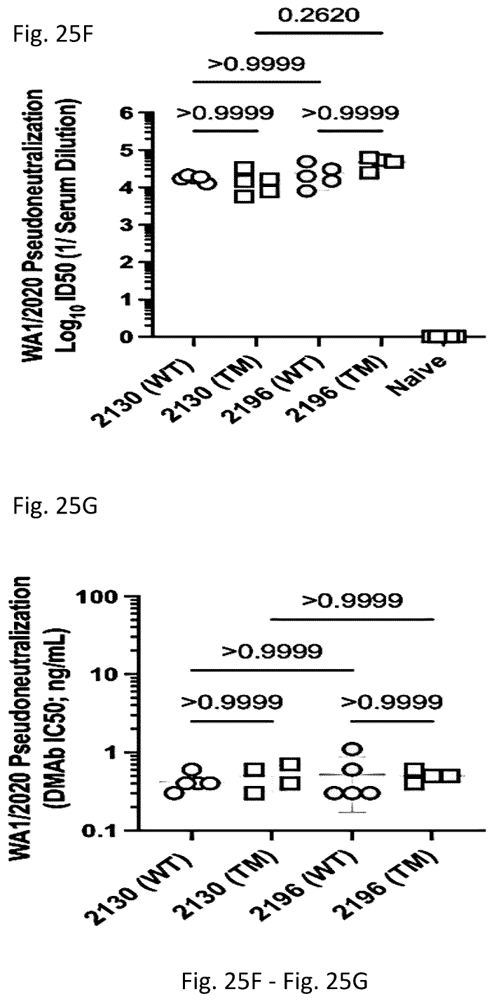

Numerous SARS-CoV-2 lineages bearing mutations in the spike protein have emerged, including B.1.1.7/alpha (Rambaut, A. et al., 2020, Virological.org 1-9), B.1.351/beta (Tegally, H. et al. 2021, Nature 592:438-443), P.1/gamma (Naveca et al., 2021, Virological.org 28:1-6), B. 1.526/iota (Annavajhala et al., 2021, Nature 597:703-708), B.1.617.2/delta (European Centre for Disease Prevention and Control. 2021, Threat assessment brief: emergence of SARS-CoV-2 B.1.617 variants in India and situation in the EU/EEA) (FIG. 24A). Mutations in S-RBD are more likely to interrupt binding by neutralizing mAbs and confer therapeutic resistance (Starr et al., 2021, Science, 371:850-85; Greaney et al., 2021, Cell Host Microbe, 29:44-57.e9). The relative binding of these DMAbs to mutant RBDs was evaluated compared to the parental RBD (USA-WA1/2020) (FIG. 24B). 2130-based DMAbs retained similar recognition of all mutant RBDs while 2196-derived DMAbs showed modest reduction in binding to the E484K single RBD mutant. Consistent with binding assays (FIG. 24B), 2130-based DMAbs were equally potent against these early pseudotyped virus variants (<3-fold reduction in ID50) (FIG. 25A-E). 2196-derived DMAbs demonstrated mild (5-to-8 fold) reduction in activity against B.1.351 while retaining activity against other major variants (FIG. 25A-E). Reproducible serum potency of 1-2 ng/ml was observed in all individual sera samples, which was unaffected by the Fc framework (WT vs TM) (FIG. 25F-G and FIG. 24C).

### DMAb Prophylaxis Protects Mice Against SARS-CoV-2 (USA WA1/2020) Lethal Challenge

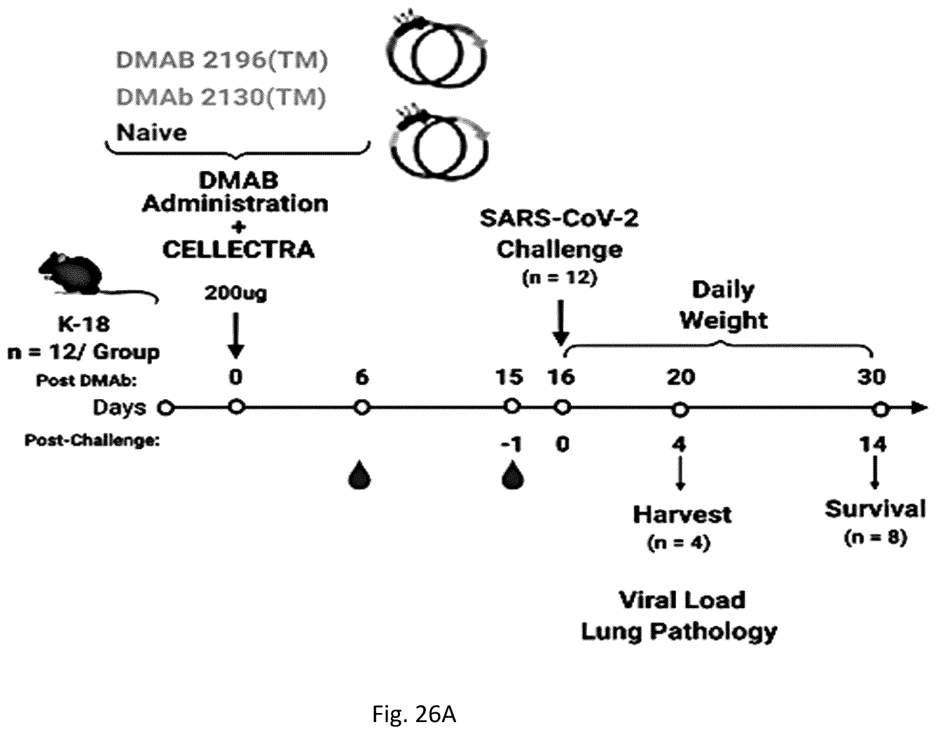

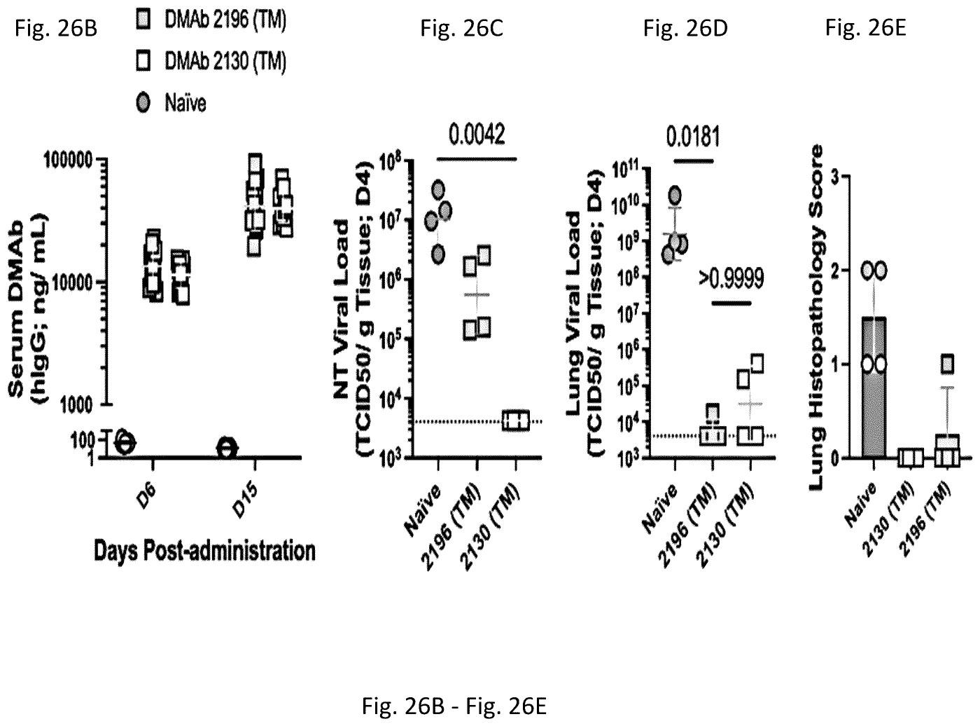

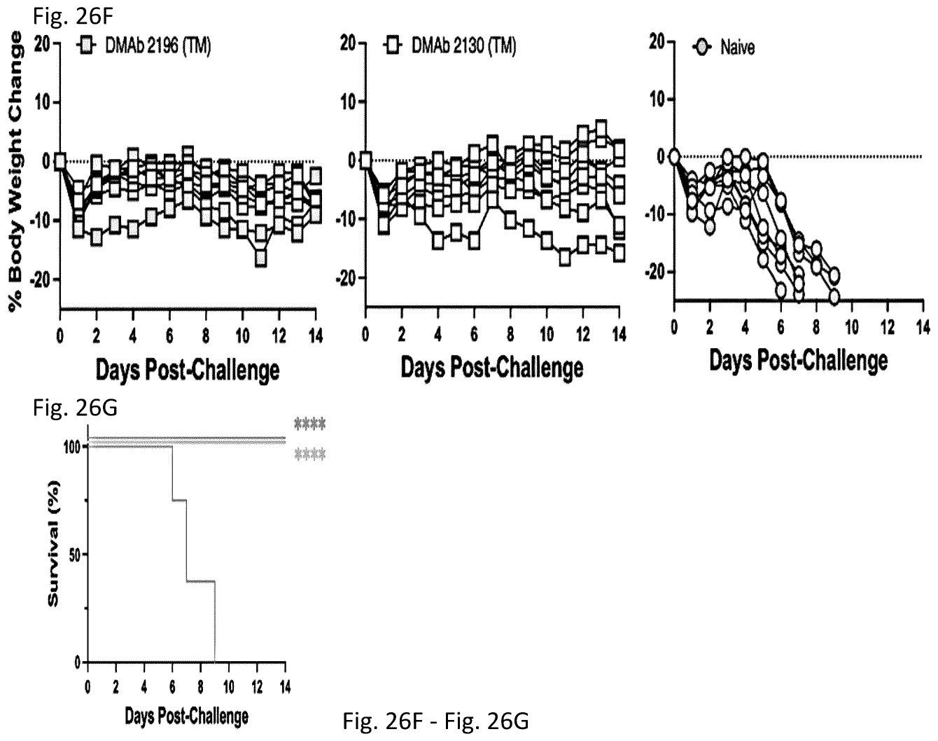

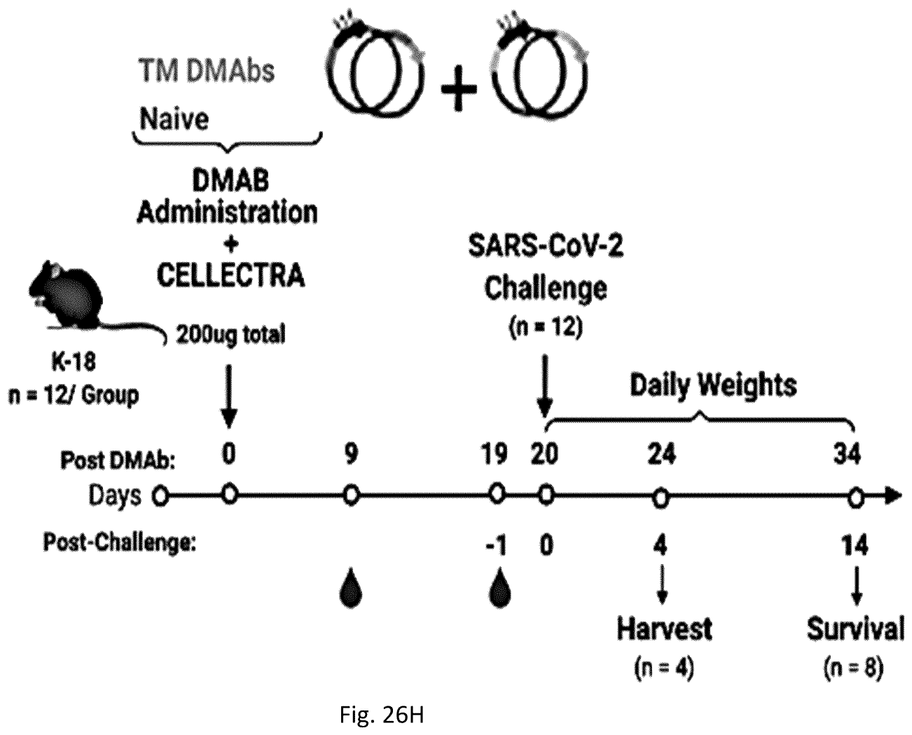

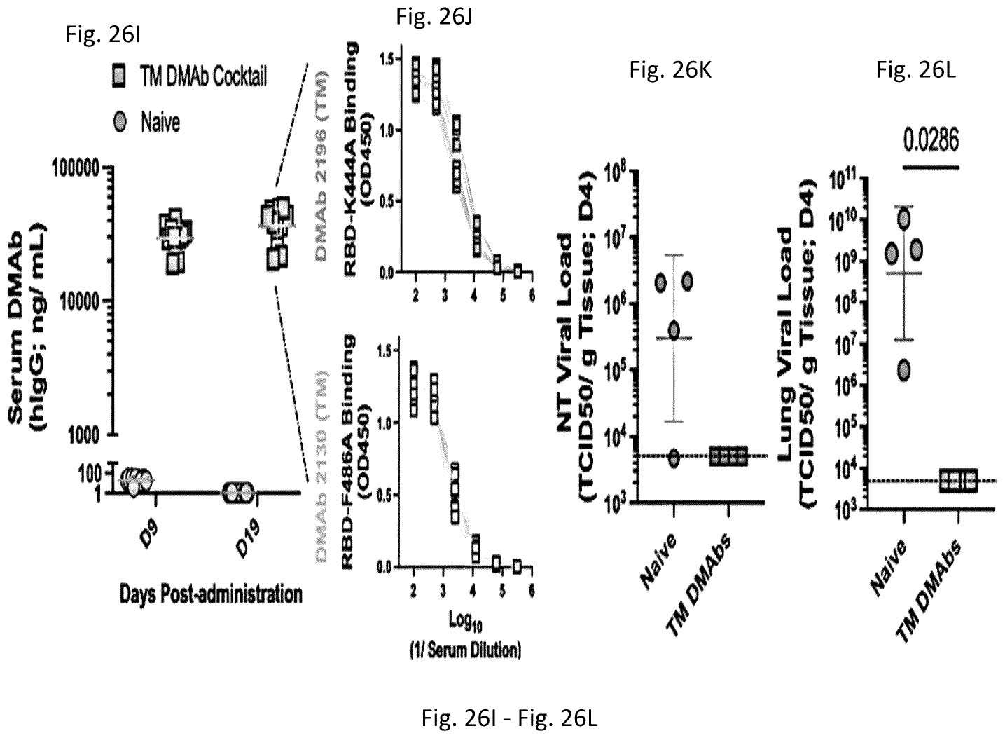

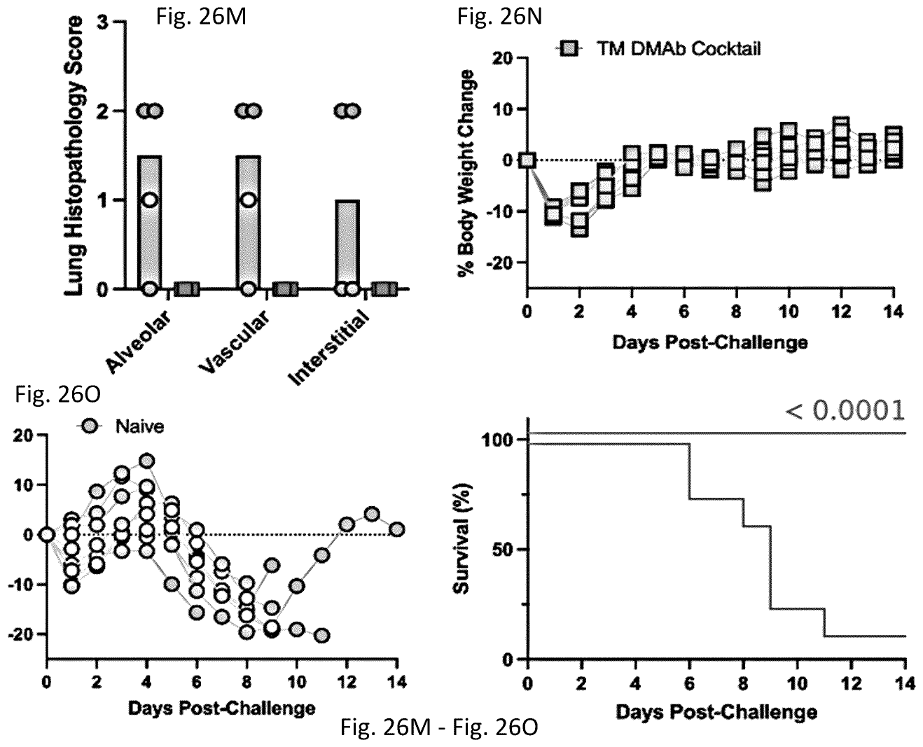

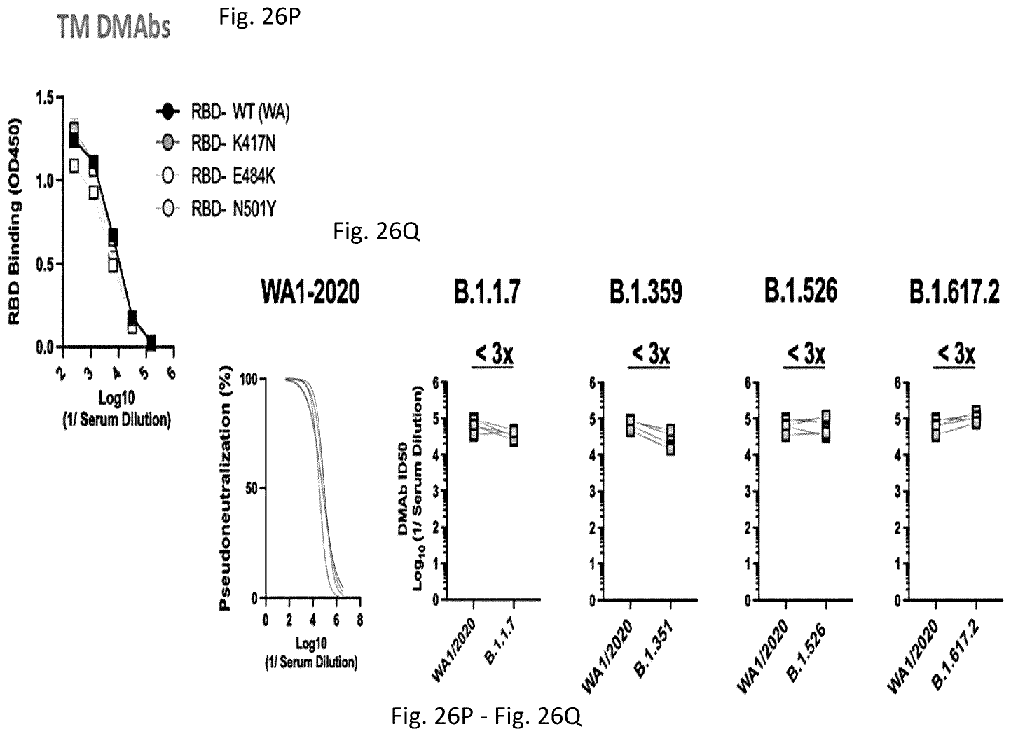

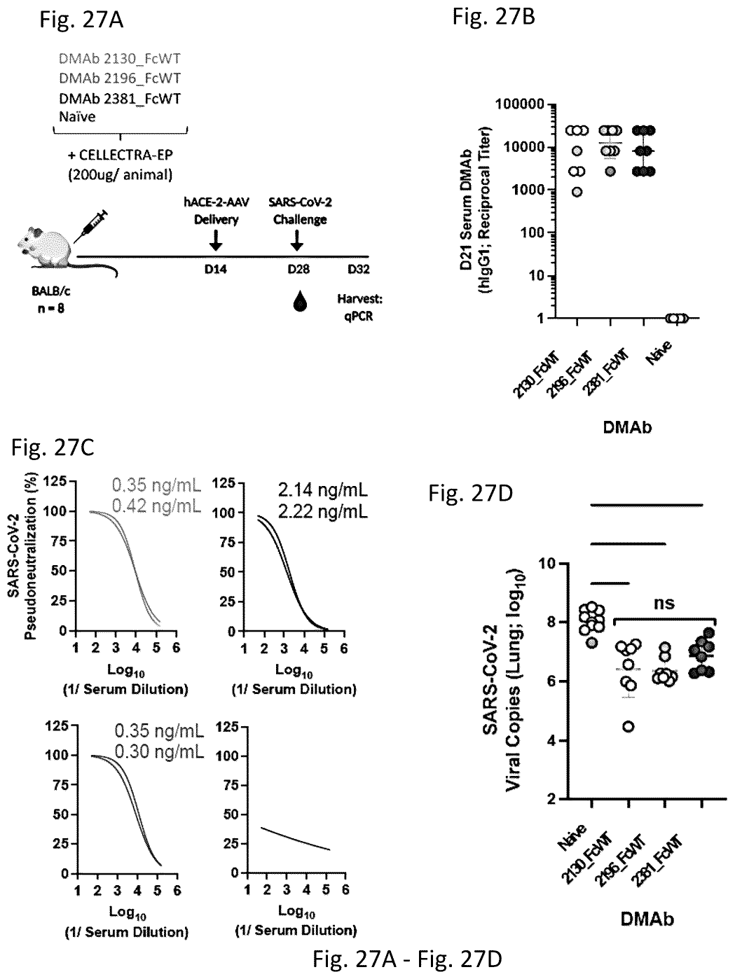

The in vivo efficacy of optimized DMAbs was evaluated using the validated SARS-CoV-2 lethal challenge model (FIG. 26A) (McCray et al., 2007, J. Virol., 81:813-82; Winkler et al., 2020, Nat. Immunol., 21:1327-1335). K-18 mice were administered DMAb 2196(TM) or DMAb 2130(TM) and serum expression was measured, reaching high and comparable levels of 30-40 μg/mL at the time of challenge (D15) (FIG. 26B). At Day 4 (D4) post-challenge, viral titers in the nasal turbinates (NT; FIG. 26C) and lungs (FIG. 26D) were reduced in DMAb groups compared to control mice; in the lung, this was a similar reduction of >4-6 logs in both DMAb groups. DMAb animals also had decreased lung pathology relative to controls (FIG. 26E) and were protected from progressive weight loss (FIG. 26F). 100% of treated animals survived while all control animals succumbed to infection (FIG. 26G). Efficacy afforded by the DMAb WT variants (2196 (WT) or DMAb 2130 (WT)) was also confirmed in a non-lethal AAV6.2FF-hACE-2-transduced murine challenge model (Gary et al., 2021, iScience, 24) in which DMAb-expressing animals had similar and significant reductions in lung viral burden (1-2 logs) compared to naïve animals (FIG. 27). These data demonstrate the ability of in vivo-launched 2130 and 2196-based DMAb variants to mediate viral reduction, prevent lung inflammation/pathology, and protect animals from severe disease and death when administered as monotherapies.

Previous reports demonstrated the complimentary and synergistic nature of mAbs 2130 and 2196 (Dong, J. et al. 2021, Nat. Microbiol., 6:1233-1244; Zost et al., 2020, Nature, 584:443-449). The protective efficacy following co-delivery of DMAbs 2196(TM) and 2130(TM) (TM DMAb cocktail), as found in AZD7442 (FIG. 26H) was evaluated. Cocktail-treated animals had robust levels (average of 37 μg/mL) of serum DMAbs (FIG. 26I) that recognized both epitope-specific RBD mutants (K444A and F486A), indicating concurrent in vivo expression of 2196 and 2130-derived DMAbs (FIG. 26J). Post-challenge, DMAb-expressing animals had viral titers below the limit of detection in NT (FIG. 26K) and lungs (FIG. 26L), representing a >5-log reduction in lung viral loads compared to control animals. No SARS-CoV-2-induced lung pathology was observed in DMAb-expressing mice, while most control mice (75%) exhibited inflammation (FIG. 26M). DMAb-treated animals exhibited minimal weight loss (FIG. 26N) and complete survival (FIG. 26O) while progressive weight loss was observed in all control mice leading to significant (88%) death. Pre-challenge sera pools containing the TM DMAb cocktail bound early RBD mutants and maintained neutralizing activity against historical SARS-CoV-2 lineages (FIG. 26P-Q). These data verify the in vivo efficacy and functional activity of plasmid-launched DMAbs against multiple SARS-CoV-2 variants following co-delivery.

### Example 5: Fc-Engineered DMAb Cocktail(s) Exhibit Equivalent Neutralizing Potency and In Vivo Efficacy Relative to Protein IgG in Murine and Hamster Challenge Models

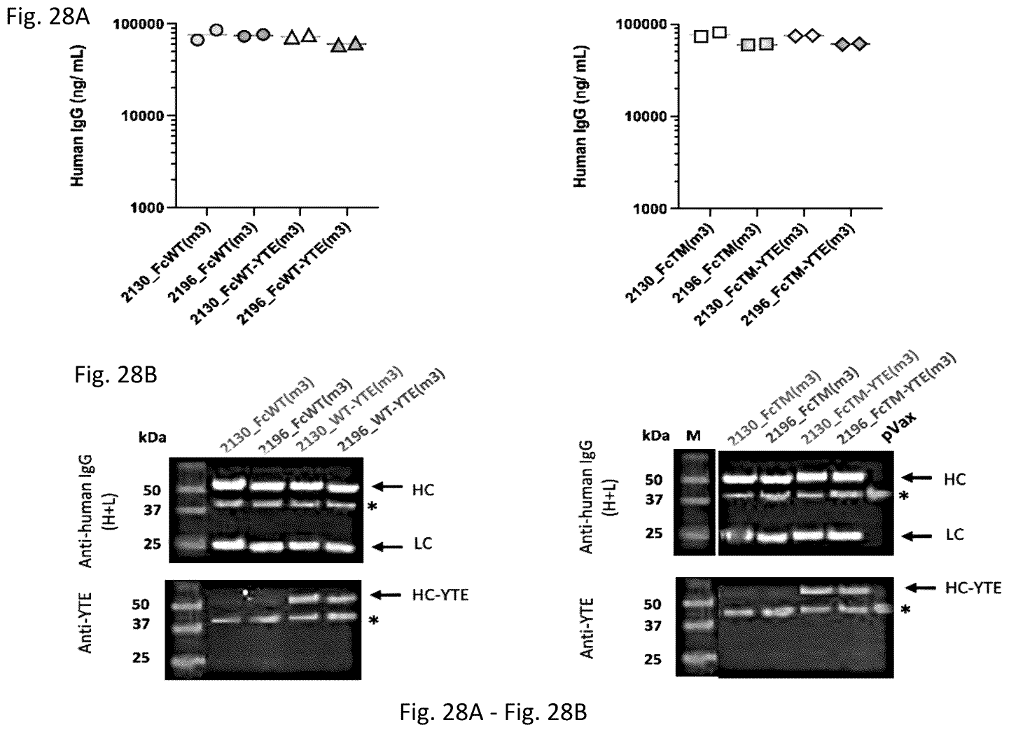

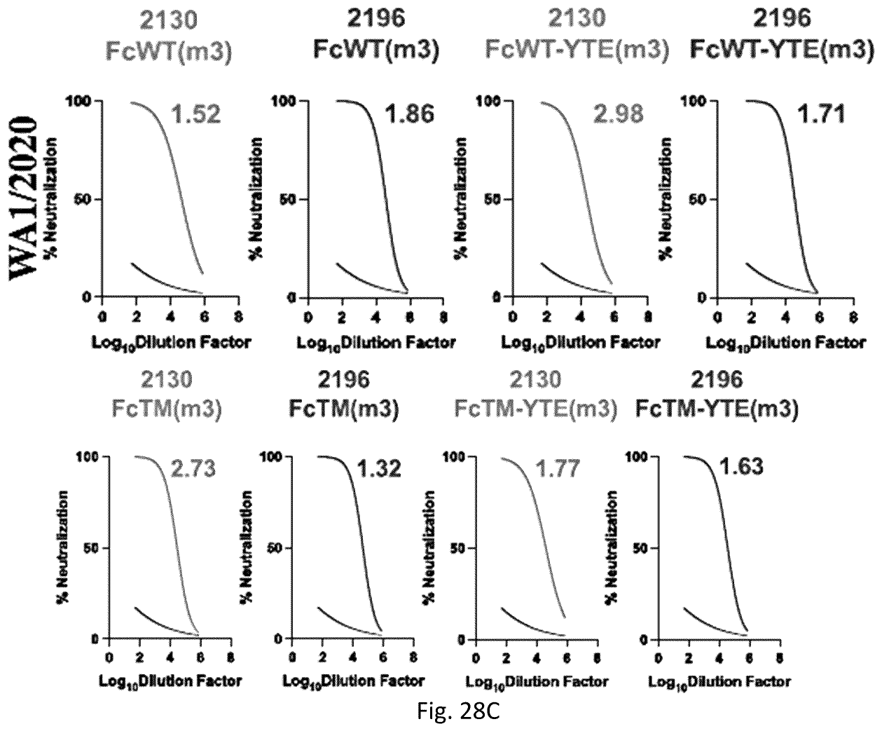

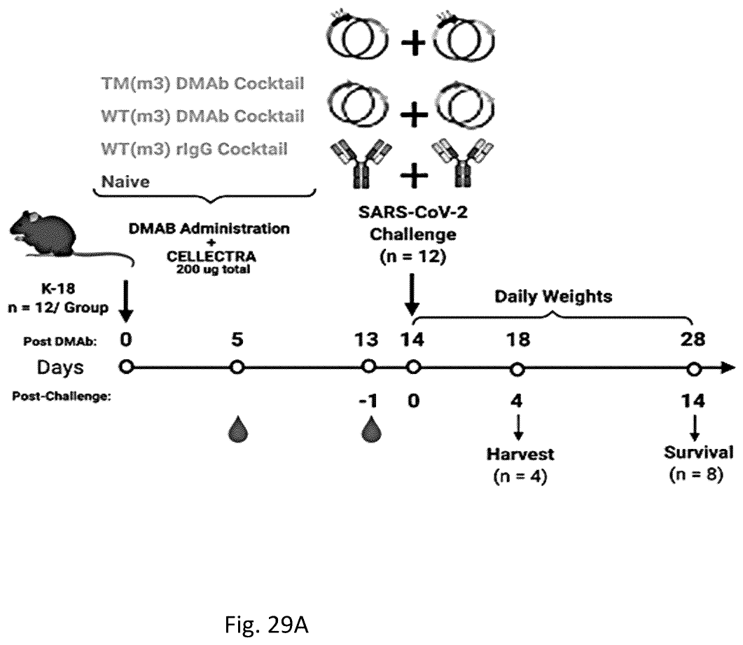

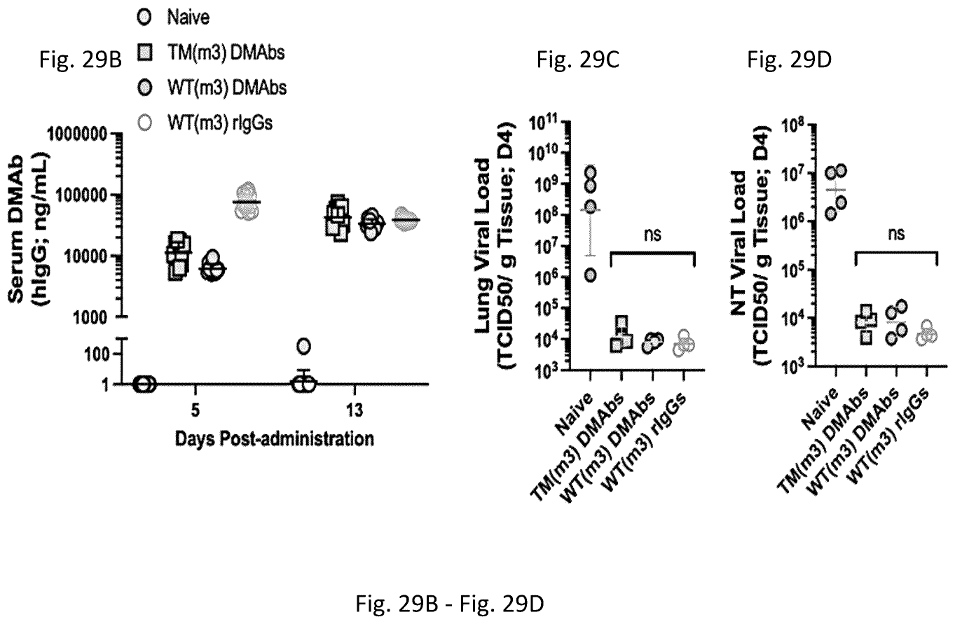

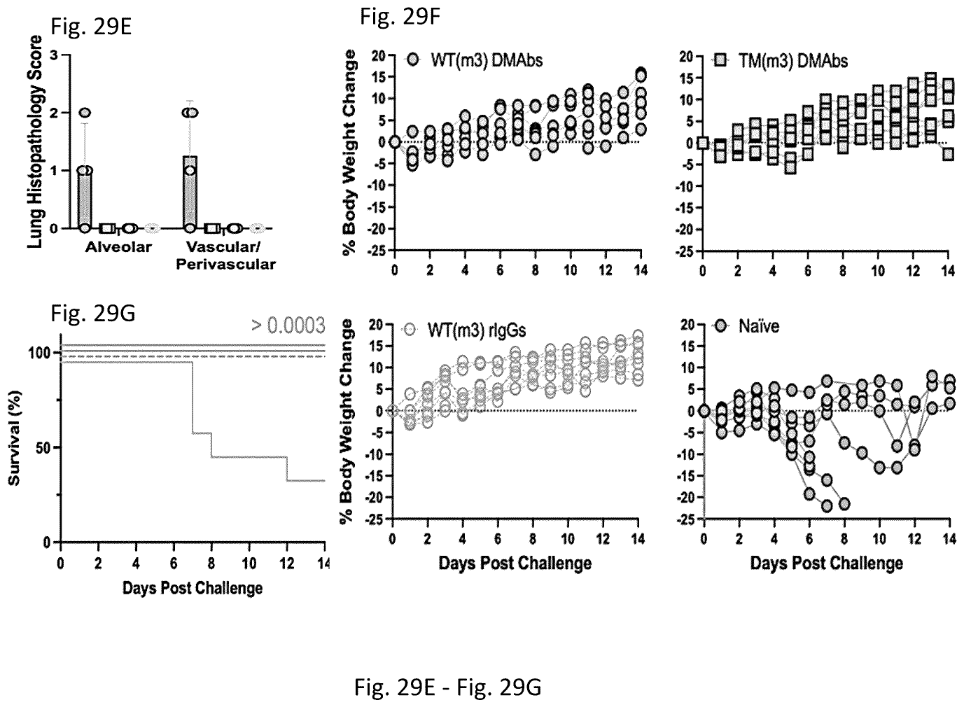

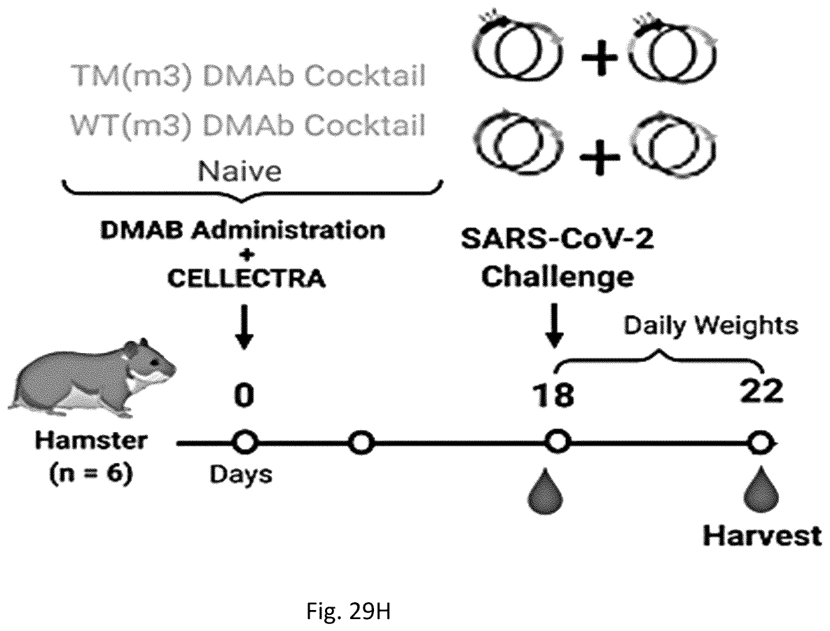

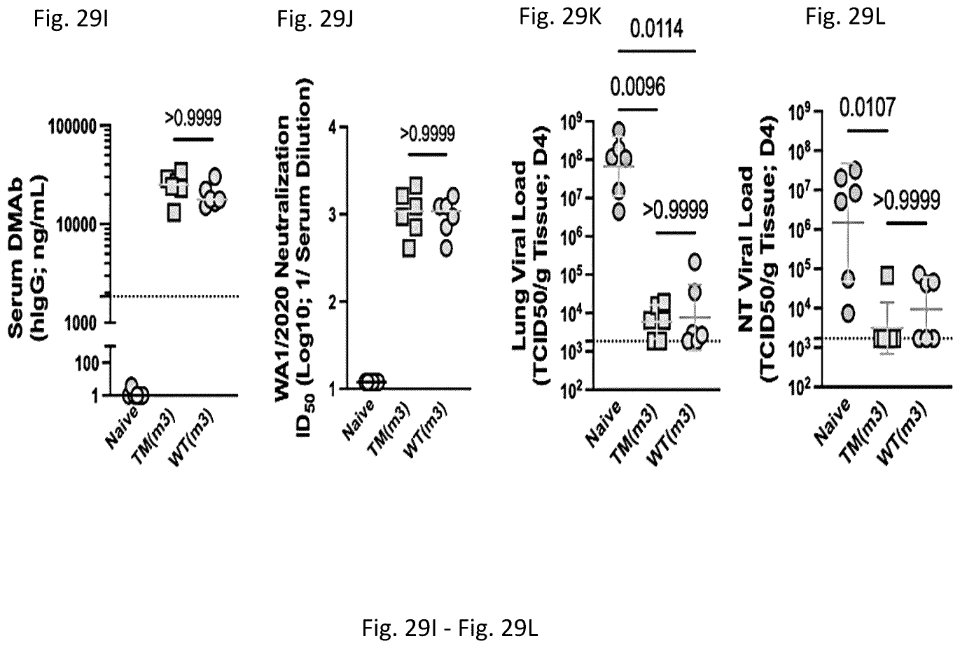

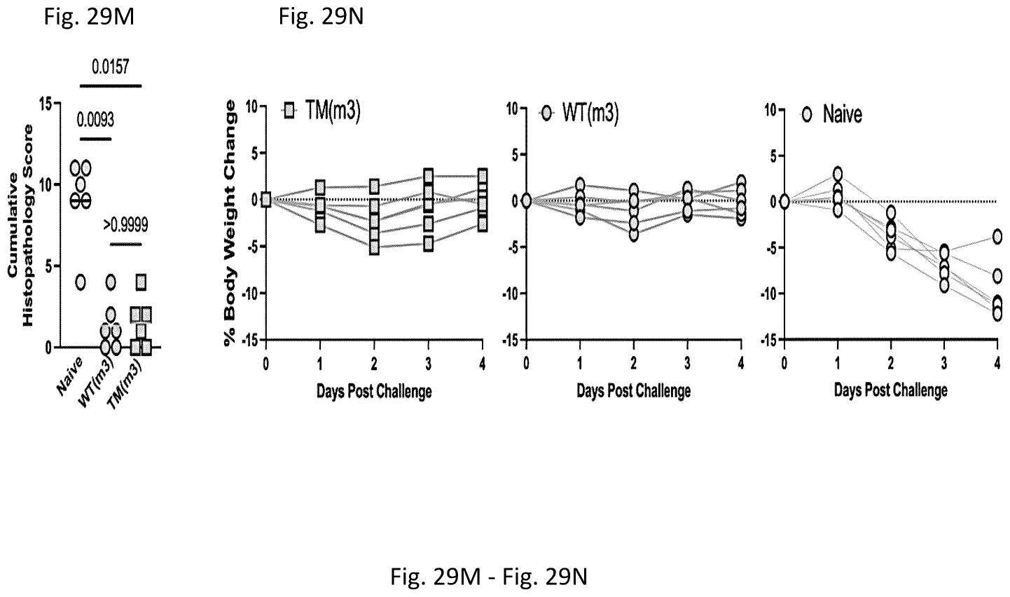

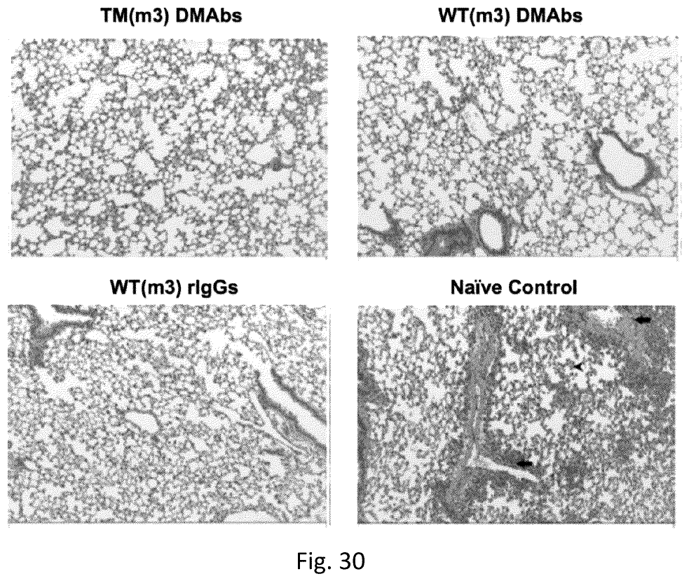

Numerous approaches to improve antibody-based therapeutics in patients have been described, including hIgG allotype selection. To facilitate clinical translation, the 2130) and 2196 DMAb plasmids (WT and TM) were modified from the human G1m1 to the G1m3 allotype framework (WT(m3) and TM(m3) constructs) utilized in AZD7442 and validated in vitro (FIG. 28). To directly investigate the potential value of effector engagement in vivo, an additional efficacy study in K-18 mice was conducted comparing TM(m3) or WT(m3) DMAb cocktails (FIG. 29A). As a benchmark standard, an additional group received the WT(m3) rIgG cocktail (purified IgG administered IP). Serum levels of both DMAb cocktails and the rIgG cocktail converged just prior to challenge (FIG. 29B). Both DMAb-administered groups were protected compared to control mice, resulting in >4-log and >2-log reduction in the lung (FIG. 29C) and NT (FIG. 29D), respectively. Viral control between the Fc-modified DMAb groups was indistinguishable from the rIgG cocktail group. Importantly, no immune pathology was detected in the lungs of antibody-treated mice, regardless of variant or mode of delivery (FIG. 29E and FIG. 30). Both DMAb cocktails conferred protection against weight loss (FIG. 29F) and mortality (FIG. 29G), compared to naïve control groups (37% survival: 3/8 animals). This protection was as potent as that observed with WT(m3) rIgG cocktail, further validating the in vivo potency and functionality of plasmid-launched antibodies relative to bioprocessed IgG.

The relative in vivo efficacy of effector-engaging and effector-null DMAb cocktails was further validated in a hamster challenge model of SARS-CoV-2 (FIG. 29H). Delivery of DMAb cocktails to Syrian golden hamsters resulted in similar and robust serum levels (FIG. 29I) of functional antibodies with activity against authentic SARS-CoV-2 (FIG. 29J). Both DMAb cocktails reduced viral loads in the lungs (FIG. 29K) and NT (FIG. 29L), protected against lung pathology (FIG. 29M) and prevented weight loss following challenge (FIG. 29N). Similar to the mouse model (FIG. 29A-G), no significant difference between in-vivo expression, in-vitro neutralization, body weight loss, pathology or reduction in viral loads were detected between the two DMAb cocktail groups. These data validate the continued efficacy of prophylactic DMAb delivery in an additional challenge model of SARS-CoV-2 and suggest that, at these concentrations, additional immune-engagement are not required for protection.

### Half-Life Extension Modifications Lead to Improved DMAb PK Profiles In Vivo Relative to Protein IgG

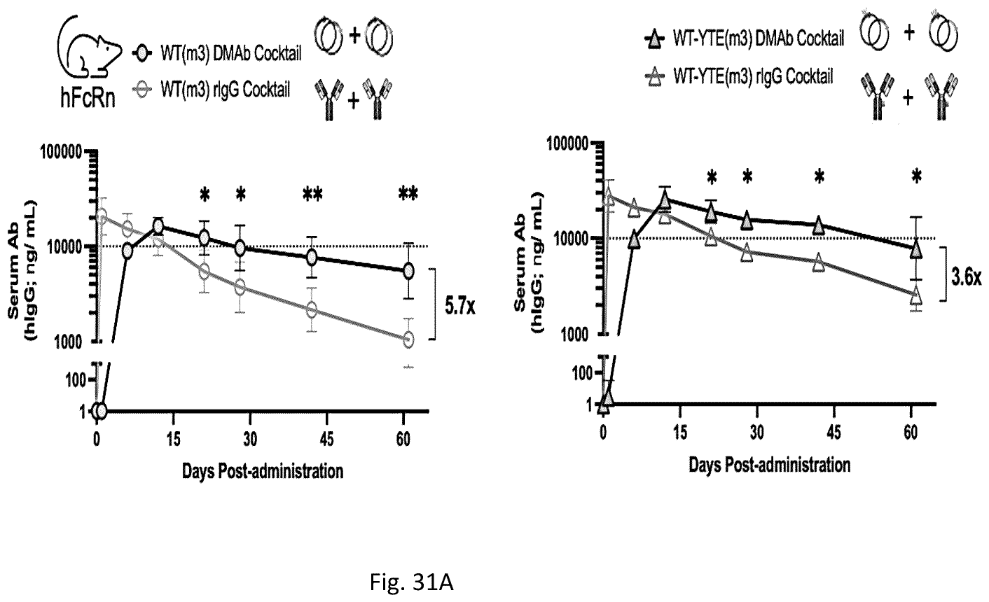

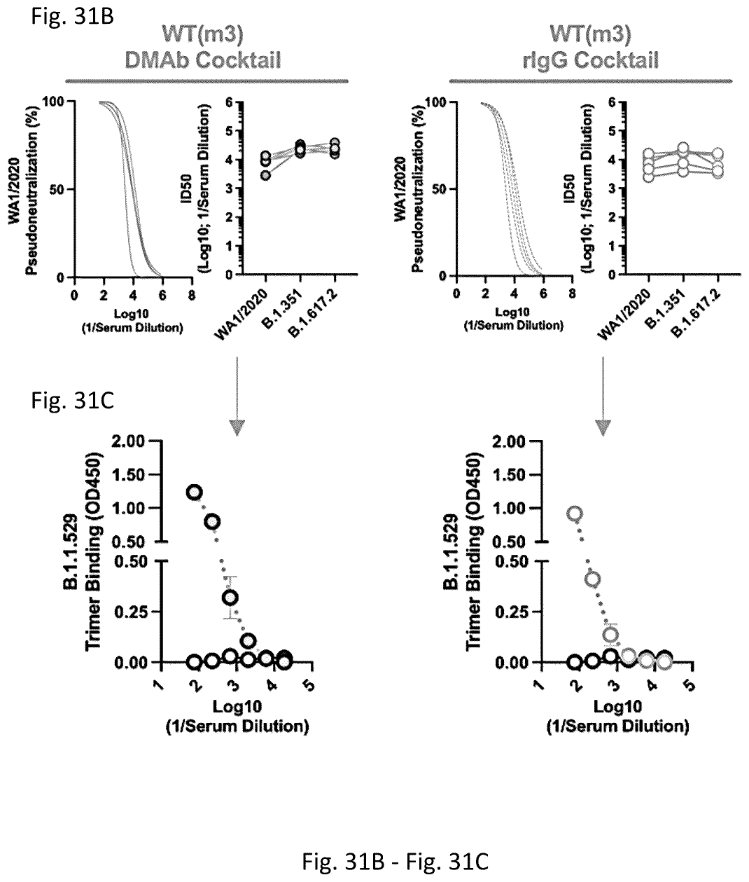

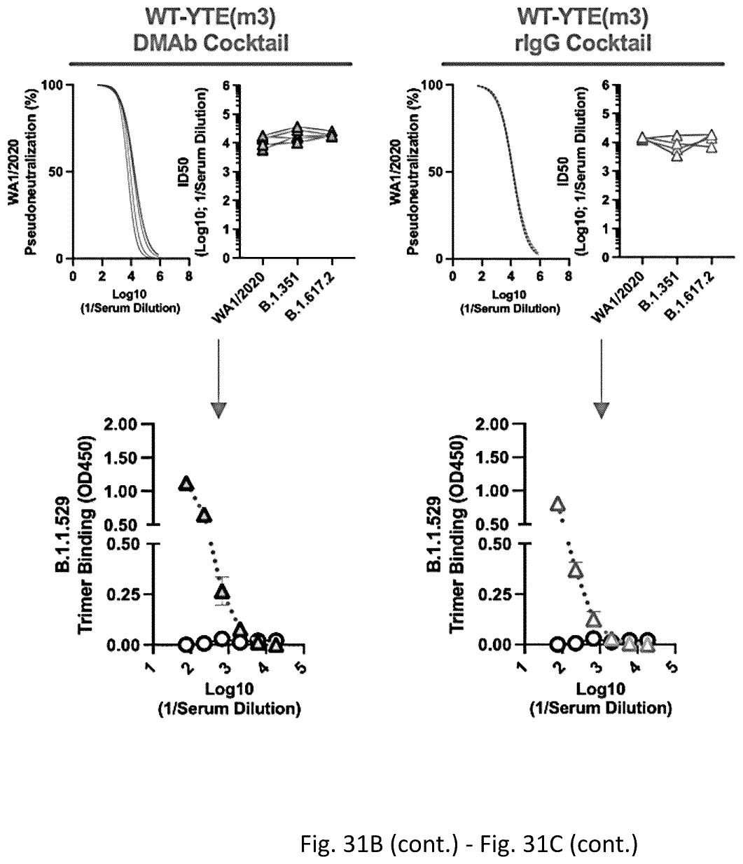

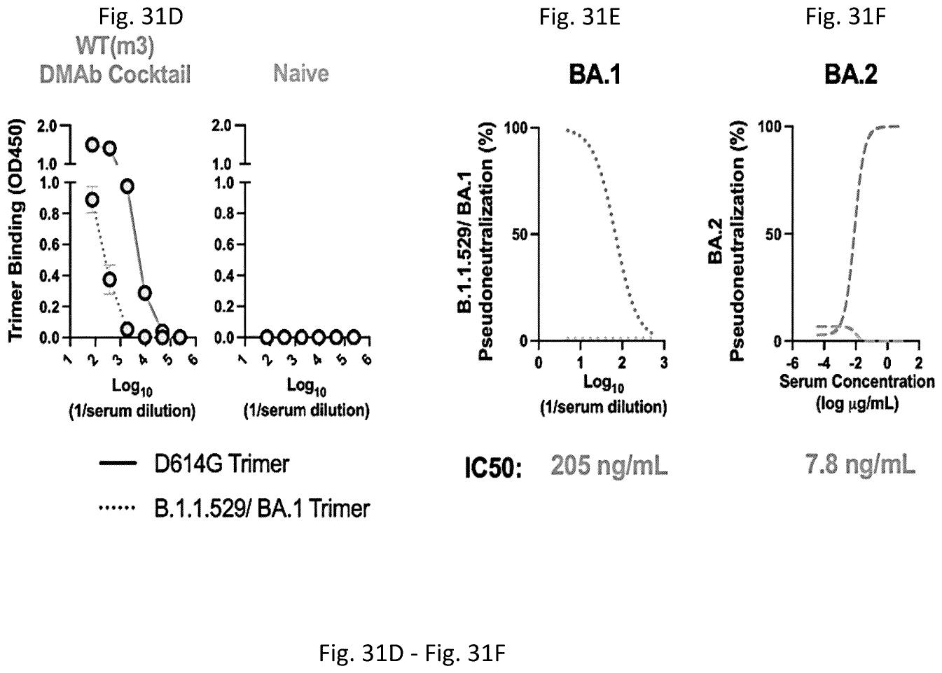

To further improve in vivo DMAb half-life, additional variants of WT(m3) and TM(m3) were generated that contain a triple Fc modification (M252Y/S254T/T256E; “YTE”) known to promote FcRn-mediated recycling of IgG into circulation (WT-YTE(m3) and TM-YTE(m3)) (FIG. 21E). These constructs were validated in vitro, achieving similar expression levels and antiviral potency as their non-YTE counterparts (FIG. 28). The effect of the YTE modification on in vivo DMAb PK was assessed using transgenic mice expressing human FcRn (hFcRn) (FIG. 31). DMAb cocktails containing WT(m3) or WT-YTE(m3) variants were delivered to mice and compared to groups that received corresponding recombinant IgG protein cocktails. Peak levels of recombinant WT mAbs were immediately detected (D1) while groups administered the WT DMAb cocktail had a more gradual accumulation of human IgG in the sera (FIG. 31A, left). Levels in the DMAb and rIgG-treated groups converged between D6-D12. While the amount of rIgG decayed over time, DMAb-treated mice maintained significantly higher levels over the following months. Parallel groups that received YTE-containing DMAb or protein rIgG cocktails exhibited similar acute kinetics that once again converged by D12 post-administration (FIG. 31A, right) and were maintained at higher levels in the DMAb group over time. YTE-containing constructs appear to be better maintained compared to their non-YTE counterparts. This is the first study to combine in vivo production and YTE function, both of which contribute to sustained expression and longer impact.

### 2196/2130 Cocktails Retain Recognition and Antiviral Activity Against SARS-CoV-2 Omicron Lineages

Sera from hFcRn mice containing DMAb or rIgG cocktails (+/−YTE) retained neutralizing activity against USA-WA1/2020 and earlier variants B.1.351 and B.1.617.2 VoC (FIG. 31B). SARS-CoV-2 strain B.1.1.529/BA. 1 subsequently emerged with increased transmissibility (Chen et al., 2022, J Chem Inf Model, 62:412-422). Due to its exceptionally high number of spike mutations, BA.1 evades vaccine-induced responses and the majority of clinically-validated mAb therapies (VanBlargan et al., 2022, Nat Med 28:490-495). The reactivity of pooled sera from DMAb-treated hFcRn (FIG. 31C) and BALB/c (FIG. 31D) mice was assessed against the BA.1 spike trimer. Strong, specific and similar binding was observed for both DMAb and rIgG groups, indicating that 2196/2130-based cocktails recognize BA. 1. This serum also neutralized B.1.1.529/BA. 1 pseudotyped virus at ng/ml levels (FIG. 31E). BA.2, a second Omicron lineage, has also evolved and recently surpassed BA. 1 in terms of global prevalence. WT DMAb cocktails retain high potency against BA.2 pseudovirus, comparable to the activity measured against the WA1/2020 strain (FIG. 31F). These data verify the sustained cross-reactivity of 2196/2130 against these dominant and difficult-to-neutralize lineages, supporting their continued use.

### Structural Profiling and Predictive Modeling of In Vivo-Launched DMAbs

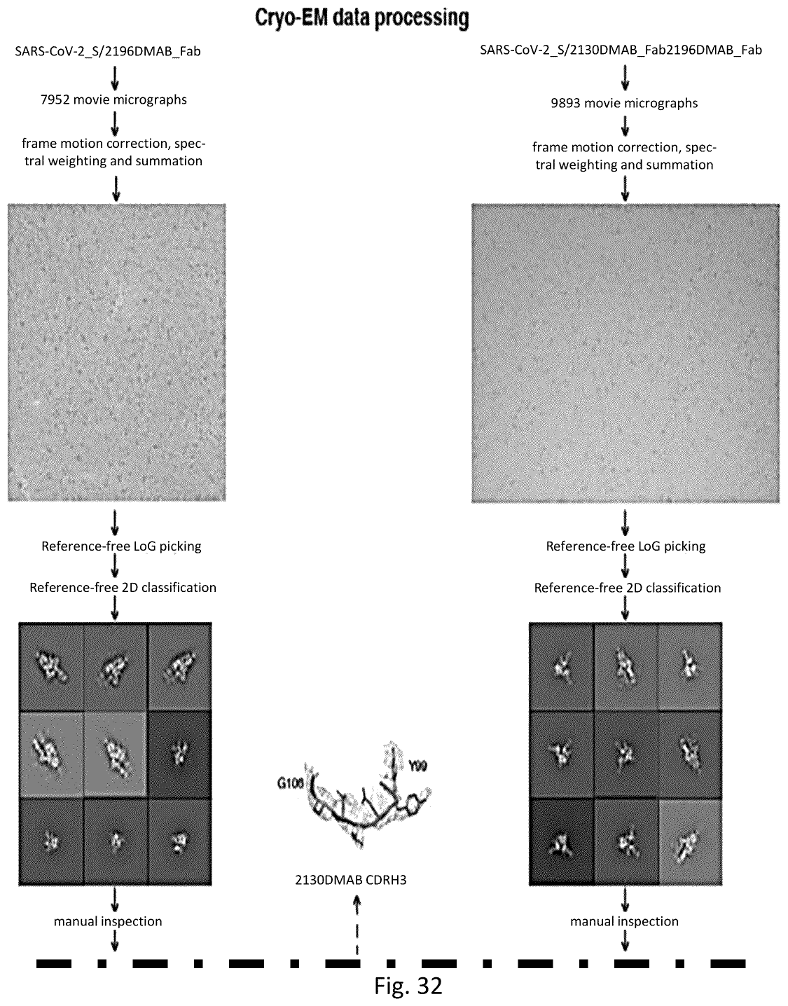

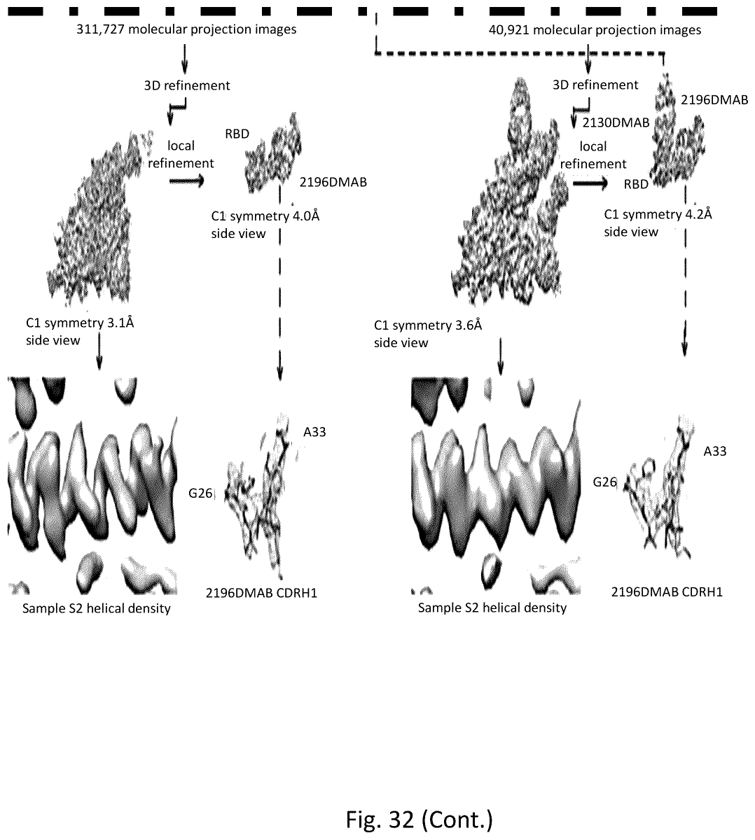

To better visualize the structural profile of in vivo-launched 2196 and 2130-based DMAbs and their interaction with SARS-CoV-2 spike, cryo-EM analysis was performed on serum-derived DMAbs. Mice were administered DMAbs 2196 TM(m3) and 2130 TM(m3) in combination or 2196 TM(m3) alone. Total IgG was purified from sera pools, digested and isolated Fabs were complexed with stabilized spike trimer from SARS-CoV-2 (USA-WA1/2020; 6P stabilization) (FIG. 32).

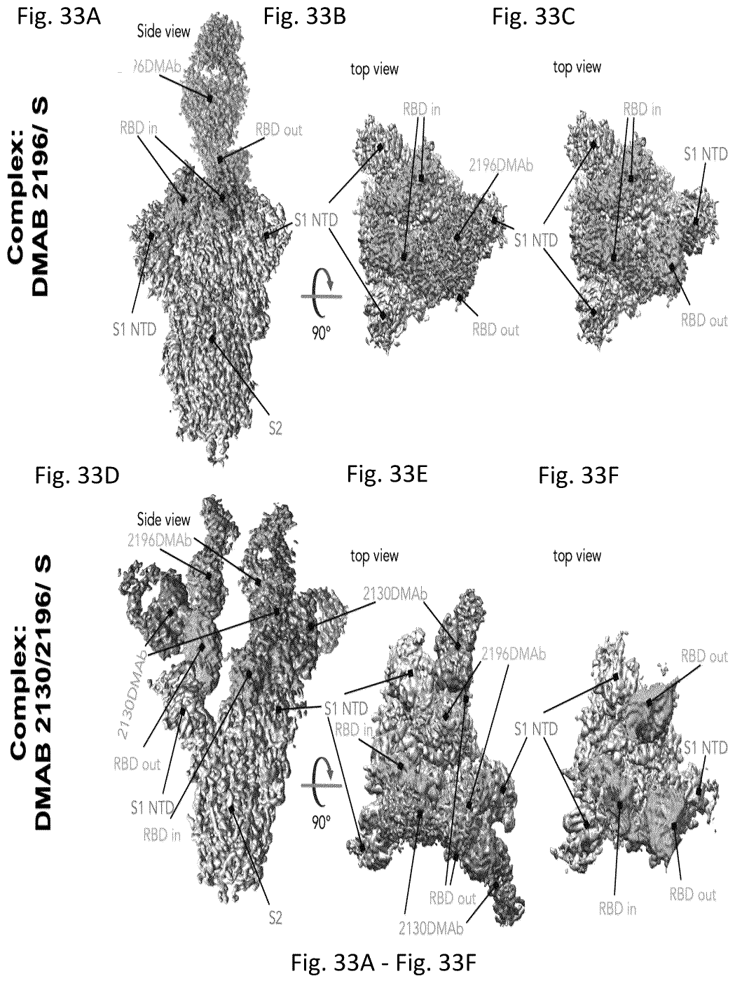

Two structures outline the overall interaction of 2196 DMAb alone (FIG. 33A-C) or the DMAb cocktail of 2196/2130 (FIG. 33D-F) with SARS-CoV-2 spike trimer. A global density map of DMAb 2196/S was generated with a resolution of 3.1 Å. Only one 2196 Fab was present in the 2196/S complex, bound to the single RBD in the “out” position; the other two RBDs were in the “in” position (FIG. 33A-B). The epitope of 2196 is accessible only in the “out” configuration. Interestingly, the global density map of the 2196/2130/S complex at 3.6 Å revealed the concurrent binding of two 2196 Fabs and three 2130 Fabs per trimer (FIG. 33D-F). Here, two RBDs were in the ‘out’ position, presenting accessible epitopes to 2196 Fabs while the third maintained the “in” conformation, sterically occluding the binding of 2196. DMAb 2130) Fabs bound to all three RBDs regardless of configuration.

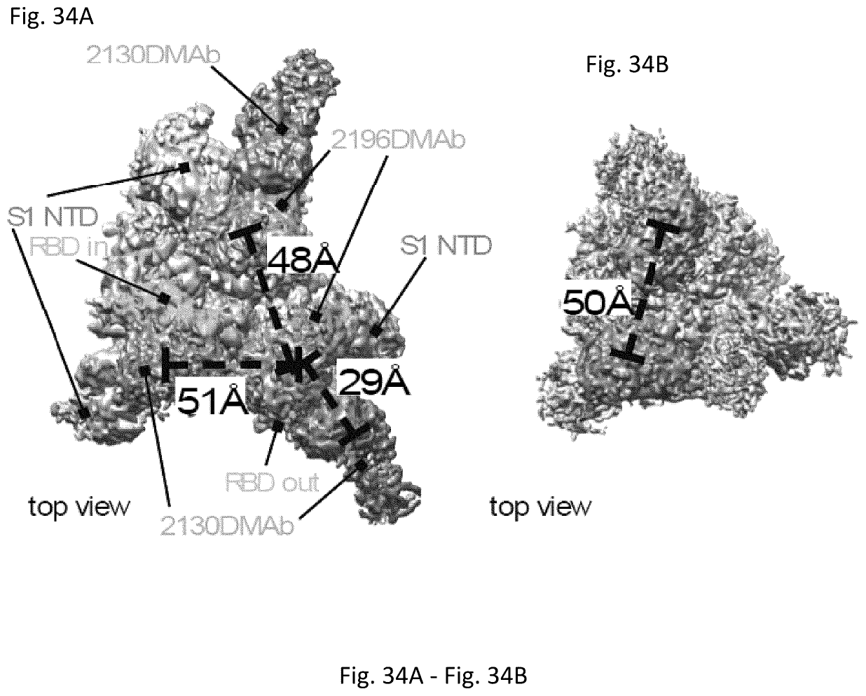

As these clones are known to be functionally synergistic, the relative spatial distance between bound DMAbs was measured as an indication of their potential to interact with one another. Distance between the center of two 2196 Fabs complexed with spike (FIG. 33D-F; FIG. 34A) was 48 Å, allowing both Fabs to recognize spike epitopes simultaneously and facilitating 2196 IgG avidity binding effects. Similarly. 2130 Fabs complexed to RBD ‘in’ were separated by 50 Å to allow 2130 IgG avidity effects (FIG. 34B). The distance between 2130 and 2196 complexed to the same “out” RBD was only 29 Å and the distance between 2130 complexed to ‘in’ RBD and 2196 complexed to ‘out’ RBD was 51A. Each of these distances allows non-covalent interactions between bound IgGs that contribute to overall cooperative binding to the viral spike trimer.

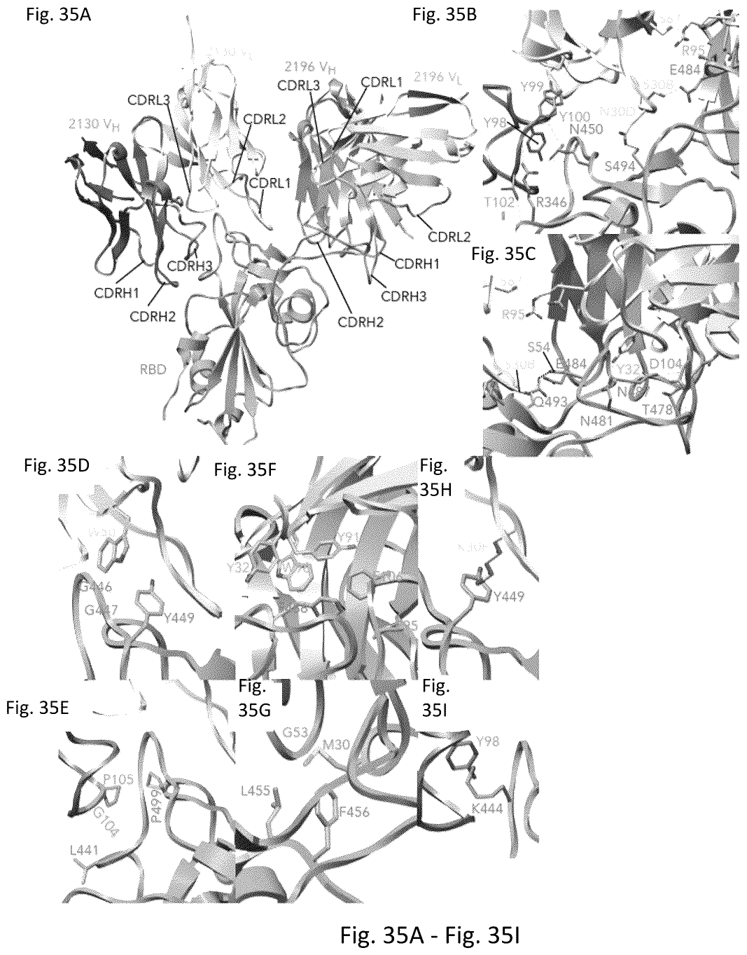

Subsequent detailed analysis focused on the DMAb 2196/2130/spike structure (FIG. 35). Here the paratope/epitope interface of both DMAbs with USA-WA1/2020 spike revealed multiple modes of interaction (FIG. 35A) including direct hydrogen bond (h-bond) partners. For DMAb 2130, many of these are relatively resistant to viral mutations since they engage RBD main chain partners, including CDRH3 T102 to RBD R346 peptide bond, RBD 346 to CDRH3 Y100 peptide bond, CDRH3 Y98 top to RBD V445 peptide bond, and RBD N450 to CDRH3 Y100 peptide bond. Other patterns depend on specific side chain interactions, including CHRL1 N30 to RBD S494 and CDRL1 to S30B to RBD 484E (FIG. 35B). H-bonding patterns on the paratope/epitope interface of DMAb 2196 and spike were also extensive, including RBD Q493 with CDRH2 S54, RBD N481 with CDRL1 Y32, RBD N487 with CHRL3 D104 and RBD T478 with CHRL3 D104 (FIG. 35C).

Supporting the notion of DMAb cooperative binding effects (FIG. 33D-F), h-bond patterns between DMAb 2196 and 2130 Fabs were observed: 2130 light chain S67 engaged 2196 light chain R95 (FIG. 35B-C), along with additional potential h-bond partners in the vicinity to strengthen this cooperativity. Further support of Fab-to-Fab h-bonding was demonstrated in the density maps. The 2196/spike RBD/Fab portion of the map was rather noisy (FIG. 33A-C) reflecting the inherent flexibility in RBD position and flexibility of Fab with respect to RBD. In comparison, the 2196/2130/spike RBD/Fab density map portions appeared much more ordered and defined (FIG. 33D-E) consistent with the observed Fab-to-Fab stabilizing interactions.

In addition to h-bonding, both DMAbs displayed numerous hydrophobic/van der Waal's interactions with RBD; DMAb 2130 CDRL2 W50 packs against RBD G446, G447 and Y449 (FIG. 35D) and CDRH3 G104-P105 packs against RBD L441 and P499 (FIG. 35E). DMAb 2130 also exhibited multiple cation-pi interactions including CDRL1 30F to RBD Y449; (FIG. 35H) and CDRH3 Y98 to RBD K444 (FIG. 35I). Likewise, DMAb 2196 participated in an unusual level of hydrophobic interactions, including the formation of a 5-member hydrophobic cage (composed of CDRL1 Y32, CDRL3 Y91 and W96, CDRH3 P95 and F106) engaged with RBD F486 (FIG. 35F). Additional hydrophobic interactions of DMAb 2196 include CDRH1 M30, CDRH2 G53 and RBD L455 and L456 (FIG. 35G).

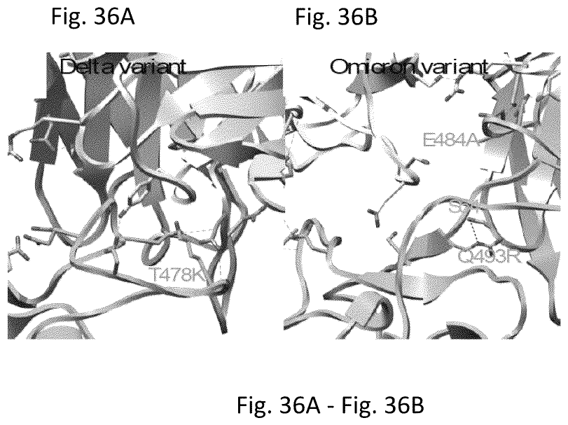

DMAb 2130/2196/S structure was used as a framework to model the impact of recent emerging variants (FIG. 36). The B. 1.617.2 variant contains a T478K mutation which is relevant to the 2196 epitope (FIG. 35C); however, this did not significantly modulate 2196-mediated neutralization (FIG. 25). Loss of the h-bond between T478 and 2196 CHRH3 D104 (FIG. 35C) could be partly recovered by h-bonding between 2196 CHRH3 D104 and the peptide bond of K478, explaining its maintained activity (FIG. 36A). The B.1.1.529 variant contains an additional two potentially consequential mutations: Q493R and E484A, both of which engaged in h-bonding in the pre-omicron isolates (FIG. 35C). As the Q493R mutation enables multiple h-bonding partners with the 2196 DMAb Fab (FIG. 36B; RBD S54 and N56), it is expected to be well-tolerated. Introduction of E484A shortens and changes the chemical properties of the side chain, breaking the hydrogen bond to 2130 CHRL1 S30B (FIG. 35B). However, the binding and neutralization data illustrate that the DMAb 2196/2130 cocktail can mitigate this mutation (FIG. 31). These structural studies not only provide the first visualization of in vivo-produced antibodies and their interaction with target antigen, but also support the in vitro and in vivo data indicating that the DMAb 2196/2130 cocktail retains recognition and activity against current SARS-CoV-2 variants.

### Example 6: In Vivo Delivery of Engineered Synthetic DNA-Encoded SARS-CoV-2 Monoclonal Antibodies and Preventative Efficacy in Non-Human Primates

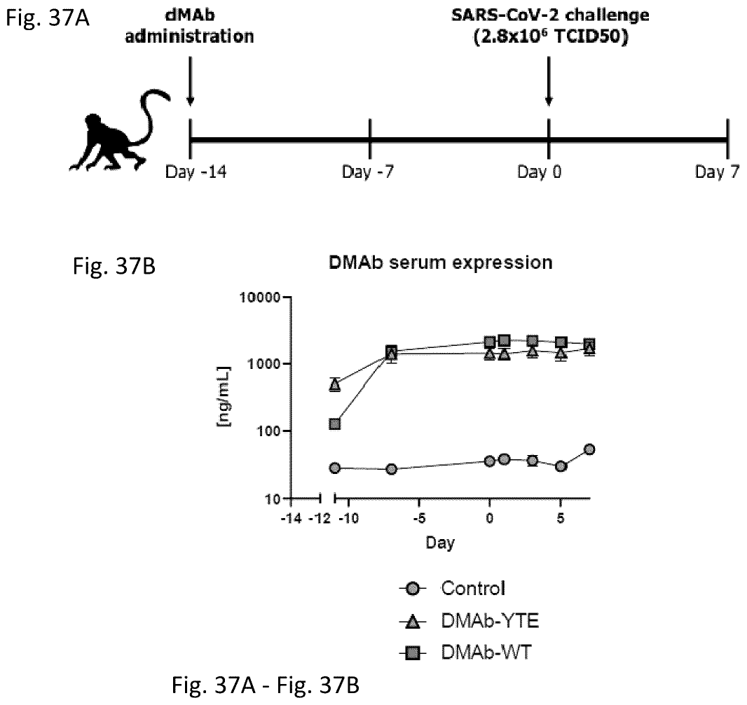

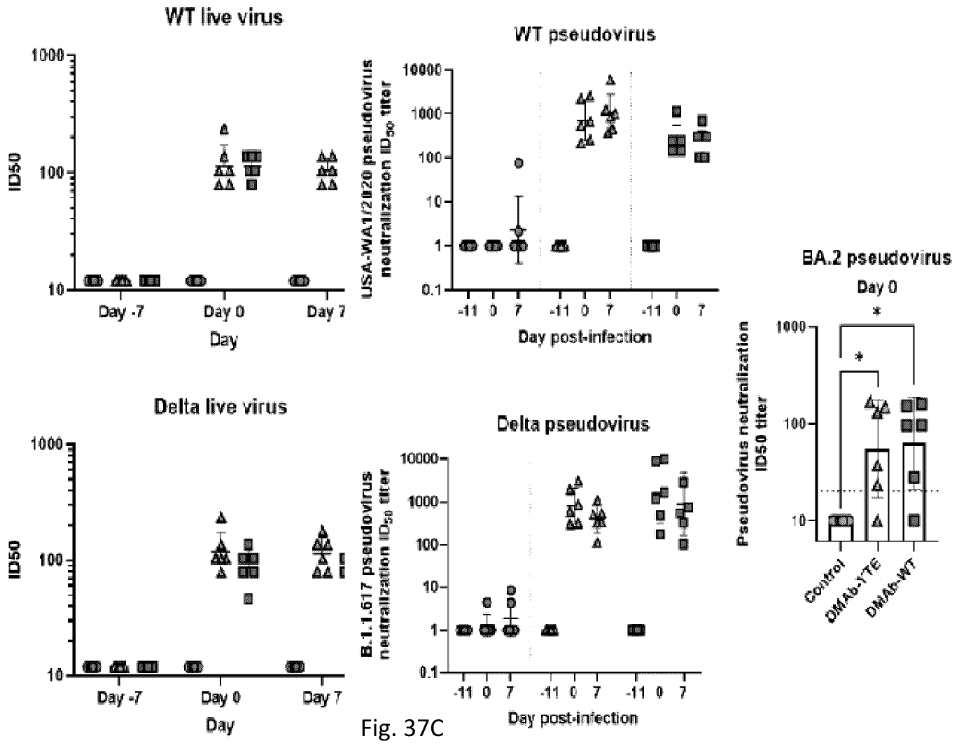

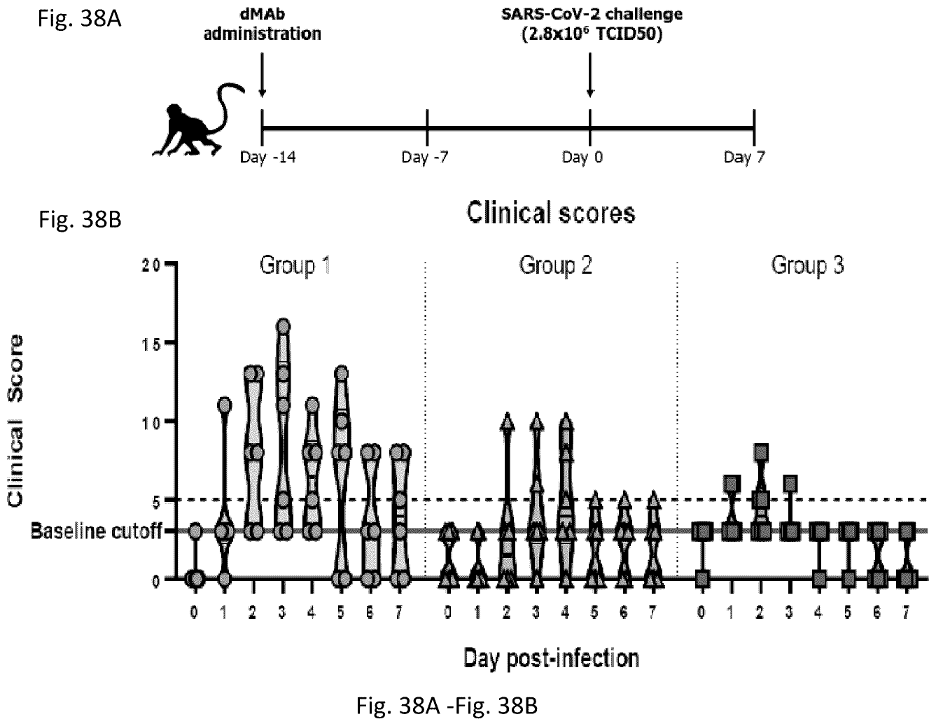

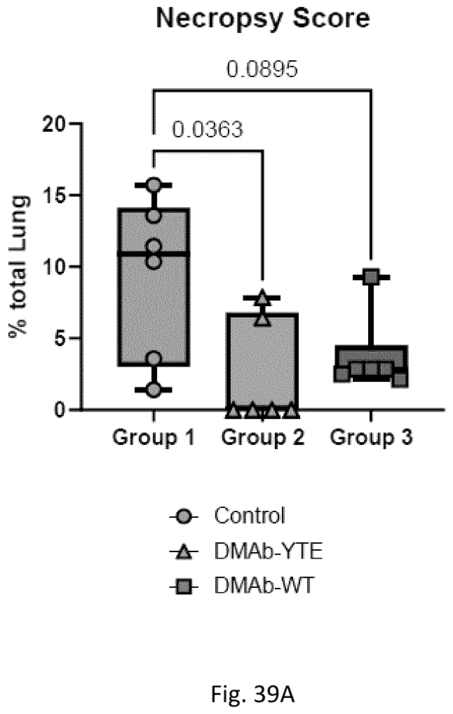

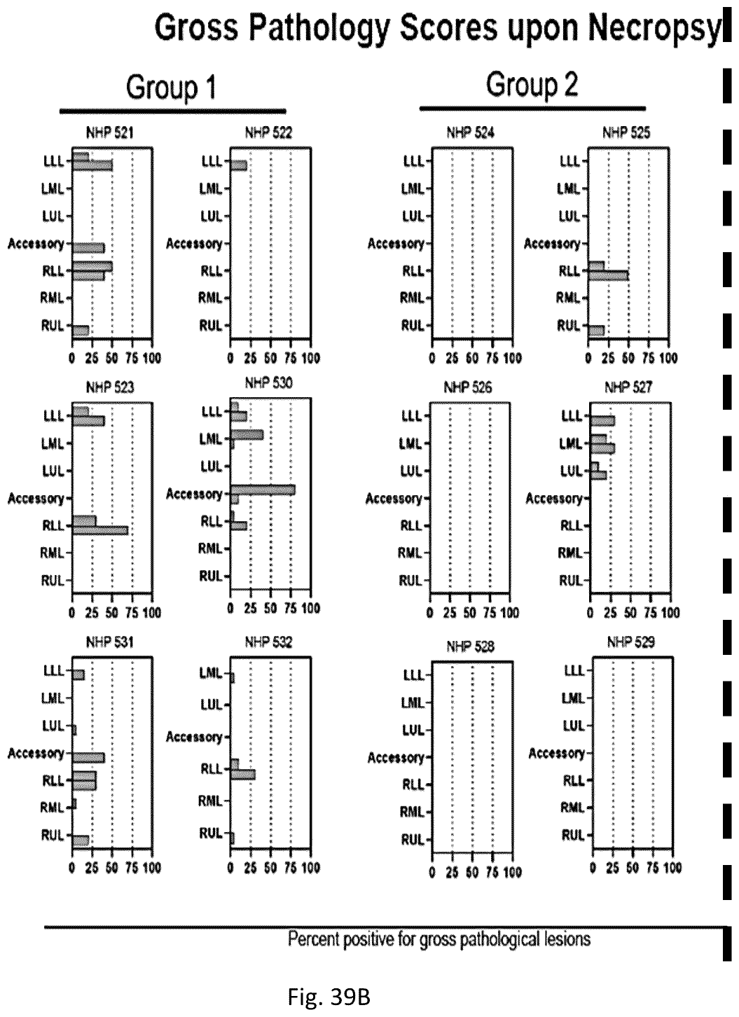

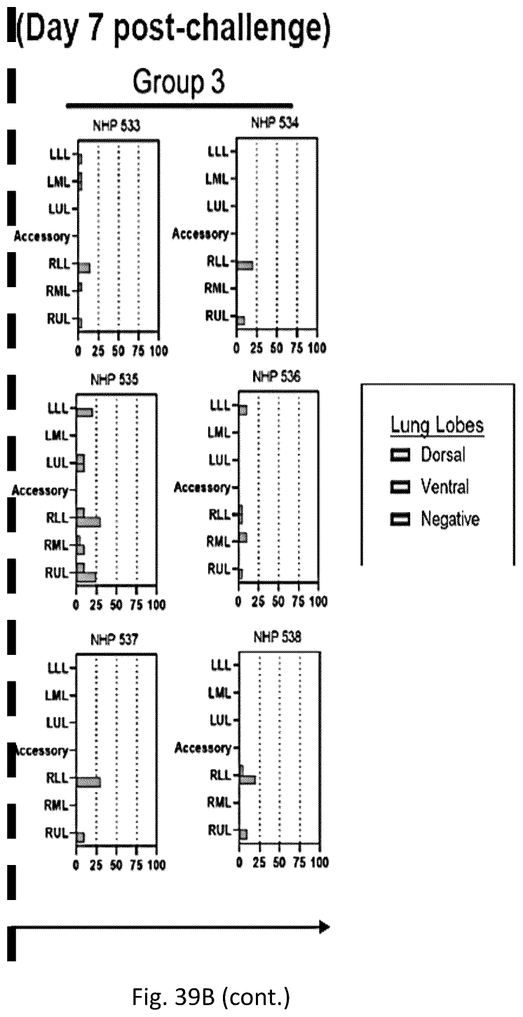

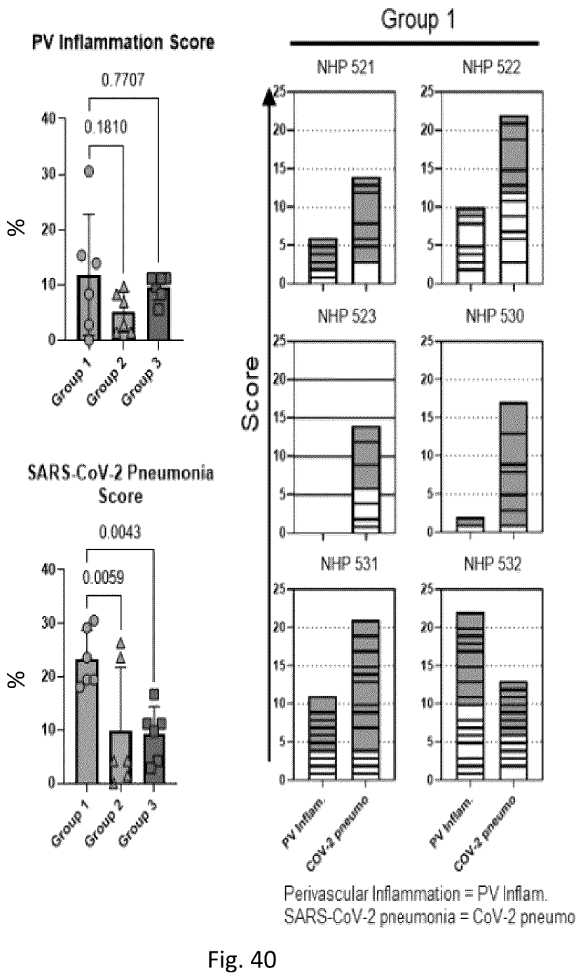

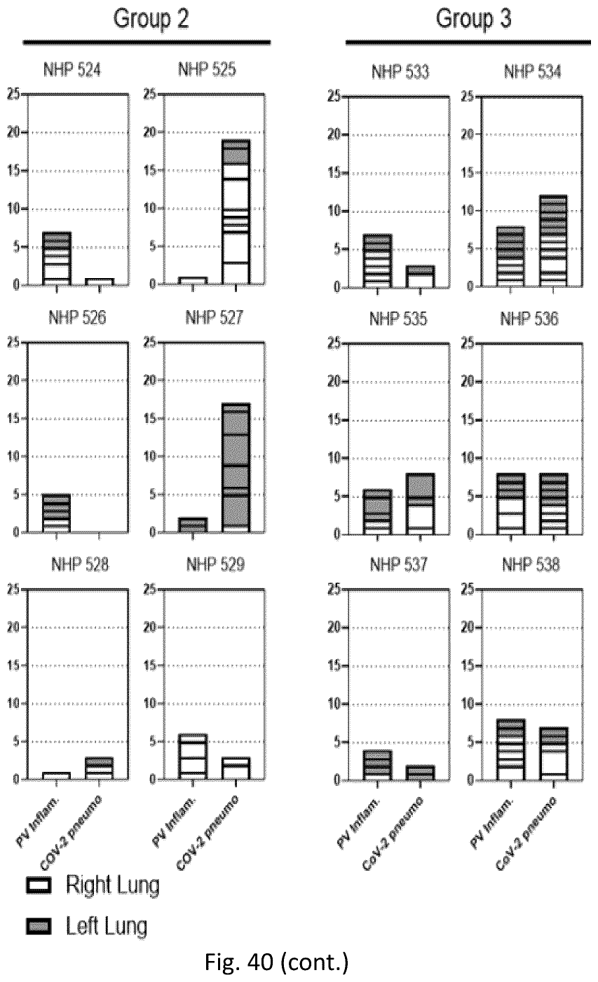

DNA-encoded antibodies have been engineered to encode recombinant IgG mAbs. Engineered DMAbs show comparable protection in mouse models as well as comparable neutralizing activity to standard mAbs. A YTE half-life extension modified DMAb has been designed that shows expression in mice (see Example 5) and NHPs (FIG. 37). Rhesus macaques receiving the DMAb cocktails were protected against severe signs of COVID-19 associated disease (FIG. 38). Significantly lower signs of disease were observed in the lungs for the DMAb-YTE cocktail (FIG. 39 and FIG. 40). Reduced signs of perivascular inflammation and SARS-CoV-2 pneumonia were observed in DMAb groups (FIG. 39 and FIG. 40.) Overall, the data support DMAbs as potential additional delivery modality for pre-exposure prophylaxis for the prevention of symptomatic COVID-19. These data have implications for human translation of DNA encoded mAbs for the prevention of COVID-19 and other infectious diseases.

### Example 7: Fc-Engineering Strategies to Optimize Potency of mAb Biologics

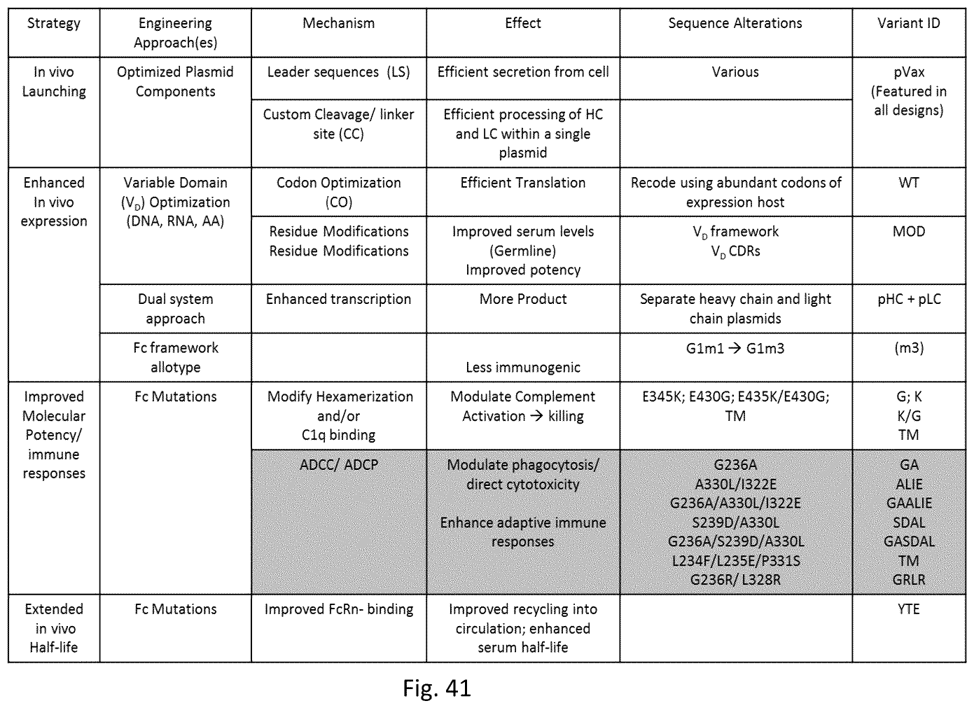

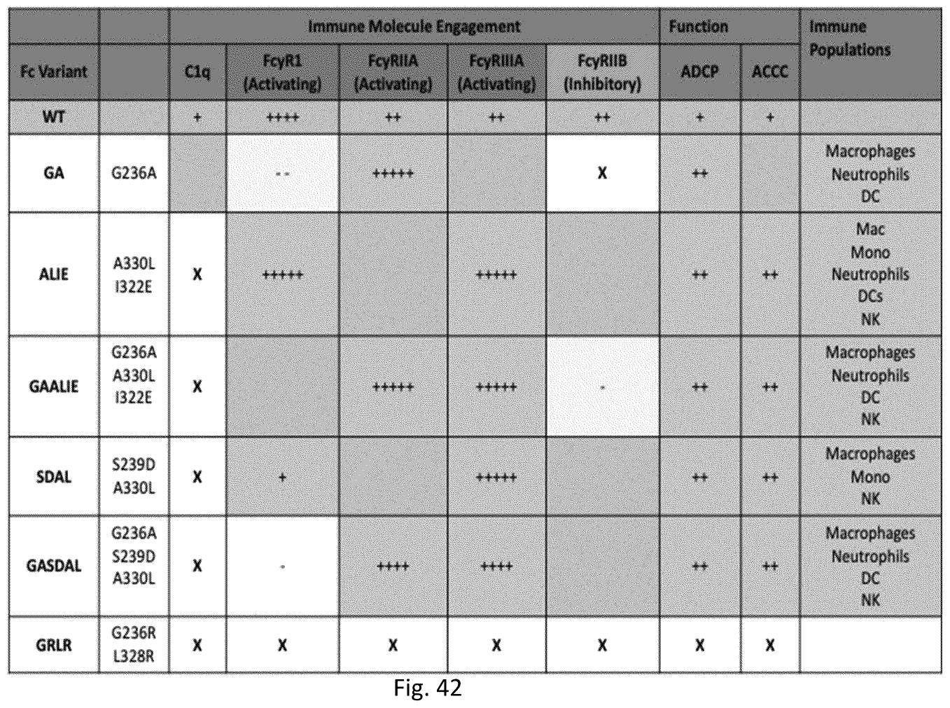

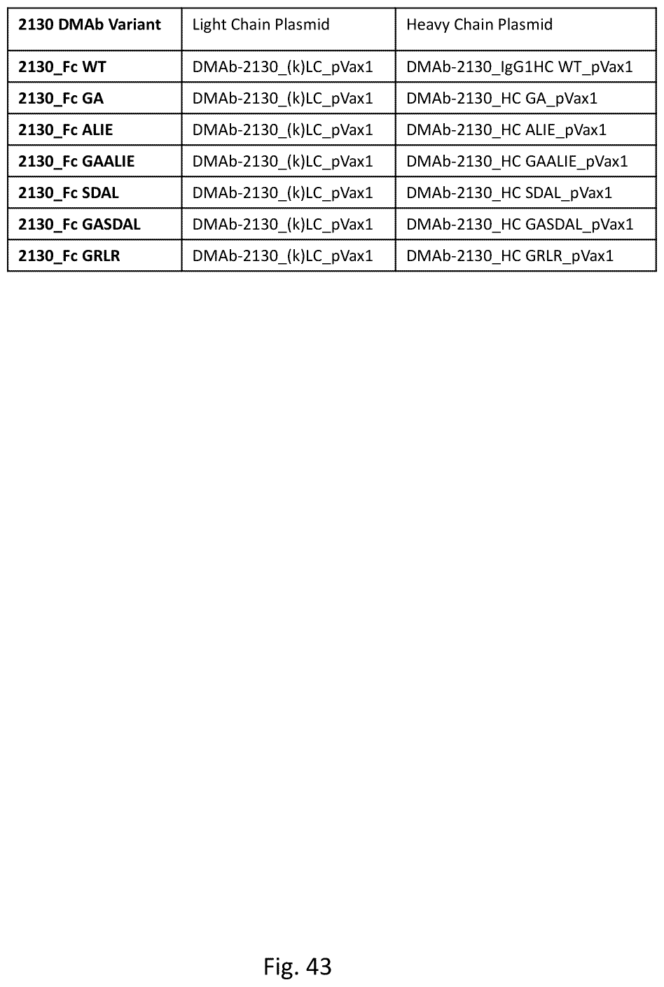

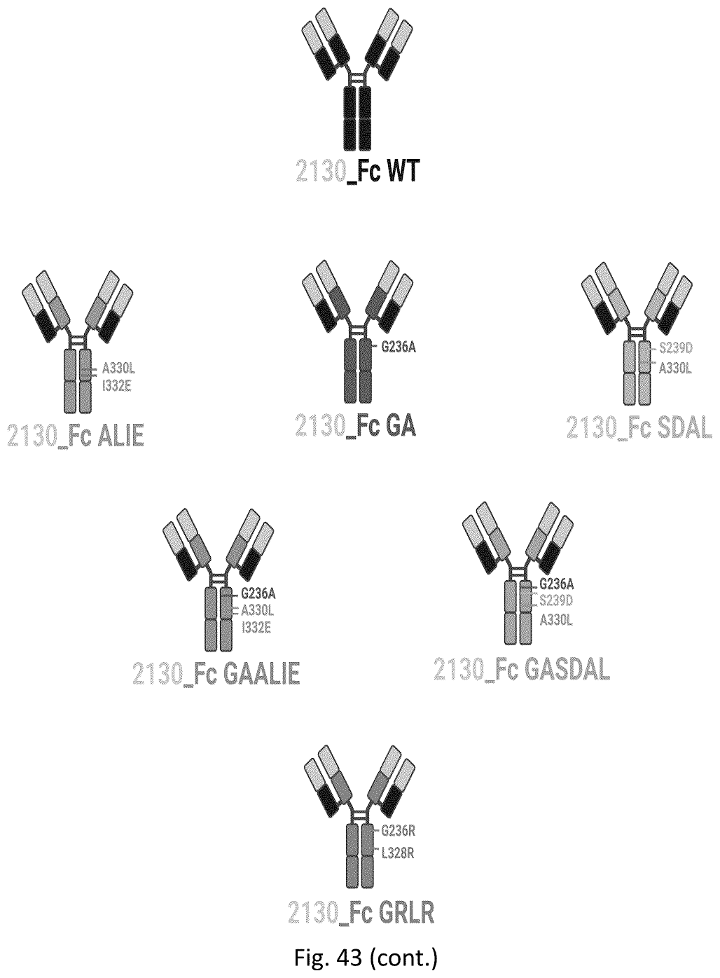

Modified antibodies were developed to further improve the in vivo potency of antibodies by engaging additional mechanisms of action including phagocytosis, cytolytic killing, etc. FIGS. 41 and 42 show the Fc modifications and predicted functionality. FIG. 43 shows the design of plasmid constructs encoding the indicated 2130 DMAb Fc variants.

Validated anti-SARS-CoV-2 mAb 2130 was used as the model clone. The indicated 2130 DMAb variants were created using a dual plasmid approach: Single light chain plasmid paired with various heavy chain plasmids. Sequence optimization of Ig insert sequences was performed and the sequences were cloned into the pVax1 expression vector.

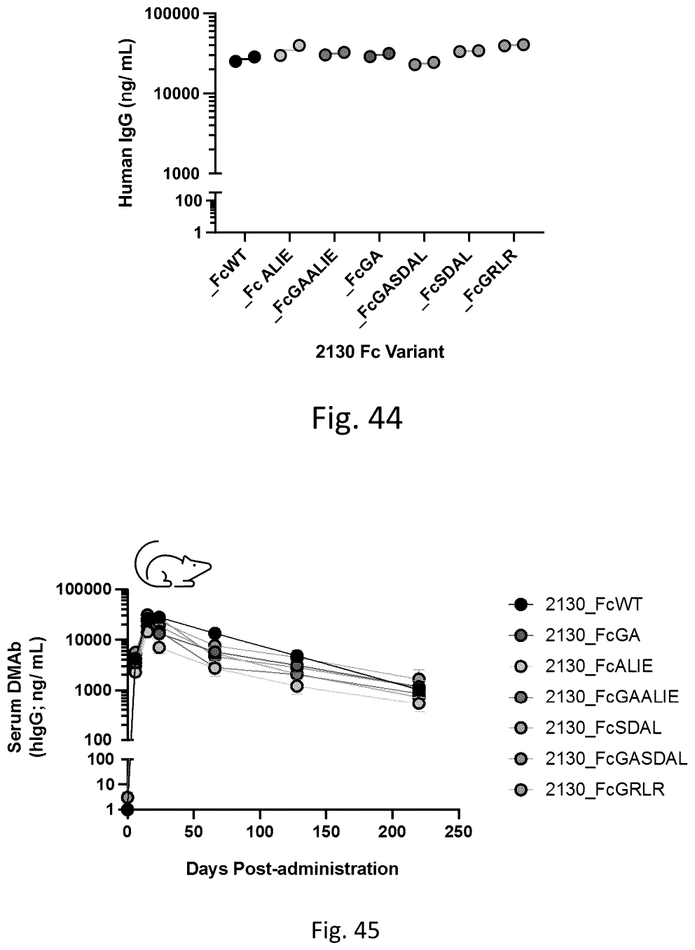

FIG. 44 shows the expression of the indicated 2130 DMAb variants in vitro, and FIG. 45 shows the in vivo expression kinetics of Fc-engineered 2130 DMAb variants.

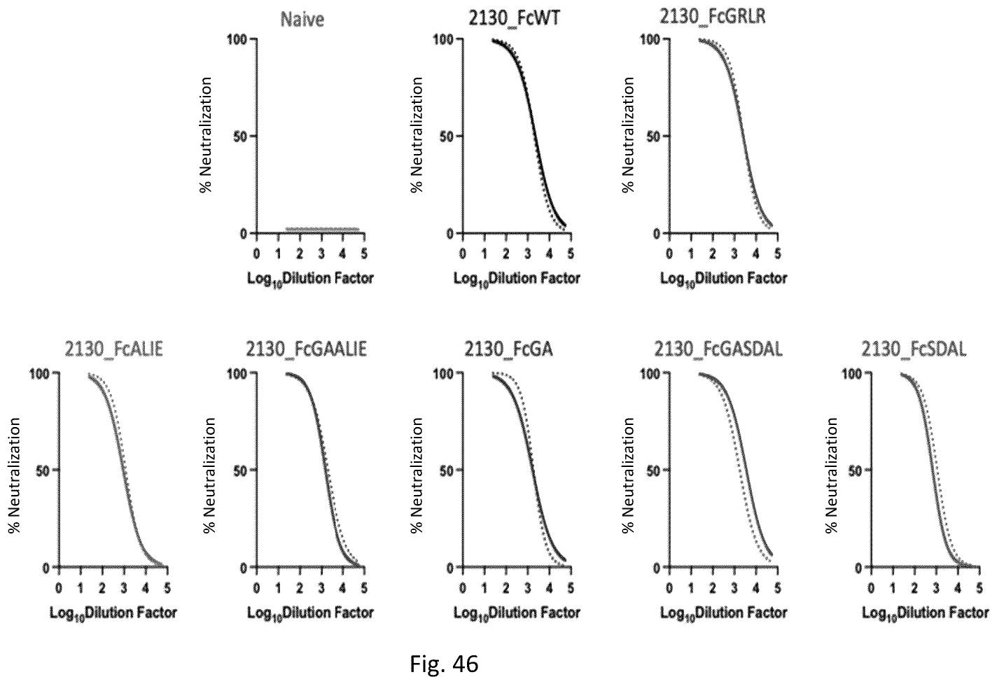

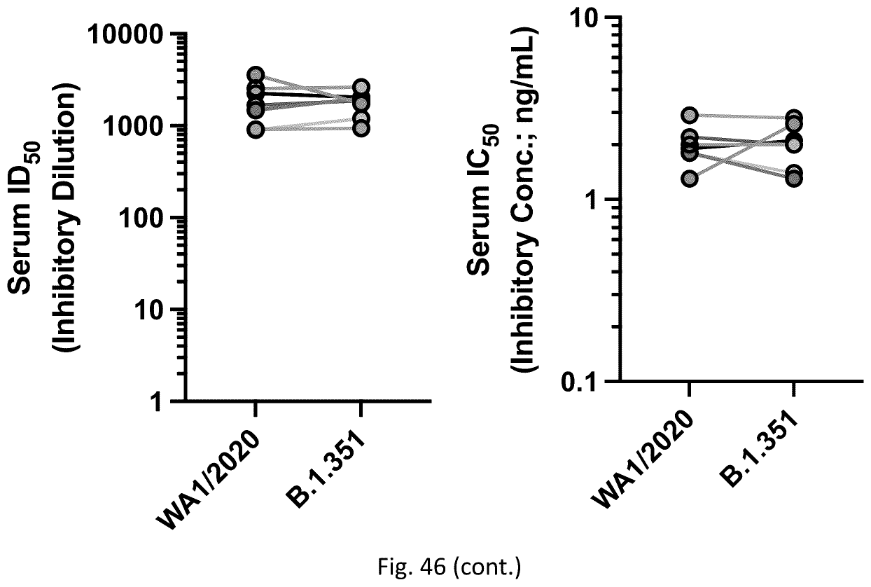

Experiments were preformed to analyze the viral neutralization of the modified antibodies. FIG. 46 shows that in vivo-launched 2130 DMAb variants retain comparable antiviral/neutralizing activity against SARSpCoV-2 pseudotyped virus (strains WA1/2020 and B.1.351).

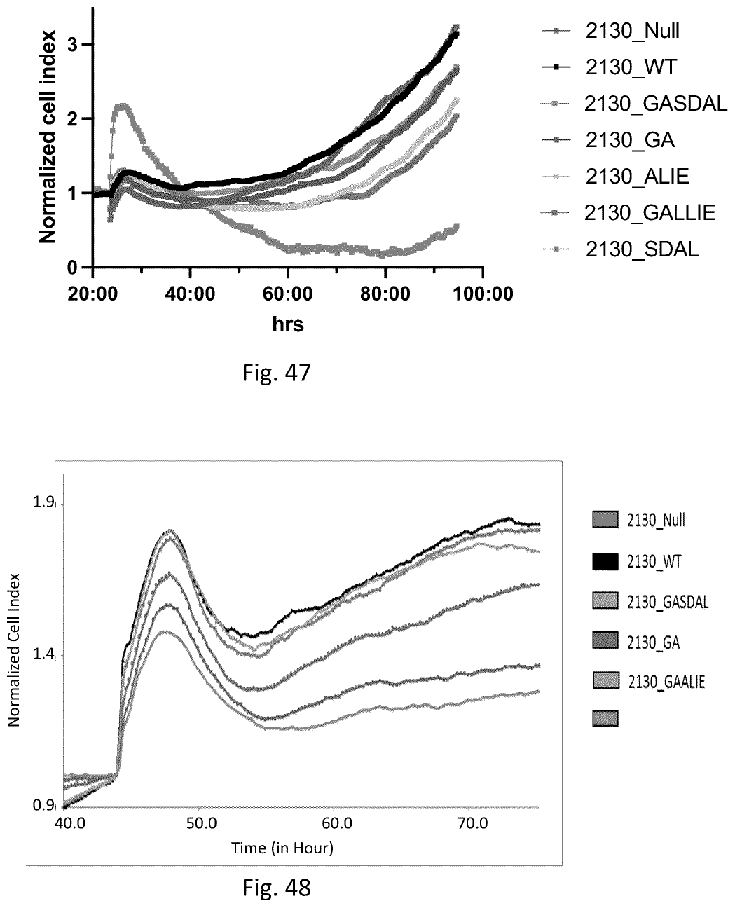

Functional assays were performed to analyze the cytolytic capacity against spike-expressing target cells (HEK293T-Spike). FIG. 47 depicts data demonstrating that the effector-enhanced 2130 DMAb variants demonstrate improved in vitro elimination of HEK293 cells expressing SARS-CoV-2 spike protein compared the parental IgG1 WT construct. This is particularly pronounced for the SDAL variant. The reduced impedance/cell index indicates direct killing/elimination of target cells (HEK293T-Spike). The loss of impedance is more pronounced in the presence of effector-enhanced variants compared to the parental WT DMAB. The data indicates that many of these variants are able to facilitate target elimination via effector cell engagement (human PMBCs).

FIG. 48 depicts data demonstrating that the effector-enhanced 2130 DMAb variants demonstrate improved in vitro elimination of Hela cells expressing SARS-CoV-2 spike protein compared the parental IgG1 WT construct. This is particularly pronounced for the SDAL variant. This data demonstrates that there is reproducible killing activity using a different target cell line.

### Clinical Sequences:

Clinical Name: AZD8076 Heavy Chain
pGX93322 PLASMID
Original Name: 36. DMAb AZD8895_HC_WT/YTE
pGX93322 PLASMID: DNA Sequence
SEQ ID NO: 99
Clinical Name: AZD8076 Heavy Chain
pGX93322 PLASMID
Original Name: 36. DMAb AZD8895_HC_WT/YTE
pGX93322 PLASMID: AA (Insert)
SEQ ID NO: 76
Clinical Name: AZD8076 Light Chain
Original Name: 22.DMAb AZD8895CLIN_LC_pVax1
pGX93315 PLASMID: DNA Sequence
SEQ ID NO: 100
Clinical Name: AZD8076 Light Chain
pGX93315 PLASMID
Original Name: 22.DMAb AZD8895CLIN_LC_pVax1
pGX93315 PLASMID: AA Sequence (Insert)
SEQ ID NO: 66
Clinical Name: AZD5396 Heavy Chain
pGX93321 PLASMID
Original Name: 35. DMAB AZD1061_HC_WT/YTE
pGX93321 PLASMID: DNA Sequence
SEQ ID NO: 101
Clinical Name: AZD5396 Heavy Chain
pGX93321 PLASMID
Original Name: 35. DMAB AZD1061_HC_WT/YTE
pGX93321 PLASMID: AA Sequence (Insert)
SEQ ID NO: 74
Clinical Name: AZD5396 Light Chain
pGX93311 PLASMID
Original Name: 18.DMAB AZ1061CLIN_LC_pVax1
pGX93311 PLASMID: DNA Sequence
SEQ ID NO: 102
Clinical Name: AZD5396 Light Chain
pGX93311 PLASMID
Original Name: 18.DMAB AZ1061CLIN_LC_pVax1
pGX93311 PLASMID: AA Sequence (Insert)
SEQ ID NO: 58

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

1. A nucleic acid molecule encoding an antibody or fragment thereof wherein the Fc domain of the heavy chain has been modified to include at least one selected from the group consisting of: a half-life extending variation, a stability increasing variation, a variation to alter complement binding, a variation to alter dimerization and a variation to alter the level of Fc-FcγR interaction.

2. The nucleic acid molecule of claim 1, wherein the nucleic acid molecule encodes a Fc domain of the heavy chain comprising at least one variation selected from the group consisting of M252Y, S254T, T256E, L234F, L235E, P331S, E430G, S239D, I332E, G236A, A330L, G236R, L328R, L235Q, and K322Q.

3. The nucleic acid molecule of claim 1, wherein the nucleic acid molecule encodes a Fc domain of the heavy chain comprising a combination of variations selected from the group consisting of:
a) M252Y/S254T/T256E (YTE);
b) L234F/L235E/P331S (TM);
c) S239D/I332E (DE);
d) S239D/I332E/E430G (DEG);
e) G236A/I332E (AE);
f) A330L/I332E (ALIE);
g) G236A/A330L/I332E (GAALIE);
h) S239D/A330L (SDAL);
i) G236A/S239D/A330L (GASDAL); and
j) G236R/L328R (GRLR).

4. The nucleic acid molecule of claim 1, further comprising a nucleotide sequence encoding a cleavage domain.

5. The nucleic acid molecule of claim 1, wherein the nucleotide sequence encodes a leader sequence.

6. The nucleic acid molecule of claim 1, wherein the nucleic acid molecule comprises an expression vector.

7. The nucleic acid molecule of claim 1 comprising a nucleotide sequence encoding a heavy chain of an anti-SARS-CoV-2 spike antigen synthetic antibody; and a nucleotide sequence encoding a light chain of an anti-SARS-CoV-2 spike antigen synthetic antibody.

8. The nucleic acid molecule of claim 7, wherein the nucleic acid molecule encodes an antibody sequence selected from the group consisting of SEQ ID NO:6, SEQ ID NO: 12, SEQ ID NO:18, SEQ ID NO:24, SEQ ID NO:26, SEQ ID NO:28, SEQ ID NO: 60, SEQ ID NO:62, SEQ ID NO:68, SEQ ID NO: 72, SEQ ID NO: 74, SEQ ID NO:88, SEQ ID NO: 90, SEQ ID NO:92, SEQ ID NO:94, SEQ ID NO:96 and SEQ ID NO:98.

9. The nucleic acid molecule of claim 8, wherein the nucleic acid molecule comprises a nucleotide sequence selected from the group consisting of SEQ ID NO:5, SEQ ID NO:11, SEQ ID NO:17, SEQ ID NO:23, SEQ ID NO:25, SEQ ID NO:27, SEQ ID NO: 59, SEQ ID NO:61, SEQ ID NO: 67, SEQ ID NO:71, SEQ ID NO: 73, SEQ ID NO:87, SEQ ID NO: 89, SEQ ID NO:91, SEQ ID NO: 93, SEQ ID NO:95, and SEQ ID NO:97.

10. A composition comprising at least one nucleic acid molecule encoding an antibody comprising an Fc variation or fragment thereof of claim 1.

11. The composition of claim 10 comprising a first nucleic acid molecule comprising a nucleotide sequence encoding a heavy chain of a synthetic antibody comprising at least one Fc variation; and a second nucleic acid molecule comprising a nucleotide sequence encoding a light chain of a synthetic antibody.

12. The composition of claim 10, further comprising a pharmaceutically acceptable excipient.

13. A method of preventing or treating a disease in a subject, the method comprising administering to the subject the nucleic acid molecule of claim 1.

14. The method of claim 13, wherein the disease is COVID-19.

15. A method of inducing an immune response against a disease or disorder in a subject, the method comprising administering to the subject the nucleic acid molecule of claim 1.

16. The method of claim 15, wherein the disease is COVID-19.

17. A method of preventing or treating a disease in a subject, the method comprising administering to the subject a composition of claim 10.

18. The method of claim 17, wherein the disease is COVID-19.

19. A method of inducing an immune response against a disease or disorder in a subject, the method comprising administering to the subject a composition of claim 10.

20. The method of claim 17, wherein the disease is COVID-19.


SEQUENCE LISTING (102 sequences)

SEQ ID NO: 1 - DNA, 714 bp, synthetic construct
  source 1..714: mol_type = other DNA
atggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctaacaccctgacactgagcaaggccgattacgagaagcacaaggtgtatgcctgcgaggtgacccatcaggggctgtcttctccagtgaccaaatccttcaatcgcggggaatgttgataa

SEQ ID NO: 2 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 3 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagaaggtggagccaaagtcttgcgataagacccacacatgccctccctgtccagcacctgagctgctgggcggcccaagcgtgttcctgtttccacccaagcccaaggacaccctgatgatcagcaggaccccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggacgagctgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 4 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagaaggtggagccaaagtcttgcgataagacccacacatgccctccctgtccagcacctgagttcgagggcggcccaagcgtgttcctgtttccacccaagcccaaggacaccctgatgatcagcaggaccccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagccagcatcgagaagaccatcagcaaggcaaagggacagccacgcgagccacaggtgtatacactgcctccatctagggacgagctgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 6 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 723 bp, synthetic construct
  source 1..723: mol_type = other DNA
atggtgctgcagacccaggtgtttatctccctgctgctgtggatctctggcgcctacggcgacatcgtgatgacccagtctcctgatagcctggccgtgtctctgggagagagggcaacaatcaactgtaagtctagccagagcgtgctgtattcctctaacaataagaattatctggcatggtaccagcagaagccaggacagccacccaagctgctgatgtactgggcatctacccgggagagcggagtgcctgaccgcttctctggcagcggctccggagcagagtttaccctgacaatcagctccctgcaggccgaggatgtggccatctattactgccagcagtattacagcaccctgaccttcggcggcggcaccaaggtggagatcaagaggacagtggccgcccctagcgtgttcatctttcctccaagcgacgagcagctgaagtccggcaccgcctctgtggtgtgcctgctgaacaacttctacccaagagaggccaaggtgcagtggaaggtggataacgccctgcagtccggcaattctcaggagagcgtgaccgagcaggactccaaggattctacatactctctgagcaacaccctgacactgagcaaggccgactatgagaagcacaaggtgtacgcctgcgaagtgacccaccaggggctgagcagtccagtgaccaagtctttcaatcggggagaatgctgataa

SEQ ID NO: 8 - protein, 239 aa, synthetic construct
MVLQTQVFISLLLWISGAYGDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 9 - DNA, 1446 bp, synthetic construct
  source 1..1446: mol_type = other DNA
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagaaggtggagccaaagagctgtgataagacccacacatgccctccctgtccagcaccagagctgctgggcggcccatccgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctgccccaatcgagaagaccatctccaaggcaaagggacagcccagggagcctcaggtgtatacactgcctccaagcagagacgagctgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagtgataa

SEQ ID NO: 10 - protein, 480 aa, synthetic construct
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1446 bp, synthetic construct
  source 1..1446: mol_type = other DNA
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagaaggtggagccaaagagctgtgataagacccacacatgccctccctgtccagcaccagagttcgagggcggcccatccgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctgccagcatcgagaagaccatctccaaggcaaagggacagcccagggagcctcaggtgtatacactgcctccaagcagagacgagctgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagtgataa

SEQ ID NO: 12 - protein, 480 aa, synthetic construct
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 714 bp, synthetic construct
  source 1..714: mol_type = other DNA
atggtgctgcagacccaggtgttcatcagcctgctgctgtggatctccggagcatacggagaggtggtgctgacccagtccccaggcacactgtccctgtctcctggagagagagccaccctgagctgcagggcaagccagtccgtgagctcctcttacctggcctggtatcagcagaagcctggacaggccccacgcctgctgatctatggagccagctcccgcgccaccggcatccccgacaggttttctggcagcggctccggcacagatttcaccctgacaatctccaggctggagcctgaggacttcgccgtgtaccactgtcagcactttggctctagctcccagtggaccttcggccagggcacaaaggtggagatcaagagaaccgtggccgccccatccgtgttcatctttccaccctctgacgagcagctgaagagcggcacagcctccgtggtgtgcctgctgaacaatttctatcccagggaggccaaggtgcagtggaaggtggataacgccctgcagtccggcaattctcaggagagcgtgaccgagcaggactccaaggattctacatactctctgagcaacaccctgacactgagcaaggccgattacgagaagcacaaggtgtatgcctgcgaggtcactcaccaggggctgtccagtcccgtcactaagtctttcaatagaggcgaatgttgataa

SEQ ID NO: 14 - protein, 236 aa, synthetic construct
MVLQTQVFISLLLWISGAYGEVVLTQSPGTLSLSPGERATLSCRASQSVSSSYLAWYQQKPGQAPRLLIYGASSRATGIPDRFSGSGSGTDFTLTISRLEPEDFAVYHCQHFGSSSQWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 15 - DNA, 1422 bp, synthetic construct
  source 1..1422: mol_type = other DNA
atggactggacttggagaatcctgtttctggtcgccgccgctactggaacccacgctcagatgcagctggtgcagagtggacccgaagtgaagaagcccggcaccagcgtgaaggtgtcctgtaaggcctctggcttcacctttacaagctccgccgtgcagtgggtgaggcaggccagaggccagcggctggagtggatcggatggatcgcagtgggctccggaaacaccaattacgcccagaagtttcaggagcgcgtgtctatcacaagggacatgtctaccagcacagcctatatggagctgtctagcctgagatccgaggataccgccgtgtactattgcgccgccccttactgttcccggacatcttgccacgacgcctttgatatctggggccagggcaccaaggtgacagtgtcctctgcctctacaaagggaccaagcgtgttcccactggcacctagctccaagtccacctctggcggcacagccgccctgggctgtctggtgaaggactactttcctgagccagtgaccgtgagctggaactccggcgccctgacctccggagtgcacacattccctgccgtgctgcagtctagcggcctgtacagcctgtcctctgtggtgaccgtgccaagctcctctctgggcacccagacatatatctgtaacgtgaatcacaagccttccaatacaaaggtggacaagaaggtggagccaaagtcttgcgataagacccacacatgccctccctgtccagcacctgagctgctgggcggcccaagcgtgttcctgtttccacccaagcccaaggacaccctgatgatcagccggaccccagaggtgacatgcgtggtggtggacgtgtcccacgaggaccccgaggtgaagttcaactggtacgtggatggcgtggaggtgcacaatgccaagaccaagcccagagaggagcagtacaactccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggacgagctgaccaagaaccaggtgagcctgacatgcctggtgaagggcttttaccccagcgatatcgccgtggagtgggagtccaatggccagcctgagaacaattacaagaccacaccccctgtgctggactctgatggcagcttctttctgtattctaagctgaccgtggacaagagccgctggcagcagggcaacgtgttcagctgttccgtgatgcacgaggccctgcacaatcactatacccagaagtctctgagcctgtcccctggcaagtgataa

SEQ ID NO: 16 - protein, 472 aa, synthetic construct
MDWTWRILFLVAAATGTHAQMQLVQSGPEVKKPGTSVKVSCKASGFTFTSSAVQWVRQARGQRLEWIGWIAVGSGNTNYAQKFQERVSITRDMSTSTAYMELSSLRSEDTAVYYCAAPYCSRTSCHDAFDIWGQGTKVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 1422 bp, synthetic construct
  source 1..1422: mol_type = other DNA
atggactggacttggagaatcctgtttctggtcgccgccgctactggaacccacgctcagatgcagctggtgcagagtggacccgaagtgaagaagcccggcaccagcgtgaaggtgtcctgtaaggcctctggcttcacctttacaagctccgccgtgcagtgggtgaggcaggccagaggccagcggctggagtggatcggatggatcgcagtgggctccggaaacaccaattacgcccagaagtttcaggagcgcgtgtctatcacaagggacatgtctaccagcacagcctatatggagctgtctagcctgagatccgaggataccgccgtgtactattgcgccgccccttactgttcccggacatcttgccacgacgcctttgatatctggggccagggcaccaaggtgacagtgtcctctgcctctacaaagggaccaagcgtgttcccactggcacctagctccaagtccacctctggcggcacagccgccctgggctgtctggtgaaggactactttcctgagccagtgaccgtgagctggaactccggcgccctgacctccggagtgcacacattccctgccgtgctgcagtctagcggcctgtacagcctgtcctctgtggtgaccgtgccaagctcctctctgggcacccagacatatatctgtaacgtgaatcacaagccttccaatacaaaggtggacaagaaggtggagccaaagtcttgcgataagacccacacatgccctccctgtccagcacctgagttcgagggcggcccaagcgtgttcctgtttccacccaagcccaaggacaccctgatgatcagccggaccccagaggtgacatgcgtggtggtggacgtgtcccacgaggaccccgaggtgaagttcaactggtacgtggatggcgtggaggtgcacaatgccaagaccaagcccagagaggagcagtacaactccacctatcgggtggtgtctgtgctgacagtgctgcaccaggactggctgaacggcaaggagtacaagtgtaaggtgtctaataaggccctgccagccagcatcgagaagaccatcagcaaggcaaagggacagccacgcgagccacaggtgtatacactgcctccatctagggacgagctgaccaagaaccaggtgagcctgacatgcctggtgaagggcttttaccccagcgatatcgccgtggagtgggagtccaatggccagcctgagaacaattacaagaccacaccccctgtgctggactctgatggcagcttctttctgtattctaagctgaccgtggacaagagccgctggcagcagggcaacgtgttcagctgttccgtgatgcacgaggccctgcacaatcactatacccagaagtctctgagcctgtcccctggcaagtgataa

SEQ ID NO: 18 - protein, 472 aa, synthetic construct
MDWTWRILFLVAAATGTHAQMQLVQSGPEVKKPGTSVKVSCKASGFTFTSSAVQWVRQARGQRLEWIGWIAVGSGNTNYAQKFQERVSITRDMSTSTAYMELSSLRSEDTAVYYCAAPYCSRTSCHDAFDIWGQGTKVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 699 bp, synthetic construct
  source 1..699: mol_type = other DNA
atggtgctgcagacccaggtgttcatctctctgctgctgtggatcagcggagcatacggagagatcgtgctgacccagagccctggcacactgagcctgtccccaggagagagggccacactgtcctgcagagcctctcagagcgtgagctcctcttacctggcctggtatcagcagaagccaggacaggcccccaggctgctgatctatggagccagctcccgggccaccggcatccccgaccgcttctccggctctggcagcggcacagacttcaccctgacaatctctaggctggagcctgaggacttcgccgtgtactattgccagcagcacgataccagcctgacatttggccagggcaccaaggtggagatcaagagaacagtggccgccccatccgtgttcatctttcccccttctgacgagcagctgaagagcggaaccgcatccgtggtgtgcctgctgaacaatttctacccccgggaggccaaggtgcagtggaaggtggataacgccctgcagtccggcaattctcaggagagcgtgaccgagcaggacaaggattccacatattctctgtctagcaccctgacactgtccaaggccgactacgagaagcacaaggtgtatgcatgcgaggtgacccaccagggactgtcctctcccgtgacaaagagctttaaccgcggcgagtgt

SEQ ID NO: 20 - protein, 233 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQQHDTSLTFGQGTKVEIKRTVAAPSVFIFPPSDEQLKSGTASVVCLLNNFYPREAKVQWKVDNALQSGNSQESVTEQDKDSTYSLSSTLTLSKADYEKHKVYACEVTHQGLSSPVTKSFNRGEC

SEQ ID NO: 21 - DNA, 1431 bp, synthetic construct
  source 1..1431: mol_type = other DNA
atggactggacctggagaatcctgttcctggtggcagcagcaaccggaacacacgcacaggtgcagctggtgcagtccggagcagaggtgaagaagcctggagcctctgtgaaggtgagctgcaaggcctccggctacacctttacatcttatggaatcagctgggtgaggcaggcaccaggacagggactggagtggatgggctggatcagcgcctacaacggcaatacaaactatgcccagaagctgcagggcagagtgaccatgaccacagacaccagcacatccaccgcctacatggagctgaggtctctgagaagcgacgatacagccgtgtactattgcgcccgggactatacccgcggcgcctggttcggagagtctctgatcggcggatttgataattggggccagggcacactggtgaccgtgtctgccagcacaaagggaccaagcgtgttcccactggcacccagctccaagtccacatctggcggcaccgccgccctgggatgtctggtgaaggattacttcccagagcccgtgaccgtgtcctggaattctggcgccctgacaagcggcgtgcacacctttccagccgtgctgcagtctagcggcctgtactccctgtcctctgtggtgacagtgcccagctcctctctgggcacacagacctatatctgcaatgtgaaccacaagccaagcaacaccaaggtggacaagaaggtggagcccaagtcctgtgataagacacacacctgccctccctgtcctgcaccagagctgctgggcggcccatccgtgttcctgtttccacccaagcctaaggacaccctgatgatctctcggacacccgaggtgacctgcgtggtggtggacgtgagccacgaggaccccgaggtgaagtttaattggtacgtggatggcgtggaggtgcacaacgccaagaccaagcccagggaggagcagtacaactccacatatagagtggtgtctgtgctgaccgtgctgcaccaggactggctgaatggcaaggagtataagtgcaaggtgtccaacaaggccctgcccgcccctatcgagaagacaatctctaaggcaaagggacagcctcgggagccacaggtgtacaccctgcctccatcccgcgacgagctgacaaagaatcaggtgtctctgacctgtctggtgaagggcttctatccttctgatatcgcagtggagtgggagagcaacggacagccagagaacaattacaagaccacaccccctgtgctggacagcgatggctccttctttctgtatagcaagctgacagtggataagtcccgctggcagcagggcaacgtgttcagctgttccgtgatgcacgaggccctgcacaaccactacacccagaagtctctgagcctgtcccctggcaagtgataa

SEQ ID NO: 22 - protein, 581 aa, synthetic construct
MDWTWRILFLVAAATGTHAQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYTRGAWFGESLIGGFDNWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 1431 bp, synthetic construct; identical to SEQ ID NO: 21
  source 1..1431: mol_type = other DNA

SEQ ID NO: 24 - protein, 475 aa, synthetic construct
MDWTWRILFLVAAATGTHAQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYTRGAWFGESLIGGFDNWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 25 - DNA, 1431 bp, synthetic construct
  source 1..1431: mol_type = other DNA
atggactggacctggagaatcctgttcctggtggcagcagcaaccggaacacacgcacaggtgcagctggtgcagtccggagcagaggtgaagaagcctggagcctctgtgaaggtgagctgcaaggcctccggctacacctttacatcttatggaatcagctgggtgaggcaggcaccaggacagggactggagtggatgggctggatcagcgcctacaacggcaatacaaactatgcccagaagctgcagggcagagtgaccatgaccacagacaccagcacatccaccgcctacatggagctgaggtctctgagaagcgacgatacagccgtgtactattgcgcccgggactatacccgcggcgcctggttcggagagtctctgatcggcggatttgataattggggccagggcacactggtgaccgtgtctgccagcacaaagggaccaagcgtgttcccactggcacccagctccaagtccacatctggcggcaccgccgccctgggatgtctggtgaaggattacttcccagagcccgtgaccgtgtcctggaattctggcgccctgacaagcggcgtgcacacctttccagccgtgctgcagtctagcggcctgtactccctgtcctctgtggtgacagtgcccagctcctctctgggcacacagacctatatctgcaatgtgaaccacaagccaagcaacaccaaggtggacaagaaggtggagcccaagtcctgtgataagacacacacctgccctccctgtcctgcaccagagctgctgggcggcccagacgtgttcctgtttccacccaagcctaaggacaccctgatgatctctcggacacccgaggtgacctgcgtggtggtggacgtgagccacgaggaccccgaggtgaagtttaattggtacgtggatggcgtggaggtgcacaacgccaagaccaagcccagggaggagcagtacaactccacatatagagtggtgtctgtgctgaccgtgctgcaccaggactggctgaatggcaaggagtataagtgcaaggtgtccaacaaggccctgcccgcccctgaggagaagacaatctctaaggcaaagggacagcctcgggagccacaggtgtacaccctgcctccatcccgcgacgagctgacaaagaatcaggtgtctctgacctgtctggtgaagggcttctatccttctgatatcgcagtggagtgggagagcaacggacagccagagaacaattacaagaccacaccccctgtgctggacagcgatggctccttctttctgtatagcaagctgacagtggataagtcccgctggcagcagggcaacgtgttcagctgttccgtgatgcacgaggccctgcacaaccactacacccagaagtctctgagcctgtcccctggcaagtgataa

SEQ ID NO: 26 - protein, 475 aa, synthetic construct
MDWTWRILFLVAAATGTHAQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYTRGAWFGESLIGGFDNWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 1431 bp, synthetic construct
  source 1..1431: mol_type = other DNA
atggactggacctggagaatcctgttcctggtggcagcagcaaccggaacacacgcacaggtgcagctggtgcagtccggagcagaggtgaagaagcctggagcctctgtgaaggtgagctgcaaggcctccggctacacctttacatcttatggaatcagctgggtgaggcaggcaccaggacagggactggagtggatgggctggatcagcgcctacaacggcaatacaaactatgcccagaagctgcagggcagagtgaccatgaccacagacaccagcacatccaccgcctacatggagctgaggtctctgagaagcgacgatacagccgtgtactattgcgcccgggactatacccgcggcgcctggttcggagagtctctgatcggcggatttgataattggggccagggcacactggtgaccgtgtctgccagcacaaagggaccaagcgtgttcccactggcacccagctccaagtccacatctggcggcaccgccgccctgggatgtctggtgaaggattacttcccagagcccgtgaccgtgtcctggaattctggcgccctgacaagcggcgtgcacacctttccagccgtgctgcagtctagcggcctgtactccctgtcctctgtggtgacagtgcccagctcctctctgggcacacagacctatatctgcaatgtgaaccacaagccaagcaacaccaaggtggacaagaaggtggagcccaagtcctgtgataagacacacacctgccctccctgtcctgcaccagagctgctgggcggcccagacgtgttcctgtttccacccaagcctaaggacaccctgatgatctctcggacacccgaggtgacctgcgtggtggtggacgtgagccacgaggaccccgaggtgaagtttaattggtacgtggatggcgtggaggtgcacaacgccaagaccaagcccagggaggagcagtacaactccacatatagagtggtgtctgtgctgaccgtgctgcaccaggactggctgaatggcaaggagtataagtgcaaggtgtccaacaaggccctgcccgcccctgaggagaagacaatctctaaggcaaagggacagcctcgggagccacaggtgtacaccctgcctccatcccgcgacgagctgacaaagaatcaggtgtctctgacctgtctggtgaagggcttctatccttctgatatcgcagtggagtgggagagcaacggacagccagagaacaattacaagaccacaccccctgtgctggacagcgatggctccttctttctgtatagcaagctgacagtggataagtcccgctggcagcagggcaacgtgttcagctgttccgtgatgcacggcgccctgcacaaccactacacccagaagtctctgagcctgtcccctggcaagtgataa

SEQ ID NO: 28 - protein, 475 aa, synthetic construct
MDWTWRILFLVAAATGTHAQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDYTRGAWFGESLIGGFDNWGQGTLVTVSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 29 - DNA, 723 bp, synthetic construct
  source 1..723: mol_type = other DNA
atggtgctgcagacacaggtgttcatcagcctgctgctgtggatctccggcgcctacggcgacatcgtgatgacacagtctccactgagcctgccagtgaccctgggacagccagcctccatctcttgccggagctcccagagcctggtgtactccgatggcaacaccttcctgaattggtttcagcagcgccctggccagagcccaaggagactgatctatcaggtgtctaacagggacagcggagtgccagatcgcttcagcggctccggctctggaaccgactttacactgaagatctccagggtggaggccgaggatgtgggcgtgtactattgcatgcagggcacccactggcccaggacattcggccagggcaccaagctggagatcaagagaacagtggccgcccctagcgtgttcatctttccccctagcgacgagcagctgaagtccggcaccgcctctgtggtgtgcctgctgaacaacttctaccctagggaggccaaggtgcagtggaaggtggataacgccctgcagtccggcaattctcaggagagcgtgacagagcaggactccaaggattctacctatagcctgtccaacaccctgacactgtccaaggccgactacgagaagcacaaggtgtatgcctgcgaggtgacacaccagggcctgtctagccccgtgaccaagtcttttaatagaggcgagtgttgataa

SEQ ID NO: 30 - protein, 238 aa, synthetic construct
MVLQTQVFISLLLWISGAYGDIVMTQSPLSLPVTLGQPASISCRSSQSLVYSDGNTFLNWFQQRPGQSPRRLIYQVSNRDSGVPDRFSGSGSGTDFTLKISRVEAEDVGVYYCMQGTHWPRTFGQGTKLEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGE

SEQ ID NO: 31 - DNA, 758 bp, synthetic construct
  source 1..758: mol_type = other DNA
atggactggacctggagaatcctgttcctggtggcagcagcaaccggaacacacgcacaggtgcagctgctggagagcggcggcggcctggtgcagcccggcggctctctgcggctgagctgcgccgcctccggcttcacctttagctcctacgcaatgtcctgggtgcgccaggcacctggcaagggactggagtgggcatctgccatctccggctctggcggcagcacatactatgccgactccgtggagggcaggttcaccatctctagagataacagcaagaatacactgtacctgcagatgaacagcctgagggccgaggacaccgccgtgtactattgcgccaaggatacaggctactgcggcgacgattgttatatcaagctgatcagaggcggccctgactattggggacagggcaccctggtgacagtgtctagcgcctccaccaagggaccaagcgtgttcccactggcaccctcctctaagagcacctccggcggcacagccgccctgggctgtctggtgaaggattatttcccagagcccgtgacagtgtcctggaactctggcgccctgacctccggagtgcacacatttccagccgtgctgcagagctccggcctgtacagcctgtctagcgtggtgaccgtgccctcctctagcctgggcacccagacatatatctgcaacgtgaatcacaagccatccaatacaaaggtggacaagaaggtggagcccaagtcttgtgataa

SEQ ID NO: 32 - protein, 478 aa, synthetic construct
MDWTWRILFLVAAATGTHAQVQLLESGGGLVQPGGSLRLSCAASGFTFSSYAMSWVRQAPGKGLEWASAISGSGGSTYYADSVEGRFTISRDNSKNTLYLQMNSLRAEDTAVYYCAKDTGYCGDDCYIKLIRGGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 714 bp, synthetic construct
  source 1..714: mol_type = other DNA
atggcctggacaccactgttcctgtttctgctgacctgctgtcccggcggcagcaactcccagtctgccctgacacagccagcaagcgtgtccggctctcctggacagtctatcaccatcagctgcaccggcacaagctccgacgtgggcggctacaattatgtgagctggtaccagcagcacccaggcaaggcccccaagctgatgatctatgacgtgagcaagaggccatccggcgtgtctaacaggttcagcggctccaagtctggcaataccgcctccctgacaatctctggcctgcagagcgaggacgaggccgattactattgcaacagcctgaccagcatctccacatgggtgttcggcggcggaaccaagctgacagtgctgggccagcctaaggccaacccaaccgtgacactgtttcccccttctagcgaggagctgcaggccaataaggccaccctggtgtgcctgatctctgacttctacccaggagcagtgaccgtggcatggaaggcagatggctcccctgtgaaggcaggagtggagaccacaaagccaagcaagcagtccaacaataagtacgccgcctcctcttatctgtccctgacacccgagcagtggaagtctcacaggtcttatagctgccaggtgacccacgagggcagcaccgtggagaagacagtggcccctaccgagtgttcctgataa

SEQ ID NO: 34 - protein, 236 aa, synthetic construct
MAWTPLFLFLLTCCPGGSNSQSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 35 - DNA, 1413 bp, synthetic construct
  source 1..1413: mol_type = other DNA
atggactggacctggagaatcctgttcctggtggcagcagcaaccggaacacacgcacaggtgcagctggtggagagcggcggcggcgtggtgcagcctggcaggagcctgagactgtcctgcgcagcatctggcttcacctttagcaactacgccatgtattgggtgaggcaggcaccaggcaagggactggagtgggtggccgtgatcagctacgacggctccaataagtactatgccgattccgtgaagggccggttcaccatctctcgcgacaacagcaagaatacactgtatctgcagatgaacagcctgagaaccgaggatacagccgtgtactattgcgcctctggcagcgactacggcgattatctgctggtgtactggggccagggcaccctggtgacagtgagctccgcctccaccaagggaccaagcgtgttcccactggcaccctctagcaagtccacctctggcggcacagccgccctgggctgtctggtgaaggattacttcccagagcccgtgacagtgtcttggaacagcggcgccctgacctctggagtgcacacatttccagccgtgctgcagtcctctggcctgtacagcctgagctccgtggtgaccgtgccctctagctccctgggcacccagacatatatctgcaacgtgaatcacaagccatctaatacaaaggtggacaagaaggtggagcccaagagctgtgataagacccacacatgccctccctgtcctgcaccagagctgctgggcggcccatccgtgttcctgtttccacccaagcctaaggacaccctgatgatctcccggaccccagaggtgacatgcgtggtggtggacgtgtctcacgaggaccccgaggtgaagtttaactggtacgtggatggcgtggaggtgcacaatgccaagaccaagcccagggaggagcagtacaacagcacctatagagtggtgtccgtgctgacagtgctgcaccaggactggctgaacggcaaggagtataagtgcaaggtgagcaataaggccctgcccgcccctatcgagaagaccatctccaaggcaaagggacagcctcgggagccacaggtgtacacactgcctccaagccgcgacgagctgaccaagaaccaggtgtccctgacatgtctggtgaagggcttctatccttccgatatcgccgtggagtgggagtctaatggccagccagagaacaattacaagaccacaccccctgtgctggactccgatggctctttctttctgtattctaagctgaccgtggataagagccgctggcagcagggcaacgtgtttagctgttccgtgatgcacgaggccctgcacaatcactacacacagaagtctctgagcctgtcccctggcaagtgataa

SEQ ID NO: 36 - protein, 469 aa, synthetic construct
MDWTWRILFLVAAATGTHAQVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 708 bp, synthetic construct
  source 1..708: mol_type = other DNA
atggtgctgcagacccaggtgttcatcagcctgctgctgtggatctccggcgcctacggcgacatccagatgacacagagcccaagctccctgtccgcctctgtgggcgacagggtgaccatcacatgccaggcctcccaggatatcaccaactacctgaattggtatcagcagaagccaggcaaggcccccaagctgctgatctacgcagcatccaacctggagaccggagtgccctctcgcttcagcggctccggctctggcacagatttcacctttacaatcagcggcctgcagcctgaggacatcgccacctactattgccagcagtatgataatctgccactgaccttcggcggcggcacaaaggtggagatcaagaggaccgtggccgcccccagcgtgttcatctttccccctagcgacgagcagctgaagtccggcacagcctctgtggtgtgcctgctgaacaatttctaccctagagaggccaaggtgcagtggaaggtggataacgccctgcagtctggcaatagccaggagtccgtgaccgagcaggactctaaggatagcacatatagcctgtccaacaccctgacactgtctaaggccgactacgagaagcacaaggtgtatgcctgcgaggtgacccaccagggcctgtctagccccgtgacaaagtcctttaataggggcgagtgttgataa

SEQ ID NO: 38 - protein, 234 aa, synthetic construct
MVLQTQVFISLLLWISGAYGDIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 39 - DNA, 1413 bp, synthetic construct
  source 1..1413: mol_type = other DNA
atggattggacctggagaatcctgttcctggtggcagcagcaaccggaacacacgcacaggtgcagctggtggagagcggcggcggcctggtgaagcccggcggctctctgaggctgagctgcgcagcatccggcttcaccttttctgactactatatgagctggatcagacaggcacctggcaagggactggagtgggtgtcctacatcacctattccggctctacaatctactatgccgattccgtgaagggccggtttaccatctctcgcgacaacgccaagagctccctgtacctgcagatgaatagcctgcgcgccgaggacacagccgtgtactattgcgccagggatagaggcaccacaatggtgcctttcgactattggggccagggcaccctggtgacagtgtctagcgcctccaccaagggaccaagcgtgttcccactggcaccctcctctaagagcacctccggcggcacagccgccctgggctgtctggtgaaggattacttcccagagcccgtgacagtgtcctggaactctggcgccctgacctccggagtgcacacatttccagccgtgctgcagagctccggcctgtacagcctgtctagcgtggtgaccgtgccctcctctagcctgggcacccagacatatatctgcaacgtgaatcacaagccatccaatacaaaggtggataagaaggtggagcccaagtcttgtgacaagacccacacatgccctccctgtcctgcaccagagctgctgggcggcccaagcgtgttcctgtttccacccaagcctaaggataccctgatgatcagcaggacccccgaggtgacatgcgtggtggtggacgtgtcccacgaggaccccgaggtgaagttcaactggtacgtggacggcgtggaggtgcacaatgccaagaccaagccaagggaggagcagtacaacagcacctatagagtggtgtccgtgctgacagtgctgcaccaggattggctgaacggcaaggagtataagtgcaaggtgtctaataaggccctgcccgcccctatcgagaagaccatcagcaaggcaaagggacagcctcgggagccacaggtgtacacactgcctccatctcgcgatgagctgaccaagaaccaggtgagcctgacatgtctggtgaagggcttctatcctagcgacatcgccgtggagtgggagtccaatggccagccagagaacaattacaagaccacaccccctgtgctggacagcgatggctccttctttctgtattctaagctgaccgtggacaagagcagatggcagcagggcaacgtgttttcttgtagcgtgatgcacgaggccctgcacaatcactacacacagaagtccctgtctctgagccctggcaagtgataa

SEQ ID NO: 40 - protein, 469 aa, synthetic construct
MDWTWRILFLVAAATGTHAQVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - DNA, 714 bp, synthetic construct
  source 1..714: mol_type = other DNA
atggcctggacaccactgttcctgtttctgctgacctgctgtcccggcggctctaacagccagtccgccctgacacagccagcatctgtgagcggctcccctggacagtctatcaccatcagctgcaccggcacaagctccgacgtgggcagctacaatctggtgtcctggtatcagcagcacccaggcaaggcccccaagctgatgatctacgagggctccaagaggccatccggcgtgtctaacagattttctggcagcaagtccggcaataccgcctccctgacaatctctggactgcaggcagaggacgaggcagattactattgctgtagctatgcaggctctagcaccctggtgttcggcggcggaaccaagctgacagtgctgggccagcctaaggccaacccaaccgtgacactgtttcccccttcctctgaggagctgcaggccaataaggccaccctggtgtgcctgatctccgacttctacccaggagcagtgaccgtggcatggaaggcagatggcagccctgtgaaggcaggagtggagaccacaaagccaagcaagcagtccaacaataagtacgccgccagctcctatctgtctctgacacccgagcagtggaagagccacaggtcttatagctgccaggtgacccacgagggctccaccgtggagaagacagtggcccctaccgagtgttcttgataa

SEQ ID NO: 42 - protein, 236 aa, synthetic construct
MAWTPLFLFLLTCCPGGSNSQSALTQPASVSGSPGQSITISCTGTSSDVGSYNLVSWYQQHPGKAPKLMIYEGSKRPSGVSNRFSGSKSGNTASLTISGLQAEDEADYYCCSYAGSSTLVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 43 - DNA, 1440 bp, synthetic construct
  source 1..1440: mol_type = other DNA
atggactggacctggagaatcctgttcctggtggcagcagcaaccggaacacacgcacaggtgcagctggtgcagtccggagcagaggtgaagaagccaggagcctctgtgaaggtgagctgcaaggcctccggctacaccttcacaggctactatatgcactgggtgagacaggcaccaggacagggactggagtggatgggctggatctctcctgtgagcggcggcaccaactacgcccagaagtttcagggccgggtgaccatgacacgcgacacctctatcagcacagcctatatggagctgtctaggctgagaagcgacgataccgccgtgtactattgcgccagggccccactgttcccaacaggcgtgctggcaggcgactactattactatggcatggacgtgtggggccagggcaccacagtgaccgtgagctccgcctccacaaagggaccaagcgtgttccctctggcaccatctagcaagtccacctctggcggcacagccgccctgggctgtctggtgaaggattacttcccagagcccgtgaccgtgtcctggaactctggcgccctgacctccggagtgcacacatttcccgccgtgctgcagtcctctggcctgtacagcctgagctccgtggtgaccgtgccttctagctccctgggcacccagacatatatctgcaacgtgaatcacaagccttctaatacaaaggtggacaagaaggtggagccaaagagctgtgataagacccacacatgccctccctgtcctgcaccagagctgctgggcggcccaagcgtgttcctgtttccacccaagcccaaggacaccctgatgatctcccggacccctgaggtgacatgcgtggtggtggacgtgtctcacgaggaccccgaggtgaagtttaactggtacgtggatggcgtggaggtgcacaatgccaagacaaagcctagggaggagcagtacaatagcacctatagagtggtgtccgtgctgacagtgctgcaccaggactggctgaacggcaaggagtataagtgcaaggtgtccaataaggccctgcccgcccctatcgagaagaccatcagcaaggcaaagggacagccccgggagcctcaggtgtacacactgcctccaagccgcgacgagctgaccaagaaccaggtgtccctgacatgtctggtgaagggcttctatccatccgatatcgccgtggagtgggagtctaatggccagcccgagaacaattacaagaccacaccccctgtgctggacagcgatggctccttctttctgtattctaagctgaccgtggataagagccgctggcagcagggcaacgtgtttagctgttccgtgatgcacgaggccctgcacaatcactacacacagaagtctctgagcctgtcccccggcaagtgataa

SEQ ID NO: 44 - protein, 478 aa, synthetic construct
MDWTWRILFLVAAATGTHAQVQLVQSGAEVKKPGASVKVSCKASGYTFTGYYMHWVRQAPGQGLEWMGWISPVSGGTNYAQKFQGRVTMTRDTSISTAYMELSRLRSDDTAVYYCARAPLFPTGVLAGDYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - DNA, 708 bp, synthetic construct
  source 1..708: mol_type = other DNA
atggtgctgcagacccaggtgttcatcagcctgctgctgtggatctccggcgcctacggcgacatccagatgacccagagcccatccacactgtctgccagcgtgggcgatagggtgaccatcacatgcagagcctcccagtctatcagcaactggctggcctggtttcagcagaagccaggcaaggcccccaagctgctgatctatgaggccagctccctggagtctggagtgcccagcaggttctccggctctggcagcggaaccgagtttaccctgacaatctctagcctgcagcctgacgatttcgccacatactattgccagcagtacaattcctatccatggacctttggccagggcacaaaggtggagatcaagaggaccgtggcagcaccaagcgtgttcatctttccccctagcgacgagcagctgaagtccggcacagcctctgtggtgtgcctgctgaacaatttctaccctcgcgaggccaaggtgcagtggaaggtggataacgccctgcagagcggcaattcccaggagtctgtgaccgagcaggacagcaaggattccacatattccctgtctaacaccctgacactgtccaaggccgactacgagaagcacaaggtgtatgcatgcgaggtgacccaccagggactgtcctctcccgtgacaaagagctttaatcggggcgagtgttgataa

SEQ ID NO: 46 - protein, 234 aa, synthetic construct
MVLQTQVFISLLLWISGAYGDIQMTQSPSTLSASVGDRVTITCRASQSISNWLAWFQQKPGKAPKLLIYEASSLESGVPSRFSGSGSGTEFTLTISSLQPDDFATYYCQQYNSYPWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 47 - DNA, 1410 bp, synthetic construct
  source 1..1410: mol_type = other DNA
atggattggacctggagaatcctgttcctggtggcagcagcaaccggaacacacgcacaggtgcagctggtggagagcggcggcggcgtggtgcagccaggcaggagcctgagactgtcctgcgccgcctctggcttcacctttagctcctacgcaatgcactgggtgaggcaggcaccaggcaagggactggagtgggtggccgtgatccccttcgacggcagaaacaagtactatgccgattccgtgaccggccggtttacaatcagccgcgacaactccaagaataccctgtatctgcagatgaatagcctgcgcgccgaggatacagccgtgtactattgcgcctctagctccggctacctgttccactccgactattggggccagggcaccctggtgacagtgtctagcgcctctaccaagggccctagcgtgtttccactggccccctcctctaagtccacctctggcggcacagccgccctgggctgtctggtgaaggactacttcccagagcccgtgacagtgtcttggaacagcggcgccctgaccagcggagtgcacacatttcctgccgtgctgcagagctccggcctgtactccctgtctagcgtggtgaccgtgccatcctctagcctgggcacccagacatatatctgcaacgtgaatcacaagcctagcaatacaaaggtggacaagaaggtggagccaaagtcctgtgataagacccacacatgccctccctgtcctgcaccagagctgctgggcggcccaagcgtgttcctgtttccacccaagcctaaggataccctgatgatctctaggaccccagaggtgacatgcgtggtggtggacgtgagccacgaggaccccgaggtgaagtttaactggtacgtggacggcgtggaggtgcacaatgccaagaccaagccaagggaggagcagtacaactccacctatagagtggtgtctgtgctgacagtgctgcaccaggattggctgaacggcaaggagtataagtgcaaggtgtccaataaggccctgcccgcccctatcgagaagaccatctctaaggcaaagggacagcctcgggagccacaggtgtacacactgcctccatcccgcgatgagctgaccaagaaccaggtgtctctgacatgtctggtgaagggcttctatccctctgacatcgccgtggagtgggagagcaatggccagcctgagaacaattacaagaccacaccccctgtgctggactccgatggctctttctttctgtatagcaagctgaccgtggacaagtccagatggcagcagggcaacgtgtttagctgttccgtgatgcacgaggccctgcacaatcactacacacagaagtctctgagcctgtcccctggcaagtgataa

SEQ ID NO: 48 - protein, 468 aa, synthetic construct
MDWTWRILFLVAAATGTHAQVQLVESGGGVVQPGRSLRLSCAASGFTFSSYAMHWVRQAPGKGLEWVAVIPFDGRNKYYADSVTGRFTISRDNSKNTLYLQMNSLRAEDTAVYYCASSSGYLFHSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - DNA, 720 bp, synthetic construct
  source 1..720: mol_type = other DNA
atggcctggacaccactgttcctgtttctgctgacctgctgtcccggcggctctaacagccagtccgccctgacacagccagcatctgtgagcggctcccctggacagtctatcaccatcagctgcaccggcacaagctccgacgtgggcggctacaattatgtgagctggtaccagcagcacccaggcaaggcccccaagctgatgatctatgacgtgagcaacaggccatccggcgtgtctaatagattttctggcagcaagtccggcaacaccgcctccctgacaatctctggactgcaggcagaggacgaggcagattactattgctctagctacacctcctctagcacacgggtgttcggcaccggcacaaaggtgaccgtgctgggccagcctaaggccaacccaaccgtgacactgtttcccccttcctctgaggagctgcaggccaataaggccaccctggtgtgcctgatctccgacttctatccaggagcagtgaccgtggcatggaaggcagatggctctcctgtgaaggccggcgtggagaccacaaagccaagcaagcagtccaacaataagtacgccgccagctcctatctgagcctgacacccgagcagtggaagtcccaccgctcttacagctgccaggtgacccacgagggctctaccgtggagaagacagtggcccctaccgagtgtagctgataactcgag

SEQ ID NO: 50 - protein, 236 aa, synthetic construct
MAWTPLFLFLLTCCPGGSNSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 51 - DNA, 1446 bp, synthetic construct
  source 1..1446: mol_type = other DNA
atggactggacctggagaatcctgttcctggtggcagcagcaaccggaacacacgcagaggtgcagctggtggagagcggcggcggcctgatccagcctggcggctctctgaggctgagctgcgcagcatccggctttaccgtgtccaacaattacatgtcttgggtgcgccaggcaccaggcaagggactggagtgggtgagcgtgatctatagcggcggctccacatactatgccgactccgtgaagggcaggttcaccatctctagagataagagcaagaacacactgtacctgcagatgaataggctgagagccgaggacaccgccgtgtactattgcgccagagagggcgaggtggagggctacaacgatttctggtctggctatagccgggaccgctactattttgattattggggccagggcaccctggtgacagtgagctccgccagcaccaagggaccatccgtgtttccactggcaccctctagcaagtctaccagcggcggcacagccgccctgggatgtctggtgaaggattacttcccagagcccgtgacagtgtcctggaactctggcgccctgacctccggagtgcacacatttccagccgtgctgcagtcctctggcctgtacagcctgagctccgtggtgaccgtgccctctagctccctgggcacccagacatatatctgcaacgtgaatcacaagccatccaatacaaaggtggacaagaaggtggagcccaagtcttgtgataagacccacacatgccctccctgtcctgcaccagagctgctgggcggcccaagcgtgttcctgtttccacccaagcctaaggacaccctgatgatctctcggacccccgaggtgacatgcgtggtggtggacgtgagccacgaggaccccgaggtgaagttcaactggtacgtggatggcgtggaggtgcacaatgccaagaccaagccccgggaggagcagtacaactctacctatcgcgtggtgagcgtgctgacagtgctgcaccaggactggctgaacggcaaggagtataagtgcaaggtgtccaataaggccctgcccgcccctatcgagaagaccatcagcaaggccaagggccagcctagggagccacaggtgtacacactgcctccatctagagacgagctgaccaagaaccaggtgagcctgacatgtctggtgaagggcttctatcctagcgatatcgccgtggagtgggagtccaatggccagccagagaacaattacaagaccacaccccctgtgctggacagcgatggctccttctttctgtattccaagctgaccgtggataagtctcgctggcagcagggcaacgtgttttcctgttctgtgatgcacgaggccctgcacaatcactacacacagaagagcctgtccctgtctcctggcaagtgataa

SEQ ID NO: 52 - protein, 480 aa, synthetic construct
MDWTWRILFLVAAATGTHAEVQLVESGGGLIQPGGSLRLSCAASGFTVSNNYMSWVRQAPGKGLEWVSVIYSGGSTYYADSVKGRFTISRDKSKNTLYLQMNRLRAEDTAVYYCAREGEVEGYNDFWSGYSRDRYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - DNA, 708 bp, synthetic construct
  source 1..708: mol_type = other DNA
atggtgctgcagacccaggtgttcatctccctgctgctgtggatctctggcgcctacggcgacatccagatgacacagagcccaagctccctgagcgcctccgtgggcgacagggtgaccatcacatgccaggccagccaggatatctccaactacctgaattggtatcagcagaagccaggcaaggcccccaagctgctgatctacgacgcctctaacctggagaccggagtgcccagccgcttctctggcagcggctccggaacagatttcacctttacaatctctagcctgcagcctgaggacatcgccacctactattgccagcagtatgataatctgccactgaccttcggcggcggcacaaaggtggagatcaagaggaccgtggccgcccccagcgtgttcatctttcccccttccgacgagcagctgaagtctggcacagccagcgtggtgtgcctgctgaacaatttctaccctagagaggccaaggtgcagtggaaggtggataacgccctgcagtccggcaattctcaggagagcgtgaccgagcaggactccaaggattctacatattctctgagcaacaccctgacactgtccaaggccgattacgagaagcacaaggtgtatgcatgcgaggtgacccaccagggactgtcctctcccgtgacaaagtcttttaataggggcgagtgttgataa

SEQ ID NO: 54 - protein, 234 aa, synthetic construct
MVLQTQVFISLLLWISGAYGDIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 55 - DNA, 708 bp, synthetic construct; identical to SEQ ID NO: 53
  source 1..708: mol_type = other DNA

SEQ ID NO: 56 - protein, 468 aa, synthetic construct
MDWTWRILFLVAAATGTHAQVQLVESGGGLVKPGGSLRLSCAASGFTFSDYYMSWIRQAPGKGLEWVSYISSSVSTIYYADSVKGRFTISRDNAKNSLYLQMNSLRAEDTAVYYCARDGVIPPRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - DNA, 726 bp, synthetic construct
  source 1..726: mol_type = other DNA
atggtgctgcagacccaggtgtttatctccctgctgctgtggatctctggcgcctacggcgacatcgtgatgacccagtctcctgatagcctggccgtgtctctgggagagagggcaacaatcaactgtaagtctagccagagcgtgctgtattcctctaacaataagaattatctggcatggtaccagcagaagccaggacagccacccaagctgctgatgtactgggcatctacccgggagagcggagtgcctgaccgcttctctggcagcggctccggagcagagtttaccctgacaatcagctccctgcaggccgaggatgtggccatctattactgccagcagtattacagcaccctgaccttcggcggcggcaccaaggtggagatcaagaggacagtggccgcccctagcgtgttcatctttcctccaagcgacgagcagctgaagtccggcaccgcctctgtggtgtgcctgctgaacaacttctacccaagagaggccaaggtgcagtggaaggtggataacgccctgcagtccggcaattctcaggagagcgtgaccgagcaggactccaaggattctacatactctctgagcagcaccctgacactgagcaaggccgactatgagaagcacaaggtgtacgcctgcgaagtgacccaccaggggctgagcagtccagtgaccaagtctttcaatcggggagaatgctgataactc

SEQ ID NO: 58 - protein, 239 aa, synthetic construct
MVLQTQVFISLLLWISGAYGDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - DNA, 1446 bp, synthetic construct
  source 1..1446: mol_type = other DNA
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagagggtggagccaaagagctgtgataagacccacacatgccctccctgtccagcaccagagttcgagggcggcccatccgtgttcctgtttccacccaagcccaaggacacactgtacatcacccgggagccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctgccagcatcgagaagaccatctccaaggcaaagggacagcccagggagcctcaggtgtatacactgcctccaagcagagaggagatgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagtgataa

SEQ ID NO: 60 - protein, 480 aa, synthetic construct
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - DNA, 1446 bp, synthetic construct
  source 1..1446: mol_type = other DNA
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagagggtggagccaaagagctgtgataagacccacacatgccctccctgtccagcaccagagttcgagggcggcccatccgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctgccagcatcgagaagaccatctccaaggcaaagggacagcccagggagcctcaggtgtatacactgcctccaagcagagaggagatgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagtgataa

SEQ ID NO: 62 - protein, 480 aa, synthetic construct
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - DNA, 1449 bp, synthetic construct
  source 1..1449: mol_type = other DNA
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagagggtggagccaaagagctgtgataagacccacacatgccctccctgtccagcaccagagctgctgggcggcccatccgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctgccccaatcgagaagaccatctccaaggcaaagggacagcccagggagcctcaggtgtatacactgcctccaagcagagaggagatgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagtgataactc

SEQ ID NO: 64 - protein, 480 aa, synthetic construct
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 714 bp, synthetic construct
  source 1..714: mol_type = other DNA
atggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctagcaccctgacactgagcaaggccgattacgagaagcacaaggtgtatgcctgcgaggtgacccatcaggggctgtcttctccagtgaccaaatccttcaatcgcggggaatgttgataa

SEQ ID NO: 66 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagagggtggagccaaagtcttgcgataagacccacacatgccctccctgtccagcacctgagttcgagggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagccagcatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 68 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagagggtggagccaaagtcttgcgataagacccacacatgccctccctgtccagcacctgagctgctgggcggcccaagcgtgttcctgtttccacccaagcccaaggacaccctgatgatcagcaggaccccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 70 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagagggtggagccaaagtcttgcgataagacccacacatgccctccctgtccagcacctgagttcgagggcggcccaagcgtgttcctgtttccacccaagcccaaggacaccctgatgatcagcaggaccccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagccagcatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 72 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - DNA, 1446 bp, synthetic construct
  source 1..1446: mol_type = other DNA
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagagggtggagccaaagagctgtgataagacccacacatgccctccctgtccagcaccagagctgctgggcggcccatccgtgttcctgtttccacccaagcccaaggacacactgtacatcacccgggagccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctgccccaatcgagaagaccatctccaaggcaaagggacagcccagggagcctcaggtgtatacactgcctccaagcagagaggagatgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagtgataa

SEQ ID NO: 74 - protein, 480 aa, synthetic construct
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 76 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - DNA, 2247 bp, synthetic construct
  source 1..2247: mol_type = other DNA
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagagggtggagccaaagagctgtgataagacccacacatgccctccctgtccagcaccagagctgctgggcggcccatccgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctgccccaatcgagaagaccatctccaaggcaaagggacagcccagggagcctcaggtgtatacactgcctccaagcagagaggagatgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagaggggaaggaagaggagaagcggctccggcgccacaaacttcagcctgctgaagcaggccggcgatgtggaggagaatcctggcccaatggtgctgcagacccaggtgtttatctccctgctgctgtggatctctggcgcctacggcgacatcgtgatgacccagtctcctgatagcctggccgtgtctctgggagagagggcaacaatcaactgtaagtctagccagagcgtgctgtattcctctaacaataagaattatctggcatggtaccagcagaagccaggacagccacccaagctgctgatgtactgggcatctacccgggagagcggagtgcctgaccgcttctctggcagcggctccggagcagagtttaccctgacaatcagctccctgcaggccgaggatgtggccatctattactgccagcagtattacagcaccctgaccttcggcggcggcaccaaggtggagatcaagaggacagtggccgcccctagcgtgttcatctttcctccaagcgacgagcagctgaagtccggcaccgcctctgtggtgtgcctgctgaacaacttctacccaagagaggccaaggtgcagtggaaggtggataacgccctgcagtccggcaattctcaggagagcgtgaccgagcaggactccaaggattctacatactctctgagcagcaccctgacactgagcaaggccgactatgagaagcacaaggtgtacgcctgcgaagtgacccaccaggggctgagcagtccagtgaccaagtctttcaatcggggagaatgctga

SEQ ID NO: 78 - protein, 748 aa, synthetic construct
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - DNA, 2247 bp, synthetic construct
  source 1..2247: mol_type = other DNA
atggtgctgcagacccaggtgtttatctccctgctgctgtggatctctggcgcctacggcgacatcgtgatgacccagtctcctgatagcctggccgtgtctctgggagagagggcaacaatcaactgtaagtctagccagagcgtgctgtattcctctaacaataagaattatctggcatggtaccagcagaagccaggacagccacccaagctgctgatgtactgggcatctacccgggagagcggagtgcctgaccgcttctctggcagcggctccggagcagagtttaccctgacaatcagctccctgcaggccgaggatgtggccatctattactgccagcagtattacagcaccctgaccttcggcggcggcaccaaggtggagatcaagaggacagtggccgcccctagcgtgttcatctttcctccaagcgacgagcagctgaagtccggcaccgcctctgtggtgtgcctgctgaacaacttctacccaagagaggccaaggtgcagtggaaggtggataacgccctgcagtccggcaattctcaggagagcgtgaccgagcaggactccaaggattctacatactctctgagcagcaccctgacactgagcaaggccgactatgagaagcacaaggtgtacgcctgcgaagtgacccaccaggggctgagcagtccagtgaccaagtctttcaatcggggagaatgcaggggaaggaagaggagaagcggctccggcgccacaaacttcagcctgctgaagcaggccggcgatgtggaggagaatcctggcccaatggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagagggtggagccaaagagctgtgataagacccacacatgccctccctgtccagcaccagagctgctgggcggcccatccgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctgccccaatcgagaagaccatctccaaggcaaagggacagcccagggagcctcaggtgtatacactgcctccaagcagagaggagatgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagtga

SEQ ID NO: 80 - protein, 748 aa, synthetic construct
MVLQTQVFISLLLWISGAYGDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECRGRKRRSGSGATNFSLLKQAGDVEENPGPMDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 737 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 82 - DNA, 2217 bp, synthetic construct
  source 1..2217: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagaaggtggagccaaagtcttgcgataagacccacacatgccctccctgtccagcacctgagctgctgggcggcccaagcgtgttcctgtttccacccaagcccaaggacaccctgatgatcagcaggaccccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggacgagctgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagaggggaaggaagaggagatccggctctggcgccacaaacttcagcctgctgaagcaggccggcgatgtggaggagaatcctggcccaatggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctaacaccctgacactgagcaaggccgattacgagaagcacaaggtgtatgcctgcgaggtgacccatcaggggctgtcttctccagtgaccaaatccttcaatcgcggggaatgttgataa

SEQ ID NO: 83 - protein, 748 aa, synthetic construct
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 84 - DNA, 2250 bp, synthetic construct
  source 1..2250: mol_type = other DNA
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagaaggtggagccaaagagctgtgataagacccacacatgccctccctgtccagcaccagagctgctgggcggcccatccgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctgccccaatcgagaagaccatctccaaggcaaagggacagcccagggagcctcaggtgtatacactgcctccaagcagagacgagctgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagaggggaaggaagaggagaagcggctccggcgccacaaacttcagcctgctgaagcaggcaggcgatgtggaggagaatccaggacctatggtgctgcagacccaggtgtttatctccctgctgctgtggatctctggcgcctacggcgacatcgtgatgacccagtctcctgatagcctggccgtgtctctgggagagagggcaacaatcaactgtaagtctagccagagcgtgctgtattcctctaacaataagaattatctggcatggtaccagcagaagccaggacagccacccaagctgctgatgtactgggcatctacccgggagagcggagtgcctgaccgcttctctggcagcggctccggagcagagtttaccctgacaatcagctccctgcaggccgaggatgtggccatctattactgccagcagtattacagcaccctgaccttcggcggcggcaccaaggtggagatcaagaggacagtggccgcccctagcgtgttcatctttcctccaagcgacgagcagctgaagtccggcaccgcctctgtggtgtgcctgctgaacaacttctacccaagagaggccaaggtgcagtggaaggtggataacgccctgcagtccggcaattctcaggagagcgtgaccgagcaggactccaaggattctacatactctctgagcaacaccctgacactgagcaaggccgactatgagaagcacaaggtgtacgcctgcgaagtgacccaccaggggctgagcagtccagtgaccaagtctttcaatcggggagaatgctgataa

SEQ ID NO: 85 - protein, 737 aa, synthetic construct
MDWTWRILFLVAAATGTHAQMQLVQSGPEVKKPGTSVKVSCKASGFTFTSSAVQWVRQARGQRLEWIGWIAVGSGNTNYAQKFQERVSITRDMSTSTAYMELSSLRSEDTAVYYCAAPYCSRTSCHDAFDIWGQGTKVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGEVVLTQSPGTLSLSPGERATLSCRASQSVSSSYLAWYQQKPGQAPRLLIYGASSRATGIPDRFSGSGSGTDFTLTISRLEPEDFAVYHCQHFGSSSQWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 86 - DNA, 2217 bp, synthetic construct
  source 1..2217: mol_type = other DNA
atggactggacttggagaatcctgtttctggtcgccgccgctactggaacccacgctcagatgcagctggtgcagagtggacccgaagtgaagaagcccggcaccagcgtgaaggtgtcctgtaaggcctctggcttcacctttacaagctccgccgtgcagtgggtgaggcaggccagaggccagcggctggagtggatcggatggatcgcagtgggctccggaaacaccaattacgcccagaagtttcaggagcgcgtgtctatcacaagggacatgtctaccagcacagcctatatggagctgtctagcctgagatccgaggataccgccgtgtactattgcgccgccccttactgttcccggacatcttgccacgacgcctttgatatctggggccagggcaccaaggtgacagtgtcctctgcctctacaaagggaccaagcgtgttcccactggcacctagctccaagtccacctctggcggcacagccgccctgggctgtctggtgaaggactactttcctgagccagtgaccgtgagctggaactccggcgccctgacctccggagtgcacacattccctgccgtgctgcagtctagcggcctgtacagcctgtcctctgtggtgaccgtgccaagctcctctctgggcacccagacatatatctgtaacgtgaatcacaagccttccaatacaaaggtggacaagaaggtggagccaaagtcttgcgataagacccacacatgccctccctgtccagcacctgagctgctgggcggcccaagcgtgttcctgtttccacccaagcccaaggacaccctgatgatcagccggaccccagaggtgacatgcgtggtggtggacgtgtcccacgaggaccccgaggtgaagttcaactggtacgtggatggcgtggaggtgcacaatgccaagaccaagcccagagaggagcagtacaactccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggacgagctgaccaagaaccaggtgagcctgacatgcctggtgaagggcttttaccccagcgatatcgccgtggagtgggagtccaatggccagcctgagaacaattacaagaccacaccccctgtgctggactctgatggcagcttctttctgtattctaagctgaccgtggacaagagccgctggcagcagggcaacgtgttcagctgttccgtgatgcacgaggccctgcacaatcactatacccagaagtctctgagcctgtcccctggcaagaggggaaggaagaggagatctggcagcggcgccacaaactttagcctgctgaagcaggccggcgatgtggaggagaatcctggcccaatggtgctgcagacccaggtgttcatcagcctgctgctgtggatctccggagcatacggagaggtggtgctgacccagtccccaggcacactgtccctgtctcctggagagagagccaccctgagctgcagggcaagccagtccgtgagctcctcttacctggcctggtatcagcagaagcctggacaggccccacgcctgctgatctatggagccagctcccgcgccaccggcatccccgacaggttttctggcagcggctccggcacagatttcaccctgacaatctccaggctggagcctgaggacttcgccgtgtaccactgtcagcactttggctctagctcccagtggaccttcggccagggcacaaaggtggagatcaagagaaccgtggccgccccatccgtgttcatctttccaccctctgacgagcagctgaagagcggcacagcctccgtggtgtgcctgctgaacaatttctatcccagggaggccaaggtgcagtggaaggtggataacgccctgcagtccggcaattctcaggagagcgtgaccgagcaggactccaaggattctacatactctctgagcaacaccctgacactgagcaaggccgattacgagaagcacaaggtgtatgcctgcgaggtcactcaccaggggctgtccagtcccgtcactaagtctttcaatagaggcgaatgttgataa

SEQ ID NO: 87 - DNA, 1446 bp, synthetic construct
  source 1..1446: mol_type = other DNA
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagaaggtggagccaaagagctgtgataagacccacacatgccctccctgtccagcaccagagctgctggccggcccatccgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctgccccaatcgagaagaccatctccaaggcaaagggacagcccagggagcctcaggtgtatacactgcctccaagcagagacgagctgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagtgataa

SEQ ID NO: 88 - protein, 480 aa, synthetic construct
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 1446 bp, synthetic construct
  source 1..1446: mol_type = other DNA
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagaaggtggagccaaagagctgtgataagacccacacatgccctccctgtccagcaccagagctgctgggcggcccatccgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctctgccagaggagaagaccatctccaaggcaaagggacagcccagggagcctcaggtgtatacactgcctccaagcagagacgagctgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagtgataa

SEQ ID NO: 90 - protein, 480 aa, synthetic construct
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1446 bp, synthetic construct
  source 1..1446: mol_type = other DNA
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagaaggtggagccaaagagctgtgataagacccacacatgccctccctgtccagcaccagagctgctggccggcccatccgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctctgccagaggagaagaccatctccaaggcaaagggacagcccagggagcctcaggtgtatacactgcctccaagcagagacgagctgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagtgataa

SEQ ID NO: 92 - protein, 480 aa, synthetic construct
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - DNA, 1446 bp, synthetic construct
  source 1..1446: mol_type = other DNA
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagaaggtggagccaaagagctgtgataagacccacacatgccctccctgtccagcaccagagctgctgggcggcccagacgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctctgccaatcgagaagaccatctccaaggcaaagggacagcccagggagcctcaggtgtatacactgcctccaagcagagacgagctgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagtgataa

SEQ ID NO: 94 - protein, 480 aa, synthetic construct
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - DNA, 1446 bp, synthetic construct
  source 1..1446: mol_type = other DNA
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagaaggtggagccaaagagctgtgataagacccacacatgccctccctgtccagcaccagagctgctggccggcccagacgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctctgccaatcgagaagaccatctccaaggcaaagggacagcccagggagcctcaggtgtatacactgcctccaagcagagacgagctgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagtgataa

SEQ ID NO: 96 - protein, 480 aa, synthetic construct
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - DNA, 1446 bp, synthetic construct
  source 1..1446: mol_type = other DNA
atggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagaaggtggagccaaagagctgtgataagacccacacatgccctccctgtccagcaccagagctgctgaggggcccatccgtgttcctgtttccacccaagcccaaggacacactgatgatctcccggaccccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccaggcctgccccaatcgagaagaccatctccaaggcaaagggacagcccagggagcctcaggtgtatacactgcctccaagcagagacgagctgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagtgataa

SEQ ID NO: 98 - protein, 480 aa, synthetic construct
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - DNA, 4379 bp, synthetic construct
  source 1..4379: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccgccaccatggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 100 - DNA, 3671 bp, synthetic construct
  source 1..3671: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccgccaccatggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctagcaccctgacactgagcaaggccgattacgagaagcacaaggtgtatgcctgcgaggtgacccatcaggggctgtcttctccagtgaccaaatccttcaatcgcggggaatgttgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 101 - DNA, 4403 bp, synthetic construct
  source 1..4403: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccgccaccatggactggacatggagaatcctgttcctggtcgccgccgcaactgggactcacgccgaagtgcagctggtcgaatctgggggaggcctggtgaagccaggcggcagcctgaggctgtcctgcgcagcatctggcttcacctttagggacgtgtggatgtcctgggtgagacaggcaccaggcaagggcctggagtgggtgggacgcatcaagagcaagatcgacggcggaaccacagattatgccgcccccgtgaagggcaggttcaccatcagcagagacgattccaagaacacactgtacctgcagatgaattccctgaagaccgaggatacagccgtgtactattgcaccacagccggctcttactattacgacacagtgggaccaggcctgccagagggcaagttcgattattggggccagggcaccctggtgacagtgagctccgccagcaccaagggcccttccgtgtttcctctggccccatctagcaagtctaccagcggcggcacagccgccctgggatgtctggtgaaggactacttccccgagcctgtgaccgtgtcctggaactctggcgccctgacctccggagtgcacacatttcctgccgtgctgcagtcctctggcctgtatagcctgagctccgtggtgaccgtgccatctagctccctgggcacccagacatacatctgcaacgtgaatcacaagccttctaatacaaaggtggacaagagggtggagccaaagagctgtgataagacccacacatgccctccctgtccagcaccagagctgctgggcggcccatccgtgttcctgtttccacccaagcccaaggacacactgtacatcacccgggagccagaggtgacatgcgtggtggtggacgtgtctcacgaggaccccgaggtgaagttcaactggtacgtggatggcgtggaggtgcacaatgccaagaccaagccccgggaggagcagtataactctacctaccgcgtggtgagcgtgctgacagtgctgcaccaggattggctgaacggcaaggagtacaagtgcaaggtgagcaataaggccctgcctgccccaatcgagaagaccatctccaaggcaaagggacagcccagggagcctcaggtgtatacactgcctccaagcagagaggagatgaccaagaaccaggtgtccctgacatgtctggtgaagggcttctacccctccgatatcgccgtggagtgggagtctaatggccagcctgagaacaattataagaccacaccccctgtgctggacagcgatggctccttctttctgtacagcaagctgaccgtggacaagtcccggtggcagcagggcaacgtgttttcctgctctgtgatgcacgaggccctgcacaatcactacacccagaagagcctgtccctgtctccaggcaagtgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 102 - DNA, 3680 bp, synthetic construct
  source 1..3680: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccgccaccatggtgctgcagacccaggtgtttatctccctgctgctgtggatctctggcgcctacggcgacatcgtgatgacccagtctcctgatagcctggccgtgtctctgggagagagggcaacaatcaactgtaagtctagccagagcgtgctgtattcctctaacaataagaattatctggcatggtaccagcagaagccaggacagccacccaagctgctgatgtactgggcatctacccgggagagcggagtgcctgaccgcttctctggcagcggctccggagcagagtttaccctgacaatcagctccctgcaggccgaggatgtggccatctattactgccagcagtattacagcaccctgaccttcggcggcggcaccaaggtggagatcaagaggacagtggccgcccctagcgtgttcatctttcctccaagcgacgagcagctgaagtccggcaccgcctctgtggtgtgcctgctgaacaacttctacccaagagaggccaaggtgcagtggaaggtggataacgccctgcagtccggcaattctcaggagagcgtgaccgagcaggactccaaggattctacatactctctgagcagcaccctgacactgagcaaggccgactatgagaagcacaaggtgtacgcctgcgaagtgacccaccaggggctgagcagtccagtgaccaagtctttcaatcggggagaatgctgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt